(19) United States
(12) Patent Application Publication
(10) Pub. No.: US 2026/0043029 A1
(43) Pub. Date: Feb. 12, 2026

(54) RNAi Agents for Inhibiting Expression of Thymic Stromal Lymphopoietin (TSLP), Compositions Thereof, and Methods of Use

(71) Applicant: Arrowhead Pharmaceuticals, Inc., Pasadena, CA (US)

(72) Inventors: Casi Schienebeck, Deerfield, WI (US); Szymon Klossowski, Middleton, WI (US); Tingting Yuan, Madison, WI (US); Tao Pei, Middleton, WI (US); Jeffrey Carlson, Madison, WI (US); Agnieszka Glebocka, Madison, WI (US); Mark Majewski, Rockton, IL (US)

(21) Appl. No.: 19/299,635

(22) Filed: Aug. 14, 2025

Related U.S. Application Data
(63) Continuation of application No. PCT/US24/15753, filed on Feb. 14, 2024.
(60) Provisional application No. 63/485,081, filed on Feb. 15, 2023.
(60) Provisional application No. 63/516,300, filed on Jul. 28, 2023.
(60) Provisional application No. 63/625,543, filed on Jan. 26, 2024.

(51) Int. Cl. C12N 15/113; A61P 11/06

(52) U.S. Cl. CPC C12N 15/1136; A61P 11/06; C12N 2310/14; C12N 2310/321; C12N 2310/322; C12N 2310/351; C12N 2320/32; C12N 2320/35

(57) ABSTRACT

Described are RNAi agents, compositions that include RNAi agents, and methods for inhibition of a thymic stromal lymphopoietin (TSLP) gene. The TSLP RNAi agents and RNAi agent conjugates disclosed herein inhibit the expression of an TSLP gene. Pharmaceutical compositions that include one or more TSLP RNAi agents, optionally with one or more additional therapeutics, are also described. Delivery of the described TSLP RNAi agents to pulmonary cells, in vivo, provides for inhibition of TSLP gene expression, which can provide a therapeutic benefit to subjects, including human subjects, for the treatment of various diseases including pulmonary inflammation diseases such as asthma, including allergic asthma.

### CROSS REFERENCE TO RELATED APPLICATIONS

This application is a continuation application of International Application No. PCT/US2024/015753, filed on Feb. 14, 2024, which claims the benefit of priority of U.S. Provisional Patent Application Ser. No. 63/485,081, filed on 15 Feb. 2023, U.S. Provisional Patent Application Ser. No. 63/516,300, filed on 28 Jul. 2023, and U.S. Provisional Patent Application Ser. No. 63/625,543, filed on 26 Jan. 2024, the contents of which are incorporated herein by reference in their entirety.

### SEQUENCE LISTING

This application contains a Sequence Listing (in compliance with Standard ST26), which has been submitted in xml format and is hereby incorporated by reference in its entirety. The xml sequence listing file is named 30723-US1_SeqListing.xml, created Aug. 12, 2025, and is 3,329,866 bytes in size.

### FIELD OF THE INVENTION

The present disclosure relates to RNA interference (RNAi) agents, e.g., double stranded oligonucleotide RNAi agents, for inhibition of Thymic Stromal Lymphopoietin (“TSLP”) gene expression, compositions that include TSLP RNAi agents, and methods of use thereof.

### BACKGROUND

Thymic Stromal Lymphopoietin (“TSLP”) is an epithelial cell-derived cytokine implicated in the initiation and persistence of inflammatory pathways in asthma (Parnes, et al. 2022). TSLP is a member of the 4-helix-bundle cytokine family and a distant paralog of interleukin (IL)-7, which is expressed by human epithelial cells in the thymus, lung, intestine, skin, and stroma, as well as in tonsils and mast cells (Hu, et al. 2017). TSLP affects various cell types through a heterodimeric receptor consisting of the IL-7 receptor chain (IL-7Ra) and a specific subunit, TSLP-specific receptor (TSLPR) (Pandey, et al. 2000).

Two variants (short and long) of human TSLP have been identified so far. Short-form TSLP (“sfTSLP”) (60 amino acids) is constitutively expressed and maintains homeostatic conditions in the skin, gut, oral epithelium, and salivary glands, and is downregulated in inflammatory conditions. In contrast, long-form TSLP (“lfTSLP”) (159 amino acids) is inducible, and it can be massively upregulated in inflammatory diseases like atopic dermatitis (AD) and allergic asthma (AA) (Adhikary, et al. 2021, Pelaia, et al. 2021). sfTSLP does not bind to the TSLPR and is incapable of blocking the binding of lfTSLP to this receptor (Adhikary, Tan et al. 2021).

TSLP stimulates dendritic cells to guide the differentiation of naïve Th cells towards the Th2 lineage, but can also promote Th17 commitment (Gauvreau, Sehmi et al. 2020). Moreover, TSLP activates ILC2, mast cells, and basophils, induces eosinophil survival and transmigration, and also affects the functions of airway structural cells such as fibroblasts and airway smooth muscle cells (Gauvreau, Sehmi et al. 2020). In allergic asthma, via activation of dendritic cells, TSLP promotes the differentiation of Th2 lymphocytes secreting IL-4, IL-5, IL-9, and IL-13, which target B cells, eosinophils, mast cells, and airway smooth muscle cells, respectively (Pelaia, et al. 2021). Given its position at the top of the inflammatory cascade, TSLP can exert broad influence over airway inflammation through its impact on multiple cell types and pathways. Therefore, treatments that are able to target TSLP would provide a novel approach to treat inflammation in asthma.

TSLP overexpression can be detected in both outer and inner surfaces of bronchial epithelial biopsies, as well as in serum, induced sputum, bronchoalveolar lavage fluid (BALF), and exhaled breath condensate of asthmatic patients and in mice with asthma (Al-Shami et al. 2005; Ying, et al. 2005; Zhou et al. 2005). Moreover, airway expression levels of TSLP are correlated with asthma severity and airflow (Ying, et al. 2008; Gauvreau, et al. 2020).

Genomic studies have shown that some single-nucleotide polymorphisms (SNPs) of the TSLP gene are associated with the risk of developing asthma (Torgerson, et al. 2011).

Tezepelumab is an anti-TSLP human monoclonal antibody for the treatment of asthma. In the PATHWAY phase 2b (NCT02054130) and NAVIGATOR phase 3 (NCT03347279) studies, tezepelumab significantly reduced exacerbation rates versus placebo in patients with severe, uncontrolled asthma (Corren, et al. 2017; Menzies-Gow, et al. 2021). Reported clinical benefits were associated with reductions in levels of a broad spectrum of cytokines (e.g., interleukin [IL]-5, IL-13) and baseline biomarkers (e.g., blood eosinophils, immunoglobulin [Ig]E, fractional exhaled nitric oxide [FeNO]) and were observed across a range of severe asthma phenotypes (including eosinophilic and non-eosinophilic)(Diver, et al. 2021; Puzzovio, et al. 2022). TSLP neutralizing antibody has also been reported to alleviate airway inflammation in different asthmatic models including mouse house dust mite (HDM), ovalbumin (OVA) and Toluene-diisocyanate (TDI)-induced models (Li, et al. 2010; Chen, et al. 2018; Yu, et al. 2019). However, tezepelumab requires administration of a subcutaneous injection every 4 weeks. A sufficiently safe, potent, and active RNA interference agent therapeutic targeting TSLP would provide an alternative therapy option for patients, and particularly if the RNAi agent can be administered through inhaled administration and/or on a less frequent (e.g., quarterly or bimonthly) basis, it could provide for an improved and more desirable therapy option for patients.

### SUMMARY

There exists a need for novel RNA interference (RNAi) agents (termed RNAi agents, RNAi triggers, or triggers), e.g., double stranded RNAi agents, that are able to selectively and efficiently inhibit the expression of a TSLP gene, including for use as a therapeutic or medicament. Further, there exists a need for compositions of novel TSLP-specific RNAi agents for the treatment of diseases or disorders associated with pulmonary inflammation such as asthma (specifically including allergic asthma) and/or disorders that can be mediated at least in part by a reduction in TSLP gene expression.

The nucleotide sequences and chemical modifications of the TSLP RNAi agents disclosed herein, as well as their combination with certain specific targeting ligands suitable for selectively and efficiently delivering the TSLP RNAi agents to relevant pulmonary cells in vivo, differ from what is previously disclosed or known in the art. The TSLP RNAi agents disclosed herein provide for highly potent and efficient inhibition of the expression of a TSLP gene.

In general, the present disclosure features TSLP gene-specific RNAi agents, compositions that include TSLP RNAi agents, and methods for inhibiting expression of a TSLP gene in vitro and/or in vivo using the TSLP RNAi agents and compositions that include TSLP RNAi agents described herein. The TSLP RNAi agents described herein are able to selectively and efficiently decrease expression of a TSLP gene, and thereby inhibiting the translation of TSLP proteins or cytokines that are at the beginning of the inflammatory cascade resulting in a reduction of airway inflammation.

The described TSLP RNAi agents can be used in methods for therapeutic treatment (including preventative or prophylactic treatment) of symptoms and diseases including, but not limited to, asthma including but not limited to allergic asthma, chronic obstructive pulmonary disease including but not limited to chronic bronchitis and emphysema, pulmonary inflammatory disorders, interstitial lung diseases (ILD), cystic fibrosis, various other types of fibrosis, infectious diseases (for example, SARS-COV-2), acute lung injury (for example, acute respiratory distress syndrome (ARDS)), pulmonary hypertension, various pulmonary cancers, chronic rhinosinutis either with or without nasal polyps, autoimmune disorders including but not limited to systemic sclerosis (SSc), and multiple inflammatory diseases including but not limited to atopic dermatitis, chronic spontaneous urticaria, and eosinophilic esophagitis.

In one aspect, the disclosure features RNAi agents for inhibiting expression of a TSLP gene, wherein the RNAi agent includes a sense strand (also referred to as a passenger strand) and an antisense strand (also referred to as a guide strand). The sense strand and the antisense strand can be partially, substantially, or fully complementary to each other. The length of the RNAi agent sense strands described herein each can be 12 to 49 nucleotides in length. The length of the RNAi agent antisense strands described herein each can be 18 to 30 nucleotides in length. In some embodiments, the sense and antisense strands are independently 18 to 26 nucleotides in length. The sense and antisense strands can be either the same length or different lengths. In some embodiments, the sense and antisense strands are independently 21 to 26 nucleotides in length. In some embodiments, the sense and antisense strands are independently 21 to 24 nucleotides in length. In some embodiments, both the sense strand and the antisense strand are 21 nucleotides in length. In some embodiments, the antisense strands are independently 18, 19, 20, 21, 22, 23, 24, 25, 26, 27, 28, 29, or 30 nucleotides in length. In some embodiments, the sense strands are independently 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides in length. The RNAi agents described herein, upon delivery to a cell expressing TSLP such as a pulmonary cell, inhibit the expression of one or more TSLP gene variants in vivo and/or in vitro.

The TSLP RNAi agents disclosed herein target a human TSLP gene (see, e.g., SEQ ID NO:1). In some embodiments, the TSLP RNAi agents disclosed herein target a portion of a TSLP gene having the sequence of any of the sequences disclosed in Table 1.

In another aspect, the disclosure features compositions, including pharmaceutical compositions, that include one or more of the disclosed TSLP RNAi agents that are able to selectively and efficiently decrease expression of an TSLP gene. The compositions that include one or more TSLP RNAi agents described herein can be administered to a subject, such as a human or animal subject, for the treatment (including prophylactic treatment or inhibition) of symptoms and diseases including, but not limited to, asthma including but not limited to allergic asthma, chronic obstructive pulmonary disease including but not limited to chronic bronchitis and emphysema, pulmonary inflammatory disorders, interstitial lung diseases (ILD), cystic fibrosis, various other types of fibrosis, infectious diseases (for example, SARS-COV-2), acute lung injury (for example, acute respiratory distress syndrome (ARDS)), pulmonary hypertension, various pulmonary cancers, chronic rhinosinutis either with or without nasal polyps, autoimmune disorders including but not limited to systemic sclerosis (SSc), and multiple inflammatory diseases including but not limited to atopic dermatitis, chronic spontaneous urticaria, and eosinophilic esophagitis.

Examples of TSLP RNAi agent sense strands and antisense strands that can be used in a TSLP RNAi agent are provided in Tables 3, 4, 5, and 6. Examples of TSLP RNAi agent duplexes are provided in Tables 7A, 7B, 8, 9, and 10. Examples of 19-nucleotide core stretch sequences that may consist of or may be included in the sense strands and antisense strands of certain TSLP RNAi agents disclosed herein, are provided in Table 2.

In another aspect, the disclosure features methods for delivering TSLP RNAi agents to epithelial cells in a subject, such as a mammal, in vivo. Also described herein are compositions for use in such methods. In some embodiments, disclosed herein are methods for delivering TSLP RNAi agents to pulmonary cells (epithelial cells, macrophages, smooth muscle, endothelial cells) to a subject in vivo. In some embodiments, the subject is a human subject.

The methods disclosed herein include the administration of one or more TSLP RNAi agents to a subject, e.g., a human or animal subject, by any suitable means known in the art. The pharmaceutical compositions disclosed herein that include one or more TSLP RNAi agents can be administered in a number of ways depending upon whether local or systemic treatment is desired. Administration can be, but is not limited to, for example, intravenous, intraarterial, subcutaneous, intraperitoneal, subdermal (e.g., via an implanted device), and intraparenchymal administration. In some embodiments, the pharmaceutical compositions described herein are administered by inhalation (such as dry powder inhalation or aerosol inhalation) or through use of a nebulizer, intranasal administration, intratracheal administration, or oropharyngeal aspiration administration.

In some embodiments, it is desired that the TSLP RNAi agents described herein inhibit the expression of an TSLP gene in the pulmonary epithelium, for which the administration is by inhalation (e.g., by an inhaler device, such as a metered-dose inhaler, or a nebulizer such as a jet or vibrating mesh nebulizer, or a soft mist inhaler).

The one or more TSLP RNAi agents can be delivered to target cells or tissues using any oligonucleotide delivery technology known in the art. In some embodiments, a TSLP RNAi agent is delivered to cells or tissues by covalently linking the RNAi agent to a targeting group. In some embodiments, the targeting group can include a cell receptor ligand, such as an integrin targeting ligand. Integrins are a family of transmembrane receptors that facilitate cell-extracellular matrix (ECM) adhesion. In particular, integrin alpha-v-beta-6 (αvβ6) is an epithelial-specific integrin that is known to be a receptor for ECM proteins and the TGF-beta latency-associated peptide (LAP), and is expressed in various cells and tissues. Integrin αvβ6 is known to be highly upregulated in injured pulmonary epithelium. In some embodiments, the TSLP RNAi agents described herein are linked to an integrin targeting ligand that has affinity for integrin αvβ6. As referred to herein, an “αvβ6 integrin targeting ligand” is a compound that has affinity for integrin αvβ6, which can be utilized as a ligand to facilitate the targeting and delivery of an RNAi agent to which it is attached to the desired cells and/or tissues (i.e., to cells expressing integrin αvβ6). In some embodiments, multiple αvβ6 integrin targeting ligands or clusters of αvβ6 integrin targeting ligands are linked to a TSLP RNAi agent. In some embodiments, the TSLP RNAi agent-αvβ6 integrin targeting ligand conjugates are selectively internalized by lung epithelial cells, either through receptor-mediated endocytosis or by other means.

Examples of targeting groups useful for delivering TSLP RNAi agents that include αvβ6 integrin targeting ligands are disclosed, for example, in International Patent Application Publication No. WO 2018/085415 and International Patent Application Publication No. WO 2019/089765, the contents of each of which are incorporated by reference herein in their entirety.

A targeting group can be linked to the 3′ or 5′ end of a sense strand or an antisense strand of a TSLP RNAi agent. In some embodiments, a targeting group is linked to the 3′ or 5′ end of the sense strand. In some embodiments, a targeting group is linked to the 5′ end of the sense strand. In some embodiments, a targeting group is linked internally to a nucleotide on the sense strand and/or the antisense strand of the RNAi agent. In some embodiments, one or more targeting ligands are linked internally to one or more nucleotides on the sense strand of the RNAi agent. In some embodiments, a targeting group is linked to the RNAi agent via a linker.

In another aspect, the disclosure features compositions that include one or more TSLP RNAi agents that have the duplex structures disclosed in Tables 7A, 7B, 8, 9, and 10.

The use of TSLP RNAi agents provides methods for therapeutic (including prophylactic) treatment of diseases or disorders for which a reduction in TSLP can provide a therapeutic benefit. The TSLP RNAi agents disclosed herein can be used to treat various diseases such as asthma including but not limited to allergic asthma, chronic obstructive pulmonary disease including but not limited to chronic bronchitis and emphysema, pulmonary inflammatory disorders, interstitial lung diseases (ILD), cystic fibrosis, various other types of fibrosis, infectious diseases (for example, SARS-COV-2), acute lung injury (for example, acute respiratory distress syndrome (ARDS)), pulmonary hypertension, various pulmonary cancers, chronic rhinosinutis either with or without nasal polyps, autoimmune disorders including but not limited to systemic sclerosis (SSc), and multiple inflammatory diseases including but not limited to atopic dermatitis, chronic spontaneous urticaria, and eosinophilic esophagitis. In some embodiments, the TSLP RNAi agents disclosed herein can be used to treat a pulmonary inflammatory disease or condition. In some embodiments, the TSLP RNAi agents disclosed herein can be used to treat asthma. TSLP RNAi agents can be used to treat, for example, allergic asthma. Such methods of treatment include administration of a TSLP RNAi agent to a human being or animal for which a reduction in TSLP levels is desired.

### Definitions

As used herein, the terms “oligonucleotide” and “polynucleotide” mean a polymer of linked nucleosides each of which can be independently modified or unmodified.

As used herein, an “RNAi agent” (also referred to as an “RNAi trigger”) means a composition of matter that contains an RNA or RNA-like (e.g., chemically modified RNA) oligonucleotide molecule that is capable of degrading or inhibiting (e.g., degrades or inhibits under appropriate conditions) translation of messenger RNA (mRNA) transcripts of a target mRNA in a sequence specific manner. As used herein, RNAi agents may operate through the RNA interference mechanism (i.e., inducing RNA interference through interaction with the RNA interference pathway machinery (RNA-induced silencing complex or RISC) of mammalian cells), or by any alternative mechanism(s) or pathway(s). While it is believed that RNAi agents, as that term is used herein, operate primarily through the RNA interference mechanism, the disclosed RNAi agents are not bound by or limited to any particular pathway or mechanism of action. RNAi agents disclosed herein are comprised of a sense strand and an antisense strand, and include, but are not limited to: short (or small) interfering RNAs (siRNAs), double stranded RNAs (dsRNA), micro RNAs (miRNAs), short hairpin RNAs (shRNA), and dicer substrates. The antisense strand of the RNAi agents described herein is at least partially complementary to the mRNA being targeted (i.e. TSLP mRNA). RNAi agents can include one or more modified nucleotides and/or one or more non-phosphodiester linkages.

As used herein, the terms “silence,” “reduce,” “inhibit,” “down-regulate,” or “knockdown” when referring to expression of a given gene, mean that the expression of the gene, as measured by the level of RNA transcribed from the gene or the level of polypeptide, protein, or protein subunit translated from the mRNA in a cell, group of cells, tissue, organ, or subject in which the gene is transcribed, is reduced when the cell, group of cells, tissue, organ, or subject is treated with the RNAi agents described herein as compared to a second cell, group of cells, tissue, organ, or subject that has not or have not been so treated.

As used herein, the terms “sequence” and “nucleotide sequence” mean a succession or order of nucleobases or nucleotides, described with a succession of letters using standard nomenclature.

As used herein, a “base,” “nucleotide base,” or “nucleobase,” is a heterocyclic pyrimidine or purine compound that is a component of a nucleotide, and includes the primary purine bases adenine and guanine, and the primary pyrimidine bases cytosine, thymine, and uracil. A nucleobase may further be modified to include, without limitation, universal bases, hydrophobic bases, promiscuous bases, size-expanded bases, and fluorinated bases. (See. e.g., Modified Nucleosides in Biochemistry, Biotechnology and Medicine, Herdewijn, P. ed. Wiley-VCH, 2008). The synthesis of such modified nucleobases (including phosphoramidite compounds that include modified nucleobases) is known in the art.

As used herein, and unless otherwise indicated, the term “complementary,” when used to describe a first nucleobase or nucleotide sequence (e.g., RNAi agent sense strand or targeted mRNA) in relation to a second nucleobase or nucleotide sequence (e.g., RNAi agent antisense strand or a single-stranded antisense oligonucleotide), means the ability of an oligonucleotide or polynucleotide including the first nucleotide sequence to hybridize (form base pair hydrogen bonds under mammalian physiological conditions (or otherwise suitable in vivo or in vitro conditions)) and form a duplex or double helical structure under certain standard conditions with an oligonucleotide that includes the second nucleotide sequence. The person of ordinary skill in the art would be able to select the set of conditions most appropriate for a hybridization test. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification. For example, a and Af, as defined herein, are complementary to U (or T) and identical to A for the purposes of determining identity or complementarity.

As used herein, “perfectly complementary” or “fully complementary” means that in a hybridized pair of nucleobase or nucleotide sequence molecules, all (100%) of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “partially complementary” means that in a hybridized pair of nucleobase or nucleotide sequence molecules, at least 70%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “substantially complementary” means that in a hybridized pair of nucleobase or nucleotide sequence molecules, at least 85%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, the terms “complementary,” “fully complementary,” “partially complementary,” and “substantially complementary” are used with respect to the nucleobase or nucleotide matching between the sense strand and the antisense strand of an RNAi agent, or between the antisense strand of an RNAi agent and a sequence of an TSLP mRNA.

As used herein, the term “substantially identical” or “substantial identity,” as applied to a nucleic acid sequence means the nucleotide sequence (or a portion of a nucleotide sequence) has at least about 85% sequence identity or more, e.g., at least 90%, at least 95%, or at least 99% identity, compared to a reference sequence. Percentage of sequence identity is determined by comparing two optimally aligned sequences over a comparison window. The percentage is calculated by determining the number of positions at which the same type of nucleic acid base occurs in both sequences to yield the number of matched positions, dividing the number of matched positions by the total number of positions in the window of comparison and multiplying the result by 100 to yield the percentage of sequence identity. The inventions disclosed herein encompass nucleotide sequences substantially identical to those disclosed herein.

As used herein, the terms “treat,” “treatment,” and the like, mean the methods or steps taken to provide relief from or alleviation of the number, severity, and/or frequency of one or more symptoms of a disease in a subject. As used herein, “treat” and “treatment” may include the prevention, management, prophylactic treatment, and/or inhibition or reduction of the number, severity, and/or frequency of one or more symptoms of a disease in a subject.

As used herein, the phrase “introducing into a cell,” when referring to an RNAi agent, means functionally delivering the RNAi agent into a cell. The phrase “functional delivery,” means delivering the RNAi agent to the cell in a manner that enables the RNAi agent to have the expected biological activity, e.g., sequence-specific inhibition of gene expression.

Unless stated otherwise, use of the symbol

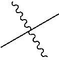

as used herein means that any group or groups may be linked thereto that is in accordance with the scope of the inventions described herein.

As used herein, the term “isomers” refers to compounds that have identical molecular formulae, but that differ in the nature or the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images are termed “enantiomers,” or sometimes optical isomers. A carbon atom bonded to four non-identical substituents is termed a “chiral center.”

As used herein, unless specifically identified in a structure as having a particular conformation, for each structure in which asymmetric centers are present and thus give rise to enantiomers, diastereomers, or other stereoisomeric configurations, each structure disclosed herein is intended to represent all such possible isomers, including their optically pure and racemic forms. For example, the structures disclosed herein are intended to cover mixtures of diastereomers as well as single stereoisomers.

As used in a claim herein, the phrase “consisting of” excludes any element, step, or ingredient not specified in the claim. When used in a claim herein, the phrase “consisting essentially of” limits the scope of a claim to the specified materials or steps and those that do not materially affect the basic and novel characteristic(s) of the claimed invention.

The person of ordinary skill in the art would readily understand and appreciate that the compounds and compositions disclosed herein may have certain atoms (e.g., N, O, or S atoms) in a protonated or deprotonated state, depending upon the environment in which the compound or composition is placed. Accordingly, as used herein, the structures disclosed herein envisage that certain functional groups, such as, for example, OH, SH, or NH, may be protonated or deprotonated. The disclosure herein is intended to cover the disclosed compounds and compositions regardless of their state of protonation based on the environment (such as pH), as would be readily understood by the person of ordinary skill in the art. Correspondingly, compounds described herein with labile protons or basic atoms should also be understood to represent salt forms of the corresponding compound. Compounds described herein may be in a free-acid, free-base, or salt form. Pharmaceutically acceptable salts of the compounds described herein should be understood to be within the scope of the invention.

As used herein, the term “linked” or “conjugated” when referring to the connection between two compounds or molecules means that two compounds or molecules are joined by a covalent bond. Unless stated, the terms “linked” and “conjugated” as used herein may refer to the connection between a first compound and a second compound either with or without any intervening atoms or groups of atoms.

As used herein, the term “including” is used to herein mean, and is used interchangeably with, the phrase “including but not limited to.” The term “or” is used herein to mean, and is used interchangeably with, the term “and/or,” unless the context clearly indicates otherwise.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other objects, features, aspects, and advantages of the invention will be apparent from the following detailed description, accompanying figures, and from the claims.

### BRIEF DESCRIPTION OF THE DRAWINGS

FIG. 1. Chemical structure representation of the tridentate αvβ6 epithelial cell targeting ligand referred to herein as Tri-SM6.1-αvb6-(TA14).

FIG. 2. Graph plotting the reduction of hTSLP protein in AAV transduced mouse lungs of certain TSLP RNAi agents tested (see also Example 5).

FIG. 3A. and FIG. 3B. Graphs plotting reduction of hTSLP protein in AAV transduced mouse lungs of the RNAi agents tested (see also Example 7). The samples were analyzed for protein expression on separate plates (plate 1 shown in FIG. 3A; plate 2 shown in FIG. 3B); the same control was used for both plates.

FIG. 4A, FIG. 4B, and FIG. 4C. Graphs plotting reduction of lung TSLP mRNA (FIG. 4A) and BAL inflammatory cell counts in rats treated with TSLP RNAi agents. The BAL samples were evaluated for eosinophils (FIG. 4B) and BAL total cells (FIG. 4C) (see also Example 10).

FIG. 5A, FIG. 5B, and FIG. 5C. Graphs plotting lung mRNA levels of TSLP (FIG. 5A), IL-13 (FIG. 5B), and IL-33 (FIG. 5C) in rats administered with rat-specific TSLP RNAi agents (see also Example 3).

FIG. 5D, FIG. 5E, and FIG. 5F. Graphs plotting BAL soluble collagen (FIG. 5D), BAL IL-5 (FIG. 5E), and BAL IL-13 (FIG. 5F) in rats administered with rat-specific TSLP RNAi agents (see also Example 3).

FIG. 6A, FIG. 6B, and FIG. 6C. Graphs plotting human TSLP mRNA in transduced mouse lungs (FIG. 6A), human TSLP protein in AAV transduced mouse lungs (FIG. 6B), and human TSLP protein in serum of AAV transduced mice (FIG. 6C) (see also Example 11).

FIG. 7. Graph plotting human TSLP protein in AAV transduced mouse lungs (see also Example 15).

FIG. 8. Graph plotting human TSLP protein in AAV transduced mouse lungs (see also Example 16).

FIG. 9A and FIG. 9B. Graph plotting human TSLP protein in AAV transduced mouse lungs (FIG. 9A) and mouse serum (FIG. 9B) (see also Example 18).

FIG. 10A and FIG. 10B. Graph plotting human TSLP protein in AAV transduced mouse lungs (FIG. 10A) and mouse serum (FIG. 10B) (see also Example 19).

FIG. 11A and FIG. 11B. Graph plotting human TSLP protein in AAV transduced mouse lungs (FIG. 11A) and mouse serum (FIG. 11B) (see also Example 25).

FIG. 12A and FIG. 12B. Graph plotting human TSLP protein in AAV transduced mouse lungs (FIG. 12A) and mouse serum (FIG. 12B) (see also Example 26).

### DETAILED DESCRIPTION

### RNAi Agents

Described herein are RNAi agents for inhibiting expression of a TSLP gene (referred to herein as TSLP RNAi agents or TSLP RNAi triggers). Each TSLP RNAi agent disclosed herein comprises a sense strand and an antisense strand. The sense strand can be 12 to 49 nucleotides in length. The antisense strand can be 18 to 49 nucleotides in length. The sense and antisense strands can be either the same length or they can be different lengths. In some embodiments, the sense and antisense strands are each independently 18 to 27 nucleotides in length. In some embodiments, both the sense and antisense strands are each 21-26 nucleotides in length. In some embodiments, the sense and antisense strands are each 21-24 nucleotides in length. In some embodiments, the sense and antisense strands are each independently 19-21 nucleotides in length. In some embodiments, the sense strand is about 19 nucleotides in length while the antisense strand is about 21 nucleotides in length. In some embodiments, the sense strand is about 21 nucleotides in length while the antisense strand is about 23 nucleotides in length. In some embodiments, a sense strand is 23 nucleotides in length and an antisense strand is 21 nucleotides in length. In some embodiments, both the sense and antisense strands are each 21 nucleotides in length. In some embodiments, the RNAi agent sense strands are each independently 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides in length. In some embodiments, the RNAi agent antisense strands are each independently 18, 19, 20, 21, 22, 23, 24, 25, 26, 27, 28, 29, or 30 nucleotides in length. In some embodiments, the RNAi agent is double stranded and has a duplex length of about 12, 13, 14, 15, 16, 17, 18, 19, 20, 21, 22, 23 or 24 nucleotides. In some embodiments, the RNAi agent is double stranded and has a duplex length of 19, 20, 21, 22, or 23 nucleotides.

Examples of nucleotide sequences used in forming TSLP RNAi agents are provided in Tables 2, 3, 4, 5, 6, and 10. Examples of RNAi agent duplexes, that include the sense strand and antisense strand sequences in Tables 2, 3, 4, 5, 6, are shown in Tables 7A, 7B, 8, 9, and 10.

In some embodiments, the region of perfect, substantial, or partial complementarity between the sense strand and the antisense strand is 16-26 (e.g., 16, 17, 18, 19, 20, 21, 22, 23, 24, 25, or 26) nucleotides in length and occurs at or near the 5′ end of the antisense strand (e.g., this region may be separated from the 5′ end of the antisense strand by 0, 1, 2, 3, or 4 nucleotides that are not perfectly, substantially, or partially complementary).

A sense strand of the TSLP RNAi agents described herein includes at least 12 consecutive nucleotides that have at least 85% identity to a core stretch sequence (also referred to herein as a “core stretch” or “core sequence”) of the same number of nucleotides in an TSLP mRNA. In some embodiments, a sense strand core stretch sequence is 100% (perfectly) complementary or at least about 85% (substantially) complementary to a core stretch sequence in the antisense strand, and thus the sense strand core stretch sequence is typically perfectly identical or at least about 85% identical to a nucleotide sequence of the same length (sometimes referred to, e.g., as a target sequence) present in the TSLP mRNA target. In some embodiments, this sense strand core stretch is 16, 17, 18, 19, 20, 21, 22, or 23 nucleotides in length. In some embodiments, this sense strand core stretch is 17 nucleotides in length. In some embodiments, this sense strand core stretch is 19 nucleotides in length. In some embodiments, this sense strand core stretch is 21 nucleotides in length.

An antisense strand of a TSLP RNAi agent described herein includes at least 15 consecutive nucleotides that have at least 85% complementarity to a core stretch of the same number of nucleotides in an TSLP mRNA and to a core stretch of the same number of nucleotides in the corresponding sense strand. In some embodiments, an antisense strand core stretch is 100% (perfectly) complementary or at least about 85% (substantially) complementary to a nucleotide sequence (e.g., target sequence) of the same length present in the TSLP mRNA target. In some embodiments, this antisense strand core stretch is 17, 18, 19, 20, 21, 22, or 23 nucleotides in length. In some embodiments, this antisense strand core stretch is 19 nucleotides in length. In some embodiments, this antisense strand core stretch is 17 nucleotides in length. A sense strand core stretch sequence can be the same length as a corresponding antisense core sequence or it can be a different length.

The TSLP RNAi agent sense and antisense strands anneal to form a duplex. A sense strand and an antisense strand of a TSLP RNAi agent can be partially, substantially, or fully complementary to each other. Within the complementary duplex region, the sense strand core stretch sequence is at least 85% complementary or 100% complementary to the antisense core stretch sequence. In some embodiments, the sense strand core stretch sequence contains a sequence of at least 16, at least 17, at least 18, at least 19, at least 20, at least 21, at least 22, or at least 23 nucleotides that is at least 85% or 100% complementary to a corresponding 16, 17, 18, 19, 20, 21, 22, or 23 nucleotide sequence of the antisense strand core stretch sequence (i.e., the sense and antisense core stretch sequences of a TSLP RNAi agent have a region of at least 16, at least 17, at least 18, at least 19, at least 20, at least 21, at least 22, or at least 23 nucleotides that is at least 85% base paired or 100% base paired.)

In some embodiments, the antisense strand of a TSLP RNAi agent disclosed herein differs by 0, 1, 2, or 3 nucleotides from any of the antisense strand sequences in Table 2 or Table 3. In some embodiments, the sense strand of a TSLP RNAi agent disclosed herein differs by 0, 1, 2, or 3 nucleotides from any of the sense strand sequences in Table 2, Table 4, Table 5, Table 6, or Table 10.

In some embodiments, the sense strand and/or the antisense strand can optionally and independently contain an additional 1, 2, 3, 4, 5, or 6 nucleotides (extension) at the 3′ end, the 5′ end, or both the 3′ and 5′ ends of the core stretch sequences. The antisense strand additional nucleotides, if present, may or may not be complementary to the corresponding sequence in the TSLP mRNA. The sense strand additional nucleotides, if present, may or may not be identical to the corresponding sequence in the TSLP mRNA. The antisense strand additional nucleotides, if present, may or may not be complementary to the corresponding sense strand's additional nucleotides, if present.

As used herein, an extension comprises 1, 2, 3, 4, 5, or 6 nucleotides at the 5′ and/or 3′ end of the sense strand core stretch sequence and/or antisense strand core stretch sequence. The extension nucleotides on a sense strand may or may not be complementary to nucleotides, either core stretch sequence nucleotides or extension nucleotides, in the corresponding antisense strand. Conversely, the extension nucleotides on an antisense strand may or may not be complementary to nucleotides, either core stretch nucleotides or extension nucleotides, in the corresponding sense strand. In some embodiments, both the sense strand and the antisense strand of an RNAi agent contain 3′ and 5′ extensions. In some embodiments, one or more of the 3′ extension nucleotides of one strand base pairs with one or more 5′ extension nucleotides of the other strand. In other embodiments, one or more of 3′ extension nucleotides of one strand do not base pair with one or more 5′ extension nucleotides of the other strand. In some embodiments, a TSLP RNAi agent has an antisense strand having a 3′ extension and a sense strand having a 5′ extension. In some embodiments, the extension nucleotide(s) are unpaired and form an overhang. As used herein, an “overhang” refers to a stretch of one or more unpaired nucleotides located at a terminal end of either the sense strand or the antisense strand that does not form part of the hybridized or duplexed portion of an RNAi agent disclosed herein (See. e.g., U.S. Pat. No. 8,362,231).

In some embodiments, a TSLP RNAi agent comprises an antisense strand having a 3′ extension of 1, 2, 3, 4, 5, or 6 nucleotides in length. In other embodiments, a TSLP RNAi agent comprises an antisense strand having a 3′ extension of 1, 2, or 3 nucleotides in length. In some embodiments, one or more of the antisense strand extension nucleotides comprise nucleotides that are complementary to the corresponding TSLP mRNA sequence. In some embodiments, one or more of the antisense strand extension nucleotides comprise nucleotides that are not complementary to the corresponding TSLP mRNA sequence.

In some embodiments, a TSLP RNAi agent comprises a sense strand having a 3′ extension of 1, 2, 3, 4, or 5 nucleotides in length. In some embodiments, one or more of the sense strand extension nucleotides comprises adenosine, uracil, or thymidine nucleotides, AT dinucleotide, or nucleotides that correspond to or are the identical to nucleotides in the TSLP mRNA sequence. In some embodiments, the 3′ sense strand extension includes or consists of one of the following sequences, but is not limited to: T, UT, TT, UU, UUT, TTT, or TTTT (each listed 5′ to 3′).

A sense strand can have a 3′ extension and/or a 5′ extension. In some embodiments, a TSLP RNAi agent comprises a sense strand having a 5′ extension of 1, 2, 3, 4, 5, or 6 nucleotides in length. In some embodiments, one or more of the sense strand extension nucleotides comprise nucleotides that correspond to or are identical to nucleotides in the TSLP mRNA sequence.

Examples of sequences used in forming TSLP RNAi agents are provided in Tables 2, 3, 4, 5, 6, and 10. In some embodiments, a TSLP RNAi agent antisense strand includes a sequence of any of the sequences in Tables 2, 3, or 10. In certain embodiments, a TSLP RNAi agent antisense strand comprises or consists of any one of the modified sequences in Table 3. In some embodiments, a TSLP RNAi agent antisense strand includes the sequence of nucleotides (from 5′ end→3′ end) 1-17, 2-15, 2-17, 1-18, 2-18, 1-19, 2-19, 1-20, 2-20, 1-21, or 2-21, of any of the sequences in Tables 2 or 3. In some embodiments, a TSLP RNAi agent sense strand includes the sequence of any of the sequences in Tables 2, 4, 5, or 6. In some embodiments, a TSLP RNAi agent sense strand includes the sequence of nucleotides (from 5′ end→3′ end) 1-18, 1-19, 1-20, 1-21, 2-19, 2-20, 2-21, 3-20, 3-21, or 4-21 of any of the sequences in Tables 2, 4, 5, or 6. In certain embodiments, a TSLP RNAi agent sense strand comprises or consists of a modified sequence of any one of the modified sequences in Table 4, 5, 6, or 10.

In some embodiments, the sense and antisense strands of the RNAi agents described herein contain the same number of nucleotides. In some embodiments, the sense and antisense strands of the RNAi agents described herein contain different numbers of nucleotides. In some embodiments, the sense strand 5′ end and the antisense strand 3′ end of an RNAi agent form a blunt end. In some embodiments, the sense strand 3′ end and the antisense strand 5′ end of an RNAi agent form a blunt end. In some embodiments, both ends of an RNAi agent form blunt ends. In some embodiments, neither end of an RNAi agent is blunt-ended. As used herein a “blunt end” refers to an end of a double stranded RNAi agent in which the terminal nucleotides of the two annealed strands are complementary (form a complementary base-pair).

In some embodiments, the sense strand 5′ end and the antisense strand 3′ end of an RNAi agent form a frayed end. In some embodiments, the sense strand 3′ end and the antisense strand 5′ end of an RNAi agent form a frayed end. In some embodiments, both ends of an RNAi agent form a frayed end. In some embodiments, neither end of an RNAi agent is a frayed end. As used herein a frayed end refers to an end of a double stranded RNAi agent in which the terminal nucleotides of the two annealed strands form a pair (i.e., do not form an overhang) but are not complementary (i.e. form a non-complementary pair). In some embodiments, one or more unpaired nucleotides at the end of one strand of a double stranded RNAi agent form an overhang. The unpaired nucleotides may be on the sense strand or the antisense strand, creating either 3′ or 5′ overhangs. In some embodiments, the RNAi agent contains: a blunt end and a frayed end, a blunt end and 5′ overhang end, a blunt end and a 3′ overhang end, a frayed end and a 5′ overhang end, a frayed end and a 3′ overhang end, two 5′ overhang ends, two 3′ overhang ends, a 5′ overhang end and a 3′ overhang end, two frayed ends, or two blunt ends. Typically, when present, overhangs are located at the 3′ terminal ends of the sense strand, the antisense strand, or both the sense strand and the antisense strand.

The TSLP RNAi agents disclosed herein may also be comprised of one or more modified nucleotides. In some embodiments, substantially all of the nucleotides of the sense strand and substantially all of the nucleotides of the antisense strand of the TSLP RNAi agent are modified nucleotides. The TSLP RNAi agents disclosed herein may further be comprised of one or more modified internucleoside linkages, e.g., one or more phosphorothioate linkages. In some embodiments, a TSLP RNAi agent contains one or more modified nucleotides and one or more modified internucleoside linkages. In some embodiments, a 2′-modified nucleotide is combined with modified internucleoside linkage.

In some embodiments, a TSLP RNAi agent is prepared or provided as a salt, mixed salt, or a free acid. In some embodiments, a TSLP RNAi agent is prepared as a pharmaceutically acceptable salt. In some embodiments, a TSLP RNAi agent is prepared as a pharmaceutically acceptable sodium salt. Such forms that are well known in the art are within the scope of the inventions disclosed herein.

### Modified Nucleotides

Modified nucleotides, when used in various oligonucleotide constructs, can preserve activity of the compound in cells while at the same time increasing the serum stability of these compounds, and can also minimize the possibility of activating interferon activity in humans upon administration of the oligonucleotide construct.

In some embodiments, a TSLP RNAi agent contains one or more modified nucleotides. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides can include, but are not limited to, deoxyribonucleotides, nucleotide mimics, abasic nucleotides, 2′-modified nucleotides, 3′-modified nucleotides (2′-internucleoside linked), inverted nucleotides, modified nucleobase-comprising nucleotides, bridged nucleotides, peptide nucleic acids (PNAs), 2′,3′-seco nucleotide mimics (unlocked nucleobase analogues), locked nucleotides, 3′-O-methoxy (2′-internucleoside linked) nucleotides, 2′-F-Arabino nucleotides, 5′-Methyl-2′-fluoro nucleotides, morpholino nucleotides (modified nucleotides with a morpholine ring), nucleotides where the typical 5-membered sugar ring of the nucleotide has been modified, vinyl phosphonate deoxyribonucleotides, vinyl phosphonate containing nucleotides, and cyclopropyl phosphonate containing nucleotides. 2′-modified nucleotides (i.e., a nucleotide with a group other than a hydroxyl group at the 2′ position of the five-membered sugar ring) include, but are not limited to, 2′-O-methyl nucleotides (also referred to as 2′-methoxy nucleotides), 2′-fluoro nucleotides (also referred as 2′-deoxy-2′-fluoro nucleotides), 2′-deoxy nucleotides, 2′-methoxyethyl (2′-O-2-methoxylethyl) nucleotides (also referred to as 2′-MOE nucleotides), 2′-amino nucleotides, 2′-halo nucleotides, and 2′-alkyl nucleotides. It is not necessary for all positions in a given compound to be uniformly modified. Conversely, more than one modification can be incorporated in a single TSLP RNAi agent or even in a single nucleotide thereof. The TSLP RNAi agent sense strands and antisense strands can be synthesized and/or modified by methods known in the art. Modification at one nucleotide is independent of modification at another nucleotide.

Modified nucleobases include synthetic and natural nucleobases, such as 5-substituted pyrimidines, 6-azapyrimidines and N-2, N-6 and O-6 substituted purines, (e.g., 2-aminopropyladenine, 5-propynyluracil, or 5-propynylcytosine), 5-methylcytosine (5-me-C), 5-hydroxymethyl cytosine, inosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulfhydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine.

In some embodiments, the 5′ and/or 3′ end of the antisense strand can include abasic residues (Ab), which can also be referred to as an “abasic site” or “abasic nucleotide.” An abasic residue (Ab) is a nucleotide or nucleoside that lacks a nucleobase at the 1′ position of the sugar moiety. (See. e.g., U.S. Pat. No. 5,998,203). In some embodiments, an abasic residue can be placed internally in a nucleotide sequence. In some embodiments, Ab or AbAb can be added to the 3′ end of the antisense strand. In some embodiments, the 5′ end of the sense strand can include one or more additional abasic residues (e.g., (Ab) or (AbAb)). In some embodiments, UUAb, UAb, or Ab are added to the 3′ end of the sense strand. In some embodiments, an abasic (deoxyribose) residue can be replaced with a ribitol (abasic ribose) residue.

In some embodiments, all or substantially all of the nucleotides of an RNAi agent are modified nucleotides. As used herein, an RNAi agent wherein substantially all of the nucleotides present are modified nucleotides is an RNAi agent having four or fewer (i.e., 0, 1, 2, 3, or 4) nucleotides in both the sense strand and the antisense strand being ribonucleotides (i.e., unmodified). As used herein, a sense strand wherein substantially all of the nucleotides present are modified nucleotides is a sense strand having two or fewer (i.e., 0, 1, or 2) nucleotides in the sense strand being unmodified ribonucleotides. As used herein, an antisense sense strand wherein substantially all of the nucleotides present are modified nucleotides is an antisense strand having two or fewer (i.e., 0, 1, or 2) nucleotides in the antisense strand being unmodified ribonucleotides. In some embodiments, one or more nucleotides of an RNAi agent is an unmodified ribonucleotide. Chemical structures for certain modified nucleotides are set forth in Table 11 herein.

### Modified Internucleoside Linkages

In some embodiments, one or more nucleotides of a TSLP RNAi agent are linked by non-standard linkages or backbones (i.e., modified internucleoside linkages or modified backbones). Modified internucleoside linkages or backbones include, but are not limited to, phosphorothioate groups (represented herein as a lower case “s”), chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some embodiments, a sense strand of a TSLP RNAi agent can contain 1, 2, 3, 4, 5, or 6 phosphorothioate linkages, an antisense strand of a TSLP RNAi agent can contain 1, 2, 3, 4, 5, or 6 phosphorothioate linkages, or both the sense strand and the antisense strand independently can contain 1, 2, 3, 4, 5, or 6 phosphorothioate linkages. In some embodiments, a sense strand of a TSLP RNAi agent can contain 1, 2, 3, or 4 phosphorothioate linkages, an antisense strand of a TSLP RNAi agent can contain 1, 2, 3, or 4 phosphorothioate linkages, or both the sense strand and the antisense strand independently can contain 1, 2, 3, or 4 phosphorothioate linkages.

In some embodiments, a TSLP RNAi agent sense strand contains at least two phosphorothioate internucleoside linkages. In some embodiments, the phosphorothioate internucleoside linkages are between the nucleotides at positions 1-3 from the 3′ end of the sense strand. In some embodiments, one phosphorothioate internucleoside linkage is at the 5′ end of the sense strand nucleotide sequence, and another phosphorothioate linkage is at the 3′ end of the sense strand nucleotide sequence. In some embodiments, two phosphorothioate internucleoside linkage are located at the 5′ end of the sense strand, and another phosphorothioate linkage is at the 3′ end of the sense strand. In some embodiments, the sense strand does not include any phosphorothioate internucleoside linkages between the nucleotides, but contains one, two, or three phosphorothioate linkages between the terminal nucleotides on both the 5′ and 3′ ends and the optionally present inverted abasic residue terminal caps. In some embodiments, the targeting ligand is linked to the sense strand via a phosphorothioate linkage.

In some embodiments, a TSLP RNAi agent antisense strand contains four phosphorothioate internucleoside linkages. In some embodiments, the four phosphorothioate internucleoside linkages are between the nucleotides at positions 1-3 from the 5′ end of the antisense strand and between the nucleotides at positions 19-21, 20-22, 21-23, 22-24, 23-25, or 24-26 from the 5′ end. In some embodiments, three phosphorothioate internucleoside linkages are located between positions 1-4 from the 5′ end of the antisense strand, and a fourth phosphorothioate internucleoside linkage is located between positions 20-21 from the 5′ end of the antisense strand. In some embodiments, a TSLP RNAi agent contains at least three or four phosphorothioate internucleoside linkages in the antisense strand.

### Capping Residues or Moieties

In some embodiments, the sense strand may include one or more capping residues or moieties, sometimes referred to in the art as a “cap,” a “terminal cap,” or a “capping residue.” As used herein, a “capping residue” is a non-nucleotide compound or other moiety that can be incorporated at one or more termini of a nucleotide sequence of an RNAi agent disclosed herein. A capping residue can provide the RNAi agent, in some instances, with certain beneficial properties, such as, for example, protection against nuclease degradation. In some embodiments, inverted abasic residues (invAb) (also referred to in the art as “inverted abasic sites”) are added as capping residues (see Table 11). (See, e.g., F. Czauderna, Nucleic Acids Res., 2003, 31(11), 2705-16). Capping residues are generally known in the art, and include, for example, inverted abasic residues as well as carbon chains such as a terminal C₃H₇ (propyl), C₆H₁₃ (hexyl), or C₁₂H₂₅ (dodecyl) groups. In some embodiments, a capping residue is present at either the 5′ terminal end, the 3′ terminal end, or both the 5′ and 3′ terminal ends of the sense strand. In some embodiments, the 5′ end and/or the 3′ end of the sense strand may include more than one inverted abasic deoxyribose moiety as a capping residue.

In some embodiments, one or more inverted abasic residues (invAb) are added to the 3′ end of the sense strand. In some embodiments, one or more inverted abasic residues (invAb) are added to the 5′ end of the sense strand. In some embodiments, one or more inverted abasic residues or inverted abasic sites are inserted between the targeting ligand and the nucleotide sequence of the sense strand of the RNAi agent. In some embodiments, the inclusion of one or more inverted abasic residues or inverted abasic sites at or near the terminal end or terminal ends of the sense strand of an RNAi agent allows for enhanced activity or other desired properties of an RNAi agent.

In some embodiments, one or more inverted abasic residues (invAb) are added to the 5′ end of the sense strand. In some embodiments, one or more inverted abasic residues can be inserted between the targeting ligand and the nucleotide sequence of the sense strand of the RNAi agent. The inverted abasic residues may be linked via phosphate, phosphorothioate (e.g., shown herein as (invAb)s)), or other internucleoside linkages. In some embodiments, the inclusion of one or more inverted abasic residues at or near the terminal end or terminal ends of the sense strand of an RNAi agent may allow for enhanced activity or other desired properties of an RNAi agent. In some embodiments, an inverted abasic (deoxyribose) residue can be replaced with an inverted ribitol (abasic ribose) residue. In some embodiments, the 3′ end of the antisense strand core stretch sequence, or the 3′ end of the antisense strand sequence, may include an inverted abasic residue. The chemical structures for inverted abasic deoxyribose residues are shown in Table 11 below.

### TSLP RNAi Agents

The TSLP RNAi agents disclosed herein are designed to target specific positions on a TSLP gene (e.g., SEQ ID NO:1 (NM_0033035.5)). As defined herein, an antisense strand sequence is designed to target a TSLP gene at a given position on the gene when the 5′ terminal nucleobase of the antisense strand is aligned with a position that is 21 nucleotides downstream (towards the 3′ end) from the position on the gene when base pairing to the gene. For example, as illustrated in Tables 1 and 2 herein, an antisense strand sequence designed to target a TSLP gene at position 571 requires that when base pairing to the gene, the 5′ terminal nucleobase of the antisense strand is aligned with position 591 of a TSLP gene.

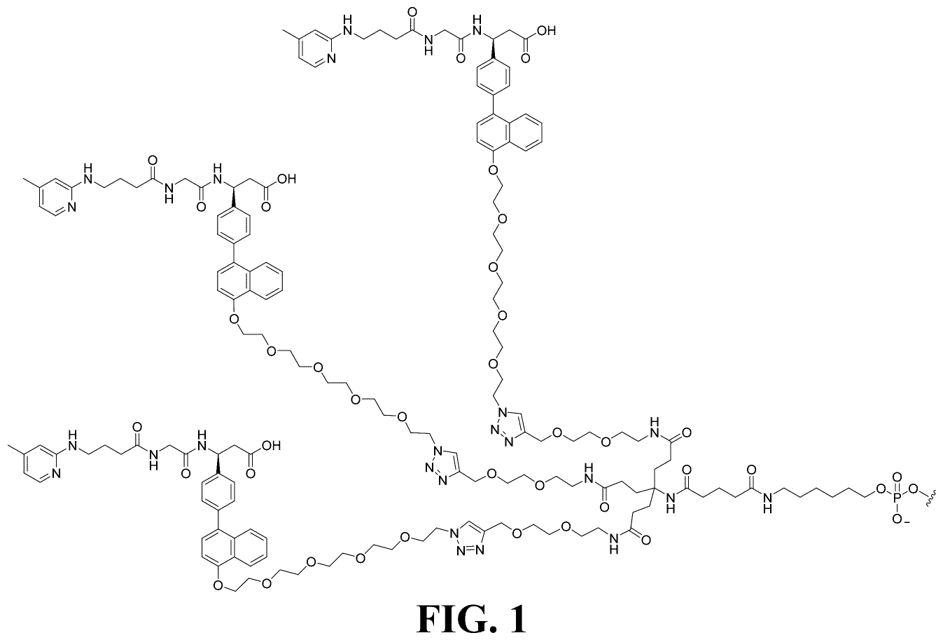

As provided herein, a TSLP RNAi agent does not require that the nucleobase at position 1(5′→3′) of the antisense strand be complementary to the gene, provided that there is at least 85% complementarity (e.g., at least 85, 86, 87, 88, 89, 90, 91, 92, 93, 94, 95, 96, 97, 98, 99, or 100% complementarity) of the antisense strand and the gene across a core stretch sequence of at least 16 consecutive nucleotides. For example, for a TSLP RNAi agent disclosed herein that is designed to target position 571 of a TSLP gene, the 5′ terminal nucleobase of the antisense strand of the of the TSLP RNAi agent must be aligned with position 591 of the gene; however, the 5′ terminal nucleobase of the antisense strand may be, but is not required to be, complementary to position 591 of a TSLP gene, provided that there is at least 85% complementarity (e.g., at least 85, 86, 87, 88, 89, 90, 91, 92, 93, 94, 95, 96, 97, 98, 99, or 100% complementarity) of the antisense strand and the gene transcript across a core stretch sequence of at least 16 consecutive nucleotides. As shown by, among other things, the various examples disclosed herein, the specific site of binding of the gene by the antisense strand of the TSLP RNAi agent (e.g., whether the TSLP RNAi agent is designed to target a TSLP gene at position 571, at position 520, at position 570, or at some other position) is an important factor to the level of inhibition achieved and the off-target effects (e.g., potential safety issues) of the TSLP RNAi agent. (See. e.g., Kamola et al., The siRNA Non-seed Region and Its Target Sequences are Auxiliary Determinants of Off-Target Effects. PLOS Computational Biology, 11(12), FIG. 1 (2015)).

In some embodiments, the TSLP RNAi agents disclosed herein target a TSLP gene at or near the positions of the TSLP sequence shown in Table 1. In some embodiments, the antisense strand of a TSLP RNAi agent disclosed herein includes a core stretch sequence that is fully, substantially, or at least partially complementary to a target TSLP 19-mer sequence disclosed in Table 1.

TABLE 1
TSLP 19-mer mRNA Target Sequences (taken from homo sapiens thymic stromal
lymphopoietin (TSLP) transcript variant 1, GenBank NM_033035.5 (SEQ ID NO: 1))

| | TSLP 19-mer | Corresponding | Targeted Gene |
|---|---|---|---|
| SEQ ID | Target Sequences | Positions of Sequence | Position (as |
| No. | (5′→3′) | on SEQ ID NO: 1 | referred to herein) |
| 2 | UUGCCUUACUGAAAUCCAG | 400-418 | 398 |
| 3 | CUGAAAUCCAGAGCCUAAC | 408-426 | 406 |
| 4 | GAAAUCCAGAGCCUAACCU | 410-428 | 408 |
| 5 | AAUCCAGAGCCUAACCUUC | 412-430 | 410 |
| 6 | CCAGAGCCUAACCUUCAAU | 415-433 | 413 |
| 7 | CAGAGCCUAACCUUCAAUC | 416-434 | 414 |
| 8 | AGAGCCUAACCUUCAAUCC | 417-435 | 415 |
| 9 | GAGCCUAACCUUCAAUCCC | 418-436 | 416 |
| 10 | AGCCUAACCUUCAAUCCCA | 419-437 | 417 |
| 11 | UUCGCCAUGAAAACUAAGG | 470-488 | 468 |
| 12 | GGCUGCCUUAGCUAUCUGG | 487-505 | 485 |
| 13 | AGGCUAUUCGGAAACUCAG | 511-529 | 509 |
| 14 | UAUUCGGAAACUCAGAUAA | 515-533 | 513 |
| 15 | AUUCGGAAACUCAGAUAAA | 516-534 | 514 |
| 16 | UUCGGAAACUCAGAUAAAU | 517-535 | 515 |
| 17 | UCGGAAACUCAGAUAAAUG | 518-536 | 516 |
| 18 | AAACUCAGAUAAAUGCUAA | 522-540 | 520 |
| 19 | GCUACUCAGGCAAUGAAGA | 536-554 | 534 |
| 20 | CUACUCAGGCAAUGAAGAA | 537-555 | 535 |
| 21 | GAAGAGGAGAAAAAGGAAA | 553-571 | 551 |
| 22 | AGGAGAAAAAGGAAAGUCA | 557-575 | 555 |
| 23 | GGAGAAAAAGGAAAGUCAC | 558-576 | 556 |
| 24 | AAAAAGGAAAGUCACAACC | 562-580 | 560 |
| 25 | AAAGGAAAGUCACAACCAA | 564-585 | 562 |
| 26 | AGGAAAGUCACAACCAAUA | 566-584 | 564 |
| 27 | GAAAGUCACAACCAAUAAA | 568-586 | 566 |
| 28 | AAAGUCACAACCAAUAAAU | 569-587 | 567 |
| 29 | AAGUCACAACCAAUAAAUG | 570-588 | 568 |
| 30 | GUCACAACCAAUAAAUGUC | 572-590 | 570 |
| 31 | UCACAACCAAUAAAUGUCU | 573-591 | 571 |
| 32 | AAUGUCUGGAACAAGUGUC | 585-603 | 583 |
| 33 | UGUCUGGAACAAGUGUCAC | 587-605 | 585 |
| 34 | UGGAACAAGUGUCACAAUU | 591-609 | 589 |
| 35 | GGAACAAGUGUCACAAUUA | 592-610 | 590 |
| 36 | GAACAAGUGUCACAAUUAC | 593-611 | 591 |
| 37 | AACAAGUGUCACAAUUACA | 594-612 | 592 |
| 38 | CUUCAAUCGACCUUUACUG | 628-646 | 626 |
| 39 | AGUAAACCAUCUUUAUUAU | 654-672 | 652 |
| 40 | AUAUUUCACAGCACCAAAA | 677-695 | 675 |
| 41 | AACAUUAACUCUAACUGUG | 721-739 | 719 |
| 42 | AGAAGAGUUUCUUAACUUA | 775-793 | 773 |
| 43 | AAGAGUUUCUUAACUUACU | 777-795 | 775 |
| 44 | ACUACUCCUCAAAUGUUGA | 838-856 | 836 |
| 45 | UCCAUAACAUUGAUGACUG | 865-883 | 863 |
| 46 | AUUGAUGACUGGCUUCAUG | 873-891 | 871 |
| 47 | AAUGAUAGCACCUAAACUU | 994-1012 | 992 |
| 48 | GACAGACAUUCCUUCUACA | 1023-1041 | 1021 |
| 49 | GACAUUCCUUCUACAUGUA | 1027-1045 | 1025 |
| 50 | CAUGUAAUGACACUUCUUG | 1040-1058 | 1038 |
| 51 | AUGUAAUGACACUUCUUGU | 1041-1059 | 1039 |
| 52 | UGUAAUGACACUUCUUGUG | 1042-1060 | 1040 |
| 53 | CAAGCAAAGUAUUGUGAAA | 1151-1169 | 1149 |
| 54 | ACAAGUAGAUCCUGAGAAG | 1220-1238 | 1218 |
| 55 | UACCUUUGUUACAGCUACU | 1239-1257 | 1237 |
| 56 | CCUUUGUAAUUGACACUAU | 1328-1346 | 1326 |

Homo sapiens thymic stromal lymphopoietin (TSLP) transcript variant 1, GenBank NM_033035.5, gene transcript (2610 bases):

| | |
|---|---|
| 1 | atcagggaga ctccaactta aggcaacagc atgggtgaat aagggcttcc tgtggactgg |
| 61 | caatgagagg caaaacctgg tgcttgagca ctggccccta aggcaggcct tacagatctc |
| 121 | ttacactcgt ggtgggaaga gtttagtgtg aaactggggt ggaattgggt gtccacgtat |
| 181 | gttccctttt gccttactat atgttctgtc agtttctttc aggaaaatct tcatcttaca |
| 241 | acttgtaggg ctggtgttaa cttacgactt cactaactgt gactttgaga agattaaagc |
| 301 | agcctatctc agtactattt ctaaagacct gattacatat atgagtggga ccaaaagtac |
| 361 | cgagttcaac aacaccgtct cttgtagcaa tcggccacat tgccttactg aaatccagag |
| 421 | cctaaccttc aatcccaccg ccggctgcgc gtcgctcgcc aaagaaatgt tcgccatgaa |
| 481 | aactaaggct gccttagcta tctggtgccc aggctattcg gaaactcaga taaatgctac |
| 541 | tcaggcaatg aagaagagga gaaaaaggaa agtcacaacc aataaatgtc tggaacaagt |
| 601 | gtcacaatta caaggattgt ggcgtcgctt caatcgacct ttactgaaac aacagtaaac |
| 661 | catctttatt atggtcatat ttcacagcac caaaataaat catctttatt aagtagatga |
| 721 | aacattaact ctaactgtga caaagaagac cacaaatagt tatcttttaa ttacagaaga |
| 781 | gtttcttaac ttacttttgt aagtttttat tgtgtaagtt tataatgcag gggaagtact |
| 841 | actcctcaaa tgttgaggga agcttccata acattgatga ctggcttcat ggcagtaatt |
| 901 | ctcggctgta gttgcataag cattgctcaa gaggaaaatc caaaagtgca gcaggagaac |
| 961 | tcttttccct gaaaaaggaa aaatattgaa ctcaatgata gcacctaaac ttacatttaa |
| 1021 | aagacagaca ttccttctac atgtaatgac acttcttgtg ttaaactaaa aatttacaag |
| 1081 | agaagaaagt gaaagcaaat ggggtttcac aaatagttgt aaatatagtg aagcaatttg |
| 1141 | aaataatttt caagcaaagt attgtgaaag tattctaagc caagttttaa atattatcta |
| 1201 | acagacaaga gtggtatata caagtagatc ctgagaagta cctttgttac agctactata |
| 1261 | aatatacata taaattatag aatctacttt aatttatttt gtgaacactt ttgaaaatgt |
| 1321 | acatgttcct ttgtaattga cactatatat ttcttaataa aataattctc aaatttgttt |
| 1381 | cttatgaatc atctctcaaa tctagttaga caatttgcac acatactttt ctaagggaca |
| 1441 | ttatcttcct tcaggttttt acctccactc atccttagag cccactgact gctccccttt |
| 1501 | atacctgttg gccctgccta taggagagaa tatttggaga taggcagctt caggatgcat |
| 1561 | tgcaatcatc cttttcttaa attatgtcac tagtctttta ttttttcccc tcttgaactt |
| 1621 | tcctcacacc tggaagaaac aaagtaggaa aaagtgaaca ggggatgtca aatcgattct |
| 1681 | tgaattcccg ctgcaagcta gagccgcagg caccctctca ctcaatttcc actcagaacc |
| 1741 | ctataaacac cagtgggaag ggcaacccac tgcacgtggg aatgcactga tttttcctag |
| 1801 | gagtagacat gttcctctaa ttactccctg agggttagtt ggggctaaac catgacagaa |
| 1861 | gtggggaagt tcaatgtcct taaatccatc ttacttgcca acaggtaaga ggaagcttac |
| 1921 | attacatgtc cagtccacat ttaaagagca cttactgtgg aacaagcctt cagccaaaca |
| 1981 | atggggatag aaaagtaggt aagactcagc ctttgtccag agaagctcag ggtatagctg |
| 2041 | aataggcagt ttcttttgtc ctgaggaaaa tcaggacatg cctgctttct aaaaatcttc |
| 2101 | ctctgaagac ctgacccaag ctcttaaatg ctattgtaag agaaatttct ttgtctatta |
| 2161 | actccatttt agtagggatt cactgactag attttactga actatgaaaa taaatacaca |
| 2221 | taatttttca caaaattttg ggcccaattc ccctaaaaga attgaggatt agggagaaag |
| 2281 | gagacaactc aaagtcatcc cattaagtgc agtttctttg aatcttctgc tttatcttta |
| 2341 | aaaatttgta taatttatat attttattct atgtgttcca tagatatctt aatgtaaaat |
| 2401 | tagtcattta aattacactg tcaattaaaa gtaatgggca agagattgca tcatactaat |
| 2461 | ttagtaagaa cgttcccaaa tgttgtaaca atgtggatca tacatctctg gttttttaaa |
| 2521 | tgtattgagg ctttcttggt ggactagtat agtatacggt cagttatgtc aatgtttcat |
| 2581 | ggtcaataaa aaggaagttg caaattgtga |

In some embodiments, a TSLP RNAi agent includes an antisense strand wherein position 19 of the antisense strand (5′→3′) is capable of forming a base pair with position 1 of a 19-mer target sequence disclosed in Table 1. In some embodiments, a TSLP agent includes an antisense strand wherein position 1 of the antisense strand (5′→3′) is capable of forming a base pair with position 19 of a 19-mer target sequence disclosed in Table 1.

In some embodiments, a TSLP agent includes an antisense strand wherein position 2 of the antisense strand (5′→3′) is capable of forming a base pair with position 18 of a 19-mer target sequence disclosed in Table 1. In some embodiments, a TSLP agent includes an antisense strand wherein positions 2 through 18 of the antisense strand (5′→3′) are capable of forming base pairs with each of the respective complementary bases located at positions 18 through 2 of the 19-mer target sequence disclosed in Table 1.

For the RNAi agents disclosed herein, the nucleotide at position 1 of the antisense strand (from 5′ end→3′ end) can be perfectly complementary to a TSLP gene, or can be non-complementary to a TSLP gene. In some embodiments, the nucleotide at position 1 of the antisense strand (from 5′ end→3′ end) is a U, A, or dT. In some embodiments, the nucleotide at position 1 of the antisense strand (from 5′ end→3′ end) forms an A:U or U:A base pair with the sense strand.

In some embodiments, a TSLP RNAi agent antisense strand comprises the sequence of nucleotides (from 5′ end→3′ end) 2-18 or 2-19 of any of the antisense strand sequences in Table 2 or Table 3. In some embodiments, a TSLP RNAi sense strand comprises the sequence of nucleotides (from 5′ end→3′ end) 1-17, 1-18, or 2-18 of any of the sense strand sequences in Table 2, Table 4, Table 5, or Table 6.

In some embodiments, a TSLP RNAi agent is comprised of (i) an antisense strand comprising the sequence of nucleotides (from 5′ end→3′ end) 2-18 or 2-19 of any of the antisense strand sequences in Table 2 or Table 3, and (ii) a sense strand comprising the sequence of nucleotides (from 5′ end→3′ end) 1-17 or 1-18 of any of the sense strand sequences in Table 2, Table 4, Table 5, or Table 6.

In some embodiments, the TSLP RNAi agents include core 19-mer nucleotide sequences shown in the following Table 2.

TABLE 2
TSLP RNAi Agent Antisense Strand and Sense Strand Core Stretch Base Sequences

| | | | | Corresponding | |
|---|---|---|---|---|---|
| | Antisense Strand Base | | Sense Strand Base | Positions of | |
| | Sequence (5′→3′) | | Sequence (5′→3′) | Identified | Targeted |
| SEQ ID | (Shown as an Unmodified | SEQ ID | (Shown as an Unmodified | Sequence on | Gene |
| NO:. | Nucleotide Sequence) | NO:. | Nucleotide Sequence) | SEQ ID NO: 1 | Position |
| 57 | CUGGAUUUCAGUAAGGCAA | 322 | UUGCCUUACUGAAAUCCAG | 400-418 | 398 |
| 58 | UUGGAUUUCAGUAACCGUU | 323 | UUGCCUUACUGAAAUCCAA | 400-418 | 398 |
| 59 | NUGGAUUUCAGUAACCGUU | 324 | UUGCCUUACUGAAAUCCAN | 400-418 | 398 |
| 60 | UUGGAUUUCAGUAACCGUN | 325 | NUGCCUUACUGAAAUCCAA | 400-418 | 398 |
| 61 | NUGGAUUUCAGUAACCGUN | 326 | NUGCCUUACUGAAAUCCAN | 400-418 | 398 |
| 62 | GUUAGGCUCUGGAUUUCAG | 327 | CUGAAAUCCAGAGCCUAAC | 408-426 | 406 |
| 63 | UUUAGGCUCUGGAUUUCAG | 328 | CUGAAAUCCAGAGCCUAAA | 408-426 | 406 |
| 64 | NUUAGGCUCUGGAUUUCAG | 329 | CUGAAAUCCAGAGCCUAAN | 408-426 | 406 |
| 65 | UUUAGGCUCUGGAUUUCAN | 330 | NUGAAAUCCAGAGCCUAAA | 408-426 | 406 |
| 66 | NUUAGGCUCUGGAUUUCAN | 331 | NUGAAAUCCAGAGCCUAAN | 408-426 | 406 |
| 67 | AGGUUAGGCUCUGGAUUUC | 332 | GAAAUCCAGAGCCUAACCU | 410-428 | 408 |
| 68 | UGGUUAGGCUCUGGAUUUC | 333 | GAAAUCCAGAGCCUAACCA | 410-428 | 408 |
| 69 | NGGUUAGGCUCUGGAUUUC | 334 | GAAAUCCAGAGCCUAACCN | 410-428 | 408 |
| 70 | NGGUUAGGCUCUGGAUUUN | 335 | NAAAUCCAGAGCCUAACCN | 410-428 | 408 |
| 71 | AGGUUAGGCUCUGGAUUUC | 336 | GAAAUCCAGAGCCUAACCU | 410-428 | 408 |
| 72 | GAAGGUUAGGCUCUGGAUU | 337 | AAUCCAGAGCCUAACCUUC | 412-430 | 410 |
| 73 | UAAGGUUAGGCUCUGGAUU | 338 | AAUCCAGAGCCUAACCUUA | 412-430 | 410 |
| 74 | NAAGGUUAGGCUCUGGAUU | 339 | AAUCCAGAGCCUAACCUUN | 412-430 | 410 |
| 75 | UAAGGUUAGGCUCUGGAUN | 340 | AAUCCAGAGCCUAACCUUN | 412-430 | 410 |
| 76 | NAAGGUUAGGCUCUGGAUN | 341 | NAUCCAGAGCCUAACCUUN | 412-430 | 410 |
| 77 | AUUGAAGGUUAGGCUCUGG | 342 | CCAGAGCCUAACCUUCAAU | 415-433 | 413 |
| 78 | UUUGAAGGUUAGGCUCUGG | 343 | CCAGAGCCUAACCUUCAAA | 415-433 | 413 |
| 79 | NUUGAAGGUUAGGCUCUGG | 344 | CCAGAGCCUAACCUUCAAN | 415-433 | 413 |
| 80 | AUUGAAGGUUAGGCUCUGN | 345 | NCAGAGCCUAACCUUCAAU | 415-433 | 413 |
| 81 | NUUGAAGGUUAGGCUCUGN | 346 | NCAGAGCCUAACCUUCAAN | 415-433 | 413 |
| 82 | GAUUGAAGGUUAGGCUCUG | 347 | CAGAGCCUAACCUUCAAUC | 416-434 | 414 |
| 83 | AAUUGAAGGUUAGGCUCUG | 348 | CAGAGCCUAACCUUCAAUU | 416-434 | 414 |
| 84 | UAUUGAAGGUUAGGCUCUG | 349 | CAGAGCCUAACCUUCAAUA | 416-434 | 414 |
| 85 | NAUUGAAGGUUAGGCUCUG | 350 | CAGAGCCUAACCUUCAAUN | 416-434 | 414 |
| 86 | NAUUGAAGGUUAGGCUCUN | 351 | NAGAGCCUAACCUUCAAUN | 416-434 | 414 |
| 87 | GGAUUGAAGGUUAGGCUCU | 352 | AGAGCCUAACCUUCAAUCC | 417-435 | 415 |
| 88 | AGAUUGAAGGUUAGGCUCU | 353 | AGAGCCUAACCUUCAAUCU | 417-435 | 415 |
| 89 | UGAUUGAAGGUUAGGCUCU | 354 | AGAGCCUAACCUUCAAUCA | 417-435 | 415 |
| 90 | NGAUUGAAGGUUAGGCUCU | 355 | AGAGCCUAACCUUCAAUCN | 417-435 | 415 |
| 91 | NGAUUGAAGGUUAGGCUCN | 356 | NGAGCCUAACCUUCAAUCN | 417-435 | 415 |
| 92 | GGGAUUGAAGGUUAGGCUC | 357 | GAGCCUAACCUUCAAUCCC | 418-436 | 416 |
| 93 | AGGAUUGAAGGUUAGGCUC | 358 | GAGCCUAACCUUCAAUCCU | 418-436 | 416 |
| 94 | UGGAUUGAAGGUUAGGCUC | 359 | GAGCCUAACCUUCAAUCCA | 418-436 | 416 |
| 95 | NGGAUUGAAGGUUAGGCUC | 360 | GAGCCUAACCUUCAAUCCN | 418-436 | 416 |
| 96 | NGGAUUGAAGGUUAGGCUN | 361 | NAGCCUAACCUUCAAUCCN | 418-436 | 416 |
| 97 | UGGGAUUGAAGGUUAGGCU | 362 | AGCCUAACCUUCAAUCCCA | 419-437 | 417 |
| 98 | NGGGAUUGAAGGUUAGGCU | 363 | AGCCUAACCUUCAAUCCCN | 419-437 | 417 |
| 99 | UGGGAUUGAAGGUUAGGCN | 364 | NGCCUAACCUUCAAUCCCA | 419-437 | 417 |
| 100 | NGGGAUUGAAGGUUAGGCN | 365 | NGCCUAACCUUCAAUCCCN | 419-437 | 417 |
| 101 | CCUUAGUUUUCAUGGCGAA | 366 | UUCGCCAUGAAAACUAAGG | 470-488 | 468 |
| 102 | ACUUAGUUUUCAUGGCGAA | 367 | UUCGCCAUGAAAACUAAGU | 470-488 | 468 |
| 103 | UCUUAGUUUUCAUGGCGAA | 368 | UUCGCCAUGAAAACUAAGA | 470-488 | 468 |
| 104 | NCUUAGUUUUCAUGGCGAA | 369 | UUCGCCAUGAAAACUAAGN | 470-488 | 468 |
| 105 | NCUUAGUUUUCAUGGCGAN | 370 | NUCGCCAUGAAAACUAAGN | 470-488 | 468 |
| 106 | CCAGAUAGCUAAGGCAGCC | 371 | GGCUGCCUUAGCUAUCUGG | 487-505 | 485 |
| 107 | ACAGAUAGCUAAGGCAGCC | 372 | GGCUGCCUUAGCUAUCUGU | 487-505 | 485 |
| 108 | UCAGAUAGCUAAGGCAGCC | 373 | GGCUGCCUUAGCUAUCUGA | 487-505 | 485 |
| 109 | NCAGAUAGCUAAGGCAGCC | 374 | GGCUGCCUUAGCUAUCUGN | 487-505 | 485 |
| 110 | NCAGAUAGCUAAGGCAGCN | 375 | NGCUGCCUUAGCUAUCUGN | 487-505 | 485 |
| 111 | CUGAGUUUCCGAAUAGCCU | 376 | AGGCUAUUCGGAAACUCAG | 511-529 | 509 |
| 112 | AUGAGUUUCCGAAUAGCCU | 377 | AGGCUAUUCGGAAACUCAU | 511-529 | 509 |
| 113 | UUGAGUUUCCGAAUAGCCU | 378 | AGGCUAUUCGGAAACUCAA | 511-529 | 509 |
| 114 | NUGAGUUUCCGAAUAGCCU | 379 | AGGCUAUUCGGAAACUCAN | 511-529 | 509 |
| 115 | NUGAGUUUCCGAAUAGCCN | 380 | NGGCUAUUCGGAAACUCAN | 511-529 | 509 |
| 116 | UUAUCUGAGUUUCCGAAUA | 381 | UAUUCGGAAACUCAGAUAA | 515-533 | 513 |
| 117 | AUAUCUGAGUUUCCGAAUA | 382 | UAUUCGGAAACUCAGAUAU | 515-533 | 513 |
| 118 | NUAUCUGAGUUUCCGAAUA | 383 | UAUUCGGAAACUCAGAUAN | 515-533 | 513 |
| 119 | NUAUCUGAGUUUCCGAAUN | 384 | NAUUCGGAAACUCAGAUAN | 515-533 | 513 |
| 120 | UUUAUCUGAGUUUCCGAAU | 385 | AUUCGGAAACUCAGAUAAA | 516-534 | 514 |
| 121 | AUUAUCUGAGUUUCCGAAU | 386 | AUUCGGAAACUCAGAUAAU | 516-534 | 514 |
| 122 | NUUAUCUGAGUUUCCGAAU | 387 | AUUCGGAAACUCAGAUAAN | 516-534 | 514 |
| 123 | NUUAUCUGAGUUUCCGAAN | 388 | NUUCGGAAACUCAGAUAAN | 516-534 | 514 |
| 124 | AUUUAUCUGAGUUUCCGAA | 389 | UUCGGAAACUCAGAUAAAU | 517-535 | 515 |
| 125 | NUUUAUCUGAGUUUCCGAA | 390 | UUCGGAAACUCAGAUAAAN | 517-535 | 515 |
| 126 | AUUUAUCUGAGUUUCCGAN | 391 | NUCGGAAACUCAGAUAAAU | 517-535 | 515 |
| 127 | NUUUAUCUGAGUUUCCGAN | 392 | NUCGGAAACUCAGAUAAAN | 517-535 | 515 |
| 128 | AUUUAUCUGAGUUUCCGAA | 393 | UUCGGAAACUCAGAUAA(A^{2N})U | 517-535 | 515 |
| 129 | NUUUAUCUGAGUUUCCGAA | 394 | UUCGGAAACUCAGAUAA(A^{2N})N | 517-535 | 515 |
| 130 | AUUUAUCUGAGUUUCCGAN | 395 | NUCGGAAACUCAGAUAA(A^{2N})U | 517-535 | 515 |
| 131 | NUUUAUCUGAGUUUCCGAN | 396 | NUCGGAAACUCAGAUAA(A^{2N})N | 517-535 | 515 |
| 132 | CAUUUAUCUGAGUUUCCGA | 397 | UCGGAAACUCAGAUAAAUG | 518-536 | 516 |
| 133 | AAUUUAUCUGAGUUUCCGA | 398 | UCGGAAACUCAGAUAAAUA | 518-536 | 516 |
| 134 | UAUUUAUCUGAGUUUCCGA | 399 | UCGGAAACUCAGAUAAAUU | 518-536 | 516 |
| 135 | NAUUUAUCUGAGUUUCCGA | 400 | UCGGAAACUCAGAUAAAUN | 518-536 | 516 |
| 136 | NAUUUAUCUGAGUUUCCGN | 401 | NCGGAAACUCAGAUAAAUN | 518-536 | 516 |
| 137 | UUAGCAUUUAUCUGAGUUU | 402 | AAACUCAGAUAAAUGCUAA | 522-540 | 520 |
| 138 | NUAGCAUUUAUCUGAGUUU | 403 | AAACUCAGAUAAAUGCUAN | 522-540 | 520 |
| 139 | UUAGCAUUUAUCUGAGUUC | 404 | GAACUCAGAUAAAUGCUAA | 522-540 | 520 |
| 140 | UUAGCAUUUAUCUGAGUUC | 405 | G(A^{2N})ACUCAGAUAAAUGCUAA | 522-540 | 520 |
| 141 | UUAGCAUUUAUCUGAGUUN | 406 | NAACUCAGAUAAAUGCUAA | 522-540 | 520 |
| 142 | NUAGCAUUUAUCUGAGUUN | 407 | NAACUCAGAUAAAUGCUAN | 522-540 | 520 |
| 143 | UUAGCAUUUAUCUGAGUUN | 408 | N(A^{2N})ACUCAGAUAAAUGCUAA | 522-540 | 520 |
| 144 | NUAGCAUUUAUCUGAGUUN | 409 | N(A^{2N})ACUCAGAUAAAUGCUAN | 522-540 | 520 |
| 145 | UCUUCAUUGCCUGAGUAGC | 410 | GCUACUCAGGCAAUGAAGA | 536-554 | 534 |
| 146 | ACUUCAUUGCCUGAGUAGC | 411 | GCUACUCAGGCAAUGAAGU | 536-554 | 534 |
| 147 | NCUUCAUUGCCUGAGUAGC | 412 | GCUACUCAGGCAAUGAAGN | 536-554 | 534 |
| 148 | NCUUCAUUGCCUGAGUAGN | 413 | NCUACUCAGGCAAUGAAGN | 536-554 | 534 |
| 149 | UUCUUCAUUGCCUGAGUAG | 414 | CUACUCAGGCAAUGAAGAA | 537-555 | 535 |
| 150 | AUCUUCAUUGCCUGAGUAG | 415 | CUACUCAGGCAAUGAAGAU | 537-555 | 535 |
| 151 | NUCUUCAUUGCCUGAGUAG | 416 | CUACUCAGGCAAUGAAGAN | 537-555 | 535 |
| 152 | NUCUUCAUUGCCUGAGUAN | 417 | NUACUCAGGCAAUGAAGAN | 537-555 | 535 |
| 153 | UUUAUUGGUUGUGACUUUC | 418 | CUACUCAGGCAAUGAAGAA | 537-555 | 535 |
| 154 | AUUUAUUGGUUGUGACUUU | 419 | GAAGAGGAGAAAAAGGAAA | 553-571 | 551 |
| 155 | UGACUUUCCUUUUUCUCCU | 420 | AGGAGAAAAAGGAAAGUCA | 557-575 | 555 |
| 156 | AGACUUUCCUUUUUCUCCU | 421 | AGGAGAAAAAGGAAAGUCN | 557-575 | 555 |
| 157 | NGACUUUCCUUUUUCUCCU | 422 | AGGAGAAAAAGGAAAGUCN | 557-575 | 555 |
| 158 | NGACUUUCCUUUUUCUCCN | 423 | NGGAGAAAAAGGAAAGUCN | 557-575 | 555 |
| 159 | UUGACUUUCCUUUUUCUCC | 424 | GGAGAAAAAGGAAAGUCAC | 558-576 | 556 |
| 160 | GGUUGUGACUUUCCUUUUU | 425 | AAAAAGGAAAGUCACAACC | 562-580 | 560 |
| 161 | AGUUGUGACUUUCCUUUUU | 426 | AAAAAGGAAAGUCACAACU | 562-580 | 560 |
| 162 | UGUUGUGACUUUCCUUUUU | 427 | AAAAAGGAAAGUCACAACA | 562-580 | 560 |
| 163 | NGUUGUGACUUUCCUUUUU | 428 | AAAAAGGAAAGUCACAACN | 562-580 | 560 |
| 164 | NGUUGUGACUUUCCUUUUN | 429 | NAAAAGGAAAGUCACAACN | 562-580 | 560 |
| 165 | UUGGUUGUGACUUUCCUUU | 430 | AAAGGAAAGUCACAACCAA | 564-585 | 562 |
| 166 | AUGGUUGUGACUUUCCUUU | 431 | AAAGGAAAGUCACAACCAU | 564-585 | 562 |
| 167 | NUGGUUGUGACUUUCCUUU | 432 | AAAGGAAAGUCACAACCAN | 564-585 | 562 |
| 168 | NUGGUUGUGACUUUCCUUN | 433 | NAAGGAAAGUCACAACCAN | 564-585 | 562 |
| 169 | UAUUGGUUGUGACUUUCCU | 434 | AGGAAAGUCACAACCAAUA | 566-584 | 564 |
| 170 | AAUUGGUUGUGACUUUCCU | 435 | AGGAAAGUCACAACCAAUU | 566-584 | 564 |
| 171 | NAUUGGUUGUGACUUUCCU | 436 | AGGAAAGUCACAACCAAUN | 566-584 | 564 |
| 172 | NAUUGGUUGUGACUUUCCN | 437 | NGGAAAGUCACAACCAAUN | 566-584 | 564 |
| 173 | UUUAUUGGUUGUGACUUUC | 438 | GAAAGUCACAACCAAUAAA | 568-586 | 566 |
| 174 | AUUAUUGGUUGUGACUUUC | 439 | GAAAGUCACAACCAAUAAU | 568-586 | 566 |
| 175 | NUUAUUGGUUGUGACUUUC | 440 | GAAAGUCACAACCAAUAAN | 568-586 | 566 |
| 176 | NUUAUUGGUUGUGACUUUN | 441 | NAAAGUCACAACCAAUAAN | 568-586 | 566 |
| 177 | AUUUAUUGGUUGUGACUUU | 442 | AAAGUCACAACCAAUAAAU | 569-587 | 567 |
| 178 | UUUUAUUGGUUGUGACUUU | 443 | AAAGUCACAACCAAUAAAA | 569-587 | 567 |
| 179 | NUUUAUUGGUUGUGACUUU | 444 | AAAGUCACAACCAAUAAAN | 569-587 | 567 |
| 180 | NUUUAUUGGUUGUGACUUN | 445 | NAAGUCACAACCAAUAAAN | 569-587 | 567 |
| 181 | CAUUUAUUGGUUGUGACUU | 446 | AAGUCACAACCAAUAAAUG | 570-588 | 568 |
| 182 | UAUUUAUUGGUUGUGACUU | 447 | AAGUCACAACCAAUAAAUA | 570-588 | 568 |
| 183 | NAUUUAUUGGUUGUGACUU | 448 | AAGUCACAACCAAUAAAUN | 570-588 | 568 |
| 184 | UAUUUAUUGGUUGUGACUN | 449 | NAGUCACAACCAAUAAAUA | 570-588 | 568 |
| 185 | NAUUUAUUGGUUGUGACUN | 450 | NAGUCACAACCAAUAAAUN | 570-588 | 568 |
| 186 | CAUUUAUUGGUUGUGACUU | 451 | AAGUCACAACCAAUAA(A^{2N})UG | 570-588 | 568 |
| 187 | UAUUUAUUGGUUGUGACUU | 452 | AAGUCACAACCAAUAA(A^{2N})UA | 570-588 | 568 |
| 188 | NAUUUAUUGGUUGUGACUU | 453 | AAGUCACAACCAAUAA(A^{2N})UN | 570-588 | 568 |
| 189 | UAUUUAUUGGUUGUGACUN | 454 | NAGUCACAACCAAUAA(A^{2N})UA | 570-588 | 568 |
| 190 | NAUUUAUUGGUUGUGACUN | 455 | NAGUCACAACCAAUAA(A^{2N})UN | 570-588 | 568 |
| 191 | GACAUUUAUUGGUUGUGAC | 456 | GUCACAACCAAUAAAUGUC | 572-590 | 570 |
| 192 | UACAUUUAUUGGUUGUGAC | 457 | GUCACAACCAAUAAAUGUA | 572-590 | 570 |
| 193 | NACAUUUAUUGGUUGUGAC | 458 | GUCACAACCAAUAAAUGUN | 572-590 | 570 |
| 194 | UACAUUUAUUGGUUGUGAN | 459 | NUCACAACCAAUAAAUGUA | 572-590 | 570 |
| 195 | NACAUUUAUUGGUUGUGAN | 460 | NUCACAACCAAUAAAUGUN | 572-590 | 570 |
| 196 | AGACAUUUAUUGGUUGUGA | 461 | UCACAACCAAUAAAUGUCU | 573-591 | 571 |
| 197 | UGACAUUUAUUGGUUGUGA | 462 | UCACAACCAAUAAAUGUCA | 573-591 | 571 |
| 198 | NGACAUUUAUUGGUUGUGA | 463 | UCACAACCAAUAAAUGUCN | 573-591 | 571 |
| 199 | UGACAUUUAUUGGUUGUGN | 464 | NCACAACCAAUAAAUGUCA | 573-591 | 571 |
| 200 | AGACAUUUAUUGGUUGUGN | 465 | NCACAACCAAUAAAUGUCU | 573-591 | 571 |
| 201 | NGACAUUUAUUGGUUGUGN | 466 | NCACAACCAAUAAAUGUCN | 573-591 | 571 |
| 202 | AGACGUUUAUUGGUUGUGA | 467 | UCACAACCAAUAAAUGUCU | 573-591 | 571 |
| 203 | UGACGUUUAUUGGUUGUGA | 468 | UCACAACCAAUAAAUGUCA | 573-591 | 571 |
| 204 | NGACGUUUAUUGGUUGUGA | 469 | UCACAACCAAUAAAUGUCN | 573-591 | 571 |
| 205 | UGACGUUUAUUGGUUGUGN | 470 | NCACAACCAAUAAAUGUCA | 573-591 | 571 |
| 206 | NGACGUUUAUUGGUUGUGN | 471 | NCACAACCAAUAAAUGUCN | 573-591 | 571 |
| 207 | AGACGUUUAUUGGUUGUGA | 472 | UCACAACCAAUAAACGUCU | 573-591 | 571 |
| 208 | UGACGUUUAUUGGUUGUGA | 473 | UCACAACCAAUAAACGUCA | 573-591 | 571 |
| 209 | AGACGUUUAUUGGUUGUGN | 474 | NCACAACCAAUAAACGUCU | 573-591 | 571 |
| 210 | NGACGUUUAUUGGUUGUGA | 475 | UCACAACCAAUAAACGUCN | 573-591 | 571 |
| 211 | NGACGUUUAUUGGUUGUGN | 476 | NCACAACCAAUAAACGUCN | 573-591 | 571 |
| 212 | GACACUUGUUCCAGACAUU | 477 | AAUGUCUGGAACAAGUGUC | 585-603 | 583 |
| 213 | UACACUUGUUCCAGACAUU | 478 | AAUGUCUGGAACAAGUGUA | 585-603 | 583 |
| 214 | AACACUUGUUCCAGACAUU | 479 | AAUGUCUGGAACAAGUGUU | 585-603 | 583 |
| 215 | NACACUUGUUCCAGACAUU | 480 | AAUGUCUGGAACAAGUGUN | 585-603 | 583 |
| 216 | NACACUUGUUCCAGACAUN | 481 | NAUGUCUGGAACAAGUGUN | 585-603 | 583 |
| 217 | GUGACACUUGUUCCAGACA | 482 | UGUCUGGAACAAGUGUCAC | 587-605 | 585 |
| 218 | UUGACACUUGUUCCAGACA | 483 | UGUCUGGAACAAGUGUCAA | 587-605 | 585 |
| 219 | AUGACACUUGUUCCAGACA | 484 | UGUCUGGAACAAGUGUCAU | 587-605 | 585 |
| 220 | NUGACACUUGUUCCAGACA | 485 | UGUCUGGAACAAGUGUCAN | 587-605 | 585 |
| 221 | NUGACACUUGUUCCAGACN | 486 | NGUCUGGAACAAGUGUCAN | 587-605 | 585 |
| 222 | AAUUGUGACACUUGUUCCA | 487 | UGGAACAAGUGUCACAAUU | 591-609 | 589 |
| 223 | UAUUGUGACACUUGUUCCA | 488 | UGGAACAAGUGUCACAAUA | 591-609 | 589 |
| 224 | NAUUGUGACACUUGUUCCA | 489 | UGGAACAAGUGUCACAAUN | 591-609 | 589 |
| 225 | NAUUGUGACACUUGUUCCN | 490 | NGGAACAAGUGUCACAAUN | 591-609 | 589 |
| 226 | UAAUUGUGACACUUGUUCC | 491 | GGAACAAGUGUCACAAUUA | 592-610 | 590 |
| 227 | AAAUUGUGACACUUGUUCC | 492 | GGAACAAGUGUCACAAUUU | 592-610 | 590 |
| 228 | NAAUUGUGACACUUGUUCC | 493 | GGAACAAGUGUCACAAUUN | 592-610 | 590 |
| 229 | NAAUUGUGACACUUGUUCN | 494 | NGAACAAGUGUCACAAUUN | 592-610 | 590 |
| 230 | GUAAUUGUGACACUUGUUC | 495 | GAACAAGUGUCACAAUUAC | 593-611 | 591 |
| 231 | UUAAUUGUGACACUUGUUC | 496 | GAACAAGUGUCACAAUUAA | 593-611 | 591 |
| 232 | AUAAUUGUGACACUUGUUC | 497 | GAACAAGUGUCACAAUUAU | 593-611 | 591 |
| 233 | NUAAUUGUGACACUUGUUC | 498 | GAACAAGUGUCACAAUUAN | 593-611 | 591 |
| 234 | NUAAUUGUGACACUUGUUN | 499 | NAACAAGUGUCACAAUUAN | 593-611 | 591 |
| 235 | UGUAAUUGUGACACUUGUU | 500 | AACAAGUGUCACAAUUACA | 594-612 | 592 |
| 236 | AGUAAUUGUGACACUUGUU | 501 | AACAAGUGUCACAAUUACU | 594-612 | 592 |
| 237 | NGUAAUUGUGACACUUGUU | 502 | AACAAGUGUCACAAUUACN | 594-612 | 592 |
| 238 | NGUAAUUGUGACACUUGUN | 503 | NACAAGUGUCACAAUUACN | 594-612 | 592 |
| 239 | CAGUAAAGGUCGAUUGAAG | 504 | CUUCAAUCGACCUUUACUG | 628-646 | 626 |
| 240 | UAGUAAAGGUCGAUUGAAG | 505 | CUUCAAUCGACCUUUACUA | 628-646 | 626 |
| 241 | AAGUAAAGGUCGAUUGAAG | 506 | CUUCAAUCGACCUUUACUU | 628-646 | 626 |
| 242 | NAGUAAAGGUCGAUUGAAG | 507 | CUUCAAUCGACCUUUACUN | 628-646 | 626 |
| 243 | NAGUAAAGGUCGAUUGAAN | 508 | NUUCAAUCGACCUUUACUN | 628-646 | 626 |
| 244 | AUAAUAAAGAUGGUUUACU | 509 | AGUAAACCAUCUUUAUUAU | 654-672 | 652 |
| 245 | UUAAUAAAGAUGGUUUACU | 510 | AGUAAACCAUCUUUAUUAA | 654-672 | 652 |
| 246 | NUAAUAAAGAUGGUUUACU | 511 | AGUAAACCAUCUUUAUUAN | 654-672 | 652 |
| 247 | NUAAUAAAGAUGGUUUACN | 512 | NGUAAACCAUCUUUAUUAN | 654-672 | 652 |
| 248 | UUUUGGUGCUGUGAAAUAU | 513 | AUAUUUCACAGCACCAAAA | 677-695 | 675 |
| 249 | AUUUGGUGCUGUGAAAUAU | 514 | AUAUUUCACAGCACCAAAU | 677-695 | 675 |
| 250 | NUUUGGUGCUGUGAAAUAU | 515 | AUAUUUCACAGCACCAAAN | 677-695 | 675 |
| 251 | NUUUGGUGCUGUGAAAUAN | 516 | NUAUUUCACAGCACCAAAN | 677-695 | 675 |
| 252 | CACAGUUAGAGUUAAUGUU | 517 | AACAUUAACUCUAACUGUG | 721-739 | 719 |
| 253 | UACAGUUAGAGUUAAUGUU | 518 | AACAUUAACUCUAACUGUA | 721-739 | 719 |
| 254 | AACAGUUAGAGUUAAUGUU | 519 | AACAUUAACUCUAACUGUU | 721-739 | 719 |
| 255 | NACAGUUAGAGUUAAUGUU | 520 | AACAUUAACUCUAACUGUN | 721-739 | 719 |
| 256 | NACAGUUAGAGUUAAUGUN | 521 | NACAUUAACUCUAACUGUN | 721-739 | 719 |
| 257 | UAAGUUAAGAAACUCUUCU | 522 | AGAAGAGUUUCUUAACUUA | 775-793 | 773 |
| 258 | AAAGUUAAGAAACUCUUCU | 523 | AGAAGAGUUUCUUAACUUU | 775-793 | 773 |
| 259 | NAAGUUAAGAAACUCUUCU | 524 | AGAAGAGUUUCUUAACUUN | 775-793 | 773 |
| 260 | NAAGUUAAGAAACUCUUCN | 525 | NGAAGAGUUUCUUAACUUN | 775-793 | 773 |
| 261 | AGUAAGUUAAGAAACUCUU | 526 | AAGAGUUUCUUAACUUACU | 777-795 | 775 |
| 262 | UGUAAGUUAAGAAACUCUU | 527 | AAGAGUUUCUUAACUUACA | 777-795 | 775 |
| 263 | AGUAAGUUAAGAAACUCUU | 528 | AAGAGUUUCUUAACUUACU | 777-795 | 775 |
| 264 | NGUAAGUUAAGAAACUCUN | 529 | NAGAGUUUCUUAACUUACN | 777-795 | 775 |
| 265 | UCAACAUUUGAGGAGUAGU | 530 | ACUACUCCUCAAAUGUUGA | 838-856 | 836 |
| 266 | ACAACAUUUGAGGAGUAGU | 531 | ACUACUCCUCAAAUGUUGU | 838-856 | 836 |
| 267 | NCAACAUUUGAGGAGUAGU | 532 | ACUACUCCUCAAAUGUUGN | 838-856 | 836 |
| 268 | NCAACAUUUGAGGAGUAGN | 533 | NCUACUCCUCAAAUGUUGN | 838-856 | 836 |
| 269 | CAGUCAUCAAUGUUAUGGA | 534 | UCCAUAACAUUGAUGACUG | 865-883 | 863 |
| 270 | UAGUCAUCAAUGUUAUGGA | 535 | UCCAUAACAUUGAUGACUA | 865-883 | 863 |
| 271 | AAGUCAUCAAUGUUAUGGA | 536 | UCCAUAACAUUGAUGACUU | 865-883 | 863 |
| 272 | NAGUCAUCAAUGUUAUGGA | 537 | UCCAUAACAUUGAUGACUN | 865-883 | 863 |
| 273 | NAGUCAUCAAUGUUAUGGN | 538 | NCCAUAACAUUGAUGACUN | 865-883 | 863 |
| 274 | CAUGAAGCCAGUCAUCAAU | 539 | AUUGAUGACUGGCUUCAUG | 873-891 | 871 |
| 275 | UAUGAAGCCAGUCAUCAAU | 540 | AUUGAUGACUGGCUUCAUA | 873-891 | 871 |
| 276 | AAUGAAGCCAGUCAUCAAU | 541 | AUUGAUGACUGGCUUCAUU | 873-891 | 871 |
| 277 | NAUGAAGCCAGUCAUCAAU | 542 | AUUGAUGACUGGCUUCAUN | 873-891 | 871 |
| 278 | NAUGAAGCCAGUCAUCAAN | 543 | NUUGAUGACUGGCUUCAUN | 873-891 | 871 |
| 279 | AAGUUUAGGUGCUAUCAUU | 544 | AAUGAUAGCACCUAAACUU | 994-1012 | 992 |
| 280 | UAGUUUAGGUGCUAUCAUU | 545 | AAUGAUAGCACCUAAACUA | 994-1012 | 992 |
| 281 | NAGUUUAGGUGCUAUCAUU | 546 | AAUGAUAGCACCUAAACUN | 994-1012 | 992 |
| 282 | NAGUUUAGGUGCUAUCAUN | 547 | NAUGAUAGCACCUAAACUN | 994-1012 | 992 |
| 283 | UGUAGAAGGAAUGUCUGUC | 548 | GACAGACAUUCCUUCUACA | 1023-1041 | 1021 |
| 284 | AGUAGAAGGAAUGUCUGUC | 549 | GACAGACAUUCCUUCUACU | 1023-1041 | 1021 |
| 285 | NGUAGAAGGAAUGUCUGUC | 550 | GACAGACAUUCCUUCUACN | 1023-1041 | 1021 |
| 286 | NGUAGAAGGAAUGUCUGUN | 551 | NACAGACAUUCCUUCUACN | 1023-1041 | 1021 |
| 287 | UACAUGUAGAAGGAAUGUC | 552 | GACAUUCCUUCUACAUGUA | 1027-1045 | 1025 |
| 288 | AACAUGUAGAAGGAAUGUC | 553 | GACAUUCCUUCUACAUGUU | 1027-1045 | 1025 |
| 289 | NACAUGUAGAAGGAAUGUC | 554 | GACAUUCCUUCUACAUGUN | 1027-1045 | 1025 |
| 290 | NACAUGUAGAAGGAAUGUN | 555 | NACAUUCCUUCUACAUGUN | 1027-1045 | 1025 |
| 291 | CAAGAAGUGUCAUUACAUG | 556 | CAUGUAAUGACACUUCUUG | 1040-1058 | 1038 |
| 292 | UAAGAAGUGUCAUUACAUG | 557 | CAUGUAAUGACACUUCUUA | 1040-1058 | 1038 |
| 293 | AAAGAAGUGUCAUUACAUG | 558 | CAUGUAAUGACACUUCUUU | 1040-1058 | 1038 |
| 294 | NAAGAAGUGUCAUUACAUG | 559 | CAUGUAAUGACACUUCUUN | 1040-1058 | 1038 |
| 295 | NAAGAAGUGUCAUUACAUN | 560 | NAUGUAAUGACACUUCUUN | 1040-1058 | 1038 |
| 296 | ACAAGAAGUGUCAUUACAU | 561 | AUGUAAUGACACUUCUUGU | 1041-1059 | 1039 |
| 297 | UCAAGAAGUGUCAUUACAU | 562 | AUGUAAUGACACUUCUUGA | 1041-1059 | 1039 |
| 298 | NCAAGAAGUGUCAUUACAU | 563 | AUGUAAUGACACUUCUUGN | 1041-1059 | 1039 |
| 299 | NCAAGAAGUGUCAUUACAN | 564 | NUGUAAUGACACUUCUUGN | 1041-1059 | 1039 |
| 300 | CACAAGAAGUGUCAUUACA | 565 | UGUAAUGACACUUCUUGUG | 1042-1060 | 1040 |
| 301 | UACAAGAAGUGUCAUUACA | 566 | UGUAAUGACACUUCUUGUA | 1042-1060 | 1040 |
| 302 | AACAAGAAGUGUCAUUACA | 567 | UGUAAUGACACUUCUUGUU | 1042-1060 | 1040 |
| 303 | NACAAGAAGUGUCAUUACA | 568 | UGUAAUGACACUUCUUGUN | 1042-1060 | 1040 |
| 304 | NACAAGAAGUGUCAUUACN | 569 | NGUAAUGACACUUCUUGUN | 1042-1060 | 1040 |
| 305 | UUUCACAAUACUUUGCUUG | 570 | CAAGCAAAGUAUUGUGAAA | 1151-1169 | 1149 |
| 306 | AUUCACAAUACUUUGCUUG | 571 | CAAGCAAAGUAUUGUGAAU | 1151-1169 | 1149 |
| 307 | NUUCACAAUACUUUGCUUG | 572 | CAAGCAAAGUAUUGUGAAN | 1151-1169 | 1149 |
| 308 | NUUCACAAUACUUUGCUUN | 573 | NAAGCAAAGUAUUGUGAAN | 1151-1169 | 1149 |
| 309 | CUUCUCAGGAUCUACUUGU | 574 | ACAAGUAGAUCCUGAGAAG | 1220-1238 | 1218 |
| 310 | UUUCUCAGGAUCUACUUGU | 575 | ACAAGUAGAUCCUGAGAAG | 1220-1238 | 1218 |
| 311 | AUUCUCAGGAUCUACUUGU | 576 | ACAAGUAGAUCCUGAGAAU | 1220-1238 | 1218 |
| 312 | NUUCUCAGGAUCUACUUGU | 577 | ACAAGUAGAUCCUGAGAAN | 1220-1238 | 1218 |
| 313 | NUUCUCAGGAUCUACUUGN | 578 | NCAAGUAGAUCCUGAGAAN | 1220-1238 | 1218 |
| 314 | AGUAGCUGUAACAAAGGUA | 579 | UACCUUUGUUACAGCUACU | 1239-1257 | 1237 |
| 315 | UGUAGCUGUAACAAAGGUA | 580 | UACCUUUGUUACAGCUACA | 1239-1257 | 1237 |
| 316 | NGUAGCUGUAACAAAGGUA | 581 | UACCUUUGUUACAGCUACN | 1239-1257 | 1237 |
| 317 | NGUAGCUGUAACAAAGGUN | 582 | NACCUUUGUUACAGCUACN | 1239-1257 | 1237 |
| 318 | AUAGUGUCAAUUACAAAGG | 583 | CCUUUGUAAUUGACACUAU | 1328-1346 | 1326 |
| 319 | UUAGUGUCAAUUACAAAGG | 584 | CCUUUGUAAUUGACACUAA | 1328-1346 | 1326 |
| 320 | NUAGUGUCAAUUACAAAGG | 585 | CCUUUGUAAUUGACACUAN | 1328-1346 | 1326 |
| 321 | NUAGUGUCAAUUACAAAGN | 586 | NCUUUGUAAUUGACACUAN | 1328-1346 | 1326 |

N = any nucleobase
I = inosine (hypoxanthine nucleobase) nucleotide
(A^{2N}) = 2-aminoadenosine nucleotide

The TSLP RNAi agent sense strands and antisense strands that comprise or consist of the nucleotide sequences in Table 2 can be modified nucleotides or unmodified nucleotides. In some embodiments, the TSLP RNAi agents having the sense and antisense strand sequences that comprise or consist of any of the nucleotide sequences in Table 2 are all or substantially all modified nucleotides.

In some embodiments, the antisense strand of a TSLP RNAi agent disclosed herein differs by 0, 1, 2, or 3 nucleotides from any of the antisense strand sequences in Table 2. In some embodiments, the sense strand of a TSLP RNAi agent disclosed herein differs by 0, 1, 2, or 3 nucleotides from any of the sense strand sequences in Table 2.

As used herein, each N listed in a sequence disclosed in Table 2 may be independently selected from any and all nucleobases (including those found on both modified and unmodified nucleotides). In some embodiments, an N nucleotide listed in a sequence disclosed in Table 2 has a nucleobase that is complementary to the N nucleotide at the corresponding position on the other strand. In some embodiments, an N nucleotide listed in a sequence disclosed in Table 2 has a nucleobase that is not complementary to the N nucleotide at the corresponding position on the other strand. In some embodiments, an N nucleotide listed in a sequence disclosed in Table 2 has a nucleobase that is the same as the N nucleotide at the corresponding position on the other strand. In some embodiments, an N nucleotide listed in a sequence disclosed in Table 2 has a nucleobase that is different from the N nucleotide at the corresponding position on the other strand.

Certain modified TSLP RNAi agent sense and antisense strands are provided in Table 3, Table 4, Table 5, Table 6, and Table 10. Certain modified TSLP RNAi agent antisense strands, as well as their underlying unmodified nucleobase sequences, are provided in Table 3. Certain modified TSLP RNAi agent sense strands, as well as their underlying unmodified nucleobase sequences, are provided in Tables 4, 5, and 6. In forming TSLP RNAi agents, each of the nucleotides in each of the underlying base sequences listed in Tables 3, 4, 5, and 6, as well as in Table 2, above, can be a modified nucleotide.

The TSLP RNAi agents described herein are formed by annealing an antisense strand with a sense strand. A sense strand containing a sequence listed in Table 2, Table 4, Table 5, or Table 6 can be hybridized to any antisense strand containing a sequence listed in Table 2 or Table 3, provided the two sequences have a region of at least 85% complementarity over a contiguous 16, 17, 18, 19, 20, or 21 nucleotide sequence.

In some embodiments, a TSLP RNAi agent antisense strand comprises a nucleotide sequence ofany of the sequences in Table 2 or Table 3.

In some embodiments, a TSLP RNAi agent comprises or consists of a duplex having the nucleobase sequences of the sense strand and the antisense strand of any of the sequences in Table 2, Table 3, Table 4, Table 5, Table 6, or Table 10.

Examples of antisense strands containing modified nucleotides are provided in Table 3. Examples of sense strands containing modified nucleotides are provided in Tables 4, 5 and 6.

As used in Tables 3, 4, 5, 6, and 10. the following notations are used to indicate modified nucleotides, targeting groups, and linking groups:

| | |
|---|---|
| A= | adenosine-3′-phosphate |
| C= | cytidine-3′-phosphate |
| G= | guanosine-3′-phosphate |
| U= | uridine-3′-phosphate |
| I= | inosine-3′-phosphate |
| a= | 2′-O-methyladenosine-3′-phosphate |
| as= | 2′-O-methyladenosine-3′-phosphorothioate |
| c= | 2′-O-methylcytidine-3′-phosphate |
| cs= | 2′-O-methylcytidine-3′-phosphorothioate |
| g= | 2′-O-methylguanosine-3′-phosphate |
| gs= | 2′-O-methylguanosine-3′-phosphorothioate |
| i= | 2′-O-methylinosine-3′-phosphate |
| is= | 2′-O-methylinosine-3′-phosphorothioate |
| t= | 2′-O-methyl-5-methyluridine-3′-phosphate |
| ts= | 2′-O-methyl-5-methyluridine-3′-phosphorothioate |
| u= | 2′-O-methyluridine-3′-phosphate |
| us= | 2′-O-methyluridine-3′-phosphorothioate |
| Af= | 2′-fluoroadenosine-3′-phosphate |
| Afs= | 2′-fluoroadenosine-3′-phosporothioate |
| Cf= | 2′-fluorocytidine-3′-phosphate |
| Cfs= | 2′-fluorocytidine-3′-phosphorothioate |
| Gf= | 2′-fluoroguanosine-3′-phosphate |
| Gfs= | 2′-fluoroguanosine-3′-phosphorothioate |
| Tf= | 2′-fluoro-5′-methyluridine-3′-phosphate |
| Tfs= | 2′-fluoro-5′-methyluridine-3′-phosphorothioate |
| Uf= | 2′-fluorouridine-3′-phosphate |
| Ufs= | 2′-fluorouridine-3′-phosphorothioate |
| dT= | 2′-deoxythymidine-3′-phosphate |
| dTs= | 2′-deoxythymidine-3′-phosphorothioate |
| dA= | 2′-deoxyadenosine-3′-phosphate |
| dAs= | 2′-deoxyadenosine-3′-phosphorothioate |
| dC= | 2′-deoxycytidine-3′-phosphate |
| dCs= | 2′-deoxycytidine-3′-phosphorothioate |
| dG= | 2′-deoxyguanosine-3′-phosphate |
| dGs= | 2′-deoxyguanosine-3′-phosphorothioate |
| A_{UNA}= | 2′,3′-seco-adenosine-3′-phosphate |
| A_{UNA}s= | 2′,3′-seco-adenosine-3′-phosphorothioate |
| C_{UNA}= | 2′,3′-seco-cytidine-3′-phosphate |
| C_{UNA}s= | 2′,3′-seco-cytidine-3′-phosphorothioate |
| G_{UNA}= | 2′,3′-seco-guanosine-3′-phosphate |
| G_{UNA}s= | 2′,3′-seco-guanosine-3′-phosphorothioate |
| U_{UNA}= | 2′,3′-seco-uridine-3′-phosphate |
| U_{UNA}s= | 2′,3′-seco-uridine-3′-phosphorothioate |
| a_2N= | 2′-O-methyl-2-aminoadenosine-3′-phosphate, |
| | see Table 11 |
| a_2Ns= | 2′-O-methyl-2-aminoadenosine-3′-phosphorothioate, |
| | see Table 11 |
| (invAb)= | inverted abasic deoxyribonucleotide-5′-phosphate, |
| | see Table 11 |
| (invAb)s= | inverted abasic deoxyribonucleotide-5′-phosphorothioate, |
| | see Table 11 |
| s= | phosphorothioate linkage |
| ss= | phosphrodithioate linkage |
| p= | terminal phosphate (as synthesized) |
| vpdN= | vinyl phosphonate deoxyribonucleotide |
| cPrpa= | 5′-cyclopropyl phosphonate-2′-O-methyladenosine-3′- |
| | phosphate (see Table 11) |
| cPrpas= | 5′-cyclopropyl phosphonate-2′-O-methyladenosine-3′- |
| | phosphorothioate (see Table 11) |
| cPrpu= | 5′-cyclopropyl phosphonate-2′-O-methyluridine-3′- |
| | phosphate (see Table 11) |
| cPrpus= | 5′-cyclopropyl phosphonate-2′-O-methyluridine-3′- |
| | phosphorothioate (see Table 11) |
| cPrpi= | 5′-cyclopropyl phosphonate-2′-O-methylinosine-3′- |
| | phosphate (see Table 11) |
| cPrpis= | 5′-cyclopropyl phosphonate-2′-O-methylinosine-3′- |
| | phosphorothioate (see Table 11) |
| (C6-SS-C6)= | see Table 11 |
| (6-SS-6)= | see Table 11 |
| (NH2-C6)= | see Table 11 |
| (NH2-C6)s= | see Table 11 |
| (TriAlk14)= | see Table 11 |
| (TriAlk14)s= | see Table 11 |
| -C6-= | see Table 11 |
| -C6s-= | see Table 11 |
| -L6-C6-= | see Table 11 |
| -L6-C6s-= | see Table 11 |
| (TA14)= | see Table 11 (structure of (TriAlk14)s after conjugation) |
| (TA14)s | see Table 11 (structure of (TriAlk14)s after conjugation) |
| TGNA | thymine glycol nucleic acid, see Table 11 |

As the person of ordinary skill in the art would readily understand, unless otherwise indicated by the sequence (such as, for example, by a phosphorothioate linkage “s”), when present in an oligonucleotide, the nucleotide monomers are mutually linked by 5′-3′-phosphodiester bonds. As the person of ordinary skill in the art would clearly understand, the inclusion of a phosphorothioate linkage as shown in the modified nucleotide sequences disclosed herein replaces the phosphodiester linkage typically present in oligonucleotides. Further, the person of ordinary skill in the art would readily understand that the terminal nucleotide at the 3′ end of a given oligonucleotide sequence would typically have a hydroxyl (—OH) group at the respective 3′ position of the given monomer instead of a phosphate moiety ex vivo. Additionally, for the embodiments disclosed herein, when viewing the respective strand 5′→3′, the inverted abasic residues are inserted such that the 3′ position of the deoxyribose is linked at the 3′ end of the preceding monomer on the respective strand (see, e.g., Table 11). Moreover, as the person of ordinary skill would readily understand and appreciate, while the phosphorothioate chemical structures depicted herein typically show the anion on the sulfur atom, the inventions disclosed herein encompass all phosphorothioate tautomers (e.g., where the sulfur atom has a double-bond and the anion is on an oxygen atom). Unless expressly indicated otherwise herein, such understandings of the person of ordinary skill in the art are used when describing the TSLP RNAi agents and compositions of TSLP RNAi agents disclosed herein.

Certain examples of targeting groups and linking groups used with the TSLP RNAi agents disclosed herein are included in the chemical structures provided below in Table 11. Each sense strand and/or antisense strand can have any targeting groups or linking groups listed herein, as well as other targeting or linking groups, conjugated to the 5′ and/or 3′ end of the sequence.

TABLE 3
TSLP RNAi Agent Antisense Strand Sequences

| | | | Underlying Base | |
|---|---|---|---|---|
| | | | Sequence (5′→3′) | |
| AS | | SEQ ID | (Shown as an Unmodified | SEQ ID |
| Strand ID | Modified Antisense Strand (5′→3′) | NO. | Nucleotide Sequence) | NO. |
| AM14179-AS | cPrpasGfsasAfuUfuGfuGfaGfgUfuUfgAfuUfsc | 587 | AGAAUUUGUGAGGUUUGAUUC | 823 |
| AM16334-AS | cPrpusAfscsCfaUfuucucUfcAfgUfuUfcasg | 588 | UACCAUUUCUCUCAGUUUCAG | 824 |
| AM18285-AS | usUfsasGfcAfuUfuAfuCfuGfaGfuUfuCfsc | 589 | UUAGCAUUUAUCUGAGUUUCC | 825 |
| AM18311-AS | usAfscsAfuUfuAfuUfgGfuUfgUfgAfcUfsu | 590 | UACAUUUAUUGGUUGUGACUU | 826 |
| AM19258-AS | cPrpusUfsgsGfaUfuUfcAfgUfaAfgGfcAfaUfsg | 591 | UUGGAUUUCAGUAAGGCAAUG | 827 |
| AM19260-AS | cPrpusAfsasGfgUfuAfgGfcUfcUfgGfaUfuUfsc | 592 | UAAGGUUAGGCUCUGGAUUUC | 828 |
| AM19262-AS | cPrpasUfsusUfaUfcUfgAfgUfuUfcCfgAfaUfsc | 593 | AUUUAUCUGAGUUUCCGAAUC | 829 |
| AM19264-AS | cPrpusAfscsAfuUfuAfuUfgGfuUfgUfgAfcUfsu | 594 | UACAUUUAUUGGUUGUGACUU | 826 |
| AM19266-AS | cPrpasGfsasCfaUfuUfaUfuGfgUfuGfuGfaCfsu | 595 | AGACAUUUAUUGGUUGUGACU | 830 |
| AM19268-AS | cPrpusAfsusUfuAfuUfgGfuUfgUfgAfcUfuUfsc | 596 | UAUUUAUUGGUUGUGACUUUC | 831 |
| AM19335-AS | cPrpusUfsasGfcAfuUfuAfuCfuGfaGfuUfuCfsc | 597 | UUAGCAUUUAUCUGAGUUUCC | 825 |
| AM19337-AS | cPrpasUfsusGfaAfgGfuUfaGfgCfuCfuGfgAfsu | 598 | AUUGAAGGUUAGGCUCUGGAU | 832 |
| AM19339-AS | cPrpusUfsusAfgGfcUfcUfgGfaUfuUfcAfgUfsa | 599 | UUUAGGCUCUGGAUUUCAGUA | 833 |
| AM19685-AS | cPrpusUfsaGfcAfuUfuAfuCfuGfaGfuUfuCfsc | 600 | UUAGCAUUUAUCUGAGUUUCC | 825 |
| AM19938-AS | cPrpuUfaGfcAfuUfuAfuCfuGfaGfuUfuCfsc | 601 | UUAGCAUUUAUCUGAGUUUCC | 825 |
| AM19939-AS | cPrpusUfsagcauuuauCfuGfaGfuuucsc | 602 | UUAGCAUUUAUCUGAGUUUCC | 825 |
| AM19941-AS | cPrpusUfsagcauuUfauCfuGfaGfuuucsc | 603 | UUAGCAUUUAUCUGAGUUUCC | 825 |
| AM19942-AS | cPrpusUfsagCfauuuauCfuGfaGfuuucsc | 604 | UUAGCAUUUAUCUGAGUUUCC | 825 |
| AM19943-AS | cPrpusUfsagcaUfuuauCfuGfaGfuuucsc | 605 | UUAGCAUUUAUCUGAGUUUCC | 825 |
| AM19944-AS | cPrpusUfsagcauUfuauCfuGfaGfuuucsc | 606 | UUAGCAUUUAUCUGAGUUUCC | 825 |
| AM19945-AS | cPrpusUfsaGfcAfUfuuauCfuGfaGfuuucsc | 607 | UUAGCAUUUAUCUGAGUUUCC | 825 |
| AM19946-AS | cPrpusdTsagcauuuauCfuGfaGfuuucsc | 608 | UTAGCAUUUAUCUGAGUUUCC | 897 |
| AM19947-AS | cPrpasGfsaCfaUfuUfaUfuGfgUfuGfuGfaCfsu | 609 | AGACAUUUAUUGGUUGUGACU | 830 |
| AM19949-AS | cPrpasGfsacauuuauuGfgUfuGfugacsu | 610 | AGACAUUUAUUGGUUGUGACU | 830 |
| AM19950-AS | cPrpasGfsacauuuaUfuGfgUfuGfugacsu | 611 | AGACAUUUAUUGGUUGUGACU | 830 |
| AM19951-AS | cPrpasGfsacauuuAfuuGfgUfuGfugacsu | 612 | AGACAUUUAUUGGUUGUGACU | 830 |
| AM19952-AS | cPrpasGfsacauUfuauuGfgUfuGfugacsu | 613 | AGACAUUUAUUGGUUGUGACU | 830 |
| AM19953-AS | cPrpasGfsacAfUfuuAfuuGfgUfuGfugacsu | 614 | AGACAUUUAUUGGUUGUGACU | 830 |
| AM19954-AS | cPrpasGfsaCfaUfUfuauuGfgUfuGfugacsu | 615 | AGACAUUUAUUGGUUGUGACU | 830 |
| AM19955-AS | cPrpaGfacauuuaUfuGfgUfuGfugacsu | 616 | AGACAUUUAUUGGUUGUGACU | 830 |
| AM20176-AS | cPrpusGfsgsGfaUfuGfaAfgGfuUfaGfgCfuCfsu | 617 | UGGGAUUGAAGGUUAGGCUCU | 834 |
| AM20299-AS | cPrpusUfsagdCauuuauCfuGfaguuucsc | 618 | UUAGCAUUUAUCUGAGUUUCC | 825 |
| AM20300-AS | cPrpusUfsagcauudTauCfuGfaguuucsc | 619 | UUAGCAUUTAUCUGAGUUUCC | 898 |
| AM20301-AS | cPrpusdTsagcauudTauCfudGaguuucsc | 620 | UTAGCAUUTAUCUGAGUUUCC | 899 |
| AM20302-AS | cPrpusdTsagcauudTauCfudGadGuuucsc | 621 | UTAGCAUUTAUCUGAGUUUCC | 899 |
| AM20303-AS | cPrpusUfsagcauuuauCfuGfaguuucsc | 622 | UUAGCAUUUAUCUGAGUUUCC | 825 |
| AM20304-AS | cPrpusUfsagcaTGNAuUfauCfuGfaGfuuucsc | 623 | UUAGCATUUAUCUGAGUUUCC | 900 |
| AM20305-AS | cPrpusUfsagcaU_{UNA}uUfauCfuGfaGfuuucsc | 624 | UUAGCAUUUAUCUGAGUUUCC | 825 |
| AM20307-AS | cPrpusUfsagcauUfuauCfuGfaGfuusc | 625 | UUAGCAUUUAUCUGAGUUC | 835 |
| AM20308-AS | cPrpusUfsagcauUfUfauCfuGfaguuucsc | 626 | UUAGCAUUUAUCUGAGUUUCC | 825 |
| AM20309-AS | cPrpusUfsagcauuUfauCfuGfaguuucsc | 627 | UUAGCAUUUAUCUGAGUUUCC | 825 |
| AM20310-AS | cPrpusUfsagCfauuUfauCfuGfaguuucsc | 628 | UUAGCAUUUAUCUGAGUUUCC | 825 |
| AM20314-AS | cPrpusdTsagcauudTauCfuGfaguuucsc | 629 | UTAGCAUUTAUCUGAGUUUCC | 899 |
| AM20315-AS | cPrpusdTsagcauudTauCfuGfadGuuucsc | 630 | UTAGCAUUTAUCUGAGUUUCC | 899 |
| AM20487-AS | cPrpusAfscAfuUfuAfuUfgGfuUfgUfgAfcUfsu | 631 | UACAUUUAUUGGUUGUGACUU | 826 |
| AM20488-AS | cPrpasGfsacAfUfuuauuGfgUfuGfugacsu | 632 | AGACAUUUAUUGGUUGUGACU | 830 |
| AM20489-AS | cPrpasGfsacAfUfuuauuGfgUfugugacsu | 633 | AGACAUUUAUUGGUUGUGACU | 830 |
| AM20490-AS | cPrpasGfsacAfuuuauuGfgUfuGfugacsu | 634 | AGACAUUUAUUGGUUGUGACU | 830 |
| AM20491-AS | cPrpusUfsagCfAfuuUfauCfuGfaGfuuucsc | 635 | UUAGCAUUUAUCUGAGUUUCC | 825 |
| AM20534-AS | cPrpasGfsaCfaUfuUfaUfuGfgUfuGfuGfaCfsc | 636 | AGACAUUUAUUGGUUGUGACC | 836 |
| AM20536-AS | cPrpusGfsaCfaUfuUfaUfuGfgUfuGfuGfaCfsc | 637 | UGACAUUUAUUGGUUGUGACC | 837 |
| AM20538-AS | cPrpusGfsaCfaUfuUfaUfuGfgUfuGfuGfaCfsu | 638 | UGACAUUUAUUGGUUGUGACU | 838 |
| AM20539-AS | cPrpusGfsacauuuaUfuGfgUfuGfugacsu | 639 | UGACAUUUAUUGGUUGUGACU | 838 |
| AM20540-AS | cPrpusGfsacauUfuauuGfgUfuGfugacsu | 640 | UGACAUUUAUUGGUUGUGACU | 838 |
| CA004207 | cPrpasGfsacauuuaUfuGfgUfuGfugacsu | 641 | AGACAUUUAUUGGUUGUGACU | 830 |
| CA004288 | cPrpusUfsagCfauuUfauCfuGfaguuucsc | 642 | UUAGCAUUUAUCUGAGUUUCC | 825 |
| CA004416 | cPrpusAfscauuuauUfgGfuUfgUfgacusu | 643 | UACAUUUAUUGGUUGUGACUU | 826 |
| CA004417 | cPrpusAfscaUfUfuaUfugGfuUfgUfgacusu | 644 | UACAUUUAUUGGUUGUGACUU | 826 |
| CA004418 | cPrpusAfscauuUfauugGfuUfgUfgacusu | 645 | UACAUUUAUUGGUUGUGACUU | 826 |
| CA004450 | cPrpusGfsacauUfuauuGfgUfuGfugacsc | 646 | UGACAUUUAUUGGUUGUGACC | 837 |
| CA004451 | cPrpasGfsacauUfuauuGfgUfuGfugacsc | 647 | AGACAUUUAUUGGUUGUGACC | 836 |
| CA004452 | cPrpusGfsacauuuaUfuGfgUfuGfugacsc | 648 | UGACAUUUAUUGGUUGUGACC | 837 |
| CA004453 | cPrpasGfsacauuuaUfuGfgUfuGfugacsc | 649 | AGACAUUUAUUGGUUGUGACC | 836 |
| CA004518 | cPrpuGfacauuuaUfuGfgUfuGfugacsu | 650 | UGACAUUUAUUGGUUGUGACU | 838 |
| CA004519 | cPrpuGfacauuuaUfuGfgUfuGfugacsc | 651 | UGACAUUUAUUGGUUGUGACC | 837 |
| CA004538 | asGfsacauuuaUfuGfgUfuGfugacsu | 652 | AGACAUUUAUUGGUUGUGACU | 830 |
| CA004830 | asGfsacauuuaUfuGfgUfuGfugacsc | 653 | AGACAUUUAUUGGUUGUGACC | 836 |
| CA004833 | asGfsacauuuAfuuGfgUfuGfugacsc | 654 | AGACAUUUAUUGGUUGUGACC | 836 |
| CA004834 | cPrpasGfsacauuuAfuuGfgUfuGfugacsc | 655 | AGACAUUUAUUGGUUGUGACC | 836 |
| CA005032 | cPrpasGfsacauU_{UNA}uaUfuGfgUfuGfugacsu | 656 | AGACAUUUAUUGGUUGUGACU | 830 |
| CA005033 | cPrpasGfsacauU_{UNA}uaUfuGfgUfuGfugacsc | 657 | AGACAUUUAUUGGUUGUGACC | 836 |
| CA005034 | cPrpasGfsacauTGNAuaUfuGfgUfuGfugacsc | 658 | AGACAUTUAUUGGUUGUGACC | 901 |
| CA005035 | cPrpusAfscauuU_{UNA}auUfgGfuUfgUfgacusu | 659 | UACAUUUAUUGGUUGUGACUU | 826 |
| CA005037 | cPrpusAfscauuU_{UNA}auUfgGfuUfgUfgacusc | 660 | UACAUUUAUUGGUUGUGACUC | 839 |
| CA005056 | isGfsacauuuaUfuGfgUfuGfugacsu | 661 | IGACAUUUAUUGGUUGUGACU | 840 |
| CA005404 | cPrpisGfsacauuuaUfuGfgUfuGfugacsu | 662 | IGACAUUUAUUGGUUGUGACU | 840 |
| CA005407 | cPrpasGfsacguuuaUfuGfgUfuGfugacsu | 663 | AGACGUUUAUUGGUUGUGACU | 841 |
| CA005410 | cPrpasGfsacauuuaUfuGfgUfuGfugacssu | 664 | AGACAUUUAUUGGUUGUGACU | 830 |
| CA005411 | cPrpaGfacauuuaUfuGfgUfuGfugascsu | 665 | AGACAUUUAUUGGUUGUGACU | 830 |
| CA005412 | cPrpaGfacauuuaUfuGfgUfuGfugacssu | 666 | AGACAUUUAUUGGUUGUGACU | 830 |
| CA005413 | cPrpasGfsacaTGNAuuaUfuGfgUfuGfugacsu | 667 | AGACATUUAUUGGUUGUGACU | 902 |
| CA005414 | cPrpasGfsacauuTGNAaUfuGfgUfuGfugacsu | 668 | AGACAUUTAUUGGUUGUGACU | 903 |
| CA005415 | cPrpasGfsacauuU_{UNA}aUfuGfgUfuGfugacsu | 669 | AGACAUUUAUUGGUUGUGACU | 830 |
| CA005630 | cPrpusCfsaGfaUfagcuaAfgGfcAfgCfcusu | 670 | UCAGAUAGCUAAGGCAGCCUU | 842 |
| CA005632 | cPrpusAfsgUfaAfaggucGfaUfuGfaAfgcsg | 671 | UAGUAAAGGUCGAUUGAAGCG | 843 |
| CA005634 | cPrpusAfscAfgUfuagagUfuAfaUfgUfuusc | 672 | UACAGUUAGAGUUAAUGUUUC | 844 |
| CA005636 | cPrpusAfsaGfuUfaagaaAfcUfcUfuCfugsu | 673 | UAAGUUAAGAAACUCUUCUGU | 845 |
| CA005638 | cPrpusCfsaAfcAfuuugaGfgAfgUfaGfuasc | 674 | UCAACAUUUGAGGAGUAGUAC | 846 |
| CA005640 | cPrpusAfsgUfcAfucaauGfuUfaUfgGfaasg | 675 | UAGUCAUCAAUGUUAUGGAAG | 847 |
| CA005642 | cPrpasAfsgUfuUfaggugCfuAfuCfaUfugsc | 676 | AAGUUUAGGUGCUAUCAUUGC | 848 |
| CA005644 | cPrpusGfsuAfgAfaggaaUfgUfcUfgUfcusu | 677 | UGUAGAAGGAAUGUCUGUCUU | 849 |
| CA005646 | cPrpusAfscAfaGfaagugUfcAfuUfaCfausg | 678 | UACAAGAAGUGUCAUUACAUG | 850 |
| CA005648 | cPrpusUfsuCfuCfaggauCfuAfcUfuGfuasu | 679 | UUUCUCAGGAUCUACUUGUAU | 851 |
| CA005746 | cPrpasGfsacauuugUfuGfgUfuGfugacsc | 680 | AGACAUUUGUUGGUUGUGACC | 852 |
| CA005749 | cPrpasGfsacguuuaUfuGfgUfuGfugacsc | 681 | AGACGUUUAUUGGUUGUGACC | 853 |
| CA005750 | cPrpasGfsacguuuaUfdTGfgUfuGfugacsc | 682 | AGACGUUUAUTGGUUGUGACC | 904 |
| CA005751 | cPrpasGfsacauuuaUfdTGfgUfuGfugacsc | 683 | AGACAUUUAUTGGUUGUGACC | 905 |
| CA005752 | cPrpasGfsacauuuadTuGfgUfuGfugacsc | 684 | AGACAUUUATUGGUUGUGACC | 906 |
| CA005958 | cPrpisGfsacguuuaUfuGfgUfuGfugacsc | 685 | IGACGUUUAUUGGUUGUGACC | 854 |
| CA005959 | cPrpisGfsacauuuaUfuGfgUfuGfugacsc | 686 | IGACAUUUAUUGGUUGUGACC | 855 |
| CA005976 | cPrpisGfsacauuuaUfuGfgUfuGfugacssc | 687 | IGACAUUUAUUGGUUGUGACC | 855 |
| CA005977 | cPrpasGfsacauuuaUfuGfgUfuGfugacssc | 688 | AGACAUUUAUUGGUUGUGACC | 836 |
| CA006068 | cPrpasGfsacguuuaUfuGfgUfuGfugacssc | 689 | AGACGUUUAUUGGUUGUGACC | 853 |
| CA006074 | cPrpisGfsacguuuaUfuGfgUfuGfugacssc | 690 | IGACGUUUAUUGGUUGUGACC | 854 |
| CA006075 | cPrpusGfsacguuuaUfuGfgUfuGfugacssc | 691 | UGACGUUUAUUGGUUGUGACC | 856 |
| CA006076 | cPrpusGfsacauuuaUfuGfgUfuGfugacssc | 692 | UGACAUUUAUUGGUUGUGACC | 837 |
| CA006343 | cPrpasGfsacauuugUfuGfgUfuGfugacssc | 693 | AGACAUUUGUUGGUUGUGACC | 852 |
| CA006345 | cPrpasGfsacauuugUfuGfgUfuGfugacssu | 694 | AGACAUUUGUUGGUUGUGACU | 857 |
| CA006347 | cPrpusGfsacauuugUfuGfgUfuGfugacssc | 695 | UGACAUUUGUUGGUUGUGACC | 858 |
| CA006350 | cPrpasGfsacguuuaUfuGfgUfuGfugacssu | 696 | AGACGUUUAUUGGUUGUGACU | 841 |
| CA006383 | cPrpusGfsacguuuaUfuGfgUfuGfugacsu | 697 | UGACGUUUAUUGGUUGUGACU | 859 |

TABLE 4
TSLP Agent Sense Strand Sequences (Shown Without Linkers, Conjugates, Capping Moieties,
or Terminal dT)

| | | | Underlying Base | |
|---|---|---|---|---|
| | | | Sequence (5′→3′) | |
| | | SEQ ID | (Shown as an Unmodified | SEQ ID |
| Strand ID | Modified Sense Strand (5′→3′) | NO. | Nucleotide Sequence) | NO. |
| AM19257-SS-NL | csa_2NuugccuUfAfCfugaaauccaa | 698 | C(A^{2N})UUGCCUUACUGAAAUCCAA | 860 |
| AM19259-SS-NL | gsa_2NaauccaGfAfGfccuaaccuua | 699 | G(A^{2N})AAUCCAGAGCCUAACCUUA | 861 |
| AM19261-SS-NL | gsauucggaAfAfCfucagauaaa_2Nu | 700 | GAUUCGGAAACUCAGAUAA(A^{2N})U | 862 |
| AM19263-SS-NL | a_2NsagucacaAfCfCfaauaaaugua | 701 | (A^{2N})AGUCACAACCAAUAAAUGUA | 863 |
| AM19265-SS-NL | asgucacaaCfCfAfauaaaugucu | 702 | AGUCACAACCAAUAAAUGUCU | 864 |
| AM19267-SS-NL | gsaaagucaCfAfAfccaauaaa_2Nua | 703 | GAAAGUCACAACCAAUAA(A^{2N})UA | 865 |
| AM19334-SS-NL | gsgaaacucAfGfAfuaaaugcuaa | 704 | GGAAACUCAGAUAAAUGCUAA | 866 |
| AM19336-SS-NL | asuccagagCfCfUfaaccuucaau | 705 | AUCCAGAGCCUAACCUUCAAU | 867 |
| AM19338-SS-NL | usa_2NcugaaaUfCfCfagagccuaaa | 706 | U(A^{2N})CUGAAAUCCAGAGCCUAAA | 868 |
| AM19940-SS-NL | gsgaaacucAfgAfuAfaaugcuaa | 707 | GGAAACUCAGAUAAAUGCUAA | 866 |
| AM19948-SS-NL | asgucacaaCfcAfaUfaaaugucu | 708 | AGUCACAACCAAUAAAUGUCU | 864 |
| AM20175-SS-NL | asgagccuaAfCfCfuucaaucuca | 709 | AGAGCCUAACCUUCAAUCUCA | 869 |
| AM20177-SS-NL | asgagccuaAfCfCfuucaauccca | 710 | AGAGCCUAACCUUCAAUCCCA | 870 |
| AM20298-SS-NL | gsgaaacucdAgdAudAaaugcuaa | 711 | GGAAACUCAGAUAAAUGCUAA | 866 |
| AM20306-SS-NL | gsa_2NacucAfGfAfuaaaugcuaa | 712 | G(A^{2N})ACUCAGAUAAAUGCUAA | 871 |
| AM20492-SS-NL | asgucacAfaCfcAfauaaaugucu | 713 | AGUCACAACCAAUAAAUGUCU | 864 |
| AM20533-SS-NL | gsgucacaaCfCfAfauaaaugucu | 714 | GGUCACAACCAAUAAAUGUCU | 872 |
| AM20535-SS-NL | gsgucacaaCfCfAfauaaauguca | 715 | GGUCACAACCAAUAAAUGUCA | 873 |
| AM20537-SS-NL | asgucacaaCfCfAfauaaauguca | 716 | AGUCACAACCAAUAAAUGUCA | 874 |
| CS004419-NL | a_2NsagucacaAfcCfaAfuaaaugua | 717 | (A^{2N})AGUCACAACCAAUAAAUGUA | 863 |
| CS004829-NL | ggucacaaCfCfAfauaaaugucu | 718 | GGUCACAACCAAUAAAUGUCU | 872 |
| CS005406-NL | asgucacaaCfCfAfauaaaugucc | 719 | AGUCACAACCAAUAAAUGUCC | 875 |
| CS005629-NL | asaggcugcCfUfUfagcuaucuga | 720 | AAGGCUGCCUUAGCUAUCUGA | 876 |
| CS005631-NL | csgcuucaaUfCfGfaccuuuacua | 721 | CGCUUCAAUCGACCUUUACUA | 877 |
| CS005633-NL | gsa_2NaacauuAfAfCfucuaacugua | 722 | G(A^{2N})AACAUUAACUCUAACUGUA | 878 |
| CS005635-NL | a_2NscagaagaGfUfUfucuuaacuua | 723 | (A^{2N})CAGAAGAGUUUCUUAACUUA | 879 |
| CS005637-NL | gsuacuacuCfCfUfcaaauguuga | 724 | GUACUACUCCUCAAAUGUUGA | 880 |
| CS005639-NL | csuuccauaAfCfAfuugaugacua | 725 | CUUCCAUAACAUUGAUGACUA | 881 |
| CS005641-NL | gscaaugauAfGfCfaccuaaacuu | 726 | GCAAUGAUAGCACCUAAACUU | 882 |
| CS005643-NL | a_2NsagacagaCfAfUfuccuucuaca | 727 | (A^{2N})AGACAGACAUUCCUUCUACA | 883 |
| CS005645-NL | csa_2NuguaauGfAfCfacuucuugua | 728 | C(A^{2N})UGUAAUGACACUUCUUGUA | 884 |
| CS005647-NL | a_2NsuacaaguAfGfAfuccugagaaa | 729 | (A^{2N})UACAAGUAGAUCCUGAGAAA | 885 |
| CS007200-NL | asgucacaaCfCfAfauaaaugucu | 730 | AGUCACAACCAAUAAAUGUCU | 864 |
| CS008694-NL | asgucacaaCfCfAfauaaaugucu | 731 | AGUCACAACCAAUAAAUGUCU | 864 |
| CS914177-NL | ggaaacucAfGfAfuaaaugcuaa | 732 | GGAAACUCAGAUAAAUGCUAA | 866 |
| CS914203-NL | agucacaaCfCfAfauaaaugucu | 733 | AGUCACAACCAAUAAAUGUCU | 864 |
| CS915060-NL | a_2NsagucacaAfCfCfaauaaaugua | 734 | (A^{2N})AGUCACAACCAAUAAAUGUA | 863 |
| CS915061-NL | asgucacaaCfCfAfauaaaugucu | 735 | AGUCACAACCAAUAAAUGUCU | 864 |
| CS915705-NL | gsgaaacucAfgAfuAfaaugcuaa | 736 | GGAAACUCAGAUAAAUGCUAA | 866 |
| CS916246-NL | gsgucacaaCfCfAfauaaaugucu | 737 | GGUCACAACCAAUAAAUGUCU | 872 |
| CS916247-NL | gsgucacaaCfCfAfauaaauguca | 738 | GGUCACAACCAAUAAAUGUCA | 873 |
| CS916248-NL | asgucacaaCfCfAfauaaauguca | 739 | AGUCACAACCAAUAAAUGUCA | 874 |

(A^{2N}) = 2-aminoadenosine nucleotide; I = hypoxanthine (inosine) nucleotide

TABLE 5
TSLP RNAi Agent Sense Strand Sequences (Shown With (TriAlk14) Linker or (NAG37)s (see Table 11 for structure information.))

| | | | Underlying Base Sequence (5′→3′) | |
|---|---|---|---|---|
| | | SEQ ID | (Shown as an Unmodified Nucleotide | SEQ ID |
| Strand ID | Modified Sense Strand (5′→3′) | NO. | Sequence) | NO. |
| AM19257-SS | (TriAlk14)csa_2NuugccuUfAfCfugaaauccaas(invAb) | 740 | C(A^{2N})UUGCCUUACUGAAAUCCAA | 860 |
| AM19259-SS | (TriAlk14)gsa_2NaauccaGfAfGfccuaaccuuas(invAb) | 741 | G(A^{2N})AAUCCAGAGCCUAACCUUA | 861 |
| AM19261-SS | (TriAlk14)gsauucggaAfAfCfucagauaaa_2Nus(invAb) | 742 | GAUUCGGAAACUCAGAUAA(A^{2N})U | 862 |
| AM19263-SS | (TriAlk14)a_2NsagucacaAfCfCfaauaaauguas(invAb) | 743 | (A^{2N})AGUCACAACCAAUAAAUGUA | 863 |
| AM19265-SS | (TriAlk14)asgucacaaCfCfAfauaaaugucus(invAb) | 744 | AGUCACAACCAAUAAAUGUCU | 864 |
| AM19267-SS | (TriAlk14)gsaaagucaCfAfAfccaauaaa_2Nuas(invAb) | 745 | GAAAGUCACAACCAAUAA(A^{2N})UA | 865 |
| AM19334-SS | (TriAlk14)gsgaaacucAfGfAfuaaaugcuaas(invAb) | 746 | GGAAACUCAGAUAAAUGCUAA | 866 |
| AM19336-SS | (TriAlk14)asuccagagCfCfUfaaccuucaaus(invAb) | 747 | AUCCAGAGCCUAACCUUCAAU | 867 |
| AM19338-SS | (TriAlk14)usa_2NcugaaaUfCfCfagagccuaaas(invAb) | 748 | U(A^{2N})CUGAAAUCCAGAGCCUAAA | 868 |
| AM19940-SS | (TriAlk14)gsgaaacucAfgAfuAfaaugcuaas(invAb) | 749 | GGAAACUCAGAUAAAUGCUAA | 866 |
| AM19948-SS | (TriAlk14)asgucacaaCfcAfaUfaaaugucus(invAb) | 750 | AGUCACAACCAAUAAAUGUCU | 864 |
| AM20175-SS | (TriAlk14)asgagccuaAfCfCfuucaaucucas(invAb) | 751 | AGAGCCUAACCUUCAAUCUCA | 869 |
| AM20177-SS | (TriAlk14)asgagccuaAfCfCfuucaaucccas(invAb) | 752 | AGAGCCUAACCUUCAAUCCCA | 870 |
| AM20298-SS | (TriAlk14)gsgaaacucdAgdAudAaaugcuaas(invAb) | 753 | GGAAACUCAGAUAAAUGCUAA | 866 |
| AM20306-SS | (TriAlk14)gsa_2NacucAfGfAfuaaaugcuaas(invAb) | 754 | G(A^{2N})ACUCAGAUAAAUGCUAA | 871 |
| AM20492-SS | (TriAlk14)asgucacAfaCfcAfauaaaugucus(invAb) | 755 | AGUCACAACCAAUAAAUGUCU | 864 |
| AM20533-SS | (TriAlk14)gsgucacaaCfCfAfauaaaugucus(invAb) | 756 | GGUCACAACCAAUAAAUGUCU | 872 |
| AM20535-SS | (TriAlk14)gsgucacaaCfCfAfauaaaugucas(invAb) | 757 | GGUCACAACCAAUAAAUGUCA | 873 |
| AM20537-SS | (TriAlk14)asgucacaaCfCfAfauaaaugucas(invAb) | 758 | AGUCACAACCAAUAAAUGUCA | 874 |
| CS004419 | (TriAlk14)a_2NsagucacaAfcCfaAfuaaauguas(invAb) | 759 | (A^{2N})AGUCACAACCAAUAAAUGUA | 863 |
| CS004829 | (NAG37)s(invAb)sggucacaaCfCfAfauaaaugucus(invAb) | 760 | GGUCACAACCAAUAAAUGUCU | 872 |
| CS005406 | (TriAlk14)asgucacaaCfCfAfauaaauguccs(invAb) | 761 | AGUCACAACCAAUAAAUGUCC | 875 |
| CS005629 | (TriAlk14)asaggcugcCfUfUfagcuaucugas(invAb) | 762 | AAGGCUGCCUUAGCUAUCUGA | 876 |
| CS005631 | (TriAlk14)csgcuucaaUfCfGfaccuuuacuas(invAb) | 763 | CGCUUCAAUCGACCUUUACUA | 877 |
| CS005633 | (TriAlk14)gsa_2NaacauuAfAfCfucuaacuguas(invAb) | 764 | G(A^{2N})AACAUUAACUCUAACUGUA | 878 |
| CS005635 | (TriAlk14)a_2NscagaagaGfUfUfucuuaacuuas(invAb) | 765 | (A^{2N})CAGAAGAGUUUCUUAACUUA | 879 |
| CS005637 | (TriAlk14)gsuacuacuCfCfUfcaaauguugas(invAb) | 766 | GUACUACUCCUCAAAUGUUGA | 880 |
| CS005639 | (TriAlk14)csuuccauaAfCfAfuugaugacuas(invAb) | 767 | CUUCCAUAACAUUGAUGACUA | 881 |
| CS005641 | (TriAlk14)gscaaugauAfGfCfaccuaaacuus(invAb) | 768 | GCAAUGAUAGCACCUAAACUU | 882 |
| CS005643 | (TriAlk14)a_2NsagacagaCfAfUfuccuucuacas(invAb) | 769 | (A^{2N})AGACAGACAUUCCUUCUACA | 883 |
| CS005645 | (TriAlk14)csa_2NuguaauGfAfCfacuucuuguas(invAb) | 770 | C(A^{2N})UGUAAUGACACUUCUUGUA | 884 |
| CS005647 | (TriAlk14)a_2NsuacaaguAfGfAfuccugagaaas(invAb) | 771 | (A^{2N})UACAAGUAGAUCCUGAGAAA | 885 |
| CS007200 | (NH2-C6)asgucacaaCfCfAfauaaaugucus(invAb) | 772 | AGUCACAACCAAUAAAUGUCU | 864 |
| CS008694 | (NH2-C6)sasgucacaaCfCfAfauaaaugucus(invAb)C6-SS-C6-dT | 773 | AGUCACAACCAAUAAAUGUCUT | 886 |
| CS914177 | (NAG37)s(invAb)sggaaacucAfGfAfuaaaugcuaas(invAb) | 774 | GGAAACUCAGAUAAAUGCUAA | 866 |
| CS914203 | (NAG37)s(invAb)sagucacaaCfCfAfauaaaugucus(invAb) | 775 | AGUCACAACCAAUAAAUGUCU | 864 |
| CS915060 | (TriAlk14)a_2NsagucacaAfCfCfaauaaauguas(invAb) | 776 | (A^{2N})AGUCACAACCAAUAAAUGUA | 863 |
| CS915061 | (TriAlk14)asgucacaaCfCfAfauaaaugucus(invAb) | 777 | AGUCACAACCAAUAAAUGUCU | 864 |
| CS915705 | (TriAlk14)gsgaaacucAfgAfuAfaaugcuaas(invAb) | 778 | GGAAACUCAGAUAAAUGCUAA | 866 |
| CS916246 | (TriAlk14)gsgucacaaCfCfAfauaaaugucus(invAb) | 779 | GGUCACAACCAAUAAAUGUCU | 872 |
| CS916247 | (TriAlk14)gsgucacaaCfCfAfauaaaugucas(invAb) | 780 | GGUCACAACCAAUAAAUGUCA | 873 |
| CS916248 | (TriAlk14)asgucacaaCfCfAfauaaaugucas(invAb) | 781 | AGUCACAACCAAUAAAUGUCA | 874 |

(A^{2N})=2-aminoadenosine nucleotide; I = hypoxanthine (inosine) nucleotide

TABLE 6
TSLP RNAi Agent Sense Strand Sequences (Shown with Targeting Ligand Conjugate. The structure of
avb6-SM6.1 is shown in Table 11, and the structure of Tri-SM6.1-avb6-TA14 is shown in FIG. 1.)

| | | | Corresponding |
|---|---|---|---|
| | | | Sense Strand |
| | | | (AM Number) |
| | | | Without Linker |
| Strand | | SEQ | or Conjugate |
| ID | Modified Sense Strand (5′→3′) | ID NO. | (See Table 4) |
| CS001922 | Tri-SM6.1-avb6-(TA14)-gsa_2NaucaaaCfCfUfcacaaauucus(invAb) | 782 | |
| CS003220 | Tri-SM6.1-avb6-(TA14)-csugaaacuGfAfGfagaaaugguas(invAb) | 783 | |
| CS003898 | Tri-SM6.1-avb6-(TA14)-csa_2NuugccuUfAfCfugaaauccaas(invAb) | 784 | AM19257-SS-NL |
| CS003900 | Tri-SM6.1-avb6-(TA14)-gsa_2NaauccaGfAfGfccuaaccuuas(invAb) | 785 | AM19259-SS-NL |
| CS003902 | Tri-SM6.1-avb6-(TA14)-gsauucggaAfAfCfucagauaaa_2Nus(invAb) | 786 | AM19261-SS-NL |
| CS003904 | Tri-SM6.1-avb6-(TA14)-a_2NsagucacaAfCfCfaauaaauguas(invAb) | 787 | AM19263-SS-NL |
| CS003906 | Tri-SM6.1-avb6-(TA14)-asgucacaaCfCfAfauaaaugucus(invAb) | 788 | AM19265-SS-NL |
| CS003908 | Tri-SM6.1-avb6-(TA14)-gsaaagucaCfAfAfccaauaaa_2Nuas(invAb) | 789 | AM19267-SS-NL |
| CS003954 | Tri-SM6.1-avb6-(TA14)-gsgaaacucAfGfAfuaaaugcuaas(invAb) | 790 | AM19334-SS-NL |
| CS003956 | Tri-SM6.1-avb6-(TA14)-asuccagagCfCfUfaaccuucaaus(invAb) | 791 | AM19336-SS-NL |
| CS003958 | Tri-SM6.1-avb6-(TA14)-usa_2NcugaaaUfCfCfagagccuaaas(invAb) | 792 | AM19338-SS-NL |
| CS004174 | Tri-SM6.1-avb6-(TA14)-gsgaaacucAfgAfuAfaaugcuaas(invAb) | 793 | AM19940-SS-NL |
| CS004205 | Tri-SM6.1-avb6-(TA14)-asgucacaaCfcAfaUfaaaugucus(invAb) | 794 | AM19948-SS-NL |
| CS004244 | Tri-SM6.1-avb6-(TA14)-asgagccuaAfCfCfuucaaucucas(invAb) | 795 | AM20175-SS-NL |
| CS004246 | Tri-SM6.1-avb6-(TA14)-asgagccuaAfCfCfuucaaucccas(invAb) | 796 | AM20177-SS-NL |
| CS004280 | Tri-SM6.1-avb6-(TA14)-gsgaaacucdAgdAudAaaugcuaas(invAb) | 797 | AM20298-SS-NL |
| CS004291 | Tri-SM6.1-avb6-(TA14)-gsa_2NacucAfGfAfuaaaugcuaas(invAb) | 798 | AM20306-SS-NL |
| CS004392 | Tri-SM6.1-avb6-(TA14)-asgucacAfaCfcAfauaaaugucus(invAb) | 799 | AM20492-SS-NL |
| CS004393 | Tri-SM6.1-avb6-(TA14)-gsgucacaaCfCfAfauaaaugucus(invAb) | 800 | AM20533-SS-NL |
| CS004395 | Tri-SM6.1-avb6-(TA14)-gsgucacaaCfCfAfauaaaugucas(invAb) | 801 | AM20535-SS-NL |
| CS004397 | Tri-SM6.1-avb6-(TA14)-asgucacaaCfCfAfauaaaugucas(invAb) | 802 | AM20537-SS-NL |
| CS004420 | Tri-SM6.1-avb6-(TA14)-a_2NsagucacaAfcCfaAfuaaauguas(invAb) | 803 | CS004419-NL |
| CS005036 | Tri-SM6.1-avb6-(TA14)-gsagucacaAfCfCfaauaaauguas(invAb) | 804 | |
| CS005405 | Tri-SM6.1-avb6-(TA14)-asgucacaaCfCfAfauaaauguccs(invAb) | 805 | CS005406-NL |
| CS005657 | Tri-SM6.1-avb6-(TA14)-asaggcugcCfUfUfagcuaucugas(invAb) | 806 | CS005629-NL |
| CS005658 | Tri-SM6.1-avb6-(TA14)-csgcuucaaUfCfGfaccuuuacuas(invAb) | 807 | CS005631-NL |
| CS005659 | Tri-SM6.1-avb6-(TA14)-gsa_2NaacauuAfAfCfucuaacuguas(invAb) | 808 | CS005633-NL |
| CS005660 | Tri-SM6.1-avb6-(TA14)-a_2NscagaagaGfUfUfucuuaacuuas(invAb) | 809 | CS005635-NL |
| CS005661 | Tri-SM6.1-avb6-(TA14)-gsuacuacuCfCfUfcaaauguugas(invAb) | 810 | CS005637-NL |
| CS005662 | Tri-SM6.1-avb6-(TA14)-csuuccauaAfCfAfuugaugacuas(invAb) | 811 | CS005639-NL |
| CS005663 | Tri-SM6.1-avb6-(TA14)-gscaaugauAfGfCfaccuaaacuus(invAb) | 812 | CS005641-NL |
| CS005664 | Tri-SM6.1-avb6-(TA14)-a_2NsagacagaCfAfUfuccuucuacas(invAb) | 813 | CS005643-NL |
| CS005665 | Tri-SM6.1-avb6-(TA14)-csa_2NuguaauGfAfCfacuucuuguas(invAb) | 814 | CS005645-NL |
| CS005666 | Tri-SM6.1-avb6-(TA14)-a_2NsuacaaguAfGfAfuccugagaaas(invAb) | 815 | CS005647-NL |
| CS005747 | Tri-SM6.1-avb6-(TA14)-gsgucacaaCfCfAfacaaaugucus(invAb) | 816 | |
| CS005748 | Tri-SM6.1-avb6-(TA14)-gsgucacaaCfCfAfauaaacgucus(invAb) | 817 | |
| CS005957 | Tri-SM6.1-avb6-(TA14)-gsgucacaaCfCfAfauaaauguccs(invAb) | 818 | |
| CS005960 | Tri-SM6.1-avb6-(TA14)-gsgucacaaCfCfAfauaaacguccs(invAb) | 819 | |
| CS006344 | Tri-SM6.1-avb6-(TA14)-asgucacaaCfCfAfacaaaugucus(invAb) | 820 | |
| CS006346 | Tri-SM6.1-avb6-(TA14)-gsgucacaaCfCfAfacaaaugucas(invAb) | 821 | |
| CS006349 | Tri-SM6.1-avb6-(TA14)-asgucacaaCfCfAfauaaacgucus(invAb) | 822 | |

The TSLP RNAi agents disclosed herein are formed by annealing an antisense strand with a sense strand. A sense strand containing a sequence listed in Table 2, Table 4, Table 5, or Table 6 can be hybridized to any antisense strand containing a sequence listed in Table 2 or Table 3, provided the two sequences have a region of at least 85% complementarity over a contiguous 16, 17, 18, 19, 20, or 21 nucleotide sequence.

As shown in Table 5 above, certain of the example TSLP RNAi agent nucleotide sequences are shown to further include reactive linking groups at one or both of the 5′ terminal end and the 3′ terminal end of the sense strand. For example, many of the TSLP RNAi agent sense strand sequences shown in Table 5 above have a (TriAlk14) linking group at the 5′ end of the nucleotide sequence. Other linking groups, such as an (NH2-C6) linking group or a (6-SS-6) or (C6-SS-C6) linking group, may be present as well or alternatively in certain embodiments. Such reactive linking groups are positioned to facilitate the linking of targeting ligands, targeting groups, and/or PK/PD modulators to the TSLP RNAi agents disclosed herein. Linking or conjugation reactions are well known in the art and provide for formation of covalent linkages between two molecules or reactants. Suitable conjugation reactions for use in the scope of the inventions herein include, but are not limited to, amide coupling reaction, Michael addition reaction, hydrazone formation reaction, inverse-demand Diels-Alder cycloaddition reaction, oxime ligation, and Copper (I)-catalyzed or strain-promoted azide-alkyne cycloaddition reaction cycloaddition reaction.

In some embodiments, targeting ligands, such as the integrin targeting ligands shown in the examples and figures disclosed herein, can be synthesized as activated esters, such as tetrafluorophenyl (TFP) esters, which can be displaced by a reactive amino group (e.g., NH₂-C₆) to attach the targeting ligand to the TSLP RNAi agents disclosed herein. In some embodiments, targeting ligands are synthesized as azides, which can be conjugated to a propargyl (e.g., TriAlk14) or DBCO group, for example, via Copper (I)-catalyzed or strain-promoted azide-alkyne cycloaddition reaction.

Additionally, certain of the nucleotide sequences can be synthesized with a dT nucleotide at the 3′ terminal end of the sense strand, followed by (3′→5′) a linker (e.g., C6-SS-C6). The linker can, in some embodiments, facilitate the linkage to additional components, such as, for example, a PK/PD modulator or one or more targeting ligands. As described herein, the disulfide bond of C6-SS-C6 is first reduced, removing the dT from the molecule, which can then facilitate the conjugation of the desired PK/PD modulator. The terminal dT nucleotide therefore is not a part of the fully conjugated construct.

In some embodiments, the antisense strand of a TSLP RNAi agent disclosed herein differs by 0, 1, 2, or 3 nucleotides from any of the antisense strand sequences in Table 3 or Table 10. In some embodiments, the sense strand of a TSLP RNAi agent disclosed herein differs by 0, 1, 2, or 3 nucleotides from any of the sense strand sequences in Table 4, Table 5, Table 6, or Table 10.

In some embodiments, a TSLP RNAi agent antisense strand comprises a nucleotide sequence of any of the sequences in Table 2 or Table 3. In some embodiments, a TSLP RNAi agent antisense strand comprises the sequence of nucleotides (from 5′ end→3′ end) 1-17, 2-17, 1-18, 2-18, 1-19, 2-19, 1-20, 2-20, 1-21, 2-21, 1-22, 2-22, 1-23, 2-23, 1-24, or 2-24 of any of the sequences in Table 2, Table 3, or Table 10. In certain embodiments, a TSLP RNAi agent antisense strand comprises or consists of a modified sequence of any one of the modified sequences in Table 3 or Table 10.

In some embodiments, a TSLP RNAi agent sense strand comprises the nucleotide sequence of any of the sequences in Table 2 or Table 4. In some embodiments, a TSLP RNAi agent sense strand comprises the sequence of nucleotides (from 5′ end→3′ end) 1-17, 2-17, 3-17, 4-17, 1-18, 2-18, 3-18, 4-18, 1-19, 2-19, 3-19, 4-19, 1-20, 2-20, 3-20, 4-20, 1-21, 2-21, 3-21, 4-21, 1-22, 2-22, 3-22, 4-22, 1-23, 2-23, 3-23, 4-23, 1-24, 2-24, 3-24, or 4-24, of any of the sequences in Table 2, Table 4, Table 5, Table 6, or Table 10. In certain embodiments, a TSLP RNAi agent sense strand comprises or consists of a modified sequence of any one of the modified sequences in Table 3 or Table 10.

For the RNAi agents disclosed herein, the nucleotide at position 1 of the antisense strand (from 5′ end→3′ end) can be perfectly complementary to a TSLP gene, or can be non-complementary to a TSLP gene. In some embodiments, the nucleotide at position 1 of the antisense strand (from 5′ end→3′ end) is a U, A, or dT (or a modified version of U, A or dT). In some embodiments, the nucleotide at position 1 of the antisense strand (from 5′ end→3′ end) forms an A:U or U:A base pair with the sense strand.

In some embodiments, a TSLP RNAi agent antisense strand comprises the sequence of nucleotides (from 5′ end→3′ end) 2-18 or 2-19 of any of the antisense strand sequences in Table 2, Table 3, or Table 10. In some embodiments, a TSLP RNAi sense strand comprises the sequence of nucleotides (from 5′ end→3′ end) 1-17 or 1-18 of any of the sense strand sequences in Table 2, Table 4, Table 5, Table 6, or Table 10.

In some embodiments, a TSLP RNAi agent includes (i) an antisense strand comprising the sequence of nucleotides (from 5′ end→3′ end) 2-18 or 2-19 of any of the antisense strand sequences in Table 2, Table 3, or Table 10, and (ii) a sense strand comprising the sequence of nucleotides (from 5′ end→3′ end) 1-17 or 1-18 of any of the sense strand sequences in Table 2, Table 4, Table 5, Table 6, or Table 10.

A sense strand containing a sequence listed in Table 2 or Table 4 can be hybridized to any antisense strand containing a sequence listed in Table 2 or Table 3 provided the two sequences have a region of at least 85% complementarity over a contiguous 16, 17, 18, 19, 20, or 21 nucleotide sequence. In some embodiments, the TSLP RNAi agent has a sense strand consisting of the modified sequence of any of the modified sequences in Table 4, Table 5, Table 6, or Table 10, and an antisense strand consisting of the modified sequence of any of the modified sequences in Table 3 or Table 10. Certain representative sequence pairings are exemplified by the Duplex ID Nos. shown in Tables 7A, 7B, 8, and 9.

In some embodiments, a TSLP RNAi agent comprises, consists of, or consists essentially of a duplex represented by any one of the Duplex ID Nos. presented herein. In some embodiments, a TSLP RNAi agent consists of any of the Duplex ID Nos. presented herein. In some embodiments, a TSLP RNAi agent comprises the sense strand and antisense strand nucleotide sequences of any of the Duplex ID Nos. presented herein. In some embodiments, a TSLP RNAi agent comprises the sense strand and antisense strand nucleotide sequences of any of the Duplex ID Nos. presented herein and a targeting group, linking group, and/or other non-nucleotide group wherein the targeting group, linking group, and/or other non-nucleotide group is covalently linked (i.e., conjugated) to the sense strand or the antisense strand. In some embodiments, a TSLP RNAi agent includes the sense strand and antisense strand modified nucleotide sequences of any of the Duplex ID Nos. presented herein. In some embodiments, a TSLP RNAi agent comprises the sense strand and antisense strand modified nucleotide sequences of any of the Duplex ID Nos. presented herein and a targeting group, linking group, and/or other non-nucleotide group, wherein the targeting group, linking group, and/or other non-nucleotide group is covalently linked to the sense strand or the antisense strand.

In some embodiments, a TSLP RNAi agent comprises an antisense strand and a sense strand having the nucleotide sequences of any of the antisense strand/sense strand duplexes of Tables 2, 7A, 7B, 8, 9, or 10, and comprises a targeting group. In some embodiments, a TSLP RNAi agent comprises an antisense strand and a sense strand having the nucleotide sequences of any of the antisense strand/sense strand duplexes of Tables 2, 7A, 7B, 8, 9, or 10, and comprises one or more αvβ6 integrin targeting ligands.

In some embodiments, a TSLP RNAi agent comprises an antisense strand and a sense strand having the nucleotide sequences of any of the antisense strand/sense strand duplexes of Tables 2, 7A, 7B, 8, 9, or 10, and comprises a targeting group that is an integrin targeting ligand. In some embodiments, a TSLP RNAi agent comprises an antisense strand and a sense strand having the nucleotide sequences of any of the antisense strand/sense strand duplexes of Tables 2, 7A, 7B, 8, 9, or 10, and comprises one or more αvβ6 integrin targeting ligands or clusters of αvβ6 integrin targeting ligands (e.g., a tridentate αvβ6 integrin targeting ligand).

In some embodiments, a TSLP RNAi agent comprises an antisense strand and a sense strand having the modified nucleotide sequences of any of the antisense strand/sense strand duplexes of Tables 7A, 7B, 8, 9, and 10.

In some embodiments, a TSLP RNAi agent comprises an antisense strand and a sense strand having the modified nucleotide sequences of any of the antisense strand/sense strand duplexes of Tables 7A, 7B, 8, 9, and 10, and comprises an integrin targeting ligand.

In some embodiments, a TSLP RNAi agent comprises, consists of, or consists essentially of any of the duplexes of Tables 7A, 7B, 8, 9, and 10.

TABLE 7A
TSLP RNAi Agent Duplexes with Corresponding Sense and Antisense Strand
ID Numbers and Sequence ID numbers for the modified and unmodified
nucleotide sequences. (Shown without Linking Agents or Conjugates)

| | | AS | AS | | SS | SS |
|---|---|---|---|---|---|---|
| | | modified | unmodified | | modified | unmodified |
| | | SEQ ID | SEQ ID | | SEQ ID | SEQ ID |
| Duplex | AS ID | NO: | NO: | SS ID | NO: | NO: |
| AD13575 | AM19258-AS | 591 | 827 | AM19257-SS-NL | 698 | 860 |
| AD13576 | AM19260-AS | 592 | 828 | AM19259-SS-NL | 699 | 861 |
| AD13577 | AM19262-AS | 593 | 829 | AM19261-SS-NL | 700 | 862 |
| AD13578 | AM19264-AS | 594 | 826 | AM19263-SS-NL | 701 | 863 |
| AD13579 | AM19266-AS | 595 | 830 | AM19265-SS-NL | 702 | 864 |
| AD13580 | AM19268-AS | 596 | 831 | AM19267-SS-NL | 703 | 865 |
| AD13581 | AM18311-AS | 590 | 826 | AM19263-SS-NL | 701 | 863 |
| AD13635 | AM19335-AS | 597 | 825 | AM19334-SS-NL | 704 | 866 |
| AD13636 | AM19337-AS | 598 | 832 | AM19336-SS-NL | 705 | 867 |
| AD13637 | AM19339-AS | 599 | 833 | AM19338-SS-NL | 706 | 868 |
| AD13942 | AM19685-AS | 600 | 825 | AM19334-SS-NL | 704 | 866 |
| AD13943 | AM18285-AS | 589 | 825 | AM19334-SS-NL | 704 | 866 |
| AD14173 | AM19938-AS | 601 | 825 | AM19334-SS-NL | 704 | 866 |
| AD14174 | AM19939-AS | 602 | 825 | AM19334-SS-NL | 704 | 866 |
| AD14175 | AM19335-AS | 597 | 825 | AM19940-SS-NL | 707 | 866 |
| AD14176 | AM19941-AS | 603 | 825 | AM19334-SS-NL | 704 | 866 |
| AD14177 | AM19942-AS | 604 | 825 | AM19334-SS-NL | 704 | 866 |
| AD14178 | AM19943-AS | 605 | 825 | AM19334-SS-NL | 704 | 866 |
| AD14179 | AM19944-AS | 606 | 825 | AM19334-SS-NL | 704 | 866 |
| AD14180 | AM19945-AS | 607 | 825 | AM19334-SS-NL | 704 | 866 |
| AD14181 | AM19946-AS | 608 | 897 | AM19334-SS-NL | 704 | 866 |
| AD14182 | AM19947-AS | 609 | 830 | AM19265-SS-NL | 702 | 864 |
| AD14183 | AM19266-AS | 595 | 830 | AM19948-SS-NL | 708 | 864 |
| AD14184 | AM19949-AS | 610 | 830 | AM19265-SS-NL | 702 | 864 |
| AD14185 | AM19950-AS | 611 | 830 | AM19265-SS-NL | 702 | 864 |
| AD14186 | AM19951-AS | 612 | 830 | AM19265-SS-NL | 702 | 864 |
| AD14187 | AM19952-AS | 613 | 830 | AM19265-SS-NL | 702 | 864 |
| AD14188 | AM19953-AS | 614 | 830 | AM19265-SS-NL | 702 | 864 |
| AD14189 | AM19954-AS | 615 | 830 | AM19265-SS-NL | 702 | 864 |
| AD14190 | AM19955-AS | 616 | 830 | AM19265-SS-NL | 702 | 864 |
| AD14378 | AM20176-AS | 617 | 834 | AM20175-SS-NL | 709 | 869 |
| AD14379 | AM20176-AS | 617 | 834 | AM20177-SS-NL | 710 | 870 |
| AD14481 | AM19941-AS | 603 | 825 | AM19940-SS-NL | 707 | 866 |
| AD14482 | AM19941-AS | 603 | 825 | AM20298-SS-NL | 711 | 866 |
| AD14483 | AM20299-AS | 618 | 825 | AM19334-SS-NL | 704 | 866 |
| AD14484 | AM20300-AS | 619 | 898 | AM19334-SS-NL | 704 | 866 |
| AD14485 | AM20301-AS | 620 | 899 | AM19334-SS-NL | 704 | 866 |
| AD14486 | AM20302-AS | 621 | 899 | AM19334-SS-NL | 704 | 866 |
| AD14487 | AM20302-AS | 621 | 899 | AM20298-SS-NL | 711 | 866 |
| AD14488 | AM20303-AS | 622 | 825 | AM19334-SS-NL | 704 | 866 |
| AD14489 | AM20304-AS | 623 | 900 | AM19334-SS-NL | 704 | 866 |
| AD14490 | AM20305-AS | 624 | 825 | AM19334-SS-NL | 704 | 866 |
| AD14491 | AM20307-AS | 625 | 835 | AM20306-SS-NL | 712 | 871 |
| AD14492 | AM20308-AS | 626 | 825 | AM19334-SS-NL | 704 | 866 |
| AD14493 | AM20309-AS | 627 | 825 | AM19334-SS-NL | 704 | 866 |
| AD14494 | AM20310-AS | 628 | 825 | AM19334-SS-NL | 704 | 866 |
| AD14500 | AM20314-AS | 629 | 899 | AM19334-SS-NL | 704 | 866 |
| AD14501 | AM20315-AS | 630 | 899 | AM19334-SS-NL | 704 | 866 |
| AD14502 | AM20315-AS | 630 | 899 | AM20298-SS-NL | 711 | 866 |
| AD14629 | AM20487-AS | 631 | 826 | AM19263-SS-NL | 701 | 863 |
| AD14630 | AM20488-AS | 632 | 830 | AM19265-SS-NL | 702 | 864 |
| AD14631 | AM20489-AS | 633 | 830 | AM19265-SS-NL | 702 | 864 |
| AD14632 | AM20490-AS | 634 | 830 | AM19265-SS-NL | 702 | 864 |
| AD14633 | AM20491-AS | 635 | 825 | AM19334-SS-NL | 704 | 866 |
| AD14634 | AM19947-AS | 609 | 830 | AM20492-SS-NL | 713 | 864 |
| AD14635 | AM19947-AS | 609 | 830 | AM19948-SS-NL | 708 | 864 |
| AD14673 | AM20534-AS | 636 | 836 | AM20533-SS-NL | 714 | 872 |
| AD14674 | AM20536-AS | 637 | 837 | AM20535-SS-NL | 715 | 873 |
| AD14675 | AM20538-AS | 638 | 838 | AM20537-SS-NL | 716 | 874 |
| AD14676 | AM20539-AS | 639 | 838 | AM20537-SS-NL | 716 | 874 |
| AD14677 | AM20540-AS | 640 | 838 | AM20537-SS-NL | 716 | 874 |
| AC003561 | CA004416 | 643 | 826 | CS915060-NL | 734 | 863 |
| AC003562 | CA004416 | 643 | 826 | CS004419-NL | 717 | 863 |
| AC003563 | CA004417 | 644 | 826 | CS915060-NL | 734 | 863 |
| AC003564 | CA004417 | 644 | 826 | CS004419-NL | 717 | 863 |
| AC003565 | CA004418 | 645 | 826 | CS004419-NL | 717 | 863 |
| AC003566 | CA004418 | 645 | 826 | CS915060-NL | 734 | 863 |
| AC003595 | CA004450 | 646 | 837 | CS916247-NL | 738 | 873 |
| AC003596 | CA004451 | 647 | 836 | CS916246-NL | 737 | 872 |
| AC003599 | CA004452 | 648 | 837 | CS916247-NL | 738 | 873 |
| AC003600 | CA004453 | 649 | 836 | CS916246-NL | 737 | 872 |
| AC003657 | CA004518 | 650 | 838 | CS916248-NL | 739 | 874 |
| AC003658 | CA004519 | 651 | 837 | CS916247-NL | 738 | 873 |
| AC003679 | CA004538 | 652 | 830 | CS914203-NL | 733 | 864 |
| AC003842 | CA004288 | 642 | 825 | CS915705-NL | 736 | 866 |
| AC003920 | CA004830 | 653 | 836 | CS004829-NL | 718 | 872 |
| AC003923 | CA004833 | 654 | 836 | CS004829-NL | 718 | 872 |
| AC003989 | CA004288 | 642 | 825 | CS914177-NL | 732 | 866 |
| AC004080 | CA005032 | 656 | 830 | CS915061-NL | 735 | 864 |
| AC004081 | CA005035 | 659 | 826 | CS915060-NL | 734 | 863 |
| AC004359 | CA005404 | 662 | 840 | CS915061-NL | 735 | 864 |
| AC004360 | CA005404 | 662 | 840 | CS005406-NL | 719 | 875 |
| AC004362 | CA005407 | 663 | 841 | CS915061-NL | 735 | 864 |
| AC004366 | CA005410 | 664 | 830 | CS915061-NL | 735 | 864 |
| AC004367 | CA005411 | 665 | 830 | CS915061-NL | 735 | 864 |
| AC004368 | CA005412 | 666 | 830 | CS915061-NL | 735 | 864 |
| AC004369 | CA005413 | 667 | 902 | CS915061-NL | 735 | 864 |
| AC004370 | CA005414 | 668 | 903 | CS915061-NL | 735 | 864 |
| AC004371 | CA005415 | 669 | 830 | CS915061-NL | 735 | 864 |
| AC004547 | CA005630 | 670 | 842 | CS005629-NL | 720 | 876 |
| AC004548 | CA005632 | 671 | 843 | CS005631-NL | 721 | 877 |
| AC004549 | CA005634 | 672 | 844 | CS005633-NL | 722 | 878 |
| AC004550 | CA005636 | 673 | 845 | CS005635-NL | 723 | 879 |
| AC004551 | CA005638 | 674 | 846 | CS005637-NL | 724 | 880 |
| AC004552 | CA005640 | 675 | 847 | CS005639-NL | 725 | 881 |
| AC004553 | CA005642 | 676 | 848 | CS005641-NL | 726 | 882 |
| AC004554 | CA005644 | 677 | 849 | CS005643-NL | 727 | 883 |
| AC004555 | CA005646 | 678 | 850 | CS005645-NL | 728 | 884 |
| AC004556 | CA005648 | 679 | 851 | CS005647-NL | 729 | 885 |
| AC004836 | CA005976 | 687 | 855 | CS916246-NL | 737 | 872 |
| AC005235 | CA006383 | 697 | 859 | CS916248-NL | 739 | 874 |
| AC005248 | CA006350 | 696 | 841 | CS915061-NL | 735 | 864 |
| AC005941 | CA004207 | 641 | 830 | CS007200-NL | 730 | 864 |
| AC005942 | CA005056 | 661 | 840 | CS915061-NL | 735 | 864 |
| AC005943 | CA004538 | 652 | 830 | CS915061-NL | 735 | 864 |
| AC007332 | CA005404 | 662 | 840 | CS008694-NL | 731 | 864 |
| AC007334 | CA004207 | 641 | 830 | CS008694-NL | 731 | 864 |

TABLE 7B
TSLP RNAi Agent Duplexes with Corresponding Sense and Antisense Strand ID Numbers
and Sequence ID numbers for the modified and unmodified nucleotide sequences.

| | | AS | | | SS | SS |
|---|---|---|---|---|---|---|
| | | modified | AS | | modified | unmodified |
| | | SEQ ID | unmodified | | SEQ ID | SEQ ID |
| Duplex | AS ID | NO: | SEQ ID NO: | SS ID | NO: | NO: |
| AD13575 | AM19258-AS | 591 | 827 | AM19257-SS | 740 | 860 |
| AD13576 | AM19260-AS | 592 | 828 | AM19259-SS | 741 | 861 |
| AD13577 | AM19262-AS | 593 | 829 | AM19261-SS | 742 | 862 |
| AD13578 | AM19264-AS | 594 | 826 | AM19263-SS | 743 | 863 |
| AD13579 | AM19266-AS | 595 | 830 | AM19265-SS | 744 | 864 |
| AD13580 | AM19268-AS | 596 | 831 | AM19267-SS | 745 | 865 |
| AD13581 | AM18311-AS | 590 | 826 | AM19263-SS | 743 | 863 |
| AD13635 | AM19335-AS | 597 | 825 | AM19334-SS | 746 | 866 |
| AD13636 | AM19337-AS | 598 | 832 | AM19336-SS | 747 | 867 |
| AD13637 | AM19339-AS | 599 | 833 | AM19338-SS | 748 | 868 |
| AD13942 | AM19685-AS | 600 | 825 | AM19334-SS | 746 | 866 |
| AD13943 | AM18285-AS | 589 | 825 | AM19334-SS | 746 | 866 |
| AD14173 | AM19938-AS | 601 | 825 | AM19334-SS | 746 | 866 |
| AD14174 | AM19939-AS | 602 | 825 | AM19334-SS | 746 | 866 |
| AD14175 | AM19335-AS | 597 | 825 | AM19940-SS | 749 | 866 |
| AD14176 | AM19941-AS | 603 | 825 | AM19334-SS | 746 | 866 |
| AD14177 | AM19942-AS | 604 | 825 | AM19334-SS | 746 | 866 |
| AD14178 | AM19943-AS | 605 | 825 | AM19334-SS | 746 | 866 |
| AD14179 | AM19944-AS | 606 | 825 | AM19334-SS | 746 | 866 |
| AD14180 | AM19945-AS | 607 | 825 | AM19334-SS | 746 | 866 |
| AD14181 | AM19946-AS | 608 | 897 | AM19334-SS | 746 | 866 |
| AD14182 | AM19947-AS | 609 | 830 | AM19265-SS | 744 | 864 |
| AD14183 | AM19266-AS | 595 | 830 | AM19948-SS | 750 | 864 |
| AD14184 | AM19949-AS | 610 | 830 | AM19265-SS | 744 | 864 |
| AD14185 | AM19950-AS | 611 | 830 | AM19265-SS | 744 | 864 |
| AD14186 | AM19951-AS | 612 | 830 | AM19265-SS | 744 | 864 |
| AD14187 | AM19952-AS | 613 | 830 | AM19265-SS | 744 | 864 |
| AD14188 | AM19953-AS | 614 | 830 | AM19265-SS | 744 | 864 |
| AD14189 | AM19954-AS | 615 | 830 | AM19265-SS | 744 | 864 |
| AD14190 | AM19955-AS | 616 | 830 | AM19265-SS | 744 | 864 |
| AD14378 | AM20176-AS | 617 | 834 | AM20175-SS | 751 | 869 |
| AD14379 | AM20176-AS | 617 | 834 | AM20177-SS | 752 | 870 |
| AD14481 | AM19941-AS | 603 | 825 | AM19940-SS | 749 | 866 |
| AD14482 | AM19941-AS | 603 | 825 | AM20298-SS | 753 | 866 |
| AD14483 | AM20299-AS | 618 | 825 | AM19334-SS | 746 | 866 |
| AD14484 | AM20300-AS | 619 | 898 | AM19334-SS | 746 | 866 |
| AD14485 | AM20301-AS | 620 | 899 | AM19334-SS | 746 | 866 |
| AD14486 | AM20302-AS | 621 | 899 | AM19334-SS | 746 | 866 |
| AD14487 | AM20302-AS | 621 | 899 | AM20298-SS | 753 | 866 |
| AD14488 | AM20303-AS | 622 | 825 | AM19334-SS | 746 | 866 |
| AD14489 | AM20304-AS | 623 | 900 | AM19334-SS | 746 | 866 |
| AD14490 | AM20305-AS | 624 | 825 | AM19334-SS | 746 | 866 |
| AD14491 | AM20307-AS | 625 | 835 | AM20306-SS | 754 | 871 |
| AD14492 | AM20308-AS | 626 | 825 | AM19334-SS | 746 | 866 |
| AD14493 | AM20309-AS | 627 | 825 | AM19334-SS | 746 | 866 |
| AD14494 | AM20310-AS | 628 | 825 | AM19334-SS | 746 | 866 |
| AD14500 | AM20314-AS | 629 | 899 | AM19334-SS | 746 | 866 |
| AD14501 | AM20315-AS | 630 | 899 | AM19334-SS | 746 | 866 |
| AD14502 | AM20315-AS | 630 | 899 | AM20298-SS | 753 | 866 |
| AD14629 | AM20487-AS | 631 | 826 | AM19263-SS | 743 | 863 |
| AD14630 | AM20488-AS | 632 | 830 | AM19265-SS | 744 | 864 |
| AD14631 | AM20489-AS | 633 | 830 | AM19265-SS | 744 | 864 |
| AD14632 | AM20490-AS | 634 | 830 | AM19265-SS | 744 | 864 |
| AD14633 | AM20491-AS | 635 | 825 | AM19334-SS | 746 | 866 |
| AD14634 | AM19947-AS | 609 | 830 | AM20492-SS | 755 | 864 |
| AD14635 | AM19947-AS | 609 | 830 | AM19948-SS | 750 | 864 |
| AD14673 | AM20534-AS | 636 | 836 | AM20533-SS | 756 | 872 |
| AD14674 | AM20536-AS | 637 | 837 | AM20535-SS | 757 | 873 |
| AD14675 | AM20538-AS | 638 | 838 | AM20537-SS | 758 | 874 |
| AD14676 | AM20539-AS | 639 | 838 | AM20537-SS | 758 | 874 |
| AD14677 | AM20540-AS | 640 | 838 | AM20537-SS | 758 | 874 |
| AC003561 | CA004416 | 643 | 826 | CS915060 | 776 | 863 |
| AC003562 | CA004416 | 643 | 826 | CS004419 | 759 | 863 |
| AC003563 | CA004417 | 644 | 826 | CS915060 | 776 | 863 |
| AC003564 | CA004417 | 644 | 826 | CS004419 | 759 | 863 |
| AC003565 | CA004418 | 645 | 826 | CS004419 | 759 | 863 |
| AC003566 | CA004418 | 645 | 826 | CS915060 | 776 | 863 |
| AC003595 | CA004450 | 646 | 837 | CS916247 | 780 | 873 |
| AC003596 | CA004451 | 647 | 836 | CS916246 | 779 | 872 |
| AC003599 | CA004452 | 648 | 837 | CS916247 | 780 | 873 |
| AC003600 | CA004453 | 649 | 836 | CS916246 | 779 | 872 |
| AC003657 | CA004518 | 650 | 838 | CS916248 | 781 | 874 |
| AC003658 | CA004519 | 651 | 837 | CS916247 | 780 | 873 |
| AC003679 | CA004538 | 652 | 830 | CS914203 | 775 | 864 |
| AC003842 | CA004288 | 642 | 825 | CS915705 | 778 | 866 |
| AC003920 | CA004830 | 653 | 836 | CS004829 | 760 | 872 |
| AC003923 | CA004833 | 654 | 836 | CS004829 | 760 | 872 |
| AC003989 | CA004288 | 642 | 825 | CS914177 | 774 | 866 |
| AC004080 | CA005032 | 656 | 830 | CS915061 | 777 | 864 |
| AC004081 | CA005035 | 659 | 826 | CS915060 | 776 | 863 |
| AC004359 | CA005404 | 662 | 840 | CS915061 | 777 | 864 |
| AC004360 | CA005404 | 662 | 840 | CS005406 | 761 | 875 |
| AC004362 | CA005407 | 663 | 841 | CS915061 | 777 | 864 |
| AC004366 | CA005410 | 664 | 830 | CS915061 | 777 | 864 |
| AC004367 | CA005411 | 665 | 830 | CS915061 | 777 | 864 |
| AC004368 | CA005412 | 666 | 830 | CS915061 | 777 | 864 |
| AC004369 | CA005413 | 667 | 902 | CS915061 | 777 | 864 |
| AC004370 | CA005414 | 668 | 903 | CS915061 | 777 | 864 |
| AC004371 | CA005415 | 669 | 830 | CS915061 | 777 | 864 |
| AC004547 | CA005630 | 670 | 842 | CS005629 | 762 | 876 |
| AC004548 | CA005632 | 671 | 843 | CS005631 | 763 | 877 |
| AC004549 | CA005634 | 672 | 844 | CS005633 | 764 | 878 |
| AC004550 | CA005636 | 673 | 845 | CS005635 | 765 | 879 |
| AC004551 | CA005638 | 674 | 846 | CS005637 | 766 | 880 |
| AC004552 | CA005640 | 675 | 847 | CS005639 | 767 | 881 |
| AC004553 | CA005642 | 676 | 848 | CS005641 | 768 | 882 |
| AC004554 | CA005644 | 677 | 849 | CS005643 | 769 | 883 |
| AC004555 | CA005646 | 678 | 850 | CS005645 | 770 | 884 |
| AC004556 | CA005648 | 679 | 851 | CS005647 | 771 | 885 |
| AC004836 | CA005976 | 687 | 855 | CS916246 | 779 | 872 |
| AC005235 | CA006383 | 697 | 859 | CS916248 | 781 | 874 |
| AC005248 | CA006350 | 696 | 841 | CS915061 | 777 | 864 |
| AC005941 | CA004207 | 641 | 830 | CS007200 | 772 | 864 |
| AC005942 | CA005056 | 661 | 840 | CS915061 | 777 | 864 |
| AC005943 | CA004538 | 652 | 830 | CS915061 | 777 | 864 |
| AC007332 | CA005404 | 662 | 840 | CS008694 | 773 | 886 |
| AC007334 | CA004207 | 641 | 830 | CS008694 | 773 | 886 |

TABLE 8
TSLP RNAi Agent Conjugate Duplexes with Corresponding Sense and Antisense
Strand ID Numbers and Sequence ID numbers for the modified and unmodified
nucleotide sequences. (Shown with Targeting Ligand Conjugates)

| | | AS | AS | | SS | SS |
|---|---|---|---|---|---|---|
| | | modified | unmodified | | modified | unmodified |
| | | SEQ ID | SEQ ID | | SEQ ID | SEQ ID |
| Duplex | AS ID | NO: | NO: | SS ID | NO: | NO: |
| AC001714 | AM14179-AS | 587 | 823 | CS001922 | 782 | 887 |
| AC002515 | AM16334-AS | 588 | 824 | CS003220 | 783 | 888 |
| AC003096 | AM19258-AS | 591 | 827 | CS003898 | 784 | 860 |
| AC003097 | AM19260-AS | 592 | 828 | CS003900 | 785 | 861 |
| AC003098 | AM19262-AS | 593 | 829 | CS003902 | 786 | 862 |
| AC003099 | AM19264-AS | 594 | 826 | CS003904 | 787 | 863 |
| AC003100 | AM19266-AS | 595 | 830 | CS003906 | 788 | 864 |
| AC003101 | AM19268-AS | 596 | 831 | CS003908 | 789 | 865 |
| AC003102 | AM18311-AS | 590 | 826 | CS003904 | 787 | 863 |
| AC003128 | AM19335-AS | 597 | 825 | CS003954 | 790 | 866 |
| AC003129 | AM19337-AS | 598 | 832 | CS003956 | 791 | 867 |
| AC003130 | AM19339-AS | 599 | 833 | CS003958 | 792 | 868 |
| AC003252 | AM18285-AS | 589 | 825 | CS003954 | 790 | 866 |
| AC003253 | AM19685-AS | 600 | 825 | CS003954 | 790 | 866 |
| AC003339 | AM19938-AS | 601 | 825 | CS003954 | 790 | 866 |
| AC003340 | AM19939-AS | 602 | 825 | CS003954 | 790 | 866 |
| AC003341 | AM19335-AS | 597 | 825 | CS004174 | 793 | 866 |
| AC003342 | AM19941-AS | 603 | 825 | CS003954 | 790 | 866 |
| AC003343 | AM19942-AS | 604 | 825 | CS003954 | 790 | 866 |
| AC003344 | AM19943-AS | 605 | 825 | CS003954 | 790 | 866 |
| AC003345 | AM19944-AS | 606 | 825 | CS003954 | 790 | 866 |
| AC003346 | AM19945-AS | 607 | 825 | CS003954 | 790 | 866 |
| AC003347 | AM19946-AS | 608 | 897 | CS003954 | 790 | 866 |
| AC003371 | AM19947-AS | 609 | 830 | CS003906 | 788 | 864 |
| AC003372 | AM19266-AS | 595 | 830 | CS004205 | 794 | 864 |
| AC003373 | AM19949-AS | 610 | 830 | CS003906 | 788 | 864 |
| AC003374 | AM19950-AS | 611 | 830 | CS003906 | 788 | 864 |
| AC003375 | AM19951-AS | 612 | 830 | CS003906 | 788 | 864 |
| AC003376 | AM19952-AS | 613 | 830 | CS003906 | 788 | 864 |
| AC003377 | AM19953-AS | 614 | 830 | CS003906 | 788 | 864 |
| AC003378 | AM19954-AS | 615 | 830 | CS003906 | 788 | 864 |
| AC003379 | AM19955-AS | 616 | 830 | CS003906 | 788 | 864 |
| AC003415 | AM20176-AS | 617 | 834 | CS004244 | 795 | 869 |
| AC003416 | AM20176-AS | 617 | 834 | CS004246 | 796 | 870 |
| AC003446 | AM19941-AS | 603 | 825 | CS004174 | 793 | 866 |
| AC003447 | AM19941-AS | 603 | 825 | CS004280 | 797 | 866 |
| AC003448 | AM20299-AS | 618 | 825 | CS003954 | 790 | 866 |
| AC003449 | AM20300-AS | 619 | 898 | CS003954 | 790 | 866 |
| AC003450 | AM20314-AS | 629 | 899 | CS003954 | 790 | 866 |
| AC003451 | AM20315-AS | 630 | 899 | CS003954 | 790 | 866 |
| AC003452 | AM20315-AS | 630 | 899 | CS004280 | 797 | 866 |
| AC003453 | AM20303-AS | 622 | 825 | CS003954 | 790 | 866 |
| AC003454 | AM20308-AS | 626 | 825 | CS003954 | 790 | 866 |
| AC003455 | AM20309-AS | 627 | 825 | CS003954 | 790 | 866 |
| AC003456 | AM20310-AS | 628 | 825 | CS003954 | 790 | 866 |
| AC003457 | AM20304-AS | 623 | 900 | CS003954 | 790 | 866 |
| AC003458 | AM20305-AS | 624 | 825 | CS003954 | 790 | 866 |
| AC003459 | AM20307-AS | 625 | 835 | CS004291 | 798 | 871 |
| AC003511 | AM20487-AS | 631 | 826 | CS003904 | 787 | 863 |
| AC003537 | AM20488-AS | 632 | 830 | CS003906 | 788 | 864 |
| AC003538 | AM20489-AS | 633 | 830 | CS003906 | 788 | 864 |
| AC003539 | AM20490-AS | 634 | 830 | CS003906 | 788 | 864 |
| AC003540 | AM20491-AS | 635 | 825 | CS003954 | 790 | 866 |
| AC003541 | AM19947-AS | 609 | 830 | CS004392 | 799 | 864 |
| AC003542 | AM19947-AS | 609 | 830 | CS004205 | 794 | 864 |
| AC003543 | AM20534-AS | 636 | 836 | CS004393 | 800 | 872 |
| AC003544 | AM20536-AS | 637 | 837 | CS004395 | 801 | 873 |
| AC003545 | AM20538-AS | 638 | 838 | CS004397 | 802 | 874 |
| AC003546 | AM20539-AS | 639 | 838 | CS004397 | 802 | 874 |
| AC003547 | AM20540-AS | 640 | 838 | CS004397 | 802 | 874 |
| AC003567 | CA004416 | 643 | 826 | CS003904 | 787 | 863 |
| AC003568 | CA004416 | 643 | 826 | CS004420 | 803 | 863 |
| AC003569 | CA004417 | 644 | 826 | CS003904 | 787 | 863 |
| AC003570 | CA004417 | 644 | 826 | CS004420 | 803 | 863 |
| AC003571 | CA004418 | 645 | 826 | CS004420 | 803 | 863 |
| AC003572 | CA004418 | 645 | 826 | CS003904 | 787 | 863 |
| AC003597 | CA004450 | 646 | 837 | CS004395 | 801 | 873 |
| AC003598 | CA004451 | 647 | 836 | CS004393 | 800 | 872 |
| AC003601 | CA004452 | 648 | 837 | CS004395 | 801 | 873 |
| AC003602 | CA004453 | 649 | 836 | CS004393 | 800 | 872 |
| AC003659 | CA004518 | 650 | 838 | CS004397 | 802 | 874 |
| AC003660 | CA004519 | 651 | 837 | CS004395 | 801 | 873 |
| AC003843 | CA004288 | 642 | 825 | CS004174 | 793 | 866 |
| AC003924 | CA004834 | 655 | 836 | CS004393 | 800 | 872 |
| AC004077 | CA005033 | 657 | 836 | CS004393 | 800 | 872 |
| AC004078 | CA005034 | 658 | 901 | CS004393 | 800 | 872 |
| AC004079 | CA005037 | 660 | 839 | CS005036 | 804 | 889 |
| AC004082 | CA005032 | 656 | 830 | CS003906 | 788 | 864 |
| AC004083 | CA005035 | 659 | 826 | CS003904 | 787 | 863 |
| AC004358 | CA005404 | 662 | 840 | CS005405 | 805 | 875 |
| AC004361 | CA005404 | 662 | 840 | CS003906 | 788 | 864 |
| AC004363 | CA005407 | 663 | 841 | CS003906 | 788 | 864 |
| AC004373 | CA005413 | 667 | 902 | CS003906 | 788 | 864 |
| AC004374 | CA005414 | 668 | 903 | CS003906 | 788 | 864 |
| AC004375 | CA005415 | 669 | 830 | CS003906 | 788 | 864 |
| AC004376 | CA005410 | 664 | 830 | CS003906 | 788 | 864 |
| AC004377 | CA005411 | 665 | 830 | CS003906 | 788 | 864 |
| AC004378 | CA005412 | 666 | 830 | CS003906 | 788 | 864 |
| AC004565 | CA005630 | 670 | 842 | CS005657 | 806 | 876 |
| AC004566 | CA005632 | 671 | 843 | CS005658 | 807 | 877 |
| AC004567 | CA005634 | 672 | 844 | CS005659 | 808 | 878 |
| AC004568 | CA005636 | 673 | 845 | CS005660 | 809 | 879 |
| AC004569 | CA005638 | 674 | 846 | CS005661 | 810 | 880 |
| AC004570 | CA005640 | 675 | 847 | CS005662 | 811 | 881 |
| AC004571 | CA005642 | 676 | 848 | CS005663 | 812 | 882 |
| AC004572 | CA005644 | 677 | 849 | CS005664 | 813 | 883 |
| AC004573 | CA005646 | 678 | 850 | CS005665 | 814 | 884 |
| AC004574 | CA005648 | 679 | 851 | CS005666 | 815 | 885 |
| AC004644 | CA005746 | 680 | 852 | CS004393 | 800 | 872 |
| AC004645 | CA005746 | 680 | 852 | CS005747 | 816 | 890 |
| AC004646 | CA005749 | 681 | 853 | CS005748 | 817 | 891 |
| AC004647 | CA005750 | 682 | 904 | CS004393 | 800 | 872 |
| AC004648 | CA005751 | 683 | 905 | CS004393 | 800 | 872 |
| AC004649 | CA005752 | 684 | 906 | CS004393 | 800 | 872 |
| AC004816 | CA005749 | 681 | 853 | CS004393 | 800 | 872 |
| AC004817 | CA005958 | 685 | 854 | CS005957 | 818 | 892 |
| AC004818 | CA005959 | 686 | 855 | CS004393 | 800 | 872 |
| AC004819 | CA005959 | 686 | 855 | CS005957 | 818 | 892 |
| AC004820 | CA005958 | 685 | 854 | CS005960 | 819 | 893 |
| AC004821 | CA005958 | 685 | 854 | CS004393 | 800 | 872 |
| AC004837 | CA005976 | 687 | 855 | CS004393 | 800 | 872 |
| AC004838 | CA005977 | 688 | 836 | CS004393 | 800 | 872 |
| AC004908 | CA006068 | 689 | 853 | CS004393 | 800 | 872 |
| AC004915 | CA006074 | 690 | 854 | CS004393 | 800 | 872 |
| AC004916 | CA006075 | 691 | 856 | CS004395 | 801 | 873 |
| AC004917 | CA006076 | 692 | 837 | CS004395 | 801 | 873 |
| AC005191 | CA006343 | 693 | 852 | CS005747 | 816 | 890 |
| AC005192 | CA006345 | 694 | 857 | CS006344 | 820 | 894 |
| AC005193 | CA006347 | 695 | 858 | CS006346 | 821 | 895 |
| AC005195 | CA005407 | 663 | 841 | CS006349 | 822 | 896 |
| AC005196 | CA006350 | 696 | 841 | CS006349 | 822 | 896 |
| AC005206 | CA005958 | 685 | 854 | CS005748 | 817 | 891 |
| AC005233 | CA006068 | 689 | 853 | CS005748 | 817 | 891 |
| AC005236 | CA006383 | 697 | 859 | CS004397 | 802 | 874 |
| AC005249 | CA006350 | 696 | 841 | CS003906 | 788 | 864 |
| AC005944 | CA005056 | 661 | 840 | CS003906 | 788 | 864 |
| AC005945 | CA004538 | 652 | 830 | CS003906 | 788 | 864 |
| AC005991 | CA006074 | 690 | 854 | CS005748 | 817 | 891 |

TABLE 9
Conjugate Duplex ID Numbers Referencing
Position Targeted On TSLP (TSLP) Gene

| | | | Targeted TSLP |
|---|---|---|---|
| Conjugated | | | Gene Position |
| Duplex | AS ID | SS ID | (of SEQ ID NO: 1) |
| AC001714 | AM14179-AS | CS001922 | N/A |
| AC002515 | AM16334-AS | CS003220 | N/A |
| AC003096 | AM19258-AS | CS003898 | 398 |
| AC003097 | AM19260-AS | CS003900 | 410 |
| AC003098 | AM19262-AS | CS003902 | 515 |
| AC003099 | AM19264-AS | CS003904 | 570 |
| AC003100 | AM19266-AS | CS003906 | 571 |
| AC003101 | AM19268-AS | CS003908 | 568 |
| AC003102 | AM18311-AS | CS003904 | 570 |
| AC003128 | AM19335-AS | CS003954 | 520 |
| AC003129 | AM19337-AS | CS003956 | 413 |
| AC003130 | AM19339-AS | CS003958 | 406 |
| AC003252 | AM18285-AS | CS003954 | 520 |
| AC003253 | AM19685-AS | CS003954 | 520 |
| AC003339 | AM19938-AS | CS003954 | 520 |
| AC003340 | AM19939-AS | CS003954 | 520 |
| AC003341 | AM19335-AS | CS004174 | 520 |
| AC003342 | AM19941-AS | CS003954 | 520 |
| AC003343 | AM19942-AS | CS003954 | 520 |
| AC003344 | AM19943-AS | CS003954 | 520 |
| AC003345 | AM19944-AS | CS003954 | 520 |
| AC003346 | AM19945-AS | CS003954 | 520 |
| AC003347 | AM19946-AS | CS003954 | 520 |
| AC003371 | AM19947-AS | CS003906 | 571 |
| AC003372 | AM19266-AS | CS004205 | 571 |
| AC003373 | AM19949-AS | CS003906 | 571 |
| AC003374 | AM19950-AS | CS003906 | 571 |
| AC003375 | AM19951-AS | CS003906 | 571 |
| AC003376 | AM19952-AS | CS003906 | 571 |
| AC003377 | AM19953-AS | CS003906 | 571 |
| AC003378 | AM19954-AS | CS003906 | 571 |
| AC003379 | AM19955-AS | CS003906 | 571 |
| AC003415 | AM20176-AS | CS004244 | 417 |
| AC003416 | AM20176-AS | CS004246 | 417 |
| AC003446 | AM19941-AS | CS004174 | 520 |
| AC003447 | AM19941-AS | CS004280 | 520 |
| AC003448 | AM20299-AS | CS003954 | 520 |
| AC003449 | AM20300-AS | CS003954 | 520 |
| AC003450 | AM20314-AS | CS003954 | 520 |
| AC003451 | AM20315-AS | CS003954 | 520 |
| AC003452 | AM20315-AS | CS004280 | 520 |
| AC003453 | AM20303-AS | CS003954 | 520 |
| AC003454 | AM20308-AS | CS003954 | 520 |
| AC003455 | AM20309-AS | CS003954 | 520 |
| AC003456 | AM20310-AS | CS003954 | 520 |
| AC003457 | AM20304-AS | CS003954 | 520 |
| AC003458 | AM20305-AS | CS003954 | 520 |
| AC003459 | AM20307-AS | CS004291 | 520 |
| AC003511 | AM20487-AS | CS003904 | 570 |
| AC003537 | AM20488-AS | CS003906 | 571 |
| AC003538 | AM20489-AS | CS003906 | 571 |
| AC003539 | AM20490-AS | CS003906 | 571 |
| AC003540 | AM20491-AS | CS003954 | 520 |
| AC003541 | AM19947-AS | CS004392 | 571 |
| AC003542 | AM19947-AS | CS004205 | 571 |
| AC003543 | AM20534-AS | CS004393 | 571 |
| AC003544 | AM20536-AS | CS004395 | 571 |
| AC003545 | AM20538-AS | CS004397 | 571 |
| AC003546 | AM20539-AS | CS004397 | 571 |
| AC003547 | AM20540-AS | CS004397 | 571 |
| AC003567 | CA004416 | CS003904 | 570 |
| AC003568 | CA004416 | CS004420 | 570 |
| AC003569 | CA004417 | CS003904 | 570 |
| AC003570 | CA004417 | CS004420 | 570 |
| AC003571 | CA004418 | CS004420 | 570 |
| AC003572 | CA004418 | CS003904 | 570 |
| AC003597 | CA004450 | CS004395 | 571 |
| AC003598 | CA004451 | CS004393 | 571 |
| AC003601 | CA004452 | CS004395 | 571 |
| AC003602 | CA004453 | CS004393 | 571 |
| AC003659 | CA004518 | CS004397 | 571 |
| AC003660 | CA004519 | CS004395 | 571 |
| AC003843 | CA004288 | CS004174 | 520 |
| AC003924 | CA004834 | CS004393 | 571 |
| AC004077 | CA005033 | CS004393 | 571 |
| AC004078 | CA005034 | CS004393 | 571 |
| AC004079 | CA005037 | CS005036 | 570 |
| AC004082 | CA005032 | CS003906 | 571 |
| AC004083 | CA005035 | CS003904 | 570 |
| AC004358 | CA005404 | CS005405 | 571 |
| AC004361 | CA005404 | CS003906 | 571 |
| AC004363 | CA005407 | CS003906 | 571 |
| AC004373 | CA005413 | CS003906 | 571 |
| AC004374 | CA005414 | CS003906 | 571 |
| AC004375 | CA005415 | CS003906 | 571 |
| AC004376 | CA005410 | CS003906 | 571 |
| AC004377 | CA005411 | CS003906 | 571 |
| AC004378 | CA005412 | CS003906 | 571 |
| AC004565 | CA005630 | CS005657 | 485 |
| AC004566 | CA005632 | CS005658 | 626 |
| AC004567 | CA005634 | CS005659 | 719 |
| AC004568 | CA005636 | CS005660 | 773 |
| AC004569 | CA005638 | CS005661 | 836 |
| AC004570 | CA005640 | CS005662 | 863 |
| AC004571 | CA005642 | CS005663 | 992 |
| AC004572 | CA005644 | CS005664 | 1021 |
| AC004573 | CA005646 | CS005665 | 1040 |
| AC004574 | CA005648 | CS005666 | 1218 |
| AC004644 | CA005746 | CS004393 | 571 |
| AC004645 | CA005746 | CS005747 | 571 |
| AC004646 | CA005749 | CS005748 | 571 |
| AC004647 | CA005750 | CS004393 | 571 |
| AC004648 | CA005751 | CS004393 | 571 |
| AC004649 | CA005752 | CS004393 | 571 |
| AC004816 | CA005749 | CS004393 | 571 |
| AC004817 | CA005958 | CS005957 | 571 |
| AC004818 | CA005959 | CS004393 | 571 |
| AC004819 | CA005959 | CS005957 | 571 |
| AC004820 | CA005958 | CS005960 | 571 |
| AC004821 | CA005958 | CS004393 | 571 |
| AC004837 | CA005976 | CS004393 | 571 |
| AC004838 | CA005977 | CS004393 | 571 |
| AC004908 | CA006068 | CS004393 | 571 |
| AC004915 | CA006074 | CS004393 | 571 |
| AC004916 | CA006075 | CS004395 | 571 |
| AC004917 | CA006076 | CS004395 | 571 |
| AC005191 | CA006343 | CS005747 | 571 |
| AC005192 | CA006345 | CS006344 | 571 |
| AC005193 | CA006347 | CS006346 | 571 |
| AC005195 | CA005407 | CS006349 | 571 |
| AC005196 | CA006350 | CS006349 | 571 |
| AC005206 | CA005958 | CS005748 | 571 |
| AC005233 | CA006068 | CS005748 | 571 |
| AC005236 | CA006383 | CS004397 | 571 |
| AC005249 | CA006350 | CS003906 | 571 |
| AC005944 | CA005056 | CS003906 | 571 |
| AC005945 | CA004538 | CS003906 | 571 |
| AC005991 | CA006074 | CS005748 | 571 |

Duplex ID Nos. AC001714 and AC002515 include a rat-specific sequence designed to target the rat TSLP transcript (NCBI GenBank XM_008772052.2) and does not have homology with the human TSLP gene.

TABLE 10
Conjugate ID Numbers With Chemically Modified Antisense and Sense Strands (including Linkers and Conjugates)

| ACID | Sense Strand (Fully Modified with Conjugated Targeting | SEQ | | SEQ |
|---|---|---|---|---|
| Number | Ligand) (5′→3′) | ID NO: | Antisense Strand (5′→3′) | ID NO: |
| AC001714 | Tri-SM6.1-avb6-(TA14)-gsa_2NaucaaaCfCfUfcacaaauucus(invAb) | 782 | cPrpasGfsasAfuUfuGfuGfaGfgUfuUfgAfuUfsc | 587 |
| AC002515 | Tri-SM6.1-avb6-(TA14)-csugaaacuGfAfGfagaaaugguas(invAb) | 783 | cPrpusAfscsCfaUfuucucUfcAfgUfuUfcasg | 588 |
| AC003096 | Tri-SM6.1-avb6-(TA14)-csa_2NuugccuUfAfCfugaaauccaas(invAb) | 784 | cPrpusUfsgsGfaUfuUfcAfgUfaAfgGfcAfaUfsg | 591 |
| AC003097 | Tri-SM6.1-avb6-(TA14)-gsa_2NaauccaGfAfGfccuaaccuuas(invAb) | 785 | cPrpusAfsasGfgUfuAfgGfcUfcUfgGfaUfuUfsc | 592 |
| AC003098 | Tri-SM6.1-avb6-(TA14)-gsauucggaAfAfCfucagauaaa_2Nus(invAb) | 786 | cPrpasUfsusUfaUfcUfgAfgUfuUfcCfgAfaUfsc | 593 |
| AC003099 | Tri-SM6.1-avb6-(TA14)-a_2NsagucacaAfCfCfaauaaauguas(invAb) | 787 | cPrpusAfscsAfuUfuAfuUfgGfuUfgUfgAfcUfsu | 594 |
| AC003100 | Tri-SM6.1-avb6-(TA14)-asgucacaaCfCfAfauaaaugucus(invAb) | 788 | cPrpasGfsasCfaUfuUfaUfuGfgUfuGfuGfaCfsu | 595 |
| AC003101 | Tri-SM6.1-avb6-(TA14)-gsaaagucaCfAfAfccaauaaa_2Nuas(invAb) | 789 | cPrpusAfsusUfuAfuUfgGfuUfgUfgAfcUfuUfsc | 596 |
| AC003102 | Tri-SM6.1-avb6-(TA14)-a_2NsagucacaAfCfCfaauaaauguas(invAb) | 787 | usAfscsAfuUfuAfuUfgGfuUfgUfgAfcUfsu | 590 |
| AC003128 | Tri-SM6.1-avb6-(TA14)-gsgaaacucAfGfAfuaaaugcuaas(invAb) | 790 | cPrpusUfsasGfcAfuUfuAfuCfuGfaGfuUfuCfsc | 597 |
| AC003129 | Tri-SM6.1-avb6-(TA14)-asuccagagCfCfUfaaccuucaaus(invAb) | 791 | cPrpasUfsusGfaAfgGfuUfaGfgCfuCfuGfgAfsu | 598 |
| AC003130 | Tri-SM6.1-avb6-(TA14)-usa_2NcugaaaUfCfCfagagccuaaas(invAb) | 792 | cPrpusUfsusAfgGfcUfcUfgGfaUfuUfcAfgUfsa | 599 |
| AC003252 | Tri-SM6.1-avb6-(TA14)-gsgaaacucAfGfAfuaaaugcuaas(invAb) | 790 | usUfsasGfcAfuUfuAfuCfuGfaGfuUfuCfsc | 589 |
| AC003253 | Tri-SM6.1-avb6-(TA14)-gsgaaacucAfGfAfuaaaugcuaas(invAb) | 790 | cPrpusUfsaGfcAfuUfuAfuCfuGfaGfuUfuCfsc | 600 |
| AC003339 | Tri-SM6.1-avb6-(TA14)-gsgaaacucAfGfAfuaaaugcuaas(invAb) | 790 | cPrpuUfaGfcAfuUfuAfuCfuGfaGfuUfuCfsc | 601 |
| AC003340 | Tri-SM6.1-avb6-(TA14)-gsgaaacucAfGfAfuaaaugcuaas(invAb) | 790 | cPrpusUfsagcauuuauCfuGfaGfuuucsc | 602 |
| AC003341 | Tri-SM6.1-avb6-(TA14)-gsgaaacucAfgAfuAfaaugcuaas(invAb) | 793 | cPrpusUfsasGfcAfuUfuAfuCfuGfaGfuUfuCfsc | 597 |
| AC003342 | Tri-SM6.1-avb6-(TA14)-gsgaaacucAfGfAfuaaaugcuaas(invAb) | 790 | cPrpusUfsagcauuUfauCfuGfaGfuuucsc | 603 |
| AC003343 | Tri-SM6.1-avb6-(TA14)-gsgaaacucAfGfAfuaaaugcuaas(invAb) | 790 | cPrpusUfsagCfauuuauCfuGfaGfuuucsc | 604 |
| AC003344 | Tri-SM6.1-avb6-(TA14)-gsgaaacucAfGfAfuaaaugcuaas(invAb) | 790 | cPrpusUfsagcaUfuuauCfuGfaGfuuucsc | 605 |
| AC003345 | Tri-SM6.1-avb6-(TA14)-gsgaaacucAfGfAfuaaaugcuaas(invAb) | 790 | cPrpusUfsagcauUfuauCfuGfaGfuuucsc | 606 |
| AC003346 | Tri-SM6.1-avb6-(TA14)-gsgaaacucAfGfAfuaaaugcuaas(invAb) | 790 | cPrpusUfsaGfcAfUfuuauCfuGfaGfuuucsc | 607 |
| AC003347 | Tri-SM6.1-avb6-(TA14)-gsgaaacucAfGfAfuaaaugcuaas(invAb) | 790 | cPrpusdTsagcauuuauCfuGfaGfuuucsc | 608 |
| AC003371 | Tri-SM6.1-avb6-(TA14)-asgucacaaCfCfAfauaaaugucus(invAb) | 788 | cPrpasGfsaCfaUfuUfaUfuGfgUfuGfuGfaCfsu | 609 |
| AC003372 | Tri-SM6.1-avb6-(TA14)-asgucacaaCfcAfaUfaaaugucus(invAb) | 794 | cPrpasGfsasCfaUfuUfaUfuGfgUfuGfuGfaCfsu | 595 |
| AC003373 | Tri-SM6.1-avb6-(TA14)-asgucacaaCfCfAfauaaaugucus(invAb) | 788 | cPrpasGfsacauuuauuGfgUfuGfugacsu | 610 |
| AC003374 | Tri-SM6.1-avb6-(TA14)-asgucacaaCfCfAfauaaaugucus(invAb) | 788 | cPrpasGfsacauuuaUfuGfgUfuGfugacsu | 611 |
| AC003375 | Tri-SM6.1-avb6-(TA14)-asgucacaaCfCfAfauaaaugucus(invAb) | 788 | cPrpasGfsacauuuAfuuGfgUfuGfugacsu | 612 |
| AC003376 | Tri-SM6.1-avb6-(TA14)-asgucacaaCfCfAfauaaaugucus(invAb) | 788 | cPrpasGfsacauUfuauuGfgUfuGfugacsu | 613 |
| AC003377 | Tri-SM6.1-avb6-(TA14)-asgucacaaCfCfAfauaaaugucus(invAb) | 788 | cPrpasGfsacAfUfuuAfuuGfgUfuGfugacsu | 614 |
| AC003378 | Tri-SM6.1-avb6-(TA14)-asgucacaaCfCfAfauaaaugucus(invAb) | 788 | cPrpasGfsaCfaUfUfuauuGfgUfuGfugacsu | 615 |
| AC003379 | Tri-SM6.1-avb6-(TA14)-asgucacaaCfCfAfauaaaugucus(invAb) | 788 | cPrpaGfacauuuaUfuGfgUfuGfugacsu | 616 |
| AC003415 | Tri-SM6.1-avb6-(TA14)-asgagccuaAfCfCfuucaaucucas(invAb) | 795 | cPrpusGfsgsGfaUfuGfaAfgGfuUfaGfgCfuCfsu | 617 |
| AC003416 | Tri-SM6.1-avb6-(TA14)-asgagccuaAfCfCfuucaaucccas(invAb) | 796 | cPrpusGfsgsGfaUfuGfaAfgGfuUfaGfgCfuCfsu | 617 |
| AC003446 | Tri-SM6.1-avb6-(TA14)-gsgaaacucAfgAfuAfaaugcuaas(invAb) | 793 | cPrpusUfsagcauuUfauCfuGfaGfuuucsc | 603 |
| AC003447 | Tri-SM6.1-avb6-(TA14)-gsgaaacucdAgdAudAaaugcuaas(invAb) | 797 | cPrpusUfsagcauuUfauCfuGfaGfuuucsc | 603 |
| AC003448 | Tri-SM6.1-avb6-(TA14)-gsgaaacucAfGfAfuaaaugcuaas(invAb) | 790 | cPrpusUfsagdCauuuauCfuGfaguuucsc | 618 |
| AC003449 | Tri-SM6.1-avb6-(TA14)-gsgaaacucAfGfAfuaaaugcuaas(invAb) | 790 | cPrpusUfsagcauudTauCfuGfaguuucsc | 619 |
| AC003450 | Tri-SM6.1-avb6-(TA14)-gsgaaacucAfGfAfuaaaugcuaas(invAb) | 790 | cPrpusdTsagcauudTauCfuGfaguuucsc | 629 |
| AC003451 | Tri-SM6.1-avb6-(TA14)-gsgaaacucAfGfAfuaaaugcuaas(invAb) | 790 | cPrpusdTsagcauudTauCfuGfadGuuucsc | 630 |
| AC003452 | Tri-SM6.1-avb6-(TA14)-gsgaaacucdAgdAudAaaugcuaas(invAb) | 797 | cPrpusdTsagcauudTauCfuGfadGuuucsc | 630 |
| AC003453 | Tri-SM6.1-avb6-(TA14)-gsgaaacucAfGfAfuaaaugcuaas(invAb) | 790 | cPrpusUfsagcauuuauCfuGfaguuucsc | 622 |
| AC003454 | Tri-SM6.1-avb6-(TA14)-gsgaaacucAfGfAfuaaaugcuaas(invAb) | 790 | cPrpusUfsagcauUfUfauCfuGfaguuucsc | 626 |
| AC003455 | Tri-SM6.1-avb6-(TA14)-gsgaaacucAfGfAfuaaaugcuaas(invAb) | 790 | cPrpusUfsagcauuUfauCfuGfaguuucsc | 627 |
| AC003456 | Tri-SM6.1-avb6-(TA14)-gsgaaacucAfGfAfuaaaugcuaas(invAb) | 790 | cPrpusUfsagCfauuUfauCfuGfaguuucsc | 628 |
| AC003457 | Tri-SM6.1-avb6-(TA14)-gsgaaacucAfGfAfuaaaugcuaas(invAb) | 790 | cPrpusUfsagcaTGNAuUfauCfuGfaGfuuucsc | 623 |
| AC003458 | Tri-SM6.1-avb6-(TA14)-gsgaaacucAfGfAfuaaaugcuaas(invAb) | 790 | cPrpusUfsagcaU_{UNA}uUfauCfuGfaGfuuucsc | 624 |
| AC003459 | Tri-SM6.1-avb6-(TA14)-gsa_2NacucAfGfAfuaaaugcuaas(invAb) | 798 | cPrpusUfsagcauUfuauCfuGfaGfuusc | 625 |
| AC003511 | Tri-SM6.1-avb6-(TA14)-a_2NsagucacaAfCfCfaauaaauguas(invAb) | 787 | cPrpusAfscAfuUfuAfuUfgGfuUfgUfgAfcUfsu | 631 |
| AC003537 | Tri-SM6.1-avb6-(TA14)-asgucacaaCfCfAfauaaaugucus(invAb) | 788 | cPrpasGfsacAfUfuuauuGfgUfuGfugacsu | 632 |
| AC003538 | Tri-SM6.1-avb6-(TA14)-asgucacaaCfCfAfauaaaugucus(invAb) | 788 | cPrpasGfsacAfUfuuauuGfgUfugugacsu | 633 |
| AC003539 | Tri-SM6.1-avb6-(TA14)-asgucacaaCfCfAfauaaaugucus(invAb) | 788 | cPrpasGfsacAfuuuauuGfgUfuGfugacsu | 634 |
| AC003540 | Tri-SM6.1-avb6-(TA14)-gsgaaacucAfGfAfuaaaugcuaas(invAb) | 790 | cPrpusUfsagCfAfuuUfauCfuGfaGfuuucsc | 635 |
| AC003541 | Tri-SM6.1-avb6-(TA14)-asgucacAfaCfcAfauaaaugucus(invAb) | 799 | cPrpasGfsaCfaUfuUfaUfuGfgUfuGfuGfaCfsu | 609 |
| AC003542 | Tri-SM6.1-avb6-(TA14)-asgucacaaCfcAfaUfaaaugucus(invAb) | 794 | cPrpasGfsaCfaUfuUfaUfuGfgUfuGfuGfaCfsu | 609 |
| AC003543 | Tri-SM6.1-avb6-(TA14)-gsgucacaaCfCfAfauaaaugucus(invAb) | 800 | cPrpasGfsaCfaUfuUfaUfuGfgUfuGfuGfaCfsc | 636 |
| AC003544 | Tri-SM6.1-avb6-(TA14)-gsgucacaaCfCfAfauaaaugucas(invAb) | 801 | cPrpusGfsaCfaUfuUfaUfuGfgUfuGfuGfaCfsc | 637 |
| AC003545 | Tri-SM6.1-avb6-(TA14)-asgucacaaCfCfAfauaaaugucas(invAb) | 802 | cPrpusGfsaCfaUfuUfaUfuGfgUfuGfuGfaCfsu | 638 |
| AC003546 | Tri-SM6.1-avb6-(TA14)-asgucacaaCfCfAfauaaaugucas(invAb) | 802 | cPrpusGfsacauuuaUfuGfgUfuGfugacsu | 639 |
| AC003547 | Tri-SM6.1-avb6-(TA14)-asgucacaaCfCfAfauaaaugucas(invAb) | 802 | cPrpusGfsacauUfuauuGfgUfuGfugacsu | 640 |
| AC003567 | Tri-SM6.1-avb6-(TA14)-a_2NsagucacaAfCfCfaauaaauguas(invAb) | 787 | cPrpusAfscauuuauUfgGfuUfgUfgacusu | 643 |
| AC003568 | Tri-SM6.1-avb6-(TA14)-a_2NsagucacaAfcCfaAfuaaauguas(invAb) | 803 | cPrpusAfscauuuauUfgGfuUfgUfgacusu | 643 |
| AC003569 | Tri-SM6.1-avb6-(TA14)-a_2NsagucacaAfCfCfaauaaauguas(invAb) | 787 | cPrpusAfscaUfUfuaUfugGfuUfgUfgacusu | 644 |
| AC003570 | Tri-SM6.1-avb6-(TA14)-a_2NsagucacaAfcCfaAfuaaauguas(invAb) | 803 | cPrpusAfscaUfUfuaUfugGfuUfgUfgacusu | 644 |
| AC003571 | Tri-SM6.1-avb6-(TA14)-a_2NsagucacaAfcCfaAfuaaauguas(invAb) | 803 | cPrpusAfscauuUfauugGfuUfgUfgacusu | 645 |
| AC003572 | Tri-SM6.1-avb6-(TA14)-a_2NsagucacaAfCfCfaauaaauguas(invAb) | 787 | cPrpusAfscauuUfauugGfuUfgUfgacusu | 645 |
| AC003597 | Tri-SM6.1-avb6-(TA14)-gsgucacaaCfCfAfauaaaugucas(invAb) | 801 | cPrpusGfsacauUfuauuGfgUfuGfugacsc | 646 |
| AC003598 | Tri-SM6.1-avb6-(TA14)-gsgucacaaCfCfAfauaaaugucus(invAb) | 800 | cPrpasGfsacauUfuauuGfgUfuGfugacsc | 647 |
| AC003601 | Tri-SM6.1-avb6-(TA14)-gsgucacaaCfCfAfauaaaugucas(invAb) | 801 | cPrpusGfsacauuuaUfuGfgUfuGfugacsc | 648 |
| AC003602 | Tri-SM6.1-avb6-(TA14)-gsgucacaaCfCfAfauaaaugucus(invAb) | 800 | cPrpasGfsacauuuaUfuGfgUfuGfugacsc | 649 |
| AC003659 | Tri-SM6.1-avb6-(TA14)-asgucacaaCfCfAfauaaaugucas(invAb) | 802 | cPrpuGfacauuuaUfuGfgUfuGfugacsu | 650 |
| AC003660 | Tri-SM6.1-avb6-(TA14)-gsgucacaaCfCfAfauaaaugucas(invAb) | 801 | cPrpuGfacauuuaUfuGfgUfuGfugacsc | 651 |
| AC003843 | Tri-SM6.1-avb6-(TA14)-gsgaaacucAfgAfuAfaaugcuaas(invAb) | 793 | cPrpusUfsagCfauuUfauCfuGfaguuucsc | 642 |
| AC003924 | Tri-SM6.1-avb6-(TA14)-gsgucacaaCfCfAfauaaaugucus(invAb) | 800 | cPrpasGfsacauuuAfuuGfgUfuGfugacsc | 655 |
| AC004077 | Tri-SM6.1-avb6-(TA14)-gsgucacaaCfCfAfauaaaugucus(invAb) | 800 | cPrpasGfsacauU_{UNA}uaUfuGfgUfuGfugacsc | 657 |
| AC004078 | Tri-SM6.1-avb6-(TA14)-gsgucacaaCfCfAfauaaaugucus(invAb) | 800 | cPrpasGfsacauTGNAuaUfuGfgUfuGfugacsc | 658 |
| AC004079 | Tri-SM6.1-avb6-(TA14)-gsagucacaAfCfCfaauaaauguas(invAb) | 804 | cPrpusAfscauuU_{UNA}auUfgGfuUfgUfgacusc | 660 |
| AC004082 | Tri-SM6.1-avb6-(TA14)-asgucacaaCfCfAfauaaaugucus(invAb) | 788 | cPrpasGfsacauU_{UNA}uaUfuGfgUfuGfugacsu | 656 |
| AC004083 | Tri-SM6.1-avb6-(TA14)-a_2NsagucacaAfCfCfaauaaauguas(invAb) | 787 | cPrpusAfscauuU_{UNA}auUfgGfuUfgUfgacusu | 659 |
| AC004358 | Tri-SM6.1-avb6-(TA14)-asgucacaaCfCfAfauaaauguccs(invAb) | 805 | cPrpisGfsacauuuaUfuGfgUfuGfugacsu | 662 |
| AC004361 | Tri-SM6.1-avb6-(TA14)-asgucacaaCfCfAfauaaaugucus(invAb) | 788 | cPrpisGfsacauuuaUfuGfgUfuGfugacsu | 662 |
| AC004363 | Tri-SM6.1-avb6-(TA14)-asgucacaaCfCfAfauaaaugucus(invAb) | 788 | cPrpasGfsacguuuaUfuGfgUfuGfugacsu | 663 |
| AC004373 | Tri-SM6.1-avb6-(TA14)-asgucacaaCfCfAfauaaaugucus(invAb) | 788 | cPrpasGfsacaTGNAuuaUfuGfgUfuGfugacsu | 667 |
| AC004374 | Tri-SM6.1-avb6-(TA14)-asgucacaaCfCfAfauaaaugucus(invAb) | 788 | cPrpasGfsacauuTGNAaUfuGfgUfuGfugacsu | 668 |
| AC004375 | Tri-SM6.1-avb6-(TA14)-asgucacaaCfCfAfauaaaugucus(invAb) | 788 | cPrpasGfsacauuU_{UNA}aUfuGfgUfuGfugacsu | 669 |
| AC004376 | Tri-SM6.1-avb6-(TA14)-asgucacaaCfCfAfauaaaugucus(invAb) | 788 | cPrpasGfsacauuuaUfuGfgUfuGfugacssu | 664 |
| AC004377 | Tri-SM6.1-avb6-(TA14)-asgucacaaCfCfAfauaaaugucus(invAb) | 788 | cPrpaGfacauuuaUfuGfgUfuGfugascsu | 665 |
| AC004378 | Tri-SM6.1-avb6-(TA14)-asgucacaaCfCfAfauaaaugucus(invAb) | 788 | cPrpaGfacauuuaUfuGfgUfuGfugacssu | 666 |
| AC004565 | Tri-SM6.1-avb6-(TA14)-asaggcugcCfUfUfagcuaucugas(invAb) | 806 | cPrpusCfsaGfaUfagcuaAfgGfcAfgCfcusu | 670 |
| AC004566 | Tri-SM6.1-avb6-(TA14)-csgcuucaaUfCfGfaccuuuacuas(invAb) | 807 | cPrpusAfsgUfaAfaggucGfaUfuGfaAfgcsg | 671 |
| AC004567 | Tri-SM6.1-avb6-(TA14)-gsa_2NaacauuAfAfCfucuaacuguas(invAb) | 808 | cPrpusAfscAfgUfuagagUfuAfaUfgUfuusc | 672 |
| AC004568 | Tri-SM6.1-avb6-(TA14)-a_2NscagaagaGfUfUfucuuaacuuas(invAb) | 809 | cPrpusAfsaGfuUfaagaaAfcUfcUfuCfugsu | 673 |
| AC004569 | Tri-SM6.1-avb6-(TA14)-gsuacuacuCfCfUfcaaauguugas(invAb) | 810 | cPrpusCfsaAfcAfuuugaGfgAfgUfaGfuasc | 674 |
| AC004570 | Tri-SM6.1-avb6-(TA14)-csuuccauaAfCfAfuugaugacuas(invAb) | 811 | cPrpusAfsgUfcAfucaauGfuUfaUfgGfaasg | 675 |
| AC004571 | Tri-SM6.1-avb6-(TA14)-gscaaugauAfGfCfaccuaaacuus(invAb) | 812 | cPrpasAfsgUfuUfaggugCfuAfuCfaUfugsc | 676 |
| AC004572 | Tri-SM6.1-avb6-(TA14)-a_2NsagacagaCfAfUfuccuucuacas(invAb) | 813 | cPrpusGfsuAfgAfaggaaUfgUfcUfgUfcusu | 677 |
| AC004573 | Tri-SM6.1-avb6-(TA14)-csa_2NuguaauGfAfCfacuucuuguas(invAb) | 814 | cPrpusAfscAfaGfaagugUfcAfuUfaCfausg | 678 |
| AC004574 | Tri-SM6.1-avb6-(TA14)-a_2NsuacaaguAfGfAfuccugagaaas(invAb) | 815 | cPrpusUfsuCfuCfaggauCfuAfcUfuGfuasu | 679 |
| AC004644 | Tri-SM6.1-avb6-(TA14)-gsgucacaaCfCfAfauaaaugucus(invAb) | 800 | cPrpasGfsacauuugUfuGfgUfuGfugacsc | 680 |
| AC004645 | Tri-SM6.1-avb6-(TA14)-gsgucacaaCfCfAfacaaaugucus(invAb) | 816 | cPrpasGfsacauuugUfuGfgUfuGfugacsc | 680 |
| AC004646 | Tri-SM6.1-avb6-(TA14)-gsgucacaaCfCfAfauaaacgucus(invAb) | 817 | cPrpasGfsacguuuaUfuGfgUfuGfugacsc | 681 |
| AC004647 | Tri-SM6.1-avb6-(TA14)-gsgucacaaCfCfAfauaaaugucus(invAb) | 800 | cPrpasGfsacguuuaUfdTGfgUfuGfugacsc | 682 |
| AC004648 | Tri-SM6.1-avb6-(TA14)-gsgucacaaCfCfAfauaaaugucus(invAb) | 800 | cPrpasGfsacauuuaUfdTGfgUfuGfugacsc | 683 |
| AC004649 | Tri-SM6.1-avb6-(TA14)-gsgucacaaCfCfAfauaaaugucus(invAb) | 800 | cPrpasGfsacauuuadTuGfgUfuGfugacsc | 684 |
| AC004816 | Tri-SM6.1-avb6-(TA14)-gsgucacaaCfCfAfauaaaugucus(invAb) | 800 | cPrpasGfsacguuuaUfuGfgUfuGfugacsc | 681 |
| AC004817 | Tri-SM6.1-avb6-(TA14)-gsgucacaaCfCfAfauaaauguccs(invAb) | 818 | cPrpisGfsacguuuaUfuGfgUfuGfugacsc | 685 |
| AC004818 | Tri-SM6.1-avb6-(TA14)-gsgucacaaCfCfAfauaaaugucus(invAb) | 800 | cPrpisGfsacauuuaUfuGfgUfuGfugacsc | 686 |
| AC004819 | Tri-SM6.1-avb6-(TA14)-gsgucacaaCfCfAfauaaauguccs(invAb) | 818 | cPrpisGfsacauuuaUfuGfgUfuGfugacsc | 686 |
| AC004820 | Tri-SM6.1-avb6-(TA14)-gsgucacaaCfCfAfauaaacguccs(invAb) | 819 | cPrpisGfsacguuuaUfuGfgUfuGfugacsc | 685 |
| AC004821 | Tri-SM6.1-avb6-(TA14)-gsgucacaaCfCfAfauaaaugucus(invAb) | 800 | cPrpisGfsacguuuaUfuGfgUfuGfugacsc | 685 |
| AC004837 | Tri-SM6.1-avb6-(TA14)-gsgucacaaCfCfAfauaaaugucus(invAb) | 800 | cPrpisGfsacauuuaUfuGfgUfuGfugacssc | 687 |
| AC004838 | Tri-SM6.1-avb6-(TA14)-gsgucacaaCfCfAfauaaaugucus(invAb) | 800 | cPrpasGfsacauuuaUfuGfgUfuGfugacssc | 688 |
| AC004908 | Tri-SM6.1-avb6-(TA14)-gsgucacaaCfCfAfauaaaugucus(invAb) | 800 | cPrpasGfsacguuuaUfuGfgUfuGfugacssc | 689 |
| AC004915 | Tri-SM6.1-avb6-(TA14)-gsgucacaaCfCfAfauaaaugucus(invAb) | 800 | cPrpisGfsacguuuaUfuGfgUfuGfugacssc | 690 |
| AC004916 | Tri-SM6.1-avb6-(TA14)-gsgucacaaCfCfAfauaaaugucas(invAb) | 801 | cPrpusGfsacguuuaUfuGfgUfuGfugacssc | 691 |
| AC004917 | Tri-SM6.1-avb6-(TA14)-gsgucacaaCfCfAfauaaaugucas(invAb) | 801 | cPrpusGfsacauuuaUfuGfgUfuGfugacssc | 692 |
| AC005191 | Tri-SM6.1-avb6-(TA14)-gsgucacaaCfCfAfacaaaugucus(invAb) | 816 | cPrpasGfsacauuugUfuGfgUfuGfugacssc | 693 |
| AC005192 | Tri-SM6.1-avb6-(TA14)-asgucacaaCfCfAfacaaaugucus(invAb) | 820 | cPrpasGfsacauuugUfuGfgUfuGfugacssu | 694 |
| AC005193 | Tri-SM6.1-avb6-(TA14)-gsgucacaaCfCfAfacaaaugucas(invAb) | 821 | cPrpusGfsacauuugUfuGfgUfuGfugacssc | 695 |
| AC005195 | Tri-SM6.1-avb6-(TA14)-asgucacaaCfCfAfauaaacgucus(invAb) | 822 | cPrpasGfsacguuuaUfuGfgUfuGfugacsu | 663 |
| AC005196 | Tri-SM6.1-avb6-(TA14)-asgucacaaCfCfAfauaaacgucus(invAb) | 822 | cPrpasGfsacguuuaUfuGfgUfuGfugacssu | 696 |
| AC005206 | Tri-SM6.1-avb6-(TA14)-gsgucacaaCfCfAfauaaacgucus(invAb) | 817 | cPrpisGfsacguuuaUfuGfgUfuGfugacsc | 685 |
| AC005233 | Tri-SM6.1-avb6-(TA14)-gsgucacaaCfCfAfauaaacgucus(invAb) | 817 | cPrpasGfsacguuuaUfuGfgUfuGfugacssc | 689 |
| AC005236 | Tri-SM6.1-avb6-(TA14)-asgucacaaCfCfAfauaaaugucas(invAb) | 802 | cPrpusGfsacguuuaUfuGfgUfuGfugacsu | 697 |
| AC005249 | Tri-SM6.1-avb6-(TA14)-asgucacaaCfCfAfauaaaugucus(invAb) | 788 | cPrpasGfsacguuuaUfuGfgUfuGfugacssu | 696 |
| AC005944 | Tri-SM6.1-avb6-(TA14)-asgucacaaCfCfAfauaaaugucus(invAb) | 788 | isGfsacauuuaUfuGfgUfuGfugacsu | 661 |
| AC005945 | Tri-SM6.1-avb6-(TA14)-asgucacaaCfCfAfauaaaugucus(invAb) | 788 | asGfsacauuuaUfuGfgUfuGfugacsu | 652 |
| AC005991 | Tri-SM6.1-avb6-(TA14)-gsgucacaaCfCfAfauaaacgucus(invAb) | 817 | cPrpisGfsacguuuaUfuGfgUfuGfugacssc | 690 |

In some embodiments, a TSLP RNAi agent is prepared or provided as a salt, mixed salt, or a free-acid. In some embodiments, a TSLP RNAi agent is prepared or provided as a pharmaceutically acceptable salt. In some embodiments, a TSLP RNAi agent is prepared or provided as a pharmaceutically acceptable sodium or potassium salt. In some embodiments, a TSLP RNAi agent is prepared or provided as a pharmaceutically acceptable sodium salt. The RNAi agents described herein, upon delivery to a cell expressing an TSLP gene, inhibit or knockdown expression of one or more TSLP genes in vivo and/or in vitro.

### Targeting Groups, Linking Groups, Pharmacokinetic/Pharmacodynamic (PK/PD) Modulators, and Delivery Vehicles

In some embodiments, a TSLP RNAi agent contains or is conjugated to one or more non-nucleotide groups including, but not limited to, a targeting group, a linking group, a pharmacokinetic/pharmacodynamic (PK/PD) modulator, a delivery polymer, or a delivery vehicle. The non-nucleotide group can enhance targeting, delivery, or attachment of the RNAi agent. The non-nucleotide group can be covalently linked to the 3′ and/or 5′ end of either the sense strand and/or the antisense strand. In some embodiments, a TSLP RNAi agent contains a non-nucleotide group linked to the 3′ and/or 5′ end of the sense strand. In some embodiments, a non-nucleotide group is linked to the 5′ end of a TSLP RNAi agent sense strand. A non-nucleotide group can be linked directly or indirectly to the RNAi agent via a linker/linking group. In some embodiments, a non-nucleotide group is linked to the RNAi agent via a labile, cleavable, or reversible bond or linker.

In some embodiments, a non-nucleotide group enhances the pharmacokinetic or biodistribution properties of an RNAi agent or conjugate to which it is attached to improve cell- or tissue-specific distribution and cell-specific uptake of the conjugate. In some embodiments, a non-nucleotide group enhances endocytosis of the RNAi agent.

Targeting groups or targeting moieties enhance the pharmacokinetic or biodistribution properties of a conjugate or RNAi agent to which they are attached to improve cell-specific (including, in some cases, organ specific) distribution and cell-specific (or organ specific) uptake of the conjugate or RNAi agent. A targeting group can be monovalent, divalent, trivalent, tetravalent, or have higher valency for the target to which it is directed. Representative targeting groups include, without limitation, compounds with affinity to cell surface molecule, cell receptor ligands, hapten, antibodies, monoclonal antibodies, antibody fragments, and antibody mimics with affinity to cell surface molecules. In some embodiments, a targeting group is linked to an RNAi agent using a linker, such as a PEG linker or one, two, or three abasic and/or ribitol (abasic ribose) residues, which in some instances can serve as linkers.

A targeting group, with or without a linker, can be attached to the 5′ or 3′ end of any of the sense and/or antisense strands disclosed in Tables 2, 3, 4, 5, 6, and 10. A linker, with or without a targeting group, can be attached to the 5′ or 3′ end of any of the sense and/or antisense strands disclosed in Tables 2, 3, 4, 5, 6, and 10.

The TSLP RNAi agents described herein can be synthesized having a reactive group, such as an amino group (also referred to herein as an amine), at the 5′-terminus and/or the 3′-terminus. The reactive group can be used subsequently to attach a targeting moiety using methods typical in the art.

For example, in some embodiments, the TSLP RNAi agents disclosed herein are synthesized having an NH₂-C₆ group at the 5′-terminus of the sense strand of the RNAi agent. The terminal amino group subsequently can be reacted to form a conjugate with, for example, a group that includes an αvβ6 integrin targeting ligand. In some embodiments, the TSLP RNAi agents disclosed herein are synthesized having one or more alkyne groups at the 5′-terminus of the sense strand of the RNAi agent. The terminal alkyne group(s) can subsequently be reacted to form a conjugate with, for example, a group that includes an αvβ6 integrin targeting ligand.

In some embodiments, a targeting group comprises an integrin targeting ligand. In some embodiments, an integrin targeting ligand is an αvβ6 integrin targeting ligand. The use of an αvβ6 integrin targeting ligand facilitates cell-specific targeting to cells having αvβ6 on its respective surface, and binding of the integrin targeting ligand can facilitate entry of the therapeutic agent, such as an RNAi agent, to which it is linked, into cells such as epithelial cells, including pulmonary epithelial cells and renal epithelial cells. Integrin targeting ligands can be monomeric or monovalent (e.g., having a single integrin targeting moiety) or multimeric or multivalent (e.g., having multiple integrin targeting moieties). The targeting group can be attached to the 3′ and/or 5′ end of the RNAi oligonucleotide using methods known in the art. The preparation of targeting groups, such as αvβ6 integrin targeting ligands, is described, for example, in International Patent Application Publication No. WO 2018/085415 and in International Patent Application Publication No. WO 2019/089765, the contents of each of which are incorporated herein in its entirety.

In some embodiments, targeting groups are linked to the TSLP RNAi agents without the use of an additional linker. In some embodiments, the targeting group is designed having a linker readily present to facilitate the linkage to a TSLP RNAi agent. In some embodiments, when two or more RNAi agents are included in a composition, the two or more RNAi agents can be linked to their respective targeting groups using the same linkers. In some embodiments, when two or more RNAi agents are included in a composition, the two or more RNAi agents are linked to their respective targeting groups using different linkers.

In some embodiments, a linking group is conjugated to the RNAi agent. The linking group facilitates covalent linkage of the agent to a targeting group, pharmacokinetic modulator, delivery polymer, or delivery vehicle. The linking group can be linked to the 3′ and/or the 5′ end of the RNAi agent sense strand or antisense strand. In some embodiments, the linking group is linked to the RNAi agent sense strand. In some embodiments, the linking group is conjugated to the 5′ or 3′ end of an RNAi agent sense strand. In some embodiments, a linking group is conjugated to the 5′ end of an RNAi agent sense strand. Examples of linking groups, include but are not limited to: C6-SS-C6, 6-SS-6, reactive groups such a primary amines (e.g., NH2-C6) and alkynes, alkyl groups, abasic residues/nucleotides, amino acids, tri-alkyne functionalized groups, ribitol, and/or PEG groups. Examples of certain linking groups are provided in Table 11.

A linker or linking group is a connection between two atoms that links one chemical group (such as an RNAi agent) or segment of interest to another chemical group (such as a targeting group, pharmacokinetic modulator, or delivery polymer) or segment of interest via one or more covalent bonds. A labile linkage contains a labile bond. A linkage can optionally include a spacer that increases the distance between the two joined atoms. A spacer may further add flexibility and/or length to the linkage. Spacers include, but are not be limited to, alkyl groups, alkenyl groups, alkynyl groups, aryl groups, aralkyl groups, aralkenyl groups, and aralkynyl groups; each of which can contain one or more heteroatoms, heterocycles, amino acids, nucleotides, and saccharides. Spacer groups are well known in the art and the preceding list is not meant to limit the scope of the description. In some embodiments, a TSLP RNAi agent is conjugated to a polyethylene glycol (PEG) moiety, or to a hydrophobic group having 12 or more carbon atoms, such as a cholesterol or palmitoyl group.

In some embodiments, a TSLP RNAi agent is linked to one or more pharmacokinetic/pharmacodynamic (PK/PD) modulators. PK/PD modulators can increase circulation time of the conjugated drug and/or increase the activity of the RNAi agent through improved cell receptor binding, improved cellular uptake, and/or other means. Various PK/PD modulators suitable for use with RNAi agents are known in the art. In some embodiments, the PK/PD modulatory can be cholesterol or cholesteryl derivatives, or in some circumstances a PK/PD modulator can be comprised of alkyl groups, alkenyl groups, alkynyl groups, aryl groups, aralkyl groups, aralkenyl groups, or aralkynyl groups, each of which may be linear, branched, cyclic, and/or substituted or unsubstituted. In some embodiments, the location of attachment for these moieties is at the 5′ or 3′ end of the sense strand, at the 2′ position of the ribose ring of any given nucleotide of the sense strand, and/or attached to the phosphate or phosphorothioate backbone at any position of the sense strand.

Any of the TSLP RNAi agent nucleotide sequences listed in Tables 2, 3, 4, 5, 6, and 10, whether modified or unmodified, can contain 3′ and/or 5′ targeting group(s), linking group(s), and/or PK/PD modulator(s). Any of the TSLP RNAi agent sequences listed in Tables 3, 4, 5, 6, and 10, or are otherwise described herein, which contain a 3′ or 5′ targeting group, linking group, and/or PK/PD modulator can alternatively contain no 3′ or 5′ targeting group, linking group, or PK/PD modulator, or can contain a different 3′ or 5′ targeting group, linking group, or pharmacokinetic modulator including, but not limited to, those depicted in Table 11. Any of the TSLP RNAi agent duplexes listed in Tables 7A, 7B, 8, 9 and 10, whether modified or unmodified, can further comprise a targeting group or linking group, including, but not limited to, those depicted in Table 11, and the targeting group or linking group can be attached to the 3′ or 5′ terminus of either the sense strand or the antisense strand of the TSLP RNAi agent duplex.

Examples of certain modified nucleotides, capping moieties, and linking groups are provided in Table 11.

TABLE 11
Structures Representing Various Modified Nucleotides, Capping Moieties, and
Linking Groups (wherein [symbol] indicates the point of connection)

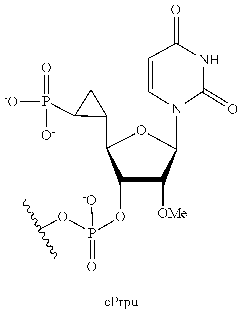

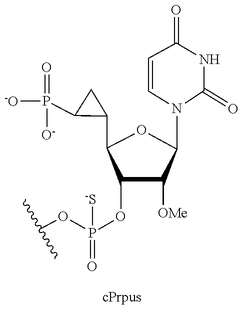

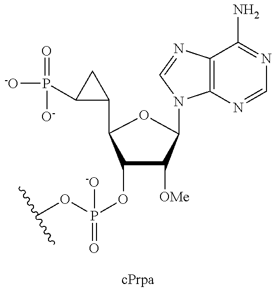

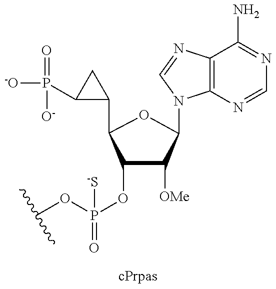

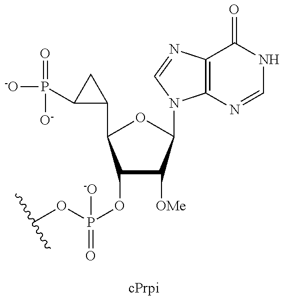

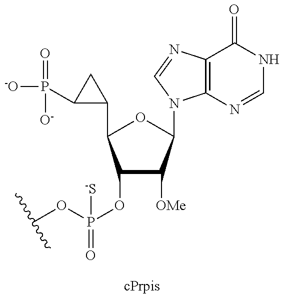

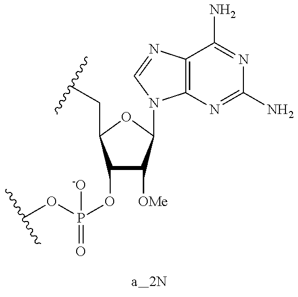

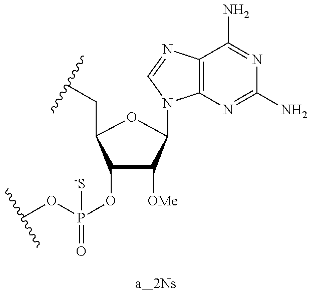

When positioned internally:

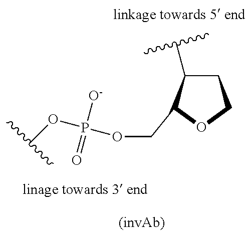

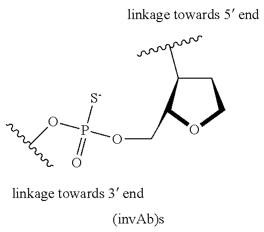

When positioned at the 3′ terminal end:

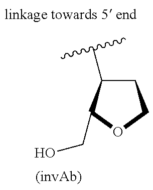

When positioned at the 3′ terminal end:

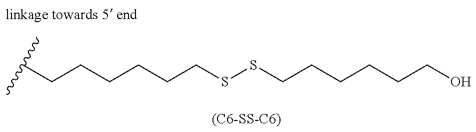 [SMILES: *CCCCCCSSCCCCCCO]

When positioned internally:

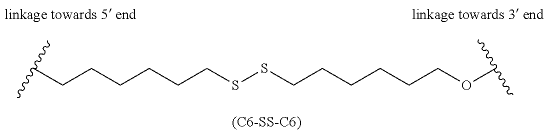 [SMILES: *CCCCCCSSCCCCCCO*]

When position at the 3′ terminal end:

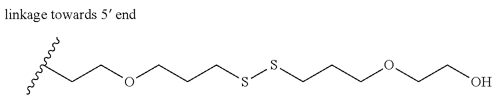 [SMILES: *CCOCCCSSCCCOCCO]

(6-SS-6)
When positioned internally:

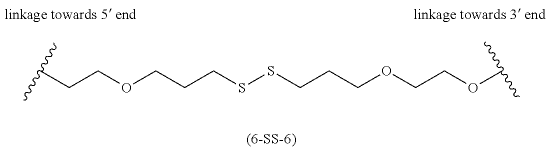 [SMILES: *CCOCCCSSCCCOCCO*]

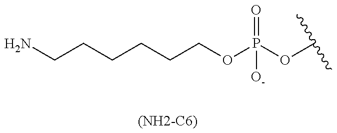

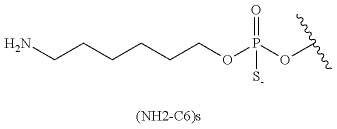

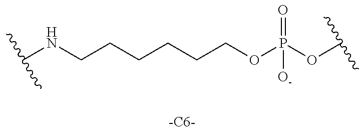

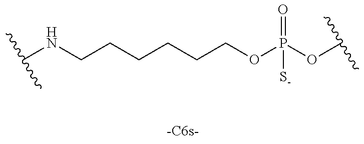

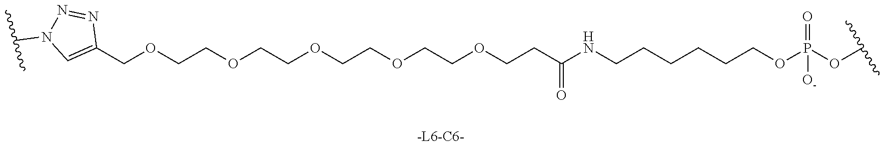

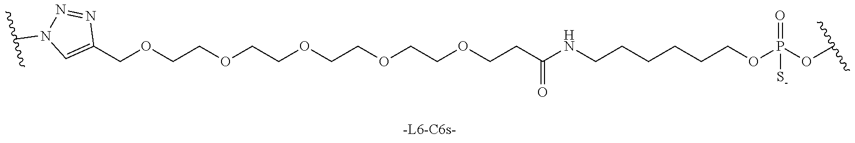

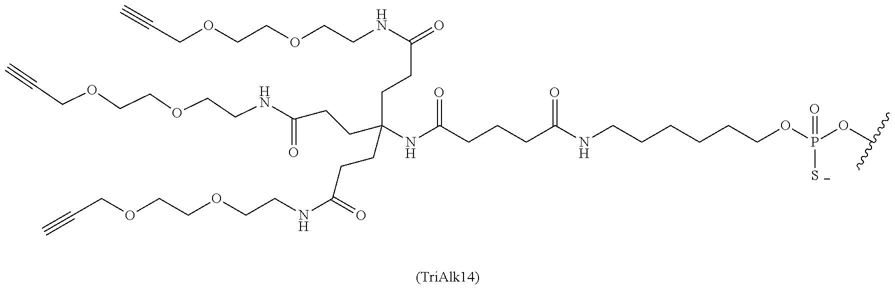 [SMILES: *OP(=O)([S-])OCCCCCCNC(=O)CCCC(=O)NC(CCC(=O)NCCOCCOCC#C)(CCC(=O)NCCOCCOCC#C)CCC(=O)NCCOCCOCC#C]

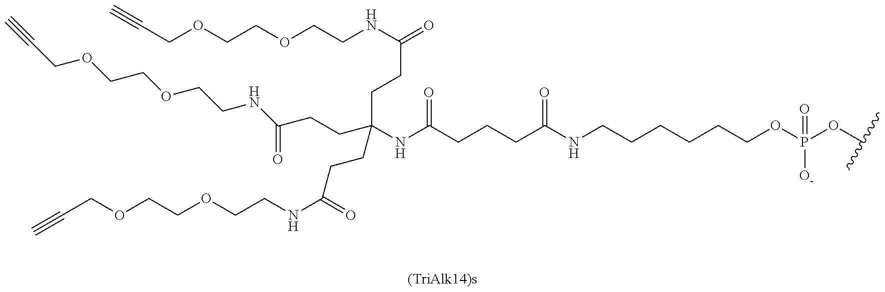

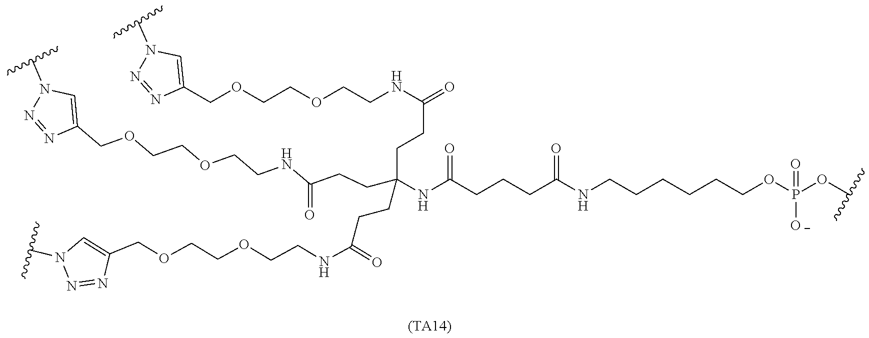

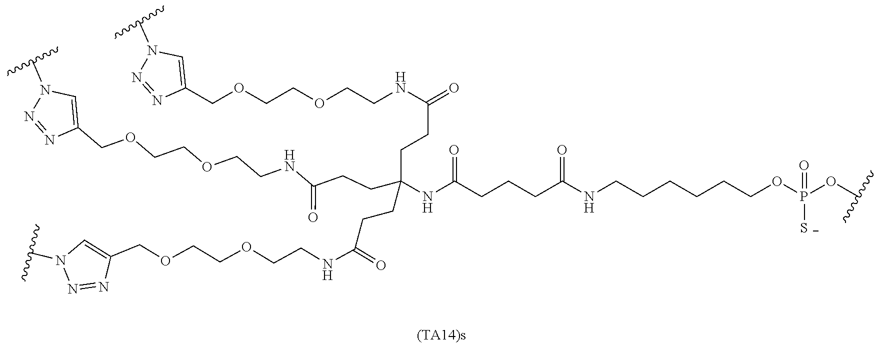 [SMILES: *OP(=O)([S-])OCCCCCCNC(=O)CCCC(=O)NC(CCC(=O)NCCOCCOCc1cn(*)nn1)(CCC(=O)NCCOCCOCc1cn(*)nn1)CCC(=O)NCCOCCOCc1cn(*)nn1]

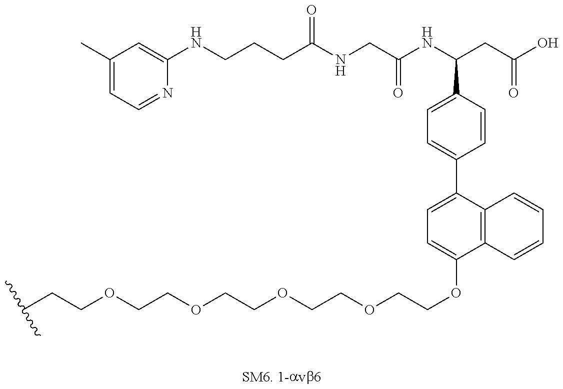 [SMILES: *CCOCCOCCOCCOCCOc1ccc(-c2ccc([C@H](CC(=O)O)NC(=O)CNC(=O)CCCNc3cc(C)ccn3)cc2)c2ccccc12]

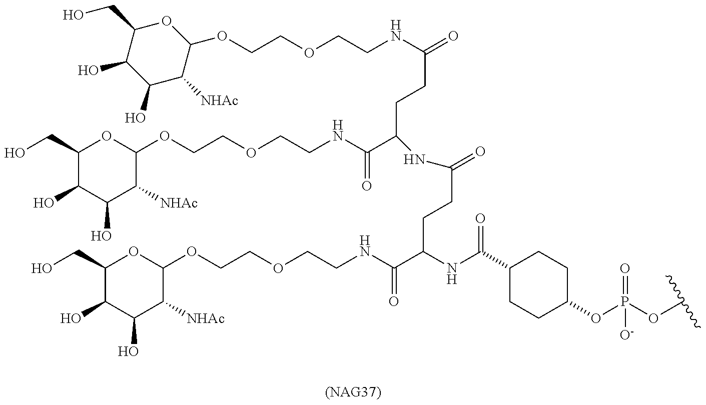 [SMILES: *OP(=O)([O-])O[C@H]1CC[C@@H](C(=O)NC(CCC(=O)NC(CCC(=O)NCCOCCOC2O[C@H](CO)[C@H](O)[C@H](O)[C@H]2NC(C)=O)C(=O)NCCOCCOC2O[C@H](CO)[C@H](O)[C@H](O)[C@H]2NC(C)=O)C(=O)NCCOCCOC2O[C@H](CO)[C@H](O)[C@H](O)[C@H]2NC(C)=O)CC1]

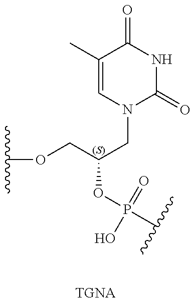

Alternatively, other linking groups known in the art may be used. In many instances, linking groups can be commercially acquired or alternatively, are incorporated into commercially available nucleotide phosphoramidites. (See. e.g., International Patent Application Publication No. WO 2019/161213, which is incorporated herein by reference in its entirety).

In some embodiments, a TSLP RNAi agent is delivered without being conjugated to a targeting ligand or pharmacokinetic/pharmacodynamic (PK/PD) modulator (referred to as being “naked” or a “naked RNAi agent”).

In some embodiments, a TSLP RNAi agent is conjugated to a targeting group, a linking group, a PK modulator, and/or another non-nucleotide group to facilitate delivery of the TSLP RNAi agent to the cell or tissue of choice, for example, to an epithelial cell in vivo. In some embodiments, a TSLP RNAi agent is conjugated to a targeting group wherein the targeting group includes an integrin targeting ligand. In some embodiments, the integrin targeting ligand is an αvβ6 integrin targeting ligand. In some embodiments, a targeting group includes one or more αvβ6 integrin targeting ligands.

In some embodiments, a delivery vehicle may be used to deliver an RNAi agent to a cell or tissue. A delivery vehicle is a compound that improves delivery of the RNAi agent to a cell or tissue. A delivery vehicle can include, or consist of, but is not limited to: a polymer, such as an amphipathic polymer, a membrane active polymer, a peptide, a melittin peptide, a melittin-like peptide (MLP), a lipid, a reversibly modified polymer or peptide, or a reversibly modified membrane active polyamine.

In some embodiments, the RNAi agents can be combined with lipids, nanoparticles, polymers, liposomes, micelles, DPCs or other delivery systems available in the art for nucleic acid delivery. The RNAi agents can also be chemically conjugated to targeting groups, lipids (including, but not limited to cholesteryl and cholesteryl derivatives), encapsulating in nanoparticles, liposomes, micelles, conjugating to polymers or DPCs (see, for example WO 2000/053722, WO 2008/022309, WO 2011/104169, and WO 2012/083185, WO 2013/032829, WO 2013/158141, each of which is incorporated herein by reference), by iontophoresis, or by incorporation into other delivery vehicles or systems available in the art such as hydrogels, cyclodextrins, biodegradable nanocapsules, bioadhesive microspheres, or proteinaceous vectors. In some embodiments the RNAi agents can be conjugated to antibodies having affinity for pulmonary epithelial cells. In some embodiments, the RNAi agents can be linked to targeting ligands that have affinity for pulmonary epithelial cells or receptors present on pulmonary epithelial cells.

### Pharmaceutical Compositions and Formulations

The TSLP RNAi agents disclosed herein can be prepared as pharmaceutical compositions (alternatively referred to as pharmaceutical formulations or medicaments). The pharmaceutical compositions disclosed herein include at least one TSLP RNAi agent. These pharmaceutical compositions are particularly useful in the inhibition of the expression of TSLP mRNA in a target cell, a group of cells, a tissue, or an organism. The pharmaceutical compositions can be used to treat a subject having a disease, disorder, or condition that would benefit from reduction in the level of the target mRNA, or inhibition in expression of the target gene. The pharmaceutical compositions can be used to treat a subject at risk of developing a disease or disorder that would benefit from reduction of the level of the target mRNA or an inhibition in expression the target gene. In one embodiment, the method includes administering a TSLP RNAi agent linked to a targeting ligand as described herein, to a subject to be treated. In some embodiments, one or more pharmaceutically acceptable excipients (including vehicles, carriers, diluents, and/or delivery polymers) are added to the pharmaceutical compositions that include a TSLP RNAi agent, thereby forming a pharmaceutical formulation or medicament suitable for in vivo delivery to a subject, including a human.

The pharmaceutical compositions that include a TSLP RNAi agent and methods disclosed herein decrease the level of the target mRNA in a cell, group of cells, group of cells, tissue, organ, or subject, including by administering to the subject a therapeutically effective amount of a herein described TSLP RNAi agent, thereby inhibiting the expression of TSLP mRNA in the subject. In some embodiments, the subject has been previously identified or diagnosed as having a disease or disorder that can be mediated at least in part by a reduction in TSLP expression. In some embodiments, the subject has been previously diagnosed with having one or more pulmonary diseases such as asthma (including allergic asthma), chronic obstructive pulmonary disease including but not limited to chronic bronchitis and emphysema, pulmonary inflammatory disorders, interstitial lung diseases (ILD), cystic fibrosis, various other types of fibrosis, infectious diseases (for example, SARS-COV-2), acute lung injury (for example, acute respiratory distress syndrome (ARDS)), pulmonary hypertension, various pulmonary cancers, chronic rhinosinutis either with or without nasal polyps, autoimmune disorders including but not limited to systemic sclerosis (SSc), and multiple inflammatory diseases including but not limited to atopic dermatitis, chronic spontaneous urticaria, and eosinophilic esophagitis.

Embodiments of the present disclosure include pharmaceutical compositions for delivering a TSLP RNAi agent to a pulmonary epithelial cell in vivo. Such pharmaceutical compositions can include, for example, a TSLP RNAi agent conjugated to a targeting group that comprises an integrin targeting ligand. In some embodiments, the integrin targeting ligand is comprised of an αvβ6 integrin ligand.

In some embodiments, the described pharmaceutical compositions including a TSLP RNAi agent are used for treating or managing clinical presentations in a subject that would benefit from the inhibition of expression of TSLP. In some embodiments, a therapeutically or prophylactically effective amount of one or more of pharmaceutical compositions is administered to a subject in need of such treatment. In some embodiments, administration of any of the disclosed TSLP RNAi agents can be used to decrease the number, severity, and/or frequency of symptoms of a disease in a subject.

In some embodiments, the described TSLP RNAi agents are optionally combined with one or more additional (i.e., second, third, etc.) therapeutics. A second therapeutic can be another TSLP RNAi agent (e.g., a TSLP RNAi agent that targets a different sequence within a TSLP gene). In some embodiments, a second therapeutic can be an RNAi agent that targets the TSLP gene. An additional therapeutic can also be a small molecule drug, antibody, antibody fragment, and/or aptamer. The TSLP RNAi agents, with or without the one or more additional therapeutics, can be combined with one or more excipients to form pharmaceutical compositions.

The described pharmaceutical compositions that include a TSLP RNAi agent can be used to treat at least one symptom in a subject having a disease or disorder that would benefit from reduction or inhibition in expression of TSLP mRNA. In some embodiments, the subject is administered a therapeutically effective amount of one or more pharmaceutical compositions that include a TSLP RNAi agent thereby treating the symptom. In other embodiments, the subject is administered a prophylactically effective amount of one or more TSLP RNAi agents, thereby preventing or inhibiting the at least one symptom.

In some embodiments, one or more of the described TSLP RNAi agents are administered to a mammal in a pharmaceutically acceptable carrier or diluent. In some embodiments, the mammal is a human.

The route of administration is the path by which a TSLP RNAi agent is brought into contact with the body. In general, methods of administering drugs, oligonucleotides, and nucleic acids, for treatment of a mammal are well known in the art and can be applied to administration of the compositions described herein. The TSLP RNAi agents disclosed herein can be administered via any suitable route in a preparation appropriately tailored to the particular route. Thus, in some embodiments, the herein described pharmaceutical compositions are administered via inhalation, intranasal administration, intratracheal administration, or oropharyngeal aspiration administration. In some embodiments, the pharmaceutical compositions can be administered by injection, for example, intravenously, intramuscularly, intracutaneously, subcutaneously, intraarticularly, intraocularly, or intraperitoneally, or topically.

The pharmaceutical compositions including a TSLP RNAi agent described herein can be delivered to a cell, group of cells, tissue, or subject using oligonucleotide delivery technologies known in the art. In general, any suitable method recognized in the art for delivering a nucleic acid molecule (in vitro or in vivo) can be adapted for use with the compositions described herein. For example, delivery can be by local administration, (e.g., direct injection, implantation, or topical administering), systemic administration, or subcutaneous, intravenous, intraperitoneal, or parenteral routes, including intracranial (e.g., intraventricular, intraparenchymal and intrathecal), intramuscular, transdermal, airway (aerosol), nasal, oral, rectal, or topical (including buccal and sublingual) administration. In some embodiments, the compositions are administered via inhalation, intranasal administration, oropharyngeal aspiration administration, or intratracheal administration.

For example, in some embodiments, it is desired that the TSLP RNAi agents described herein inhibit the expression of an TSLP gene in the pulmonary epithelium, for which administration via inhalation (e.g., by an inhaler device, such as a metered-dose inhaler, or a nebulizer such as a jet or vibrating mesh nebulizer, or a soft mist inhaler) is particularly suitable and advantageous.

In some embodiments, the pharmaceutical compositions described herein comprise one or more pharmaceutically acceptable excipients. The pharmaceutical compositions described herein are formulated for administration to a subject.

As used herein, a pharmaceutical composition includes a pharmacologically effective amount of at least one of the described therapeutic compounds and one or more pharmaceutically acceptable excipients. Pharmaceutically acceptable excipients (excipients) are substances other than the Active Pharmaceutical Ingredient (API, therapeutic product, e.g., TSLP RNAi agent) that are intentionally included in the drug delivery system. Excipients do not exert or are not intended to exert a therapeutic effect at the intended dosage. Excipients can act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during stoTSLP or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, detergents, dextran, dextrose, diluents, disintegrants, emulsifiers, extenders, fillers, flavors, glidants, humectants, lubricants, oils, polymers, preservatives, saline, salts, solvents, sugars, surfactants, suspending agents, sustained release matrices, sweeteners, thickening agents, tonicity agents, vehicles, water-repelling agents, and wetting agents.

Pharmaceutical compositions suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor® EL™ (BASF, Parsippany, NJ) or phosphate buffered saline (PBS). It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration can be in the form of a sterile aqueous preparation of the drug that can be in microcrystalline form, for example, in the form of an aqueous microcrystalline suspension. Liposomal formulations or biodegradable polymer systems can also be used to present the drug for both intra-articular and ophthalmic administration.

Formulations suitable for inhalation administration can be prepared by incorporating the active compound in the desired amount in an appropriate solvent, followed by sterile filtration. In general, formulations for inhalation administration are sterile solutions at physiological pH and have low viscosity (<5 cP). Salts may be added to the formulation to balance tonicity. In some cases, surfactants or co-solvents can be added to increase active compound solubility and improve aerosol characteristics. In some cases, excipients can be added to control viscosity in order to ensure size and distribution of nebulized droplets.

In some embodiments, pharmaceutical formulations that include the TSLP RNAi agents disclosed herein suitable for inhalation administration can be prepared in water for injection (sterile water), or an aqueous sodium phosphate buffer (for example, the TSLP RNAi agent formulated in 0.5 mM sodium phosphate monobasic, 0.5 mM sodium phosphate dibasic, in water).

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

The TSLP RNAi agents can be formulated in compositions in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the therapeutic effect to be achieved, and the limitations inherent in the art of compounding such an active compound for the treatment of individuals.

A pharmaceutical composition can contain other additional components commonly found in pharmaceutical compositions. Such additional components include, but are not limited to: anti-pruritics, astringents, local anesthetics, or anti-inflammatory agents (e.g., antihistamine, diphenhydramine, etc.). It is also envisioned that cells, tissues, or isolated organs that express or comprise the herein defined RNAi agents may be used as “pharmaceutical compositions.” As used herein, “pharmacologically effective amount,” “therapeutically effective amount,” or simply “effective amount” refers to that amount of an RNAi agent to produce a pharmacological, therapeutic, or preventive result.

In some embodiments, the methods disclosed herein further comprise the step of administering a second therapeutic or treatment in addition to administering an RNAi agent disclosed herein. In some embodiments, the second therapeutic is another TSLP RNAi agent (e.g., a TSLP RNAi agent that targets a different sequence within the TSLP target). In other embodiments, the second therapeutic can be a small molecule drug, an antibody, an antibody fragment, and/or an aptamer.

In some embodiments, described herein are compositions that include a combination or cocktail of at least two TSLP RNAi agents having different sequences. In some embodiments, the two or more TSLP RNAi agents are each separately and independently linked to targeting groups. In some embodiments, the two or more TSLP RNAi agents are each linked to targeting groups that include or consist of integrin targeting ligands. In some embodiments, the two or more TSLP RNAi agents are each linked to targeting groups that include or consist of αvβ6 integrin targeting ligands.

Described herein are compositions for delivery of TSLP RNAi agents to pulmonary epithelial cells. Furthermore, compositions for delivery of TSLP RNAi agents to cells, including renal epithelial cells and/or epithelial cells in the GI or reproductive tract and/or and ocular surface epithelial cells in the eye, in vivo, are generally described herein.

Generally, an effective amount of a TSLP RNAi agent disclosed herein will be in the range of from about 0.0001 to about 20 mg/kg of body weight/deposited dose, e.g., from about 0.001 to about 5 mg/kg of body weight/deposited dose. In some embodiments, an effective amount of a TSLP RNAi agent will be in the range of from about 0.01 mg/kg to about 3.0 mg/kg of body weight per deposited dose. In some embodiments, an effective amount of a TSLP RNAi agent will be in the range of from about 0.03 mg/kg to about 2.0 mg/kg of body weight per deposited dose. In some embodiments, an effective amount of a TSLP RNAi agent will be in the range of from about 0.01 to about 1.0 mg/kg of deposited dose per body weight. In some embodiments, an effective amount of a TSLP RNAi agent will be in the range of from about 0.50 to about 1.0 mg/kg of deposited dose per body weight. Calculating the pulmonary deposited dose (PDD) is done in accordance with methods known in the art. (See Wolff R. K., Dorato M. A., Toxicologic Testing of Inhaled Pharmaceutical Aerosols, Crit Rev Toxicol., 1993; 23(4):343-369; Tepper et al., International J. Toxicology, 2016, vol. 35(4):376-392). The amount administered will also likely depend on such variables as the overall health status of the patient, the relative biological efficacy of the compound delivered, the formulation of the drug, the presence and types of excipients in the formulation, and the route of administration. Also, it is to be understood that the initial dosage administered can be increased beyond the above upper level to rapidly achieve the desired blood-level or tissue level, or the initial dosage can be smaller than the optimum. In some embodiments, a dose is administered daily. In some embodiments, a dose is administered weekly. In further embodiments, a dose is administered bi-weekly, tri-weekly, once monthly, or once quarterly (i.e., once every three months).

For treatment of disease or for formation of a medicament or composition for treatment of a disease, the pharmaceutical compositions described herein including a TSLP RNAi agent can be combined with an excipient or with a second therapeutic agent or treatment including, but not limited to: a second or other RNAi agent, a small molecule drug, an antibody, an antibody fragment, peptide, and/or an aptamer.

The described TSLP RNAi agents, when added to pharmaceutically acceptable excipients or adjuvants, can be packaged into kits, containers, packs, or dispensers. The pharmaceutical compositions described herein can be packaged in dry powder or aerosol inhalers, other metered-dose inhalers, nebulizers, pre-filled syringes, or vials.

### Methods of Treatment and Inhibition of TSLP Expression

The TSLP RNAi agents disclosed herein can be used to treat a subject (e.g., a human or other mammal) having a disease or disorder that would benefit from administration of the RNAi agent. In some embodiments, the RNAi agents disclosed herein can be used to treat a subject (e.g., a human) that would benefit from a reduction and/or inhibition in expression of TSLP mRNA and/or a reduction in TSLP cytokine levels.

In some embodiments, the RNAi agents disclosed herein can be used to treat a subject (e.g., a human) having a disease or disorder for which the subject would benefit from reduction in TSLP cytokine levels, including but not limited to, chronic obstructive pulmonary disease including but not limited to chronic bronchitis and emphysema, pulmonary inflammatory disorders, interstitial lung diseases (ILD), cystic fibrosis, various other types of fibrosis, infectious diseases (for example, SARS-COV-2), acute lung injury (for example, acute respiratory distress syndrome (ARDS)), pulmonary hypertension, various pulmonary cancers, chronic rhinosinutis either with or without nasal polyps, autoimmune disorders including but not limited to systemic sclerosis (SSc), and multiple inflammatory diseases including but not limited to atopic dermatitis, chronic spontaneous urticaria, and eosinophilic esophagitis. In some embodiments the disease is allergic asthma. In some embodiments the subject has been previously diagnosed with having asthma, or more specifically, allergic asthma, or another pulmonary inflammatory diseases. Treatment of a subject can include therapeutic and/or prophylactic treatment. The subject is administered a therapeutically effective amount of any one or more TSLP RNAi agents described herein. The subject can be a human, patient, or human patient. The subject may be an adult, adolescent, child, or infant. Administration of a pharmaceutical composition described herein can be to a human being or animal.

Increased TSLP cytokine levels are known to contribute to aberrant epithelial cell, fibroblast, and immune cell function and have been linked to fibrosis particularly in pulmonary tissues and cells. In some embodiments, the described TSLP RNAi agents are used to treat at least one symptom mediated at least in part by a reduction in TSLP cytokine levels, in a subject. The subject is administered a therapeutically effective amount of any one or more of the described TSLP RNAi agents. In some embodiments, the subject is administered a prophylactically effective amount of any one or more of the described RNAi agents, thereby treating the subject by preventing or inhibiting the at least one symptom.

In certain embodiments, the present disclosure provides methods for treatment of diseases, disorders, conditions, or pathological states mediated at least in part by TSLP gene expression, in a patient in need thereof, wherein the methods include administering to the patient any of the TSLP RNAi agents described herein.

In some embodiments, the TSLP RNAi agents are used to treat or manage a clinical presentation or pathological state in a subject, wherein the clinical presentation or pathological state is mediated at least in part by a reduction in TSLP expression. The subject is administered a therapeutically effective amount of one or more of the TSLP RNAi agents or TSLP RNAi agent-containing compositions described herein. In some embodiments, the method comprises administering a composition comprising a TSLP RNAi agent described herein to a subject to be treated.

In a further aspect, the disclosure features methods of treatment (including prophylactic or preventative treatment) of diseases or symptoms that may be addressed by a reduction in TSLP cytokine levels, the methods comprising administering to a subject in need thereof a TSLP RNAi agent that includes an antisense strand comprising the sequence of any of the sequences in Table 2, Table 3, or Table 10. Also described herein are compositions for use in such methods.

The described TSLP RNAi agents and/or compositions that include TSLP RNAi agents can be used in methods for therapeutic treatment of disease or conditions caused by enhanced or elevated TSLP cytokine levels. Such methods include administration of a TSLP RNAi agent as described herein to a subject, e.g., a human or animal subject.

In another aspect, the disclosure provides methods for the treatment (including prophylactic treatment) of a pathological state (such as a condition or disease) mediated at least in part by TSLP expression, wherein the methods include administering to a subject a therapeutically effective amount of an RNAi agent that includes an antisense strand comprising the sequence of any of the sequences in Table 2, Table 3, or Table 10.

In some embodiments, methods for inhibiting expression of an TSLP gene are disclosed herein, wherein the methods include administering to a cell an RNAi agent that includes an antisense strand comprising the sequence of any of the sequences in Table 2, Table 3, or Table 10.

In some embodiments, methods for the treatment (including prophylactic treatment) of a pathological state mediated at least in part by TSLP expression are disclosed herein, wherein the methods include administering to a subject a therapeutically effective amount of an RNAi agent that includes a sense strand comprising the sequence of any of the sequences in Table 2, Table 4, Table 5, Table 6, or Table 10.

In some embodiments, methods for inhibiting expression of an TSLP gene are disclosed herein, wherein the methods comprise administering to a cell an RNAi agent that includes a sense strand comprising the sequence of any of the sequences in Table 2, Table 4, Table 5, Table 6, or Table 10.

In some embodiments, methods for the treatment (including prophylactic treatment) of a pathological state mediated at least in part by TSLP expression are disclosed herein, wherein the methods include administering to a subject a therapeutically effective amount of an RNAi agent that includes a sense strand comprising the sequence of any of the sequences in Table 4, Table 5, Table 6, or Table 10, and an antisense strand comprising the sequence of any of the sequences in Table 3 or Table 10.

In some embodiments, methods for inhibiting expression of a TSLP gene are disclosed herein, wherein the methods include administering to a cell an RNAi agent that includes a sense strand comprising the sequence of any of the sequences in Table 4, Table 5, Table 6, or Table 10, and an antisense strand comprising the sequence of any of the sequences in Table 3 or Table 10.

In some embodiments, methods of inhibiting expression of a TSLP gene are disclosed herein, wherein the methods include administering to a subject a TSLP RNAi agent that includes a sense strand consisting of the nucleobase sequence of any of the sequences in Table 4, Table 5, Table 6, or Table 10, and the antisense strand consisting of the nucleobase sequence of any of the sequences in Table 3 or Table 10. In other embodiments, disclosed herein are methods of inhibiting expression of a TSLP gene, wherein the methods include administering to a subject a TSLP RNAi agent that includes a sense strand consisting of the modified sequence of any of the modified sequences in Table 4, Table 5, Table 6, or Table 10, and the antisense strand consisting of the modified sequence of any of the modified sequences in Table 3 or Table 10.

In some embodiments, methods for inhibiting expression of an TSLP gene in a cell are disclosed herein, wherein the methods include administering one or more TSLP RNAi agents comprising a duplex structure of one of the duplexes set forth in Tables 7A, 7B, 8, 9, and 10.

In some embodiments, the TSLP gene expression level and/or TSLP mRNA level in certain pulmonary epithelial cells of subject to whom a described TSLP RNAi agent is administered is reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99%, relative to the subject's respective level prior to being administered the TSLP RNAi agent or to a different subject not receiving the TSLP RNAi agent. In some embodiments, the TSLP cytokine levels in certain epithelial cells or circulating TSLP cytokine levels of a subject to whom a described TSLP RNAi agent is administered is reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99%, relative to the subject prior to being administered the TSLP RNAi agent or to a different subject not receiving the TSLP RNAi agent. The gene expression level, cytokine or protein level, and/or mRNA level in the subject may be reduced in a cell, group of cells, serum, and/or tissue of the subject. In some embodiments, the TSLP cytokine levels in certain subject to whom a described TSLP RNAi agent has been administered is reduced by at least about 30%, 35%, 40%, 45%, 50%, 55%, 60%, 65%, 70%, 75%, 80%, 85%, 90%, 95%, or 98% relative to the subject prior to being administered the TSLP RNAi agent or to a subject not receiving the TSLP RNAi agent.

A reduction in gene expression, mRNA, and cytokine or protein levels can be assessed by any methods known in the art. Reduction or decrease in TSLP cytokine levels or TSLP mRNA levels are sometimes collectively referred to herein as a decrease in, reduction of, or inhibition of TSLP gene expression. The Examples set forth herein illustrate known methods for assessing inhibition of TSLP.

### Cells, Tissues, Organs, and Non-Human Organisms

Cells, tissues, organs, and non-human organisms that include at least one of the TSLP RNAi agents described herein are contemplated. The cell, tissue, organ, or non-human organism is made by delivering the RNAi agent to the cell, tissue, organ, or non-human organism.

### REFERENCES

Adhikary, P. P., et al. (2021). “TSLP as druggable target—a silver-lining for atopic diseases?” Pharmacol Ther 217: 107648.
Al-Shami, A., et al. (2005). “A role for TSLP in the development of inflammation in an asthma model.” J Exp Med 202(6): 829-839.
Chen, Z. et al. (2018). “Thymic stromal lymphopoietin contribution to the recruitment of circulating fibrocytes to the lung in a mouse model of chronic allergic asthma.” J Asthma 55(9): 975-983.
Corren, J., et al. (2017). “Tezepelumab in Adults with Uncontrolled Asthma.” N Engl J Med 377(10): 936-946.
Diver, S., et al. (2021). “Effect of tezepelumab on airway inflammatory cells, remodelling, and hyperresponsiveness in patients with moderate-to-severe uncontrolled asthma (CASCADE): a double-blind, randomised, placebo-controlled, phase 2 trial.” Lancet Respir Med 9(11): 1299-1312.
Gauvreau, G. M., et al. (2020). “Thymic stromal lymphopoietin: its role and potential as a therapeutic target in asthma.” Expert Opin Ther Targets 24(8): 777-792.
Hu, Y., et al. (2017). “TSLP signaling blocking alleviates E-cadherin dysfunction of airway epithelium in a HDM-induced asthma model.” Cell Immunol 315: 56-63.
Li, Y. L., et al. (2010). “Thymic stromal lymphopoietin promotes lung inflammation through activation of dendritic cells.” J Asthma 47(2): 117-123.
Menzies-Gow, A., et al. (2021). “Tezepelumab in Adults and Adolescents with Severe, Uncontrolled Asthma.” N Engl J Med 384(19): 1800-1809.
Pandey, A., et al. (2000). “Cloning of a receptor subunit required for signaling by thymic stromal lymphopoietin.” Nat Immunol 1(1): 59-64.
Parnes, J. R, et al. (2022). “Targeting TSLP in Asthma.” J Asthma Allergy 15: 749-765.
Pelaia, C., et al. (2021). “Tezepelumab: A Potential New Biological Therapy for Severe Refractory Asthma.” Int J Mol Sci 22(9).
Puzzovio, P. G., et al. (2022). “Tezepelumab administration in moderate-to-severe uncontrolled asthma: Is it all about eosinophils?” J Allergy Clin Immunol 149(5): 1582-1584.
Torgerson, D. G., et al. (2011). “Meta-analysis of genome-wide association studies of asthma in ethnically diverse North American populations.” Nat Genet 43(9): 887-892.
Ying, S., et al. (2008). “Expression and cellular provenance of thymic stromal lymphopoietin and chemokines in patients with severe asthma and chronic obstructive pulmonary disease.” J Immunol 181(4): 2790-2798.
Ying, S., et al. (2005). “Thymic stromal lymphopoietin expression is increased in asthmatic airways and correlates with expression of Th2-attracting chemokines and disease severity.” J Immunol 174(12): 8183-8190.
Yu, G., et al. (2019). “Thymic stromal lymphopoietin (TSLP) and Toluene-diisocyanate-induced airway inflammation: Alleviation by TSLP neutralizing antibody.” Toxicol Lett 317: 59-67.
Zhou, B., et al. (2005). “Thymic stromal lymphopoietin as a key initiator of allergic airway inflammation in mice.” Nat Immunol 6(10): 1047-1053.

### ADDITIONAL ILLUSTRATIVE EMBODIMENTS

Provided here are certain additional illustrative embodiments of the disclosed technology. These embodiments are illustrative only and do not limit the scope of the present disclosure or of the claims attached hereto.
1. An RNAi agent for inhibiting expression of a thymic stromal lymphopoietin gene, comprising:
   an antisense strand comprising at least 17 contiguous nucleotides differing by 0 or 1 nucleotides from any one of the sequences provided in Table 2 or Table 3; and
   a sense strand comprising a nucleotide sequence that is at least partially complementary to the antisense strand.
2. The RNAi agent of embodiment 1, wherein the antisense strand comprises nucleotides 2-18 of any one of the sequences provided in Table 2 or Table 3.
3. The RNAi agent of embodiment 1 or embodiment 2, wherein the sense strand comprises a nucleotide sequence of at least 17 contiguous nucleotides differing by 0 or 1 nucleotides from any one of the sequences provided in Table 2 or Table 4, and wherein the sense strand has a region of at least 85% complementarity over the 17 contiguous nucleotides to the antisense strand.
4. The RNAi agent of any one of embodiments 1-3, wherein at least one nucleotide of the TSLP RNAi agent is a modified nucleotide or includes a modified internucleoside linkage.
5. The RNAi agent of any one of embodiments 1-4, wherein all or substantially all of the nucleotides are modified nucleotides.
6. The RNAi agent of any one of embodiments 4-5, wherein the modified nucleotide is selected from the group consisting of: 2′-O-methyl nucleotide, 2′-fluoro nucleotide, 2′-deoxy nucleotide, 2′,3′-seco nucleotide mimic, locked nucleotide, 2′-F-arabino nucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, vinyl phosphonate-containing nucleotide, cyclopropyl phosphonate-containing nucleotide, and 3′-O-methyl nucleotide.
7. The RNAi agent of embodiment 5, wherein all or substantially all of the nucleotides are modified with 2′-O-methyl nucleotides, 2′-fluoro nucleotides, or combinations thereof.
8. The RNAi agent of any one of embodiments 1-7, wherein the antisense strand comprises the nucleotide sequence of any one of the modified sequences provided in Table 3.
9. The RNAi agent of any one of embodiments 1-8, wherein the sense strand comprises the nucleotide sequence of any one of the modified sequences provided in Table 4.
10. The RNAi agent of embodiment 1, wherein the antisense strand comprises the nucleotide sequence of any one of the modified sequences provided in Table 3 and the sense strand comprises the nucleotide sequence of any one of the modified sequences provided in Table 4.
11. The RNAi agent of any one of embodiments 1-10, wherein the sense strand is between 18 and 30 nucleotides in length, and the antisense strand is between 18 and 30 nucleotides in length.
12. The RNAi agent of embodiment 11, wherein the sense strand and the antisense strand are each between 18 and 27 nucleotides in length.
13. The RNAi agent of embodiment 12, wherein the sense strand and the antisense strand are each between 18 and 24 nucleotides in length.
14. The RNAi agent of embodiment 13, wherein the sense strand and the antisense strand are each 21 nucleotides in length.
15. The RNAi agent of embodiment 14, wherein the RNAi agent has two blunt ends.
16. The RNAi agent of any one of embodiments 1-15, wherein the sense strand comprises one or two terminal caps.
17. The RNAi agent of any one of embodiments 1-16, wherein the sense strand comprises one or two inverted abasic residues.
18. The RNAi agent of embodiment 1, wherein the RNAi agent is comprised of a sense strand and an antisense strand that form a duplex having the structure of any one of the duplexes in Table 7A, Table 7B, Table 8, Table 9, or Table 10.
19. The RNAi agent of embodiment 18, wherein all or substantially all of the nucleotides are modified nucleotides.
20. The RNAi agent of embodiment 1, comprising an antisense strand that consists of, consists essentially of, or comprises a nucleotide sequence that differs by 0 or 1 nucleotides from one of the following nucleotide sequences (5′→3′):

(SEQ ID NO: 836)
AGACAUUUAUUGGUUGUGACC;
(SEQ ID NO: 853)
AGACGUUUAUUGGUUGUGACC;
(SEQ ID NO: 837)
UGACAUUUAUUGGUUGUGACC;
(SEQ ID NO: 856)
UGACGUUUAUUGGUUGUGACC;
(SEQ ID NO: 196)
AGACAUUUAUUGGUUGUGA;
(SEQ ID NO: 197)
UGACAUUUAUUGGUUGUGA;
(SEQ ID NO: 137)
UUAGCAUUUAUCUGAGUUU;
(SEQ ID NO: 139)
UUAGCAUUUAUCUGAGUUC;
(SEQ ID NO: 192)
UACAUUUAUUGGUUGUGAC;
(SEQ ID NO: 830)
AGACAUUUAUUGGUUGUGACU;
(SEQ ID NO: 825)
UUAGCAUUUAUCUGAGUUUCC;
or
(SEQ ID NO: 826)
UACAUUUAUUGGUUGUGACUU.

21. The RNAi agent of embodiment 20, wherein the sense strand consists of, consists essentially of, or comprises a nucleotide sequence that differs by 0 or 1 nucleotides from one of the following nucleotide sequences (5′→3′):

(SEQ ID NO: 872)
GGUCACAACCAAUAAAUGUCU;
(SEQ ID NO: 873)
GGUCACAACCAAUAAAUGUCA;
(SEQ ID NO: 461)
UCACAACCAAUAAAUGUCU;
(SEQ ID NO: 462)
UCACAACCAAUAAAUGUCA;
(SEQ ID NO: 402)
AAACUCAGAUAAAUGCUAA;
(SEQ ID NO: 871)
G(A^{2N})ACUCAGAUAAAUGCUAA;
(SEQ ID NO: 457)
GUCACAACCAAUAAAUGUA
(SEQ ID NO: 864)
AGUCACAACCAAUAAAUGUCU;
(SEQ ID NO: 866)
GGAAACUCAGAUAAAUGCUAA;
or
(SEQ ID NO: 863)
(A^{2N})AGUCACAACCAAUAAAUGUA, wherein (A^{2N})

represents a 2-aminoadenosine nucleotide.
22. The RNAi agent of embodiment 20 or 21, wherein all or substantially all of the nucleotides are modified nucleotides.
23. The RNAi agent of embodiment 1, comprising an antisense strand that comprises, consists of, or consists essentially of a modified nucleotide sequence that differs by 0 or 1 nucleotides from one of the following nucleotide sequences (5′→3′):

(SEQ ID NO: 649)
cPrpasGfsacauuuaUfuGfgUfuGfugacsc
(SEQ ID NO: 609)
cPrpasGfsaCfaUfuUfaUfuGfgUfuGfuGfaCfsu;
(SEQ ID NO: 611)
cPrpasGfsacauuuaUfuGfgUfuGfugacsu;
(SEQ ID NO: 681)
cPrpasGfsacguuuaUfuGfgUfuGfugacsc;
(SEQ ID NO: 612)
cPrpasGfsacauuuAfuuGfgUfuGfugacsu;
(SEQ ID NO: 603)
cPrpusUfsagcauuUfauCfuGfaGfuuucsc;
(SEQ ID NO: 606)
cPrpusUfsagcauUfuauCfuGfaGfuuucsc;
or
(SEQ ID NO: 594)
cPrpusAfscsAfuUfuAfuUfgGfuUfgUfgAfcUfsu;

wherein a represents 2′-O-methyl adenosine, c represents 2′-O-methyl cytidine, g represents 2′-O-methyl guanosine, and u represents 2′-O-methyl uridine; Af, represents 2′-fluoro adenosine, Cf represents 2′-fluoro cytidine, Gf represents 2′-fluoro guanosine, and Uf represents 2′-fluoro uridine; cPrpa represents a 5′-cyclopropyl phosphonate-2′-O-methyl adenosine; cPrpu represents a 5′-cyclopropyl phosphonate-2′-O-methyl uridine; s represents a phosphorothioate linkage; and wherein all or substantially all of the nucleotides on the sense strand are modified nucleotides.
24. The RNAi agent of embodiment 1, wherein the sense strand comprises, consists of, or consists essentially of a modified nucleotide sequence that differs by 0 or 1 nucleotides from one of the following nucleotide sequences (5′→3′):

(SEQ ID NO: 714)
gsgucacaaCfCfAfauaaaugucu;
(SEQ ID NO: 702)
asgucacaaCfCfAfauaaaugucu;
(SEQ ID NO: 704)
gsgaaacucAfGfAfuaaaugcuaa;
(SEQ ID NO: 701)
a_2NsagucacaAfCfCfaauaaaugua;

wherein a represents 2′-O-methyl adenosine, c represents 2′-O-methyl cytidine, g represents 2′-O-methyl guanosine, and u represents 2′-O-methyl uridine; Af, represents 2′-fluoro adenosine, Cf represents 2′-fluoro cytidine, Gf represents 2′-fluoro guanosine, and Uf represents 2′-fluoro uridine; a 2N represents 2′-O-methyl-2-aminoadenosine; s represents a phosphorothioate linkage; and wherein all or substantially all of the nucleotides on the antisense strand are modified nucleotides.
25. The RNAi agent of any one of embodiments 20-24, wherein the sense strand further includes inverted abasic residues at the 3′ terminal end of the nucleotide sequence, at the 5′ end of the nucleotide sequence, or at both.
26. The RNAi agent of any one of embodiments 1-25, wherein the RNAi agent is linked to a targeting ligand.
27. The RNAi agent of embodiment 26, wherein the targeting ligand has affinity for a cell receptor expressed on an epithelial cell.
28. The RNAi agent of embodiment 27, wherein the targeting ligand comprises an integrin targeting ligand.
29. The RNAi agent of embodiment 28, wherein the integrin targeting ligand is an αvβ6 integrin targeting ligand.
30. The RNAi agent of embodiment 29, wherein the targeting ligand comprises the structure:

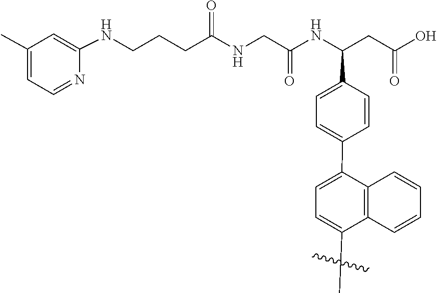 [SMILES: *c1ccc(-c2ccc([C@H](CC(=O)O)NC(=O)CNC(=O)CCCNc3cc(C)ccn3)cc2)c2ccccc12]

or a pharmaceutically acceptable salt thereof, or

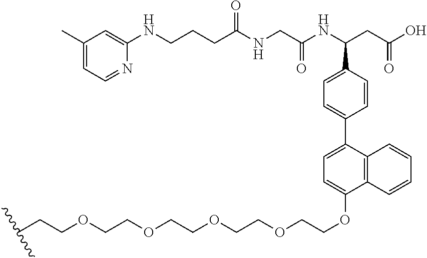 [SMILES: *CCOCCOCCOCCOCCOc1ccc(-c2ccc([C@H](CC(=O)O)NC(=O)CNC(=O)CCCNc3cc(C)ccn3)cc2)c2ccccc12]

or a pharmaceutically acceptable salt thereof,
wherein [symbol] indicates the point of connection to the RNAi agent.
31. The RNAi agent of any one of embodiments 26-29, wherein the targeting ligand has a structure selected from the group consisting of:

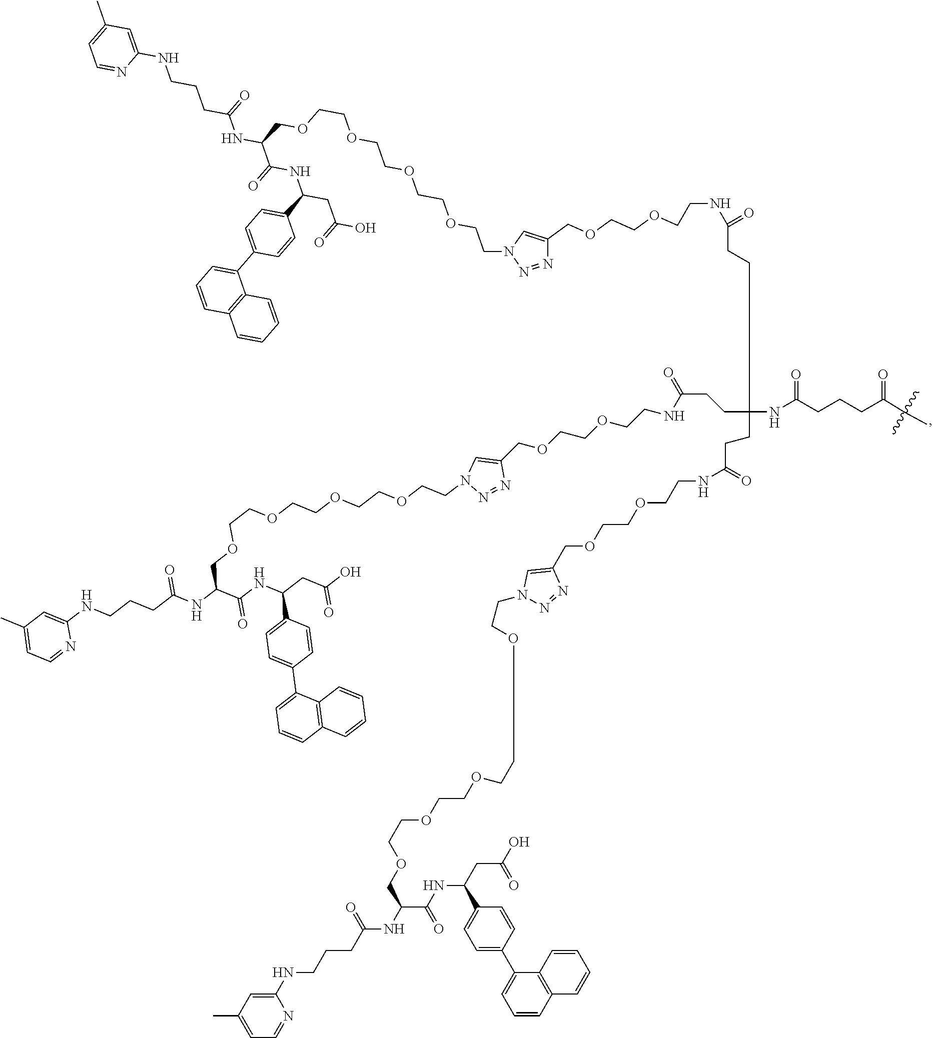 [SMILES: *C(=O)CCCC(=O)NC(CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1)(CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1)CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1]

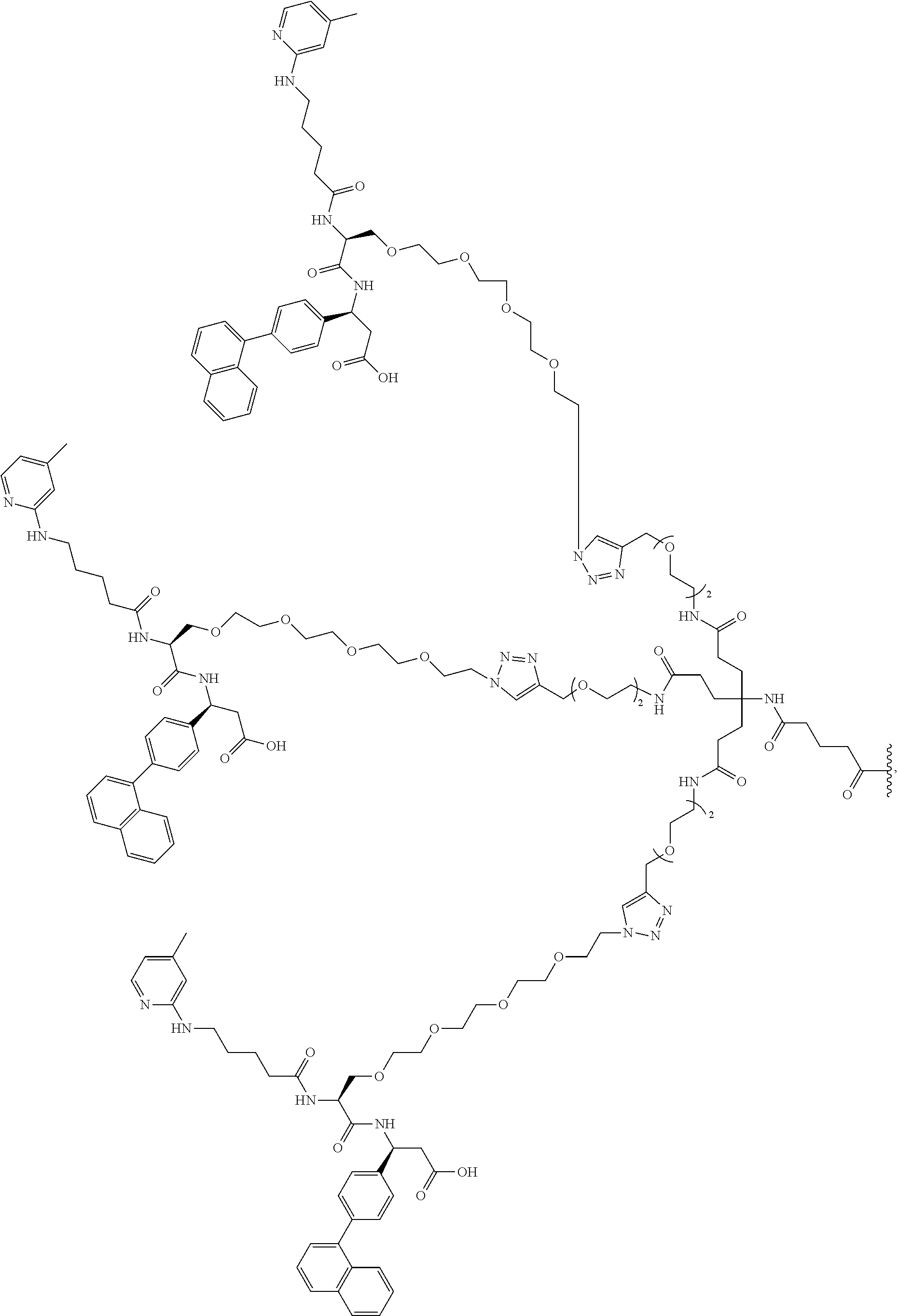

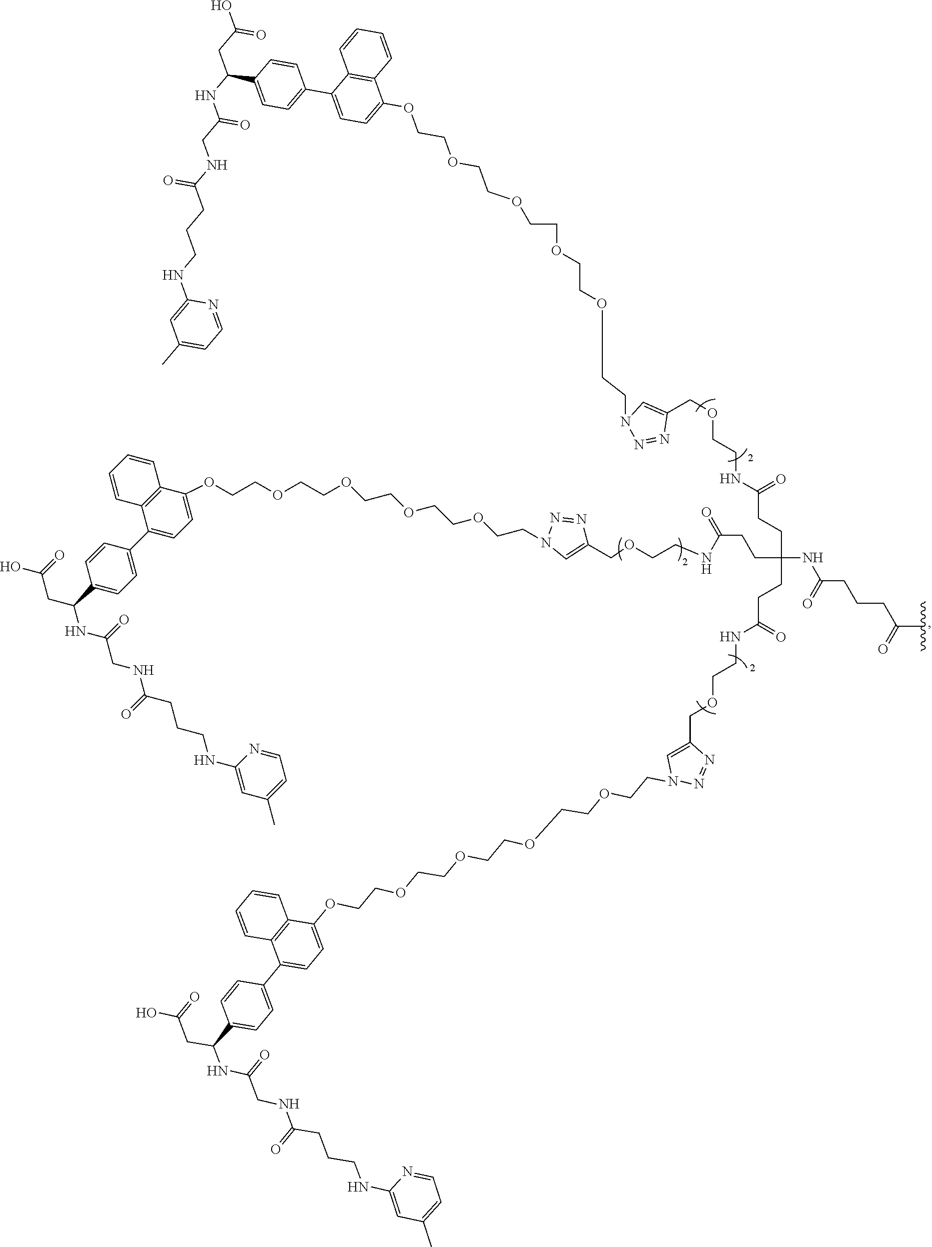

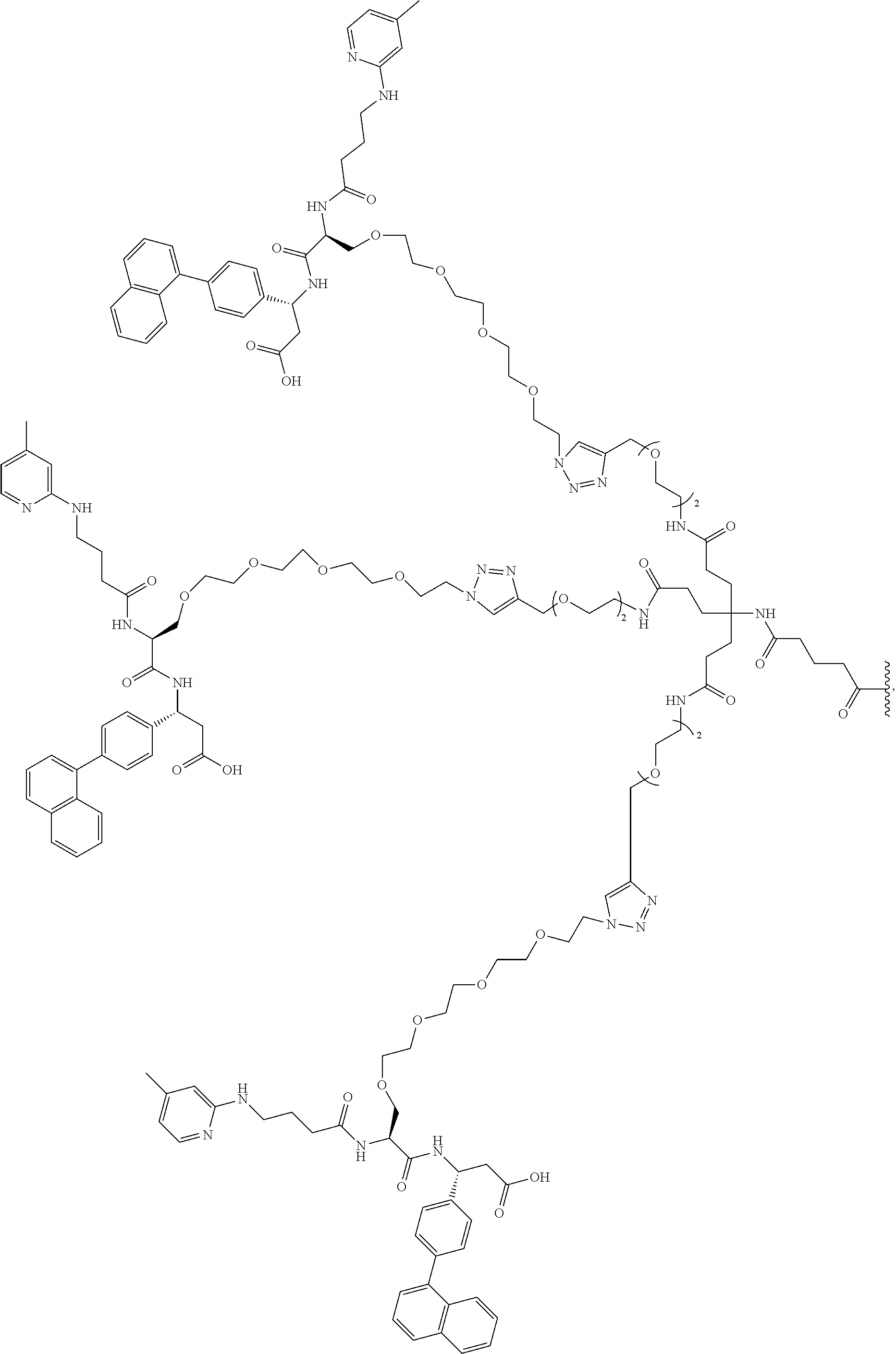

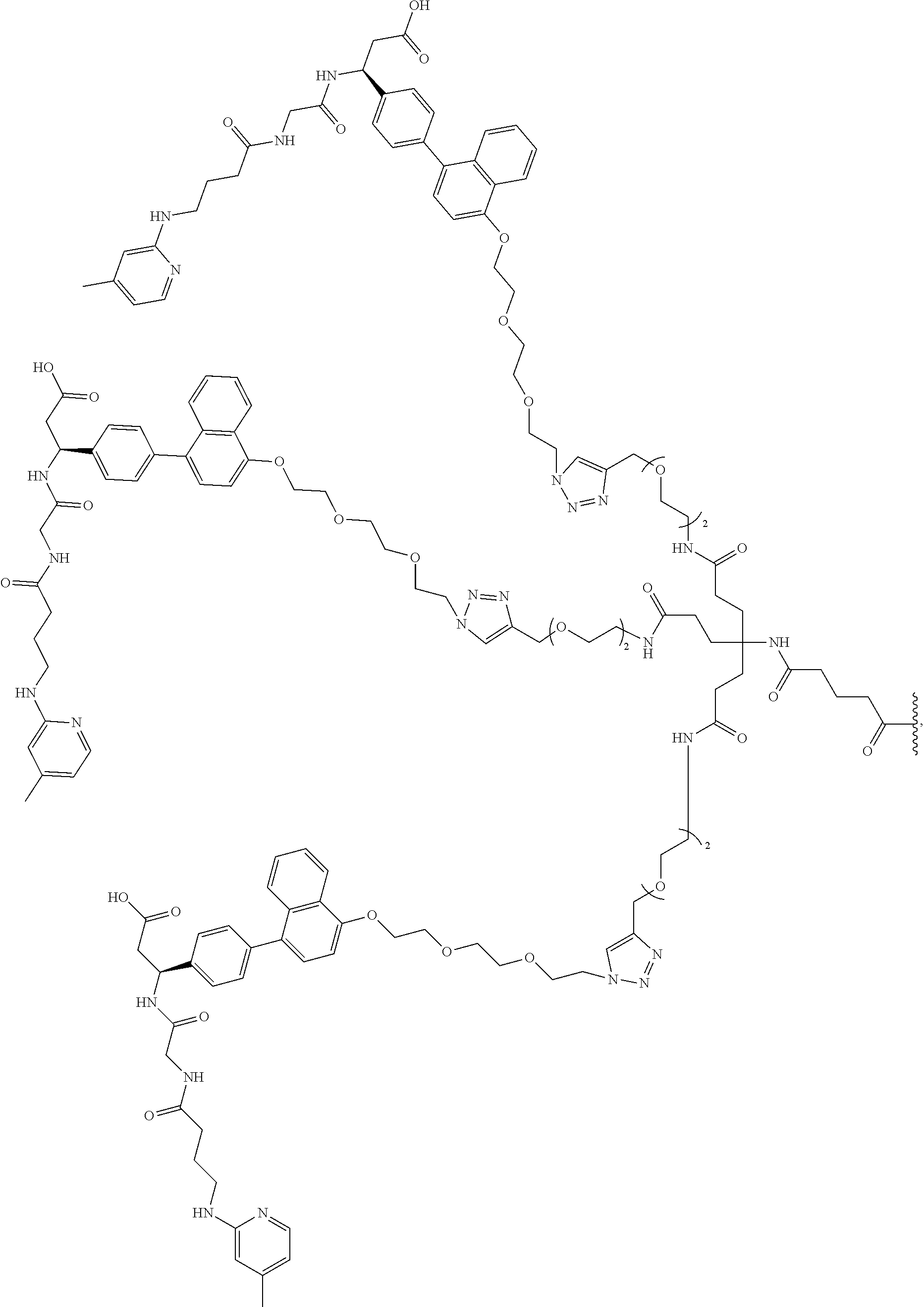

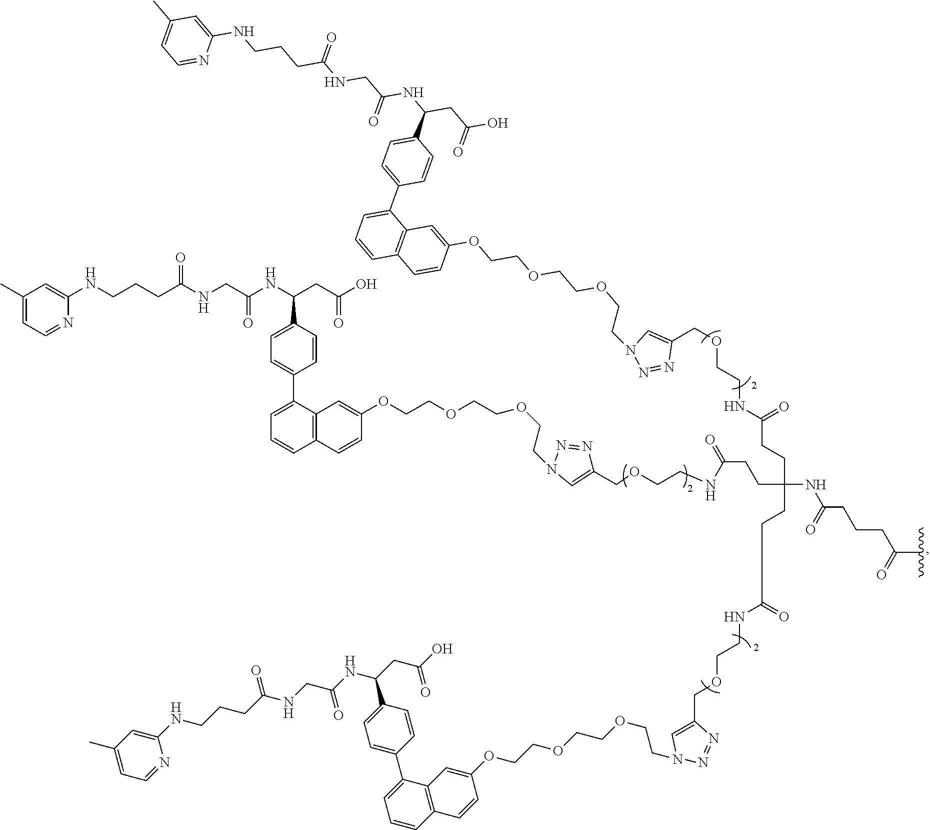

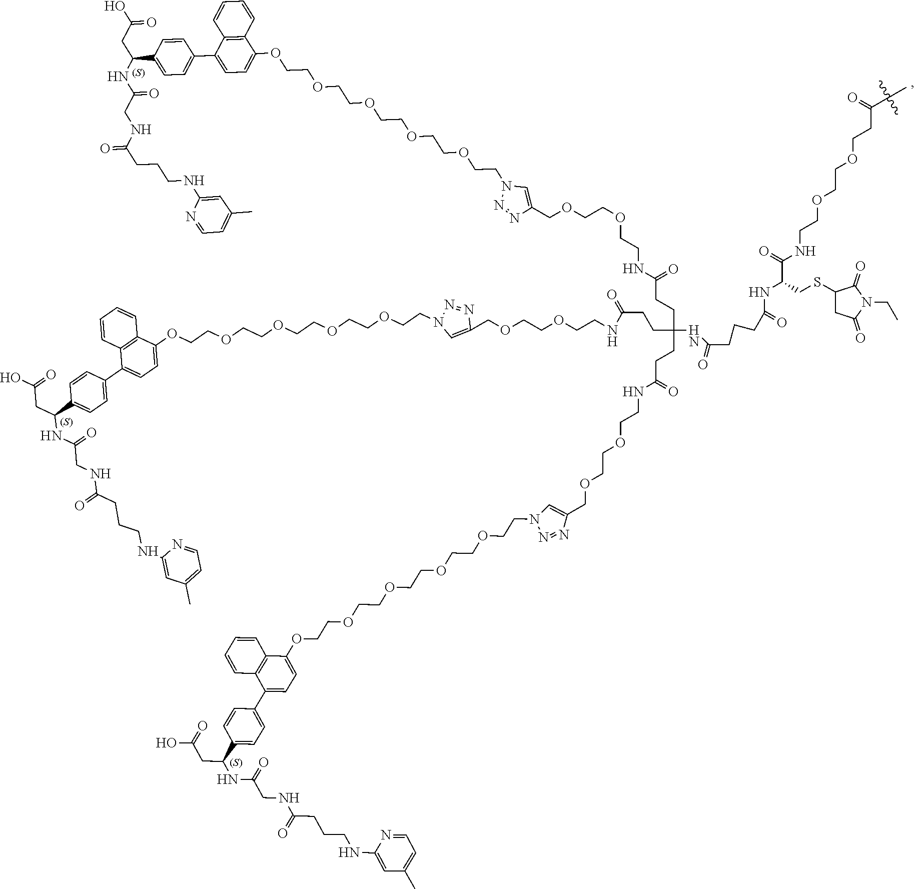

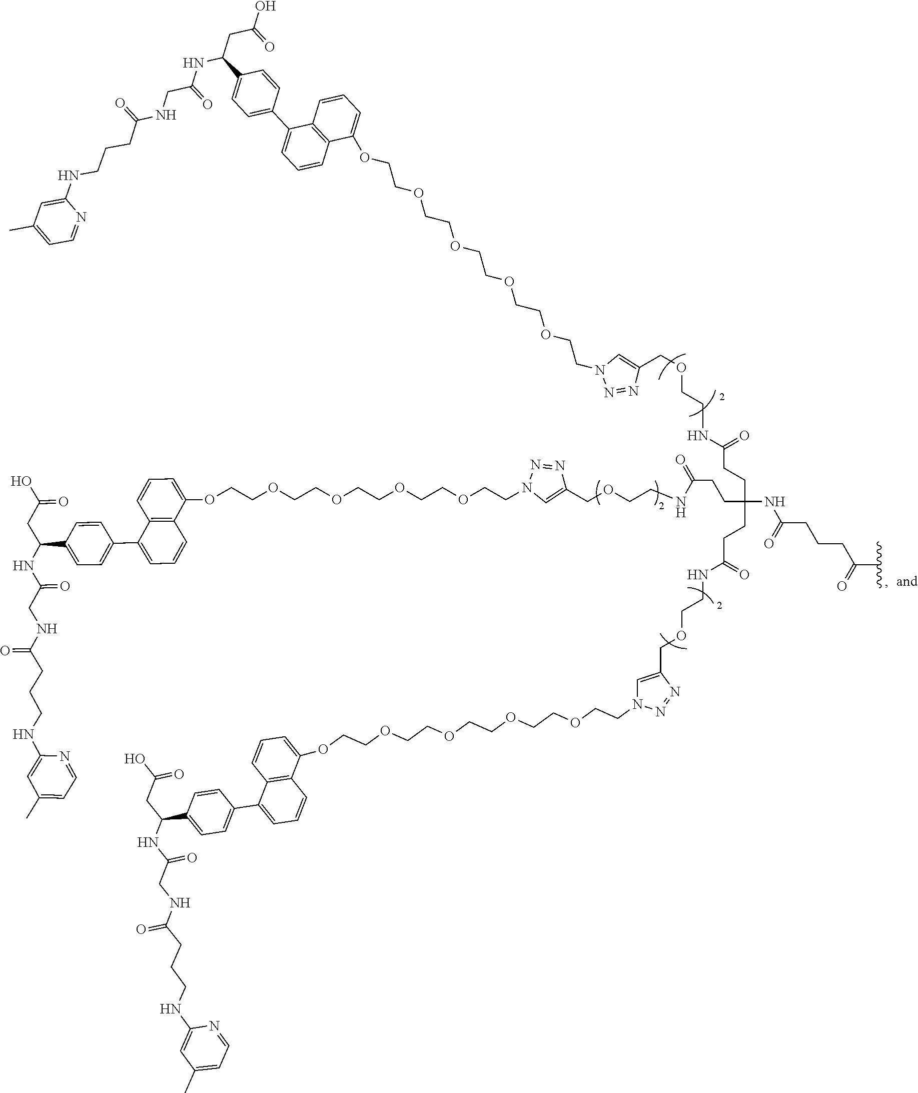

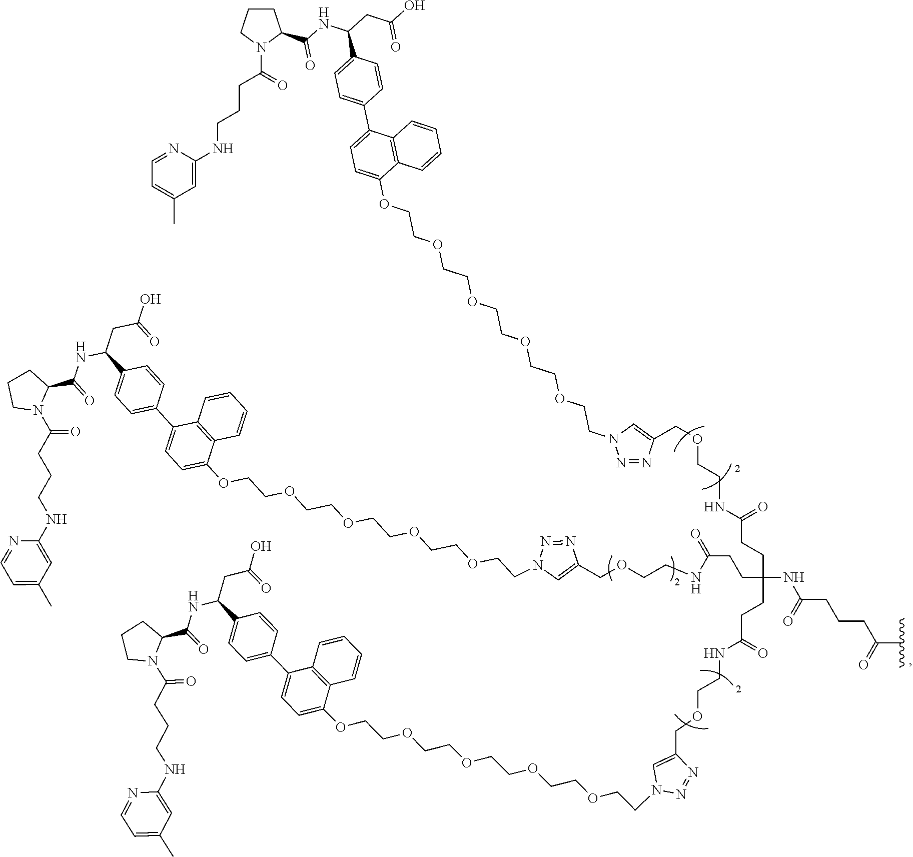

wherein [symbol] indicates the point of connection to the RNAi agent.
32. The RNAi agent of embodiment 31, wherein RNAi agent is conjugated to a targeting ligand having the following structure:

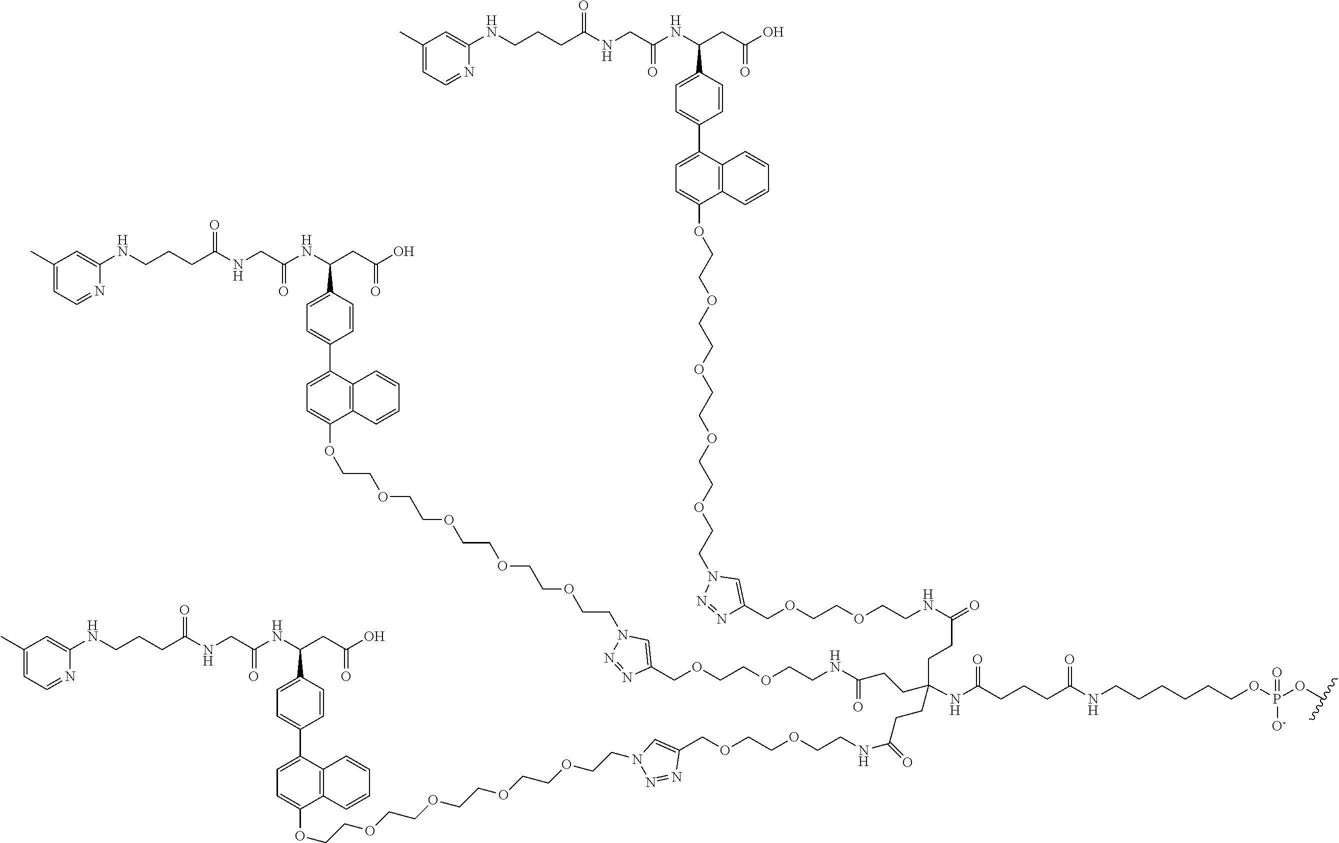 [SMILES: *OP(=O)([O-])OCCCCCCNC(=O)CCCC(=O)NC(CCC(=O)NCCOCCOCc1cn(CCOCCOCCOCCOCCOc2ccc(-c3ccc([C@H](CC(=O)O)NC(=O)CNC(=O)CCCNc4cc(C)ccn4)cc3)c3ccccc23)nn1)(CCC(=O)NCCOCCOCc1cn(CCOCCOCCOCCOCCOc2ccc(-c3ccc([C@H](CC(=O)O)NC(=O)CNC(=O)CCCNc4cc(C)ccn4)cc3)c3ccccc23)nn1)CCC(=O)NCCOCCOCc1cn(CCOCCOCCOCCOCCOc2ccc(-c3ccc([C@H](CC(=O)O)NC(=O)CNC(=O)CCCNc4cc(C)ccn4)cc3)c3ccccc23)nn1]

33. The RNAi agent of any one of embodiments 26-32, wherein the targeting ligand is 7 conjugated to the sense strand.
34. The RNAi agent of embodiment 33, wherein the targeting ligand is conjugated to the 5′ terminal end of the sense strand.
35. The RNAi agent of any one of embodiments 1-34, wherein the RNAi agent is a pharmaceutically acceptable salt.
36. The RNAi agent of any one of embodiment 35, wherein the RNAi agent is a sodium salt.
37. A composition comprising the RNAi agent of any one of embodiments 1-36, wherein the composition further comprises a pharmaceutically acceptable excipient.
38. The composition of embodiment 37, further comprising a second RNAi agent capable of inhibiting the expression of thymic stromal lymphopoietin gene expression.
39. The composition of any one of embodiments 37-38, further comprising one or more additional therapeutics.
40. The composition of any one of embodiments 37-39, wherein the composition is formulated for administration by inhalation.
41. The composition of embodiment 40, wherein the composition is delivered by a metered-dose inhaler, jet nebulizer, vibrating mesh nebulizer, or soft mist inhaler.
42. The composition of any of embodiments 37-41, wherein the RNAi agent is a sodium salt.
43. The composition of any of embodiments 37-42, wherein the pharmaceutically acceptable excipient is water for injection.
44. The composition of any of embodiments 37-42, wherein the pharmaceutically acceptable excipient is a buffered saline solution.
45. A method for inhibiting expression of a TSLP gene in a cell, the method comprising introducing into a cell an effective amount of an RNAi agent of any one of embodiments 1-35 or the composition of any one of embodiments 37-45.
46. The method of embodiment 45, wherein the cell is within a subject.
47. The method of embodiment 46, wherein the subject is a human subject.
48. The method of any one of embodiments 45-47, wherein following the administration of the RNAi agent the thymic stromal lymphopoietin gene expression is inhibited by at least about 30%.
49. A method of treating one or more symptoms or diseases associated with enhanced or elevated TSLP cytokine activity levels, the method comprising administering to a human subject in need thereof a therapeutically effective amount of the composition of any one of embodiments 37-44.
50. The method of embodiment 49, wherein the disease is asthma including but not limited to allergic asthma, chronic obstructive pulmonary disease including but not limited to chronic bronchitis and emphysema, pulmonary inflammatory disorders, interstitial lung diseases (ILD), cystic fibrosis, various other types of fibrosis, infectious diseases (for example, SARS-COV-2), acute lung injury (for example, acute respiratory distress syndrome (ARDS)), pulmonary hypertension, various pulmonary cancers, chronic rhinosinutis either with or without nasal polyps, autoimmune disorders including but not limited to systemic sclerosis (SSc), and multiple inflammatory diseases including but not limited to atopic dermatitis, chronic spontaneous urticaria, and eosinophilic esophagitis.
51. The method of embodiment 50, wherein the disease is allergic asthma.
52. The method of any one of embodiments 45-51, wherein the RNAi agent is administered at a deposited dose of about 0.01 mg/kg to about 5.0 mg/kg of body weight of the subject.
53. The method of any one of embodiments 45-52, wherein the RNAi agent is administered at a deposited dose of about 0.03 mg/kg to about 2.0 mg/kg of body weight of the subject.
54. The method of any of embodiments 45-53, wherein the RNAi agent is administered in two or more doses.
55. Use of the RNAi agent of any one of embodiments 1-36, for the treatment of a disease, disorder, or symptom that is mediated at least in part by TSLP cytokine activity and/or TSLP gene expression.
56. Use of the composition according to any one of embodiments 37-44, for the treatment of a disease, disorder, or symptom that is mediated at least in part by thymic stromal lymphopoietin cytokine activity and/or thymic stromal lymphopoietin gene expression.
57. Use of the composition according to any one of embodiments 37-44, for the manufacture of a medicament for treatment of a disease, disorder, or symptom that is mediated at least in part by thymic stromal lymphopoietin cytokine and/or thymic stromal lymphopoietin gene expression.
58. The use of any one of embodiments 55-57, wherein the disease is pulmonary inflammation.
59. A method of making an RNAi agent of any one of embodiments 1-36, comprising annealing a sense strand and an antisense strand to form a double-stranded ribonucleic acid molecule.
60. The method of embodiment 59, wherein the sense strand comprises a targeting ligand.
61. The method of embodiment 60, comprising conjugating a targeting ligand to the sense strand.

The above provided embodiments and items are now illustrated with the following, non-limiting examples.

### EXAMPLES

### Example 1. Synthesis of TSLP RNAI Agents

TSLP RNAi agent duplexes disclosed herein were synthesized in accordance with the following:

A. Synthesis. The sense and antisense strands of the TSLP RNAi agents were synthesized according to phosphoramidite technology on solid phase used in oligonucleotide synthesis. Depending on the scale, a MerMade96E® (Bioautomation), a MerMade12® (Bioautomation), or an OP Pilot 100 (GE Healthcare) was used. Syntheses were performed on a solid support made of controlled pore glass (CPG, 500 Å or 600 Å, obtained from Prime Synthesis, Aston, PA, USA). The monomer positioned at the 3′ end of the respective strand attached to the solid support was used as a starting point for synthesis and is acquired commercially. All RNA and 2′-modified RNA phosphoramidites were purchased from Thermo Fisher Scientific (Milwaukee, WI, USA). Specifically, the 2′-O-methyl phosphoramidites that were used included the following: (5′-O-dimethoxytrityl-N⁶-(benzoyl)-2′-O-methyl-adenosine-3′-O-(2-cyanoethyl-N,N-diisopropylamino) phosphoramidite, 5′-O dimethoxy-trityl-N⁴-(acetyl)-2′-O-methyl-cytidine-3′-O-(2-cyanoethyl-N,N-diisopropylamino) phosphoramidite, (5′-O-dimethoxytrityl-N²-(isobutyryl)-2′-O-methyl-guanosine-3′-O-(2-cyanoethyl-N,N-diisopropylamino) phosphoramidite, and 5′-O-dimethoxytrityl-2′-O-methyl-uridine-3′-O-(2-cyanoethyl-N,N-diisopropylamino) phosphoramidite. The 2′-deoxy-2′-fluoro-phosphoramidites carried the same protecting groups as the 2′-O-methyl RNA amidites. 5′-dimethoxytrityl-2′-O-methyl-inosine-3′-O-(2-cyanoethyl-N,N-diisopropylamino) phosphoramidites were purchased from Glen Research (Virginia). The inverted abasic (3′-O-dimethoxytrityl-2′-deoxyribose-5′-O-(2-cyanoethyl-N,N-diisopropylamino) phosphoramidites were purchased from ChemGenes (Wilmington, MA, USA). The following UNA phosphoramidites were used: 5′-(4,4′-Dimethoxytrityl)-N6-(benzoyl)-2′,3′-seco-adenosine, 2′-benzoyl-3′-[(2-cyanoethyl)-(N,N-diisopropyl)]-phosphoramidite, 5′-(4,4′-Dimethoxytrityl)-N-acetyl-2′,3′-seco-cytosine, 2′-benzoyl-3′-[(2-cyanoethyl)-(N,N-diiso-propyl)]-phosphoramidite, 5′-(4,4′-Dimethoxytrityl)-N-isobutyryl-2′,3′-seco-guanosine, 2′-benzoyl-3′-[(2-cyanoethyl)-(N,N-diisopropyl)]-phosphoramidite, and 5′-(4,4′-Dimethoxy-trityl)-2′,3′-seco-uridine, 2′-benzoyl-3′-[(2-cyanoethyl)-(N,N-diiso-propyl)]-phosphoramidite. TFA aminolink phosphoramidites were also commercially purchased (ThermoFisher). Linker L6 was purchased as propargyl-PEG5-NHS from BroadPharm (catalog #BP-20907) and coupled to the NH₂-C₆ group from an aminolink phosphoramidite to form -L6-C6-, using standard coupling conditions. In each case, phosphorothioate linkages were introduced as specified using the conditions set forth herein. The cyclopropyl phosphonate phosphoramidites were synthesized in accordance with International Patent Application Publication No. WO 2017/214112 (see also Altenhofer et. al., Chem. Communications (Royal Soc. Chem.), 57(55):6808-6811 (July 2021)).

Tri-alkyne-containing phosphoramidites were dissolved in anhydrous dichloromethane or anhydrous acetonitrile (50 mM), while all other amidites were dissolved in anhydrous acetonitrile (50 mM) and molecular sieves (3 Å) were added. 5-Benzylthio-1H-tetrazole (BTT, 250 mM in acetonitrile) or 5-Ethylthio-1H-tetrazole (ETT, 250 mM in acetonitrile) was used as activator solution. Coupling times were 10 minutes (RNA), 90 seconds (2′ O-Me), and 60 seconds (2′ F). In order to introduce phosphorothioate linkages, a 100 mM solution of 3-phenyl 1,2,4-dithiazoline-5-one (POS, obtained from PolyOrg, Inc., Leominster, MA, USA) in anhydrous acetonitrile was employed.

Alternatively, tri-alkyne moieties were introduced post-synthetically (see section E, below). For this route, the sense strand was functionalized with a 5′ and/or 3′ terminal nucleotide containing a primary amine. TFA aminolink phosphoramidite was dissolved in anhydrous acetonitrile (50 mM) and molecular sieves (3 Å) were added. 5-Benzylthio-1H-tetrazole (BTT, 250 mM in acetonitrile) or 5-Ethylthio-1H-tetrazole (ETT, 250 mM in acetonitrile) was used as activator solution. Coupling times were 10 minutes (RNA), 90 seconds (2′ O-Me), and 60 seconds (2′ F). In order to introduce phosphorothioate linkages, a 100 mM solution of 3-phenyl 1,2,4-dithiazoline-5-one (POS, obtained from PolyOrg, Inc., Leominster, MA, USA) in anhydrous acetonitrile was employed.

B. Cleavage and deprotection of support bound oligomer. After finalization of the solid phase synthesis, the dried solid support was treated with a 1:1 volume solution of 40 wt. % methylamine in water and 28% to 31% ammonium hydroxide solution (Aldrich) for 1.5 hours at 30° C. The solution was evaporated and the solid residue was reconstituted in water (see below).

C. Purification. Crude oligomers were purified by anionic exchange HPLC using a TSKgel SuperQ-5PW 13 μm column and Shimadzu LC-8 system. Buffer A was 20 mM Tris, 5 mM EDTA, pH 9.0 and contained 20% Acetonitrile and buffer B was the same as buffer A with the addition of 1.5 M sodium chloride. UV traces at 260 nm were recorded. Appropriate fractions were pooled then run on size exclusion HPLC using a GE Healthcare XK 16/40 column packed with Sephadex G-25 fine with a running buffer of 100 mM ammonium bicarbonate, pH 6.7 and 20% Acetonitrile or filtered water. Alternatively, pooled fractions were desalted and exchanged into an appropriate buffer or solvent system via tangential flow filtration.

D. Annealing. Complementary strands were mixed by combining equimolar RNA solutions (sense and antisense) in 1×PBS (Phosphate-Buffered Saline, 1×, Corning, Cellgro) to form the RNAi agents. Some RNAi agents were lyophilized and stored at −15 to −25° C. Duplex concentration was determined by measuring the solution absorbance on a UV-Vis spectrometer in 1×PBS. The solution absorbance at 260 nm was then multiplied by a conversion factor (0.050 mg/(mL·cm)) and the dilution factor to determine the duplex concentration.

E. Conjugation of Tri-alkyne linker. In some embodiments a tri-alkyne linker is conjugated to the sense strand of the RNAi agent on resin as a phosphoramidite (see Example 1G for the synthesis of an example tri-alkyne linker phosphoramidite and Example 1A for the conjugation of the phosphoramidite.). In other embodiments, a tri-alkyne linker may be conjugated to the sense strand following cleavage from the resin, described as follows: either prior to or after annealing, in some embodiments, the 5′ or 3′ amine functionalized sense strand is conjugated to a tri-alkyne linker. An example tri-alkyne linker structure that can be used in forming the constructs disclosed herein is as follows:

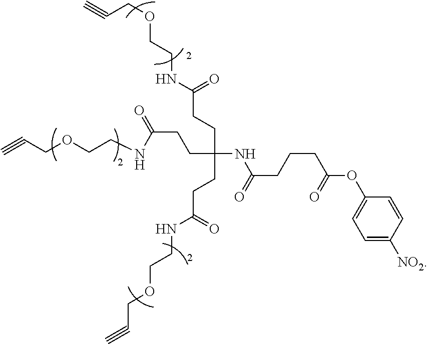

To conjugate the tri-alkyne linker to the annealed duplex, amine-functionalized duplex was dissolved in 90% DMSO/10% H₂O, at ˜50-70 mg/mL. 40 equivalents triethylamine was added, followed by 3 equivalents tri-alkyne-PNP. Once complete, the conjugate was precipitated twice in a solvent system of 1× phosphate buffered saline/acetonitrile (1:14 ratio), and dried.

### F. Synthesis of Targeting Ligand SM6.1

### ((S)-3-(4-(4-((14-azido-3,6,9,12-tetraoxatetradecyl)oxy)naphthalen-1-yl)phenyl)-3-(2-(4-((4-methylpyridin-2-yl)amino)butanamido)acetamido)propanoic Acid)

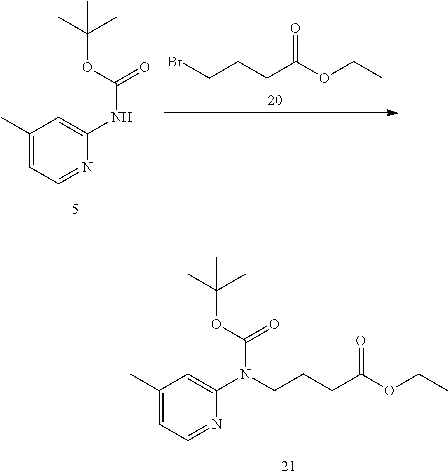

Compound 5 (tert-Butyl(4-methylpyridin-2-yl)carbamate) (0.501 g, 2.406 mmol, 1 equiv.) was dissolved in DMF (17 mL). To the mixture was added NaH (0.116 mg, 3.01 mmol, 1.25 eq, 60% dispersion in oil) The mixture stirred for 10 min before adding Compound 20 (Ethyl 4-Bromobutyrate (0.745 g, 3.82 mmol, 0.547 mL)) (Sigma 167118). After 3 hours the reaction was quenched with ethanol (18 mL) and concentrated. The concentrate was dissolved in DCM (50 mL) and washed with saturated aq. NaCl solution (1×50 mL), dried over Na₂SO₄, filtered and concentrated. The product was purified on silica column, gradient 0-5% Methanol in DCM.

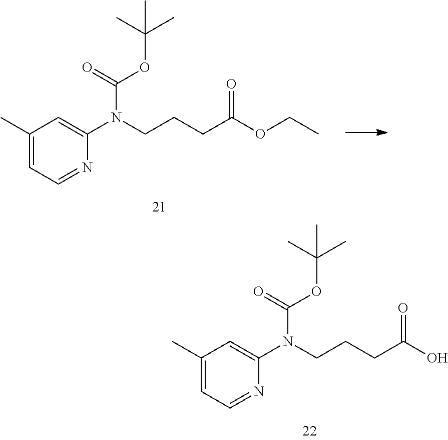

Compound 21 was dissolved (0.80 g, 2.378 mmol) in 100 mL of Acetone:0.1 M NaOH [1:1]. The reaction was monitored by TLC (5% ethyl acetate in hexane). The organics were concentrated away, and the residue was acidified to pH 3-4 with 0.3 M Citric Acid (40 mL). The product was extracted with DCM (3×75 mL). The organics were pooled, dried over Na₂SO₄, filtered and concentrated. The product was used without further purification.

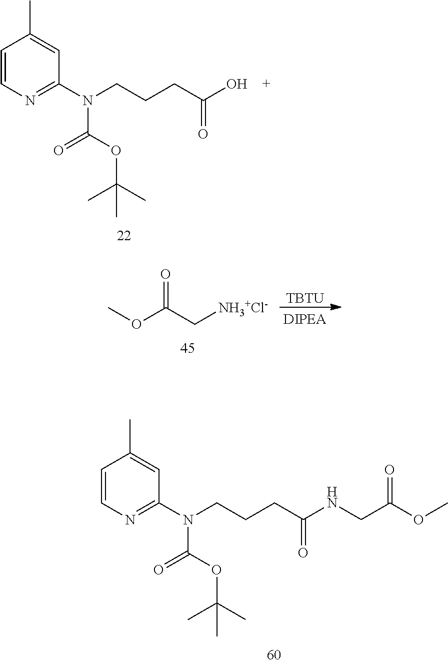

To a solution of Compound 22 (1.1 g, 3.95 mmol, 1 equiv.), Compound 45 (595 mg, 4.74 mmol, 1.2 equiv.), and TBTU (1.52 g, 4.74 mmol, 1.2 equiv.) in anhydrous DMF (10 mL) was added diisopropylethylamine (2.06 mL, 11.85 mmol, 3 equiv.) at 0° C. The reaction mixture was warmed to room temperature and stirred 3 hours. The reaction was quenched by saturated NaHCO₃ solution (10 mL). The aqueous phase was extracted with ethyl acetate (3×10 mL) and the organic phase was combined, dried over anhydrous Na₂SO₄, and concentrated. The product was separated by CombiFlash® using silica gel as the stationary phase. LC-MS: calculated [M+H]+ 366.20. found 367.

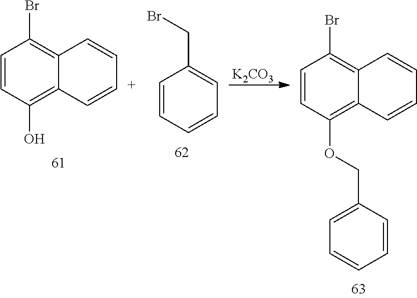

To a solution of compound 61 (2 g, 8.96 mmol, 1 equiv.), and compound 62 (2.13 ML, 17.93 mmol, 2 equiv.) in anhydrous DMF (10 mL) was added K₂CO₃ (2.48 g, 17.93 mmol, 2 equiv.) at 0° C. The reaction mixture was warmed to room temperature and stirred overnight. The reaction was quenched by water (10 mL). The aqueous phase was extracted with ethyl acetate (3×10 mL) and the organic phase was combined, dried over anhydrous Na₂SO₄, and concentrated. The product was separated by CombiFlash® using silica gel as the stationary phase.

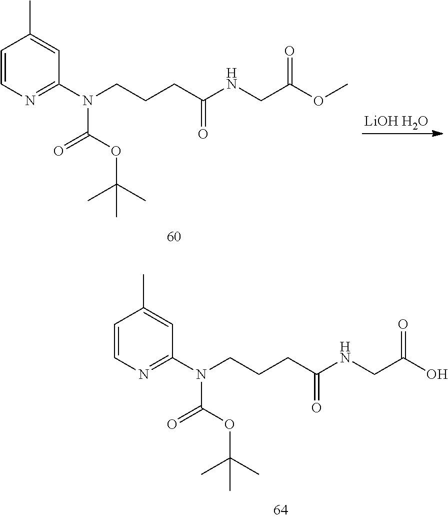 [SMILES: COC(=O)CNC(=O)CCCN(C(=O)OC(C)(C)C)c1cc(C)ccn1 ; "LiOH H2O" ; Cc1ccnc(N(CCCC(=O)NCC(=O)O)C(=O)OC(C)(C)C)c1]

To a solution of compound 60 (1.77 g, 4.84 mmol, 1 equiv.) in THF (5 mL) and H₂O (5 mL) was added lithium hydroxide monohydrate (0.61 g, 14.53 mmol, 3 equiv.) portion-wise at 0° C. The reaction mixture was warmed to room temperature. After stirring at room temperature for 3 hours, the reaction mixture was acidified by HCl (6 N) to pH 3.0. The aqueous phase was extracted with ethyl acetate (3×20 mL) and the organic layer was combined, dried over Na₂SO₄, and concentrated. LC-MS: calculated [M+H]+ 352.18. found 352.

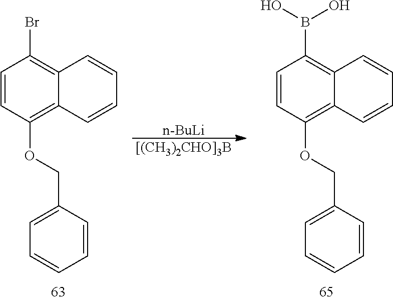

To a solution of compound 63 (1.88 g, 6.0 mmol, 1.0 equiv.) in anhydrous THF (20 mL) was added n-BuLi in hexane (3.6 mL, 9.0 mmol, 1.5 equiv.) drop-wise at −78° C. The reaction was kept at −78° C. for another 1 hour. Triisopropylborate (2.08 mL, 9.0 mmol, 1.5 equiv.) was then added into the mixture at −78° C. The reaction was then warmed up to room temperature and stirred for another 1 hour. The reaction was quenched by saturated NH₄Cl solution (20 mL) and the pH was adjusted to 3. The aqueous phase was extracted with EtOAc (3×20 mL) and the organic phase was combined, dried over Na₂SO₄, and concentrated.

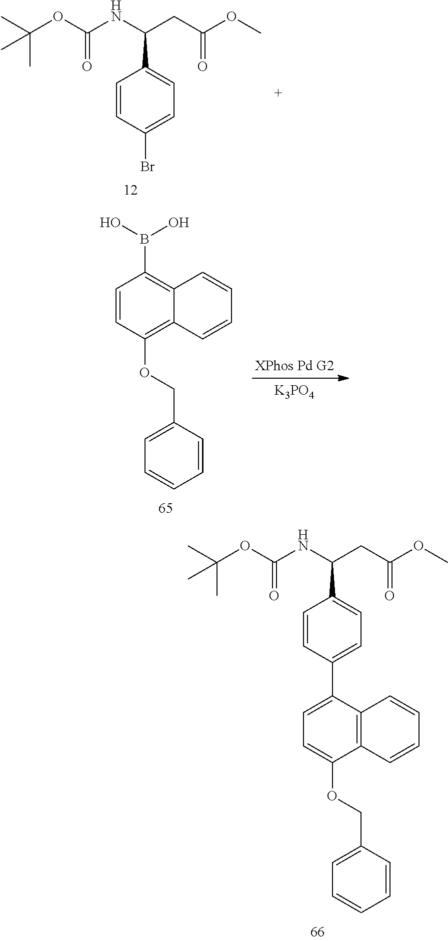

Compound 12 (300 mg, 0.837 mmol, 1.0 equiv.), Compound 65 (349 mg, 1.256 mmol, 1.5 equiv.), XPhos Pd G2 (13 mg, 0.0167 mmol, 0.02 equiv.), and K₃PO₄ (355 mg, 1.675 mmol, 2.0 equiv.) were mixed in a round-bottom flask. The flask was sealed with a screw-cap septum, and then evacuated and backfilled with nitrogen (this process was repeated a total of 3 times). Then, THF (8 mL) and water (2 mL) were added via syringe. The mixture was bubbled with nitrogen for 20 min and the reaction was kept at room temperature for overnight. The reaction was quenched with water (10 mL), and the aqueous phase was extracted with ethyl acetate (3×10 mL). The organic phase was dried over Na₂SO₄, concentrated, and purified via CombiFlash® using silica gel as the stationary phase and was eluted with 15% EtOAc in hexane. LC-MS: calculated [M+H]+ 512.24. found 512.56.

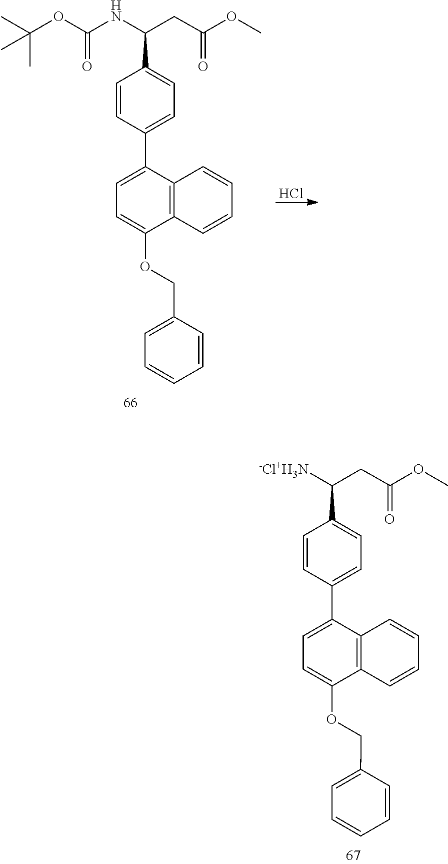

Compound 66 (858 mg, 1.677 mmol, 1.0 equiv.) was cooled by ice bath. HCl in dioxane (8.4 mL, 33.54 mmol, 20 equiv.) was added into the flask. The reaction was warmed to room temperature and stirred for another 1 hr. The solvent was removed by rotary evaporator and the product was directly used without further purification. LC-MS: calculated [M+H]+ 412.18. found 412.46.

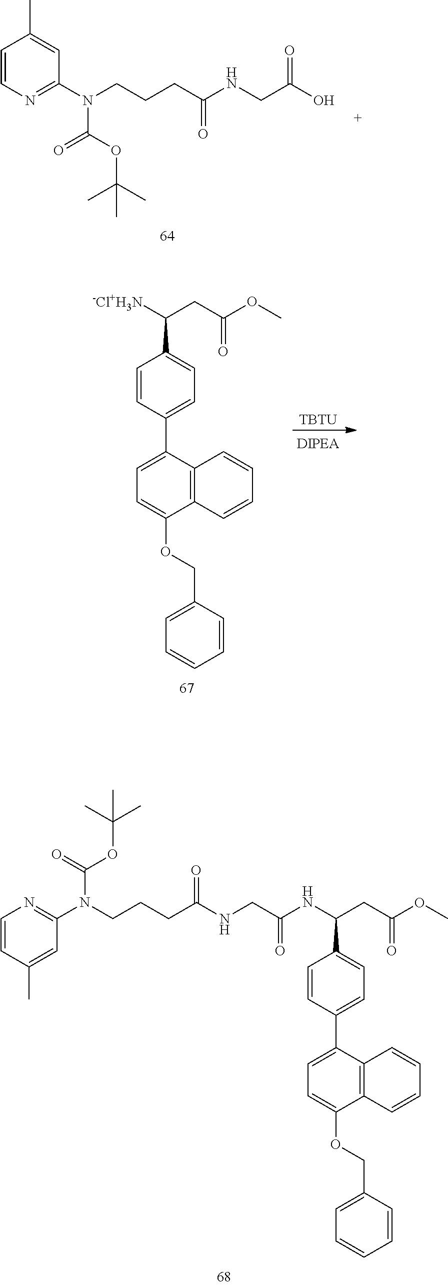

To a solution of compound 64 (500 mg, 1.423 mmol, 1 equiv.), compound 67 (669 mg, 1.494 mmol, 1.05 equiv.), and TBTU (548 mg, 0.492 mmol, 1.2 equiv.) in anhydrous DMF (15 mL) was added diisopropylethylamine (0.744 mL, 4.268 mmol, 3 equiv.) at 0° C. The reaction mixture was warmed to room temperature and stirred for another 1 hr. The reaction was quenched by saturated NaHCO₃ aqueous solution (10 mL) and the product was extracted with ethyl acetate (3×20 mL). The organic phase was combined, dried over Na₂SO₄, and concentrated. The product was purified by CombiFlash® using silica gel as the stationary phase and was eluted with 3-4% methanol in DCM. The yield was 96.23%. LC-MS: calculated [M+H]+ 745.35. found 746.08.

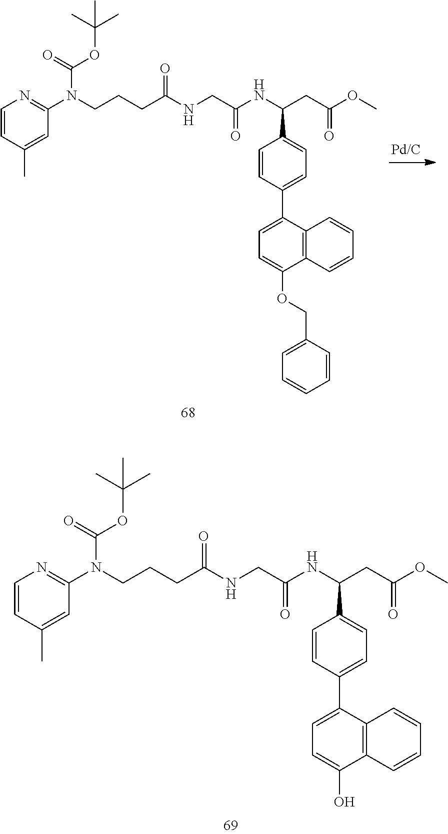 [SMILES: COC(=O)C[C@H](NC(=O)CNC(=O)CCCN(C(=O)OC(C)(C)C)c1cc(C)ccn1)c1ccc(-c2ccc(OCc3ccccc3)c3ccccc23)cc1 ; COC(=O)C[C@H](NC(=O)CNC(=O)CCCN(C(=O)OC(C)(C)C)c1cc(C)ccn1)c1ccc(-c2ccc(O)c3ccccc23)cc1]

To a solution of compound 68 (1.02 g, 1.369 mmol, 1 equiv.) in ethyl acetate (10 mL) was added 10% Pd/C (0.15 g, 50% H₂O) at room temperature. The reaction mixture was warmed to room temperature and the reaction was monitored by LC-MS. The reaction was kept at room temperature overnight. The solids were filtered through Celite® and the solvent was removed by rotary evaporator. The product was directly used without further purification. LC-MS: [M+H]+ 655.31. found 655.87.

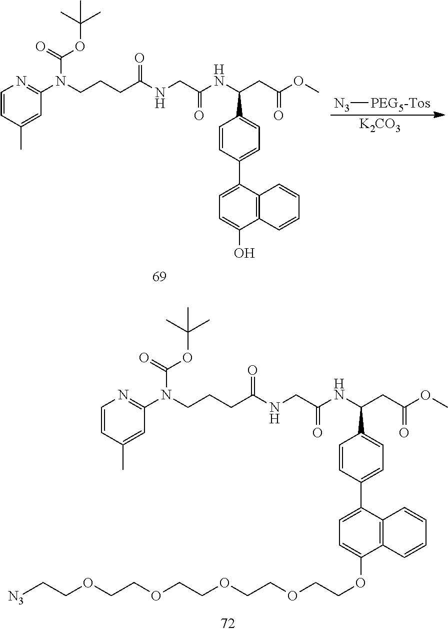

To a solution of compound 69 (100 mg, 0.152 mmol, 1 equiv.) and azido-PEG₅-OTs (128 mg, 0.305 mmol, 2 equiv.) in anhydrous DMF (2 mL) was added K₂CO₃ (42 mg, 0.305 mmol, 2 equiv.) at 0° C. The reaction mixture was stirred for 6 hours at 80° C. The reaction was quenched by saturated NaHCO₃ solution and the aqueous layer was extracted with ethyl acetate (3×10 mL). The organic phase was combined, dried over Na₂SO₄, and concentrated. LC-MS: calculated [M+H]+ 900.40. found 901.46.

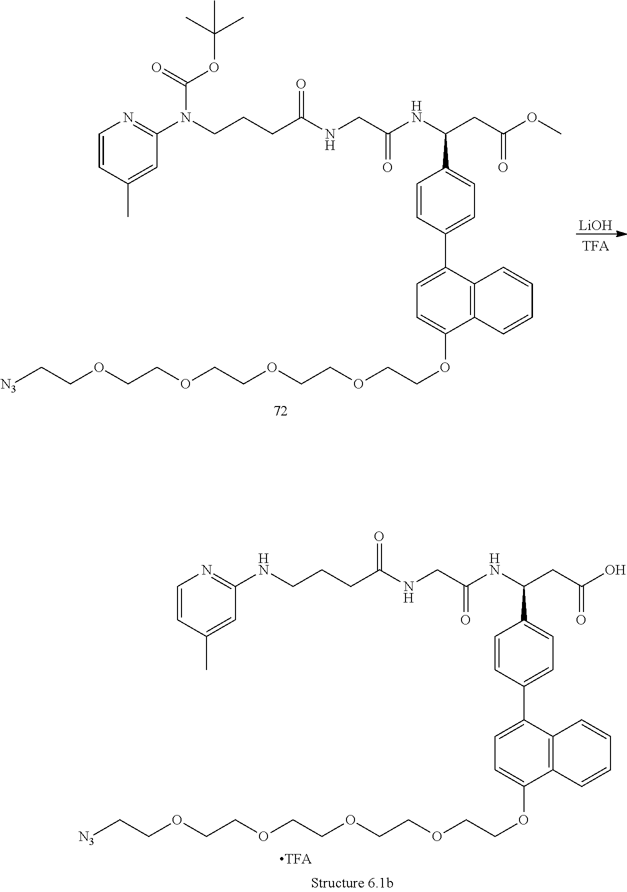 [SMILES: COC(=O)C[C@H](NC(=O)CNC(=O)CCCN(C(=O)OC(C)(C)C)c1cc(C)ccn1)c1ccc(-c2ccc(OCCOCCOCCOCCOCCN=[N+]=[N-])c3ccccc23)cc1 ; "LiOH" ; Cc1ccnc(NCCCC(=O)NCC(=O)N[C@@H](CC(=O)O)c2ccc(-c3ccc(OCCOCCOCCOCCOCCN=[N+]=[N-])c4ccccc34)cc2)c1]

To a solution of compound 72 (59 mg, 0.0656 mmol, 1.0 equiv.) in THF (2 mL) and water (2 mL) was added lithium hydroxide (5 mg, 0.197 mmol, 3.0 equiv.) at room temperature. The mixture was stirred at room temperature for another 1 hr. The pH was adjusted to 3.0 by HCl (6N) and the aqueous phase was extracted with EtOAc (3×10 mL). The organic phase was combined, dried over Na₂SO₄, and concentrated. TFA (0.5 mL) and DCM (0.5 mL) was added into the residue and the mixture was stirred at room temperature for another 3 hr. The solvent was removed by rotary evaporator. LC-MS: calculated [M+H]+ 786.37. found 786.95.

### G. Synthesis of TriAlk 14

TriAlk14 and (TriAlk14)s as shown in Table 11, above, may be synthesized using the synthetic route shown below. Compound 14 may be added to the sense strand as a phosphoramidite using standard oligonucleotide synthesis techniques, or compound 22 may be conjugated to the sense strand comprising an amine in an amide coupling reaction.

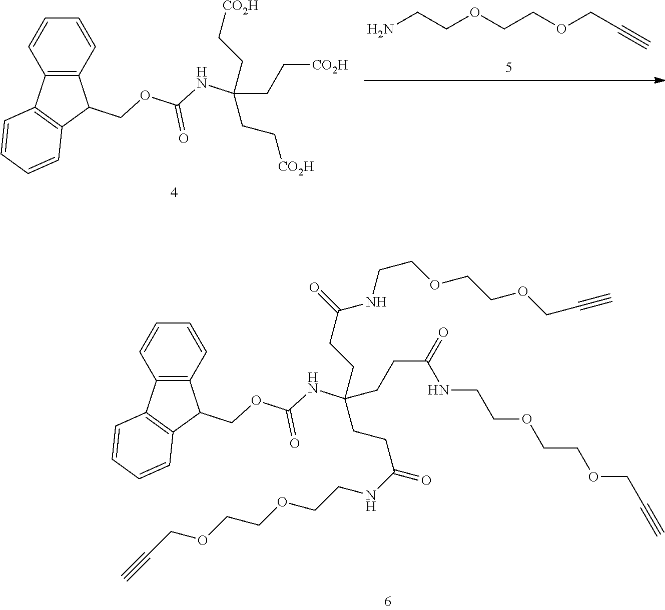

To a 3-L jacketed reactor was added 500 mL DCM and 4 (75.0 g, 0.16 mol). The internal temperature of the reaction was cooled to 0° C. and TBTU (170.0 g, 0.53 mol) was added. The suspension was then treated with the amine 5 (75.5 g, 0.53 mol) dropwise keeping the internal temperature less than 5° C. The reaction was then treated with DIPEA (72.3 g, 0.56 mol) slowly, keeping the internal temperature less than 5° C. After the addition was complete, the reaction was warmed up to 23° C. over 1 hour, and allowed to stir for 3 hours. A 10% kicker charge of all three reagents were added and allowed to stir an additional 3 hours. The reaction was deemed complete when <1% of 4 remained. The reaction mixture was washed with saturated ammonium chloride solution (2×500 mL) and once with saturated sodium bicarbonate solution (500 mL). The organic layer was then dried over sodium sulfate and concentrated to an oil. The mass of the crude oil was 188 g which contained 72% 6 by QNMR The crude oil was carried to the next step. Calculated mass for C₄₆H₆₀N₄O₁₁=845.0 m/z. Found [M+H]=846.0.

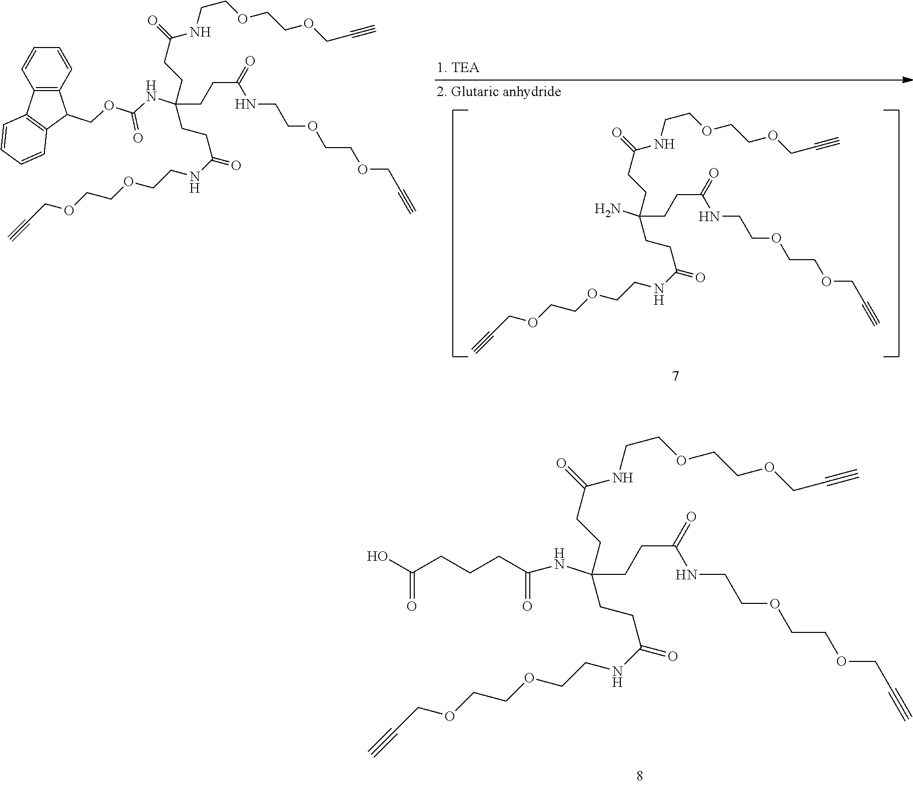

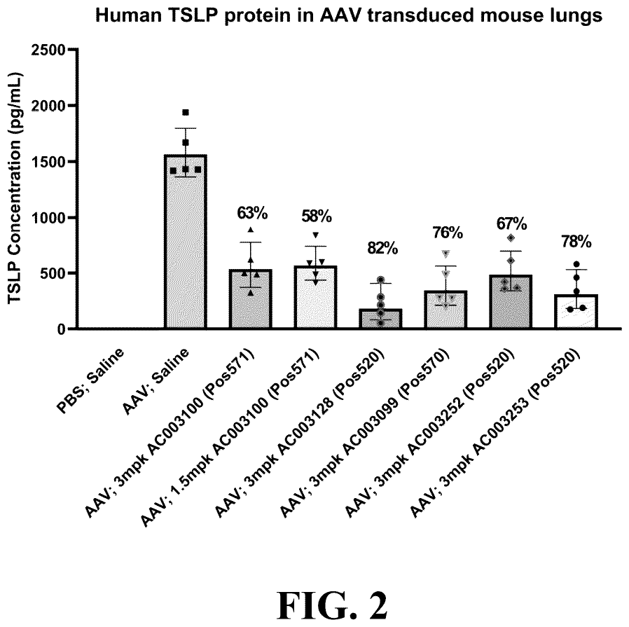

The 121.2 g of crude oil containing 72 wt % compound 6 (86.0 g, 0.10 mol) was dissolved in DMF (344 mL) and treated with TEA (86 mL, 20 v/v %), keeping the internal temperature below 23° C. The formation of dibenzofulvene (DBF) relative to the consumption of Fmoc-amine 6 was monitored via HPLC method 1 (FIG. 2) and the reaction was complete within 10 hours. To the solution was added glutaric anhydride (12.8 g, 0.11 mol) and the intermediate amine 7 was converted to compound 8 within 2 hours. Upon completion, the DMF and TEA were removed at 30° C. under reduced pressure resulting in 100 g of a crude oil. Due to the high solubility of compound 7 in water, an aqueous workup could not be used, and chromatography is the only way to remove DBF, TMU, and glutaric anhydride. The crude oil (75 g) was purified on a Teledyne ISCO Combi-Flash® purification system in three portions. The crude oil (25 g) was loaded onto a 330 g silica column and eluted from 0-20% methanol/DCM over 30 minutes resulting in 42 g of compound 8 (54% yield over 3 steps). Calculated mass for C₃₆H₅₅N₄O₁₂=736.4 m/z. Found [M+H]=737.0.

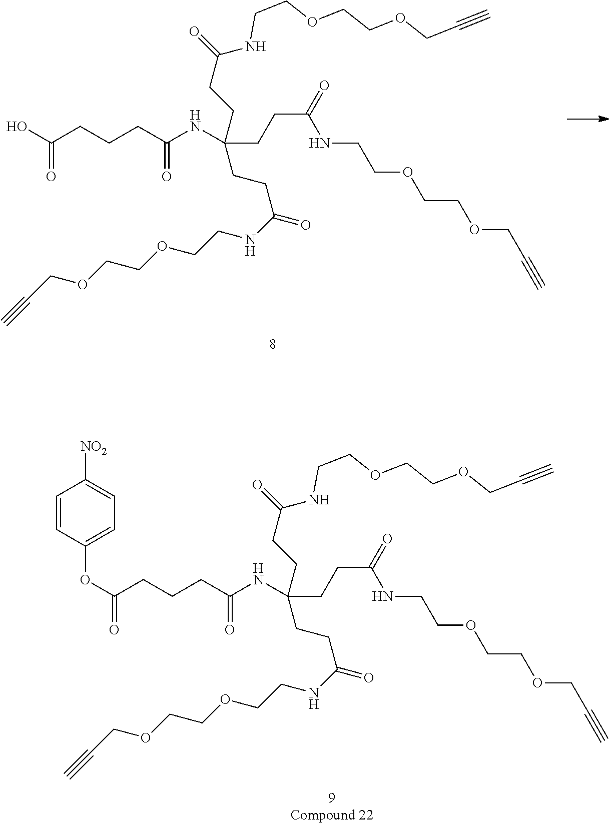 [SMILES: C#CCOCCOCCNC(=O)CCC(CCC(=O)NCCOCCOCC#C)(CCC(=O)NCCOCCOCC#C)NC(=O)CCCC(=O)O ; C#CCOCCOCCNC(=O)CCC(CCC(=O)NCCOCCOCC#C)(CCC(=O)NCCOCCOCC#C)NC(=O)CCCC(=O)Oc1ccc([N+](=O)[O-])cc1]

Compound 8 (42.0 g, 0.057 mol) was co-stripped with 10 volumes of acetonitrile prior to use to remove any residual methanol from chromatography solvents. The oil was redissolved in DMF (210 mL) and cooled to 0° C. The solution was treated with 4-nitrophenol (8.7 g, 0.063 moL) followed by EDC-hydrochloride (12.0 g, 0.063 mol) and found to reach completion within 10 hours. The solution was cooled to 0° C. and 10 volumes ethyl acetate was added followed by 10 volumes saturated ammonium chloride solution, keeping the internal temperature below 15° C. The layers were allowed to separate and the ethyl acetate layer was washed with brine. The combined aqueous layers were extracted twice with 5 volumes ethyl acetate. The combined organic layers were dried over sodium sulfate and concentrated to an oil. The crude oil (55 g) was purified on a Teledyne ISCO Combi-Flash® purification system in three portions. The crude oil (25 g) was loaded onto a 330 g silica column and eluted from 0-10% methanol/DCM over 30 minutes resulting in 22 g of pure 9 (Compound 22) (50% yield). Calculated mass for C₄₂H₅₉N₅O₁₄=857.4 m/z. Found [M+H]=858.0.

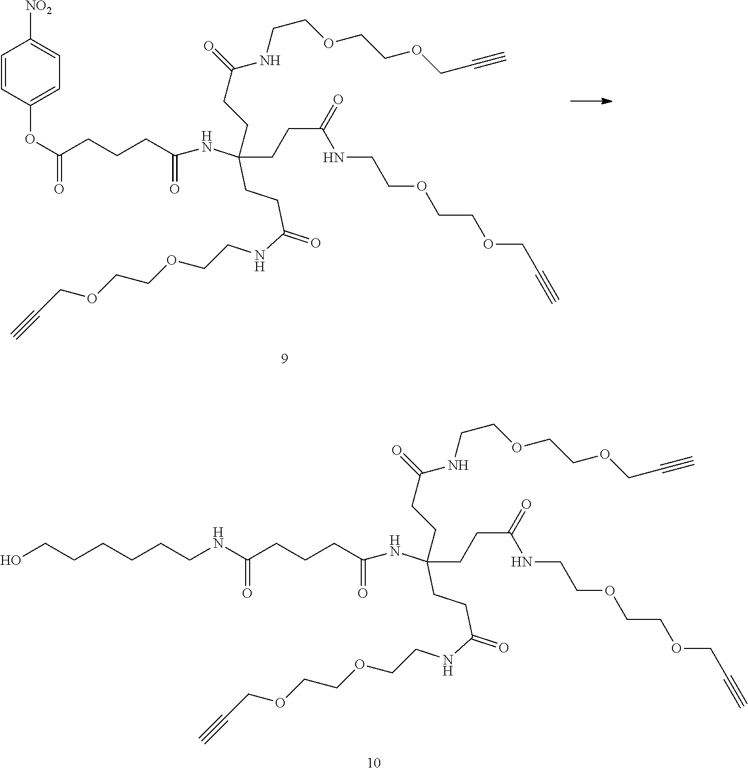 [SMILES: C#CCOCCOCCNC(=O)CCC(CCC(=O)NCCOCCOCC#C)(CCC(=O)NCCOCCOCC#C)NC(=O)CCCC(=O)Oc1ccc([N+](=O)[O-])cc1 ; C#CCOCCOCCNC(=O)CCC(CCC(=O)NCCOCCOCC#C)(CCC(=O)NCCOCCOCC#C)NC(=O)CCCC(=O)NCCCCCCO]

A solution of ester 9 (49.0 g, 57.1 mmol) and 6-amino-1-hexanol (7.36 g, 6.28 mmol) in dichloromethane (3 volumes) was treated with triethylamine (11.56 g, 111.4 mmol) dropwise. The reaction was monitored by observing the disappearance of compound 9 on HPLC Method 1 and was found to be complete in 10 minutes. The crude reaction mixture was diluted with 5 volumes dichloromethane and washed with saturated ammonium chloride (5 volumes) and brine (5 volumes). The organic layer was dried over sodium sulfate and concentrated to an oil. The crude oil was purified on a Teledyne ISCO Combi-Flash® purification system using a 330 g silica column. The 4-nitrophenol was eluted with 100% ethyl acetate and 10 was flushed from the column using 20% methanol/DCM resulting in a colorless oil (39 g, 81% yield). Calculated mass for C₄₂H₆₉N₅O₁₂=836.0 m/z. Found [M+H]837.0.

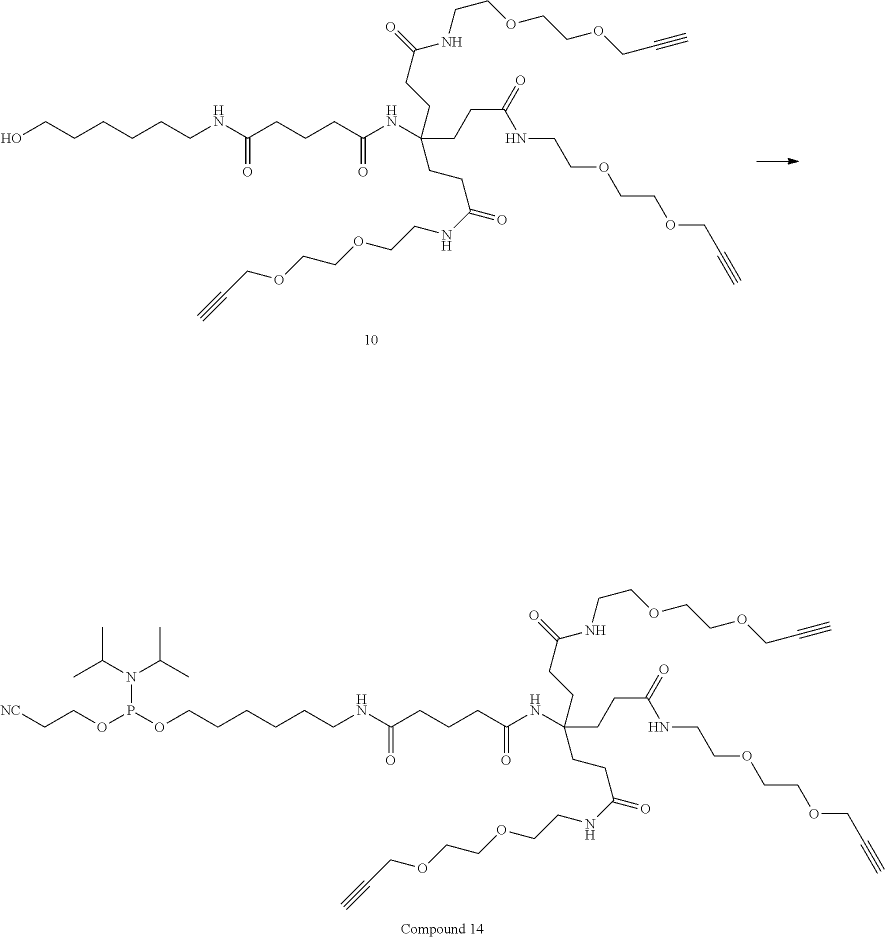 [SMILES: C#CCOCCOCCNC(=O)CCC(CCC(=O)NCCOCCOCC#C)(CCC(=O)NCCOCCOCC#C)NC(=O)CCCC(=O)NCCCCCCO ; C#CCOCCOCCNC(=O)CCC(CCC(=O)NCCOCCOCC#C)(CCC(=O)NCCOCCOCC#C)NC(=O)CCCC(=O)NCCCCCCOP(OCCC#N)N(C(C)C)C(C)C]

Alcohol 10 was co-stripped twice with 10 volumes of acetonitrile to remove any residual methanol from chromatography solvents and once more with dry dichloromethane (KF<60 ppm) to remove trace water. The alcohol 10 (2.30 g, 2.8 mmol) was dissolved in 5 volumes dry dichloromethane (KF<50 ppm) and treated with diisopropylammonium tetrazolide (188 mg, 1.1 mmol). The solution was cooled to 0° C. and treated with 2-cyanoethyl N,N,N′,N′-tetraisopropylphosphoramidite (1.00 g, 3.3 mmol) dropwise. The solution was removed from ice-bath and stirred at 20° C. The reaction was found to be complete within 3-6 hours. The reaction mixture was cooled to 0° C. and treated with 10 volumes of a 1:1 solution of saturated ammonium bicarbonate/brine and then warmed to ambient over 1 minute and allowed to stir an additional 3 minutes at 20° C. The biphasic mixture was transferred to a separatory funnel and 10 volumes of dichloromethane was added. The organic layer was separated and washed with 10 volumes of saturated sodium bicarbonate solution to hydrolyze unreacted bis-phosphorous reagent. The organic layer was dried over sodium sulfate and concentrated to an oil resulting in 3.08 g of 94 wt % Compound 14. Calculated mass for C₅₁H₈₆N₇O₁₃P=1035.6 m/z. Found [M+H]=1036.

H. Conjugation of Targeting Ligands. Either prior to or after annealing, the 5′ or 3′ tridentate alkyne functionalized sense strand is conjugated to targeting ligands. The following example describes the conjugation of targeting ligands to the annealed duplex: Stock solutions of 0.5M Tris(3-hydroxypropyltriazolylmethyl)amine (THPTA), 0.5M of Cu(II) sulfate pentahydrate (Cu(II)SO₄·5H₂O) and 2M solution of sodium ascorbate were prepared in deionized water. A 75 mg/mL solution in DMSO of targeting ligand was made. In a 1.5 mL centrifuge tube containing tri-alkyne functionalized duplex (3 mg, 75 μL, 40 mg/mL in deionized water, ˜15,000 g/mol), 25 μL of 1M Hepes pH 8.5 buffer is added. After vortexing, 35 μL of DMSO was added and the solution is vortexed. Targeting ligand was added to the reaction (6 equivalents/duplex, 2 equivalents/alkyne, ˜15 μL) and the solution is vortexed. Using pH paper, pH was checked and confirmed to be pH˜8. In a separate 1.5 mL centrifuge tube, 50 μL of 0.5M THPTA was mixed with 10 μL of 0.5M Cu(II)SO₄·5H₂O, vortexed, and incubated at room temp for 5 min. After 5 min, THPTA/Cu solution (7.2 μL, 6 equivalents 5:1 THPTA:Cu) was added to the reaction vial, and vortexed. Immediately afterwards, 2M ascorbate (5 μL, 50 equivalents per duplex, 16.7 per alkyne) was added to the reaction vial and vortexed. Once the reaction was complete (typically complete in 0.5-1h), the reaction was immediately purified by non-denaturing anion exchange chromatography.

### Example 2. In Vivo Anti-Inflammatory Effect of TSLP Knock-Down in Rat Model of Airway Inflammation, Delivery Via Intra-Tracheal Microsprayer

On study day 1 and day 3, male Sprague Dawley rats were administered a dose of 5 mg/kg of a rat-specific RNAi agent linked to a Tri-SM6.1-αvβ6 integrin targeting ligand (referred to as AC001714), or saline vehicle. Volume of 200 μL was loaded into a syringe that was connected to a microsprayer device (Penn Century, Philadelphia, PA) for intra-tracheal administration.

AC001714 includes a rat-specific sequence designed to target the rat TSLP transcript (NCBI GenBank XM_008772052.2) and does not have homology with the human TSLP gene, and was chemically modified as follows:

Modified Sense Strand (5′→3′):
(SEQ ID NO: 782)
Tri-SM6.1-avb6-(TA14)-
gsa_2NaucaaaCfCfUfcacaaauucus(invAb)
Modified Antisense Strand (5′→3′):
(SEQ ID NO: 587)
cPrpasGfsasAfuUfuGfuGfaGfgUfuUfgAfuUfsc

On day 14, rats were challenged with a single intra-tracheal dose of 400 μg/rat of Alternaria alternata prepared in phosphate buffered saline (PBS). Rats in Group 1 were administered only with PBS as control.

TABLE 12
Rat-specific TSLP RNAi Agent and Dosing for Example 2.

| | AC | Animals | Harvest/ |
|---|---|---|---|
| | Duplex | per | Sacrifice |
| Group ID | Number | Group | Day |
| Group 1 (saline IT days 1, 3) (PBS IT day 14) | N/A | 4 | Day 15 |
| Group 2 (saline IT days 1, 3) (Alternaria | N/A | 7 | Day 15 |
| IT day 14) | | | |
| Group 3 (saline IT days 1, 3) (Alternaria | N/A | 7 | Day 16 |
| IT day 14) | | | |
| Group 4 (saline IT days 1, 3) (Alternaria | N/A | 5 | Day 17 |
| IT day 14) | | | |
| Group 5 (IT dose 5.0 mg/kg AC001714 on days | AC001714 | 7 | Day 15 |
| 1, 3)/(Alternaria IT day 14) | | | |
| Group 6 (IT dose 5.0 mg/kg AC001714 on days | AC001714 | 5 | Day 16 |
| 1, 3)/(Alternaria IT day 14) | | | |
| Group 7 (IT dose 5.0 mg/kg AC001714 on days | AC001714 | 5 | Day 17 |
| 1, 3)/(Alternaria IT day 14) | | | |

After either 24, 48, or 72 hours post-administration of the Alternaria (i.e., either day 15, 16, or 17), rats were anesthetized with isoflurane/02, blood was drawn, and were euthanized by exsanguination. Days of sacrifice/euthanasia are shown in Table 12 above. Trachea was canulated and bronchoalveolar lavage (BAL) collected after washing with 2×5 mL of ice-cold PBS. BAL samples were spun down, cells resuspended with 1 mL of ice-cold PBS, and aliquot was mixed with Turk's solution (ratio 1:1), and total cell counted via hemocytomers. Cytospins were prepared, stained and differential cell counting performed. Supernatant was used for cytokine measurements. Right lung lobes were used to determine rTSLP mRNA expression and left lung lobes were collected in 4% PFA/PBS for histology (Trichrome and Sirius Red Staining, RNAscope).

Rat TSLP mRNA expression was quantitated by probe-based quantitative PCR, normalized to rat B2M expression, and expressed as fraction of vehicle control group (geometric mean, +/−95% confidence interval).

TABLE 13
Average Relative Rat TSLP mRNA Expression at Sacrifice
(i.e., Day 15, 16, or 17) in Example 2

| | Average Relative rTSLP | Low | High |
|---|---|---|---|
| Group ID | mRNA Expression | (error) | (error) |
| Group 1 (saline IT days 1, 3) (PBS IT day 14) | 1.000 | 0.228 | 0.296 |
| Group 2 (saline IT days 1, 3) (Alternaria IT | 0.787 | 0.149 | 0.183 |
| day 14) | | | |
| Group 3 (saline IT days 1, 3) (Alternaria IT | 1.072 | 0.151 | 0.176 |
| day 14) | | | |
| Group 4 (saline IT days 1, 3) (Alternaria IT | 0.828 | 0.121 | 0.142 |
| day 14) | | | |
| Group 5 (IT dose 5.0 mg/kg AC001714 on | 0.387 | 0.098 | 0.131 |
| days 1, 3)/(Alternaria IT day 14) | | | |
| Group 6 (IT dose 5.0 mg/kg AC001714 on | 0.379 | 0.102 | 0.139 |
| days 1, 3)/(Alternaria IT day 14) | | | |
| Group 7 (IT dose 5.0 mg/kg AC001714 on | 0.459 | 0.095 | 0.121 |
| days 1, 3)/(Alternaria IT day 14) | | | |

As shown in Table 13 above, the Groups administered AC001714 (i.e., Groups 5, 6 and 7) each showed reductions of approximately 45-65% of rTSLP mRNA at the respective time of sacrifice relative to the respective control groups (Groups 2, 3, and 4).

Granulocytes (both eosinophils and neutrophils) are well known markers for cellular inflammation. For the BAL samples, the total and differential cells were counted and the number of inflammatory cells were derived. The impact of rTSLP inhibition by the rat-specific TSLP RNAi agents disclosed herein on eosinophilic inflammation induced by Alternaria extract was assessed. Groups 5-7 (treated with rat-specific TSLP RNAi agent) showed significant reductions of total BAL cell counts, lymphocytes, and neutrophils across all time points when compared to their respective control. Further, a significant reduction of eosinophils at the 72 hour time point (Group 7) was observed as compared to Group 4. Moreover, BAL total protein was significantly reduced at both 24 hour and 72 hour time points (Group 5 and 7) as compared to control groups 2 and 4 respectively.

Other biomarkers, such as IL-18 and VEGF, are also indicative of cellular inflammation. For the Alternaria challenged groups, administration of the rat-specific RNAi agent (Groups 5, 6 and 7) resulted in a reductions of each of these pro-inflammatory biomarkers compared to the group in which no RNAi agent was administered. This study provides physiological support in a rat model that a reduction in TSLP gene expression of approximately 45% or more can provide a phenotype improvement to reduce pulmonary inflammation, and thus can potentially treat diseases such as allergic asthma.

### Example 3. In Vivo Anti-Inflamatory Effect of TSLP Knock-Down in Rat Model of Airway Inflammation, Delivery Via Intra-Tracheal Microsprayer

On study day 1 and day 3, male Brown-Norway rats were administered a dose of 5 mg/kg of a rat-specific RNAi agent linked to a Tri-SM6.1-αvβ6 integrin targeting ligand (referred to as AC001714 or AC002515), or saline vehicle. Additionally, a “RISC-blocked” RNAi trigger was used, which include a construct similar to AC001714, including the same targeting ligand, but included chemical modifications designed to prevent the loading of the antisense strand into RISC, thus serving as a negative control. Volume of 200 μL was loaded into a syringe that was connected to a microsprayer device (Penn Century, Philadelphia, PA) for intra-tracheal administration.

AC001714 includes a rat-specific sequence designed to target the rat TSLP transcript (NCBI GenBank XM_008772052.2) and does not have homology with the human TSLP gene, the chemical structure of which is shown above in Example 2.

AC002515 is also a rat-specific sequence designed to target a different position on the rat TSLP transcript (NCBI GenBank XM_008772052.2) that does not have homology with the human TSLP gene, and was chemically modified as follows:

Modified Sense Strand (5′→3′):
(SEQ ID NO: 783)
Tri-SM6.1-avb6-(TA14)-csugaaacuGfAfGfagaaaugguas
(invAb)
Modified Antisense Strand (5′→3′):
(SEQ ID NO: 588)
cPrpusAfscsCfaUfuucucUfcAfgUfuUfcasg

On day 14, rats were challenged with a single intra-tracheal dose of 500 μg/rat of Alternaria alternata prepared in PBS. Rats in Group 1 were administered only with PBS as a control.

TABLE 14
Rat-specific TSLP RNAi Agent and Dosing for Example 3.

| | AC | Animals | Harvest/ |
|---|---|---|---|
| | Duplex | per | Sacrifice |
| Group ID | Number | Group | Day |
| Group 1 (saline IT days 1, 3) (PBS IT day 15) | N/A | 6 | Day 16 |
| Group 2 (saline IT days 1, 3) (Alternaria IT | N/A | 6 | Day 16 |
| day 15) | | | |
| Group 3 (saline IT days 1, 3) (IT dose 5.0 | RISC-blocked | 6 | Day 16 |
| mg/kg RISC-blocked trigger on days 1, 3)/ | RNAi Trigger | | |
| (Alternaria IT day 15) | | | |
| Group 4 (IT dose 5.0 mg/kg AC001714 on days | AC001714 | 7 | Day 16 |
| 1, 3)/(Alternaria IT day 15) | | | |
| Group 5 (IT dose 5.0 mg/kg AC002515 on days | AC002515 | 7 | Day 16 |
| 1, 3)/(Alternaria IT day 15) | | | |

After 24 hours post-administration of the Alternaria (i.e., day 16), rats were anesthetized with isoflurane/02, blood was drawn, and were euthanized by exsanguination. Days of sacrifice/euthanasia are shown in Table 14 above. Trachea was canulated and bronchoalveolar lavage (BAL) collected after washing with 2×5 mL of ice-cold PBS. BAL samples were spun down, cells resuspended with 1 mL of ice-cold PBS, and aliquot was mixed with Turk's solution (ratio 1:1), and total cell counted via hemocytomers. Cytospins were prepared, stained and differential cell counting performed. Supernatant was used for cytokine measurements. Right lung lobes were used to determine rTSLP mRNA expression and left lung lobes were collected in 4% PFA/PBS for histology (Trichrome and Sirius Red Staining, RNAscope).

Rat TSLP mRNA expression was quantitated by probe-based quantitative PCR, normalized to rat B2M expression, and expressed as fraction of vehicle control group (geometric mean, +/−95% confidence interval).

TABLE 15
Average Relative Rat TSLP mRNA Expression
at Sacrifice (i.e., Day 16) in Example 3

| | Average Relative rTSLP | Low | High |
|---|---|---|---|
| Group ID | mRNA Expression | (error) | (error) |
| Group 1 (saline IT days 1, 3) (PBS IT day | 1.232 | 0.224 | 0.273 |
| 15) | | | |
| Group 2 (saline IT days 1, 3) (Alternaria | 1.000 | 0.143 | 0.167 |
| IT day 15) | | | |
| Group 3 (saline IT days 1, 3) (IT dose 5.0 | 0.953 | 0.144 | 0.170 |
| mg/kg RISC-blocked trigger on days 1, 3)/ | | | |
| (Alternaria IT day 15) | | | |
| Group 4 (IT dose 5.0 mg/kg AC001714 on | 0.382 | 0.109 | 0.153 |
| days 1, 3)/(Alternaria IT day 15) | | | |
| Group 5 (IT dose 5.0 mg/kg AC002515 on | 0.598 | 0.096 | 0.115 |
| days 1, 3)/(Alternaria IT day 15) | | | |

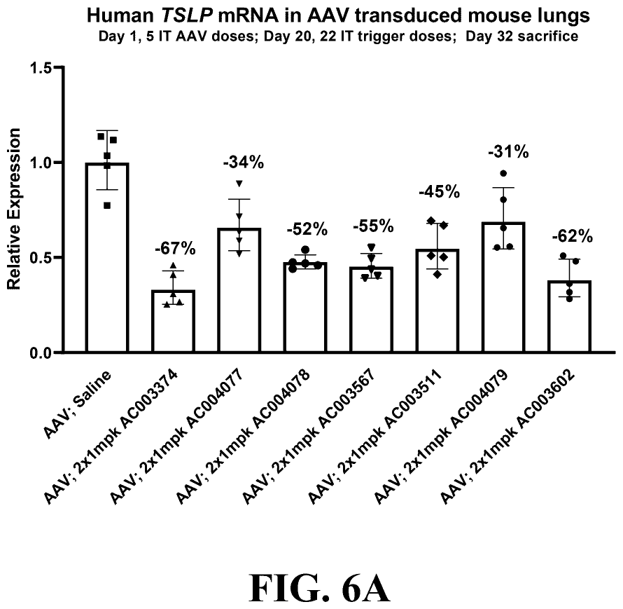

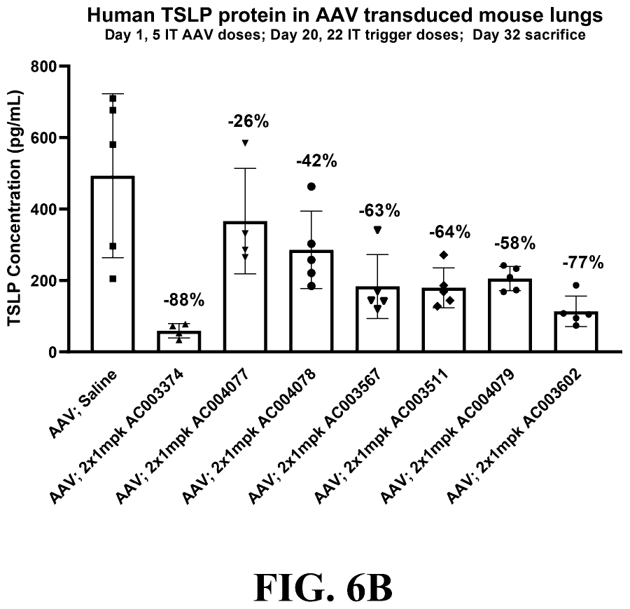

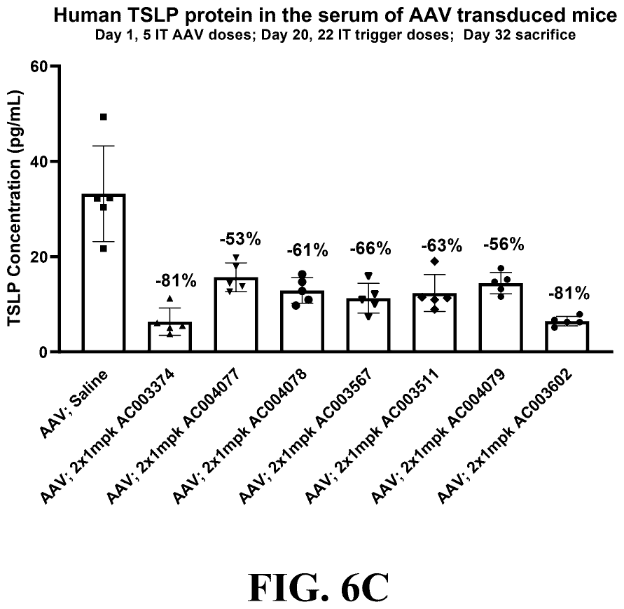

As shown in Table 15 above, the Groups administered AC001714 (Group 4) and AC002515 (Group 5) each showed reductions of TSLP mRNA, with AC001714 showing approximately 62% inhibition. This is also shown in FIG. 6A.

IL-13 and IL-33 are Th2 cytokines that are known indicators for inflammation in the lung. Rat IL-13 mRNA expression and Rat IL-33 mRNA expression were similarly quantitated by probe-based quantitative PCR, normalized to rat B2M expression, and expressed as fraction of vehicle control group (geometric mean, +/−95% confidence interval).

TABLE 16
Average Relative Rat IL-13 mRNA Expression
at Sacrifice (i.e., Day 16) in Example 3

| | Average Relative rIL-13 | Low | High |
|---|---|---|---|
| Group ID | mRNA Expression | (error) | (error) |
| Group 1 (saline IT days 1, 3) (PBS IT day | 1.000 | 0.279 | 0.387 |
| 15) | | | |
| Group 2 (saline IT days 1, 3) (Alternaria | 4.018 | 2.735 | 8.564 |
| IT day 15) | | | |
| Group 3 (saline IT days 1, 3) (IT dose 5.0 | 10.727 | 4.676 | 8.290 |
| mg/kg RISC-blocked trigger on days 1, 3)/ | | | |
| (Alternaria IT day 15) | | | |
| Group 4 (IT dose 5.0 mg/kg AC001714 on | 1.637 | 0.856 | 1.793 |
| days 1, 3)/(Alternaria IT day 15) | | | |
| Group 5 (IT dose 5.0 mg/kg AC002515 on | 1.151 | 0.652 | 1.506 |
| days 1, 3)/(Alternaria IT day 15) | | | |

TABLE 17
Average Relative Rat IL-33 mRNA Expression
at Sacrifice (i.e., Day 16) in Example 3

| | Average Relative rIL-33 | Low | High |
|---|---|---|---|
| Group ID | mRNA Expression | (error) | (error) |
| Group 1 (saline IT days 1, 3) (PBS IT day | 1.000 | 0.286 | 0.401 |
| 15) | | | |
| Group 2 (saline IT days 1, 3) (Alternaria | 1.366 | 0.395 | 0.556 |
| IT day 15) | | | |
| Group 3 (saline IT days 1, 3) (IT dose 5.0 | 1.710 | 0.215 | 0.246 |
| mg/kg RISC-blocked trigger on days 1, 3)/ | | | |
| (Alternaria IT day 15) | | | |
| Group 4 (IT dose 5.0 mg/kg AC001714 on | 0.848 | 0.297 | 0.458 |
| days 1, 3)/(Alternaria IT day 15) | | | |
| Group 5 (IT dose 5.0 mg/kg AC002515 on | 0.792 | 0.172 | 0.220 |
| days 1, 3)/(Alternaria IT day 15) | | | |

As shown in Tables 16 and 17 above, the Groups administered AC001714 (Group 4) and AC002515 (Group 5) that were challenged with Alternaria each showed cytokine levels that were maintained to levels similar to the untreated group (Group 1) that was not challenged, indicating a preventative effect. In contrast, both the Alternaria group without an RNAi treatment (Group 2) and the Alternaria group with a RISC-blocked RNAi trigger that is unable to inhibit rTSLP gene expression both showed marked increases in IL-13 and IL-33, indicative of lung inflammation. This IL-13 mRNA levels are also shown in FIG. 6B, and IL-33 shown in FIG. 6C.

Further, as noted in the prior Example, granulocytes (both eosinophils and neutrophils) are well known markers for cellular inflammation. For the BAL samples, the total and differential cells were counted and the number of inflammatory cells were derived. The impact of rTSLP inhibition by the rat-specific TSLP RNAi agents disclosed herein on eosinophilic inflammation induced by Alternaria extract was assessed. Groups 4 and 5 (treated with rat-specific TSLP RNAi agent) showed significant reductions of lymphocytes. The negative control group (Group 3) showed no such changes, confirming that the reductions are due to the reductions in TSLP mRNA. Further, a trend of reduction of BAL total protein, eosinophils, and total BAL cell counts was observed only in the two treatment Groups (Groups 4 and 5). Furthermore, soluble collagen content was significantly reduced in the two treatment Groups (Groups 4 and 5) relative to the Alternaria control (Group 2).

Other biomarkers, such as IL-13, IL-5, Leptin, MCP-1, RATES, TNF-alpha, and IP-10 are also indicative of cellular inflammation. For the Alternaria challenged groups, administration of the rat-specific RNAi agent (Group 4 and 5) resulted in a trend showing reductions of each of these pro-inflammatory biomarkers compared to the group in which no RNAi agent was administered (Group 2) and the negative control trigger group (Group 3).

As shown in FIG. 6D, rat-specific TSLP RNAi agents achieved significant reduction in BAL soluble collagen (Groups 4 and 5) in comparison with no RNAi agent Alternaria control group (Group 2) as well as negative control RISC-blocked Alternaria group (Group 3). Statistical significance is denoted * p-value is p<0.05.

As shown in FIG. 6E (BAL IL-5) and FIG. 6F (BAL IL-13), rat-specific TSLP RNAi agents (Groups 4 and 5) also achieved reduction of IL-5 and IL-13 in comparison with no RNAi agent Alternaria control group (Group 2) as well as negative control RISC-blocked Alternaria group (Group 3).

Duplex RNAscope of TSLP and ITGB6 confirmed TSLP is expressed in airway epithelium. Co-staining of TSLP RNAscope and Sftpc IHC demonstrated TSLP expression in alveolar type 2 cells.

### Example 4. AAV9-CAG-hTSLP AAV Mouse Model

The following procedure was used to evaluate TSLP RNAi agents in an AAV mouse model. To evaluate certain TSLP RNAi agents, an AAV9-CAG-hTSLP (Adeno-associated virus) mouse model was used. The transgenic sequence included human TSLP CDS with 3′UTR. Six- to eight-week-old female C57BL/6 mice were transduced with human TSLP using AAV with serotype 9 (specifically, AAV9-CAG-hTSLP) and eGFP using AAV9-CAG-eGFP. Mice were intratracheally administered AAV several weeks prior to intracheal administration of either TSLP RNAi agents or control. The genome of the AAV9-CAG-hTSLP. UTRs construct contains the human TSLP cDNA sequence (GenBank NM_033035.5). eGFP was used as a control to normalize human TSLP mRNA expression by qPCR. 2e10 GC of the respective AAV mixed in PBS in a total volume of 50 μL was intratracheally (IT) delivered into mice to create AAV-hTSLP model mice. Lung tissues were collected 2-3 weeks after the administration of RNAi agents.

The human TSLP mRNA expression was measured in the lung tissues by qPCR.

At day 1 and Day 3, each mouse was given an intratracheal (IT) administration of 50 μL AAV solutions containing 2e10 GC (genome copy) of AAV9-CAG-eGFP and 2e10 GC of AAV9-CAG-hTSLP in PBS, or vehicle control (PBS). At Day 30 and 31, each mouse was given intratracheal administration of 50 μL of different dose levels of TSLP RNAi agents formulated in isotonic saline, or vehicle control (isotonic saline with no RNAi agent), according to the following Table 18. The mice were humanely sacrificed and harvested on Day 44.

TABLE 18
Targeted Positions and Dosing Groups of Example 4.

| | Targeted TSLP | | | |
|---|---|---|---|---|
| | Gene Position | | | |
| | (within SEQ ID | | RNAi Agent | |
| | NO: 1, GenBank | AAV dose | and Dose | |
| Group | NM_033035.5) | (Day 1, 3) | (Day 30, 31) | Dosing Regimen |
| 1 | N/A | PBS | Saline (no RNAi | IT doses of PBS on |
| | | | agent) | Day 1, 3; |
| | | | | IT doses of saline on |
| | | | | day 30, 31 |
| 2 | N/A | 2e10 GC of AAV9- | Saline (no RNAi | IT doses of AAV on |
| | | CAG-eGFP and | agent) | Day 1, 3; |
| | | 2e10 GC of AAV9- | | IT doses of saline on |
| | | CAG-hTSLP | | day 30, 31 |
| 3 | 398 | 2e10 GC of AAV9- | 0.5 mg/kg | IT doses of AAV on |
| | | CAG-eGFP and | AC003096 | Day 1, 3; |
| | | 2e10 GC of AAV9- | | IT doses of RNAi |
| | | CAG-hTSLP | | agent on day 30, 31 |
| 4 | 410 | 2e10 GC of AAV9- | 0.5 mg/kg | IT doses of AAV on |
| | | CAG-eGFP and | AC003097 | Day 1, 3; |
| | | 2e10 GC of AAV9- | | IT doses of RNAi |
| | | CAG-hTSLP | | agent on day 30, 31 |
| 5 | 515 | 2e10 GC of AAV9- | 0.5 mg/kg | IT doses of AAV on |
| | | CAG-eGFP and | AC003098 | Day 1, 3; |
| | | 2e10 GC of AAV9- | | IT doses of RNAi |
| | | CAG-hTSLP | | agent on day 30, 31 |
| 6 | 570 | 2e10 GC of AAV9- | 0.5 mg/kg | IT doses of AAV on |
| | | CAG-eGFP and | AC003099 | Day 1, 3; |
| | | 2e10 GC of AAV9- | | IT doses of RNAi |
| | | CAG-hTSLP | | agent on day 30, 31 |
| 7 | 571 | 2e10 GC of AAV9- | 0.5 mg/kg | IT doses of AAV on |
| | | CAG-eGFP and | AC003100 | Day 1, 3; |
| | | 2e10 GC of AAV9- | | IT doses of RNAi |
| | | CAG-hTSLP | | agent on day 30, 31 |
| 8 | 568 | 2e10 GC of AAV9- | 0.5 mg/kg | IT doses of AAV on |
| | | CAG-eGFP and | AC003101 | Day 1, 3; |
| | | 2e10 GC of AAV9- | | IT doses of RNAi |
| | | CAG-hTSLP | | agent on day 30, 31 |
| 9 | 520 | 2e10 GC of AAV9- | 0.5 mg/kg | IT doses of AAV on |
| | | CAG-eGFP and | AC003128 | Day 1, 3; |
| | | 2e10 GC of AAV9- | | IT doses of RNAi |
| | | CAG-hTSLP | | agent on day 30, 31 |
| 10 | 413 | 2e10 GC of AAV9- | 0.5 mg/kg | IT doses of AAV on |
| | | CAG-eGFP and | AC003129 | Day 1, 3; |
| | | 2e10 GC of AAV9- | | IT doses of RNAi |
| | | CAG-hTSLP | | agent on day 30, 31 |
| 11 | 406 | 2e10 GC of AAV9- | 0.5 mg/kg | IT doses of AAV on |
| | | CAG-eGFP and | AC003130 | Day 1, 3; |
| | | 2e10 GC of AAV9- | | IT doses of RNAi |
| | | CAG-hTSLP | | agent on day 30, 31 |

Each of the TSLP RNAi agents included mod ified nucleotides that were conjugated at the 5′ terminal end of the sense strand to an αvβ6 integrin targeting ligand having the modified sequences as set forth in the duplex structures herein. (See Tables 3, 4, 5, 6, 7A, 7B, 8, 9, 10, and 11 for specific modifications and structure information related to the TSLP RNAi agents, including Tri-SM6.1-αvβ6). The TSLP RNAi agents in Groups 3-8 each included nucleotide sequences that were designed to inhibit expression of a TSLP gene by targeting specific positions of TSLP mRNA as set forth in Table 18, above. (See. e.g., SEQ ID NO:1 and Table 2 for the TSLP mRNA sequence referenced.)

Five (5) mice in each group were tested (n=5) in each group, except for Group 1 where only 4 mice were tested. TSLP mRNA expression levels were determined by qPCR Data from the experiment are shown in the following Table 19:

TABLE 19
Average Relative TSLP Normalized to Control in AAV-hTSLP Mice from Example 4.

| | Average Relative hTSLP | Low | High |
|---|---|---|---|
| Group ID | mRNA Expression | (error) | (error) |
| Group 1 (PBS IT days 1, 3) (saline IT days 30, 31) | N/A | | |
| Group 2 (AAV days 1, 3) (saline IT days 30, 31) | 1.000 | 0.097 | 0.108 |
| Group 3 (AAV IT days 1, 3) (0.5 mg/kg AC003096 IT days 30, 31) | 0.826 | 0.159 | 0.197 |
| Group 4 (AAV IT days 1, 3) (0.5 mg/kg AC003097 IT days 30, 31) | 0.737 | 0.187 | 0.250 |
| Group 5 (AAV IT days 1, 3) (0.5 mg/kg AC003098 IT days 30, 31) | 0.882 | 0.145 | 0.174 |
| Group 6 (AAV IT days 1, 3) (0.5 mg/kg AC003099 IT days 30, 31) | 0.701 | 0.050 | 0.054 |
| Group 7 (AAV IT days 1, 3) (0.5 mg/kg AC003100 IT days 30, 31) | 0.576 | 0.085 | 0.099 |
| Group 8 (AAV IT days 1, 3) (0.5 mg/kg AC003101 IT days 30, 31) | 0.930 | 0.158 | 0.191 |
| Group 9 (AAV IT days 1, 3) (0.5 mg/kg AC003128 IT days 30, 31) | 0.522 | 0.092 | 0.112 |
| Group 10 (AAV IT days 1, 3) (0.5 mg/kg AC003129 IT days 30, 31) | 0.745 | 0.093 | 0.106 |
| Group 11 (AAV IT days 1, 3) (0.5 mg/kg AC003130 IT days 30, 31) | 0.697 | 0.198 | 0.277 |

As shown in Table 19, above, the TSLP RNAi agents each showed some reductions in hTSLP expression compared to control. Of particular note, Group 7 (AC003100, targeting position 571 of the TSLP gene) showed an approximately 42% reduction (0.576) in hTSLP mRNA, and Group 9 (AC003128, targeting position 520 of the TSLP gene) showed reductions of approximately 48% (0.522) on day 44, and provided substantially more knock-down that the other TSLP RNAi agents tested.

### Example 5. AAV9-CAG-hTSLP AAV Mouse Model

To evaluate certain TSLP RNAi agents, the same AAV9-CAG-hTSLP (Adeno-associated virus) mouse model as discussed in Example 4 was used.

The human TSLP mRNA expression was measured in the mice lung tissues by qPCR

At day 1 and Day 3, each mouse was given an intratracheal (IT) administration of 50 μL AAV solutions containing 2e10 GC (genome copy) of AAV9-CAG-eGFP and 3e10 GC of AAV9-CAG-hTSLP in PBS, or vehicle control (PBS). At Day 15, each mouse was given intratracheal administration of 50 μL of different dose levels of TSLP RNAi agents formulated in isotonic saline, or vehicle control (isotonic saline with no RNAi agent), according to the following Table 20. The mice were humanely sacrificed and harvested on Day 31.

TABLE 20
Targeted Positions and Dosing Groups of Example 5.

| | Targeted TSLP | | | |
|---|---|---|---|---|
| | Gene Position | | | |
| | (within SEQ ID | | RNAi Agent | |
| | NO: 1, GenBank | AAV dose | and Dose | |
| Group | NM_033035.5) | (Day 1, 3) | (Day 15) | Dosing Regimen |
| 1 | N/A | PBS | Saline (no RNAi | IT doses of PBS on |
| | | | agent) | Day 1, 3; |
| | | | | IT dose of saline on |
| | | | | day 15 |
| 2 | N/A | 2e10 GC of AAV9- | Saline (no RNAi | IT doses of AAV on |
| | | CAG-eGFP and | agent) | Day 1, 3; |
| | | 3e10 GC of AAV9- | | IT dose of saline on |
| | | CAG-hTSLP | | day 15 |
| 3 | 571 | 2e10 GC of AAV9- | 3.0 mg/kg | IT doses of AAV on |
| | | CAG-eGFP and | AC003100 | Day 1, 3; |
| | | 3e10 GC of AAV9- | | IT dose of RNAi |
| | | CAG-hTSLP | | agent on day 15 |
| 4 | 571 | 2e10 GC of AAV9- | 1.5 mg/kg | IT doses of AAV on |
| | | CAG-eGFP and | AC003100 | Day 1, 3; |
| | | 3e10 GC of AAV9- | | IT dose of RNAi |
| | | CAG-hTSLP | | agent on day 15 |
| 5 | 571 | 2e10 GC of AAV9- | 0.75 mg/kg | IT doses of AAV on |
| | | CAG-eGFP and | AC003100 | Day 1, 3; |
| | | 3e10 GC of AAV9- | | IT dose of RNAi |
| | | CAG-hTSLP | | agent on day 15 |
| 6 | 520 | 2e10 GC of AAV9- | 3.0 mg/kg | IT doses of AAV on |
| | | CAG-eGFP and | AC003128 | Day 1, 3; |
| | | 3e10 GC of AAV9- | | IT dose of RNAi |
| | | CAG-hTSLP | | agent on day 15 |
| 7 | 520 | 2e10 GC of AAV9- | 1.5 mg/kg | IT doses of AAV on |
| | | CAG-eGFP and | AC003128 | Day 1, 3; |
| | | 3e10 GC of AAV9- | | IT dose of RNAi |
| | | CAG-hTSLP | | agent on day 15 |
| 8 | 568 | 2e10 GC of AAV9- | 0.75 mg/kg | IT doses of AAV on |
| | | CAG-eGFP and | AC003101 | Day 1, 3; |
| | | 3e10 GC of AAV9- | | IT dose of RNAi |
| | | CAG-hTSLP | | agent on day 15 |
| 9 | 570 | 2e10 GC of AAV9- | 3.0 mg/kg | IT doses of AAV on |
| | | CAG-eGFP and | AC003099 | Day 1, 3; |
| | | 3e10 GC of AAV9- | | IT dose of RNAi |
| | | CAG-hTSLP | | agent on day 15 |
| 10 | 520 | 2e10 GC of AAV9- | 3.0 mg/kg | IT doses of AAV on |
| | | CAG-eGFP and | AC003252 | Day 1, 3; |
| | | 3e10 GC of AAV9- | | IT dose of RNAi |
| | | CAG-hTSLP | | agent on day 15 |
| 11 | 520 | 2e10 GC of AAV9- | 3.0 mg/kg | IT doses of AAV on |
| | | CAG-eGFP and | AC003253 | Day 1, 3; |
| | | 3e10 GC of AAV9- | | IT dose of RNAi |
| | | CAG-hTSLP | | agent on day 15 |

Each of the TSLP RNAi agents included modified nucleotides that were conjugated at the 5′ terminal end of the sense strand to an αvβ36 integrin targeting ligand having the modified sequences as set forth in the duplex structures herein. (See Tables 3, 4, 5, 6, 7A, 7B, 8, 9, 10, and 11 for specific modifications and structure information related to the TSLP RNAi agents, including Tri-SM6.1-αvβ6). The TSLP RNAi agents in Groups 3-8 each included nucleotide sequences that were designed to inhibit expression of a TSLP gene by targeting specific positions of TSLP mRNA as set forth in Table 20, above. (See. e.g., SEQ ID NO:1 and Table 2 for the TSLP mRNA sequence referenced.)

Five (5) mice in each group were tested (n=5) in each group, except for Group 1 where only 4 mice were tested. Left lobe lungs were collected in 4% PFA for histology analysis. Lower right lobes were collected for human TSLP protein measurement by Meso Scale Discovery (MSD) Assay. All remaining right lobes were collected for TSLP mRNA expression measurement by qPCR Data from the experiment are shown in the following Table 21:

TABLE 21
Average Relative TSLP Normalized to Control in AAV-hTSLP Mice from Example 5.

| | Average Relative hTSLP | Low | High |
|---|---|---|---|
| Group ID | mRNA Expression | (error) | (error) |
| Group 1 (PBS IT days 1, 3) (saline IT day 15) | N/A | | |
| Group 2 (AAV days 1, 3) (saline IT day 15) | 1.000 | 0.077 | 0.083 |
| Group 3 (AAV IT days 1, 3) (3.0 mg/kg AC003100 IT day 15) | 0.448 | 0.077 | 0.092 |
| Group 4 (AAV IT days 1, 3) (1.5 mg/kg AC003100 IT day 15) | 0.484 | 0.055 | 0.062 |
| Group 5 (AAV IT days 1, 3) (0.75 mg/kg AC003100 IT day 15) | 0.642 | 0.078 | 0.088 |
| Group 6 (AAV IT days 1, 3) (3.0 mg/kg AC003128 IT day 15) | 0.562 | 0.098 | 0.118 |
| Group 7 (AAV IT days 1, 3) (1.5 mg/kg AC003128 IT day 15) | 0.705 | 0.149 | 0.190 |
| Group 8 (AAV IT days 1, 3) (0.75 mg/kg AC003128 IT day 15) | 0.800 | 0.074 | 0.082 |
| Group 9 (AAV IT days 1, 3) (3.0 mg/kg AC003099 IT day 15) | 0.518 | 0.100 | 0.124 |
| Group 10 (AAV IT days 1, 3) (3.0 mg/kg AC003252 IT day 15) | 0.576 | 0.124 | 0.157 |
| Group 11 (AAV IT days 1, 3) (3.0 mg/kg AC003253 IT day 15) | 0.508 | 0.127 | 0.170 |

As shown in Table 21, above, the TSLP RNAi agents each showed some reductions in hTSLP expression compared to control, and further a dose response was shown for AC003100 and AC003128. Group 2 (3.0 mg/kg of AC003100, targeting position 571 of the TSLP gene) showed an approximately 55% reduction (0.448) in hTSLP mRNA. Further, hTSLP protein expression was measured for some of the dosing groups by MSD assay from the lower right lobe of the mouse lung tissues collected, and the data from certain of the samples are shown in FIG. 2. As shown in FIG. 2, 82% reduction in hTSLP protein was seen from Group 6 (3 mg/kg AC003128), which targeted position 520 of the TSLP gene; substantial reductions in hTSLP protein were also evidenced with other groups.

### Example 6. AAV9-CAG-hTSLP AAV Mouse Model

To evaluate certain TSLP RNAi agents, the same AAV9-CAG-hTSLP (Adeno-associated virus) mouse model as discussed in Example 4 was used.

The human TSLP mRNA expression was measured in the mice lung tissues by qPCR

At day 1 and Day 3, each mouse was given an intratracheal (IT) administration of 50 μL AAV solutions containing 2e10 GC (genome copy) of AAV9-CAG-eGFP and 3e10 GC of AAV9-CAG-hTSLP in PBS, or vehicle control (PBS). At Day 17 and 20, each mouse was given intratracheal administration of 50 μL of 1.5 mg/kg of TSLP RNAi agents formulated in isotonic saline, or vehicle control (isotonic saline with no RNAi agent), according to the following Table 22. The mice were humanely sacrificed and harvested on Day 31.

TABLE 22
Targeted Positions and Dosing Groups of Example 6.

| | Targeted TSLP | | | |
|---|---|---|---|---|
| | Gene Position | | | |
| | (within SEQ ID | | RNAi Agent | |
| | NO: 1, GenBank | AAV dose | and Dose | |
| Group | NM_033035.5) | (Day 1, 3) | (Day 17, 20) | Dosing Regimen |
| 1 | N/A | PBS | Saline (no RNAi | IT doses of PBS on |
| | | | agent) | Day 1, 3; |
| | | | | IT doses of saline on |
| | | | | days 17, 20 |
| 2 | N/A | 2e10 GC of AAV9- | Saline (no RNAi | IT doses of AAV on |
| | | CAG-eGFP and | agent) | Day 1, 3; |
| | | 3e10 GC of AAV9- | | IT dose2 of saline |
| | | CAG-hTSLP | | on days 17, 20 |
| 3 | 520 | 2e10 GC of AAV9- | 1.5 mg/kg | IT doses of AAV on |
| | | CAG-eGFP and | AC003128 | Day 1, 3; |
| | | 3e10 GC of AAV9- | | IT doses of RNAi |
| | | CAG-hTSLP | | agent on days 17, 20 |
| 4 | 520 | 2e10 GC of AAV9- | 1.5 mg/kg | IT doses of AAV on |
| | | CAG-eGFP and | AC003341 | Day 1, 3; |
| | | 3e10 GC of AAV9- | | IT doses of RNAi |
| | | CAG-hTSLP | | agent on days 17, 20 |
| 5 | 520 | 2e10 GC of AAV9- | 1.5 mg/kg | IT doses of AAV on |
| | | CAG-eGFP and | AC003342 | Day 1, 3; |
| | | 3e10 GC of AAV9- | | IT doses of RNAi |
| | | CAG-hTSLP | | agent on days 17, 20 |
| 6 | 520 | 2e10 GC of AAV9- | 1.5 mg/kg | IT doses of AAV on |
| | | CAG-eGFP and | AC003343 | Day 1, 3; |
| | | 3e10 GC of AAV9- | | IT doses of RNAi |
| | | CAG-hTSLP | | agent on days 17, 20 |
| 7 | 520 | 2e10 GC of AAV9- | 1.5 mg/kg | IT doses of AAV on |
| | | CAG-eGFP and | AC003344 | Day 1, 3; |
| | | 3e10 GC of AAV9- | | IT doses of RNAi |
| | | CAG-hTSLP | | agent on days 17, 20 |
| 8 | 520 | 2e10 GC of AAV9- | 1.5 mg/kg | IT doses of AAV on |
| | | CAG-eGFP and | AC003345 | Day 1, 3; |
| | | 3e10 GC of AAV9- | | IT doses of RNAi |
| | | CAG-hTSLP | | agent on days 17, 20 |
| 9 | 520 | 2e10 GC of AAV9- | 1.5 mg/kg | IT doses of AAV on |
| | | CAG-eGFP and | AC003346 | Day 1, 3; |
| | | 3e10 GC of AAV9- | | IT doses of RNAi |
| | | CAG-hTSLP | | agent on days 17, 20 |
| 10 | 520 | 2e10 GC of AAV9- | 1.5 mg/kg | IT doses of AAV on |
| | | CAG-eGFP and | AC003347 | Day 1, 3; |
| | | 3e10 GC of AAV9- | | IT doses of RNAi |
| | | CAG-hTSLP | | agent on days 17, 20 |
| 11 | 571 | 2e10 GC of AAV9- | 1.5 mg/kg | IT doses of AAV on |
| | | CAG-eGFP and | AC003100 | Day 1, 3; |
| | | 3e10 GC of AAV9- | | IT doses of RNAi |
| | | CAG-hTSLP | | agent on days 17, 20 |

Each of the TSLP RNAi agents included modified nucleotides that were conjugated at the 5′ terminal end of the sense strand to an αvβ6 integrin targeting ligand having the modified sequences as set forth in the duplex structures herein. (See Tables 3, 4, 5, 6, 7A, 7B, 8, 9, 10, and 11 for specific modifications and structure information related to the TSLP RNAi agents, including Tri-SM6.1-αvβ6). The TSLP RNAi agents in Groups 3-8 each included nucleotide sequences that were designed to inhibit expression of a TSLP gene by targeting specific positions of TSLP mRNA as set forth in Table 22, above. (See. e.g., SEQ ID NO:1 and Table 2 for the TSLP mRNA sequence referenced.)

Five (5) mice in each group were tested (n=5) in each group, except for Group 1 where only 4 mice were tested. TSLP mRNA expression levels were determined by qPCR Data from the experiment are shown in the following Table 23:

TABLE 23
Average Relative TSLP Normalized to Control in AAV-hTSLP Mice from Example 6.

| | Average Relative hTSLP | Low | High |
|---|---|---|---|
| Group ID | mRNA Expression | (error) | (error) |
| Group 1 (PBS IT days 1, 3) (saline IT days 17, 20) | N/A | | |
| Group 2 (AAV days 1, 3) (saline IT days 17, 20) | 1.000 | 0.232 | 0.302 |
| Group 3 (AAV IT days 1, 3) (1.5 mg/kg AC003128 IT days 17, 20) | 0.685 | 0.183 | 0.249 |
| Group 4 (AAV IT days 1, 3) (1.5 mg/kg AC003341 IT days 17, 20) | 0.512 | 0.130 | 0.174 |
| Group 5 (AAV IT days 1, 3) (1.5 mg/kg AC003342 IT days 17, 20) | 0.453 | 0.077 | 0.093 |
| Group 6 (AAV IT days 1, 3) (1.5 mg/kg AC003343 IT days 17, 20) | 0.552 | 0.207 | 0.332 |
| Group 7 (AAV IT days 1, 3) (1.5 mg/kg AC003344 IT days 17, 20) | 0.495 | 0.116 | 0.151 |
| Group 8 (AAV IT days 1, 3) (1.5 mg/kg AC003345 IT days 17, 20) | 0.434 | 0.086 | 0.108 |
| Group 9 (AAV IT days 1, 3) (1.5 mg/kg AC003346 IT days 17, 20) | 0.613 | 0.188 | 0.271 |
| Group 10 (AAV IT days 1, 3) (1.5 mg/kg AC003347 IT days 17, 20) | 0.595 | 0.173 | 0.243 |
| Group 11 (AAV IT days 1, 3) (1.5 mg/kg AC003100 IT days 17, 20) | 0.847 | 0.229 | 0.313 |

As shown in Table 23, above, each of the TSLP RNAi agents tested showed reductions in hTSLP expression compared to control. In particular, Group 5 (1.5 mg/kg of AC003342, targeting position 520 of the TSLP gene) showed an approximately 55% reduction (0.453) in hTSLP mRNA, and Group 8 (1.5 mg/kg of AC003345, also targeting position 520 of the TSLP gene) showed an approximately 57% reduction (0.434) in hTSLP mRNA.

### Example 7. AAV9-CAG-hTSLP AAV Mouse Model

To evaluate certain TSLP RNAi agents, the same AAV9-CAG-hTSLP (Adeno-associated virus) mouse model as discussed in Example 4 was used.

The human TSLP mRNA expression was measured in the mice lung tissues by qPCR

At day 1 and Day 3, each mouse was given an intratracheal (IT) administration of 50 μL AAV solutions containing 2e10 GC (genome copy) of AAV9-CAG-eGFP and 3e10 GC of AAV9-CAG-hTSLP in PBS, or vehicle control (PBS). At Day 15 and 18, each mouse was given intratracheal administration of 50 μL of 1.5 mg/kg of TSLP RNAi agents formulated in isotonic saline, or vehicle control (isotonic saline with no RNAi agent), according to the following Table 22. The mice were humanely sacrificed and harvested on Day 31.

TABLE 24
Targeted Positions and Dosing Groups of Example 7.

| | Targeted TSLP | | | |
|---|---|---|---|---|
| | Gene Position | | | |
| | (within SEQ ID | | RNAi Agent | |
| | NO: 1, GenBank | AAV dose | and Dose | |
| Group | NM_033035.5) | (Day 1, 3) | (Day 15, 18) | Dosing Regimen |
| 1 | N/A | PBS | Saline (no RNAi | IT doses of PBS on |
| | | | agent) | Day 1, 3; |
| | | | | IT doses of saline on |
| | | | | days 15, 18 |
| 2 | N/A | 2e10 GC of AAV9- | Saline (no RNAi | IT doses of AAV on |
| | | CAG-eGFP and | agent) | Day 1, 3; |
| | | 3e10 GC of AAV9- | | IT doses of saline on |
| | | CAG-hTSLP | | days 15, 18 |
| 3 | 571 | 2e10 GC of AAV9- | 1.5 mg/kg | IT doses of AAV on |
| | | CAG-eGFP and | AC003100 | Day 1, 3; |
| | | 3e10 GC of AAV9- | | IT doses of RNAi |
| | | CAG-hTSLP | | agent on days 15, 18 |
| 4 | 571 | 2e10 GC of AAV9- | 1.5 mg/kg | IT doses of AAV on |
| | | CAG-eGFP and | AC003371 | Day 1, 3; |
| | | 3e10 GC of AAV9- | | IT doses of RNAi |
| | | CAG-hTSLP | | agent on days 15, 18 |
| 5 | 571 | 2e10 GC of AAV9- | 1.5 mg/kg | IT doses of AAV on |
| | | CAG-eGFP and | AC003372 | Day 1, 3; |
| | | 3e10 GC of AAV9- | | IT doses of RNAi |
| | | CAG-hTSLP | | agent on days 15, 18 |
| 6 | 571 | 2e10 GC of AAV9- | 1.5 mg/kg | IT doses of AAV on |
| | | CAG-eGFP and | AC003373 | Day 1, 3; |
| | | 3e10 GC of AAV9- | | IT doses of RNAi |
| | | CAG-hTSLP | | agent on days 15, 18 |
| 7 | 571 | 2e10 GC of AAV9- | 1.5 mg/kg | IT doses of AAV on |
| | | CAG-eGFP and | AC003374 | Day 1, 3; |
| | | 3e10 GC of AAV9- | | IT doses of RNAi |
| | | CAG-hTSLP | | agent on days 15, 18 |
| 8 | 571 | 2e10 GC of AAV9- | 1.5 mg/kg | IT doses of AAV on |
| | | CAG-eGFP and | AC003375 | Day 1, 3; |
| | | 3e10 GC of AAV9- | | IT doses of RNAi |
| | | CAG-hTSLP | | agent on days 15, 18 |
| 9 | 571 | 2e10 GC of AAV9- | 1.5 mg/kg | IT doses of AAV on |
| | | CAG-eGFP and | AC003376 | Day 1, 3; |
| | | 3e10 GC of AAV9- | | IT doses of RNAi |
| | | CAG-hTSLP | | agent on days 15, 18 |
| 10 | 571 | 2e10 GC of AAV9- | 1.5 mg/kg | IT doses of AAV on |
| | | CAG-eGFP and | AC003377 | Day 1, 3; |
| | | 3e10 GC of AAV9- | | IT doses of RNAi |
| | | CAG-hTSLP | | agent on days 15, 18 |
| 11 | 571 | 2e10 GC of AAV9- | 1.5 mg/kg | IT doses of AAV on |
| | | CAG-eGFP and | AC003378 | Day 1, 3; |
| | | 3e10 GC of AAV9- | | IT doses of RNAi |
| | | CAG-hTSLP | | agent on days 15, 18 |
| 12 | 571 | 2e10 GC of AAV9- | 1.5 mg/kg | IT doses of AAV on |
| | | CAG-eGFP and | AC003379 | Day 1, 3; |
| | | 3e10 GC of AAV9- | | IT doses of RNAi |
| | | CAG-hTSLP | | agent on days 15, 18 |

Each of the TSLP RNAi agents included modified nucleotides that were conjugated at the 5′ terminal end of the sense strand to an αvβ6 integrin targeting ligand having the modified sequences as set forth in the duplex structures herein. (See Tables 3, 4, 5, 6, 7A, 7B, 8, 9, 10, and 11 for specific modifications and structure information related to the TSLP RNAi agents, including Tri-SM6.1-αvβ6). The TSLP RNAi agents in Groups 3-8 each included nucleotide sequences that were designed to inhibit expression of a TSLP gene by targeting specific positions of TSLP mRNA as set forth in Table 24, above. (See. e.g., SEQ ID NO:1 and Table 2 for the TSLP mRNA sequence referenced.)

Five (5) mice in each group were tested (n=5) in each group, except for Group 1 where only 4 mice were tested. Left lobe lungs were collected in 4% PFA for histology analysis. Lower right lobes were collected for human TSLP protein measurement by Meso Scale Discovery (MSD) Assay. All remaining right lobes were collected for TSLP mRNA expression measurement by qPCR Data from the experiment are shown in the following Table 25:

TABLE 25
Average Relative TSLP Normalized to Control in AAV-hTSLP Mice from Example 7.

| | Average Relative hTSLP | Low | High |
|---|---|---|---|
| Group ID | mRNA Expression | (error) | (error) |
| Group 1 (PBS IT days 1, 3) (saline IT days 17, 20) | N/A | | |
| Group 2 (AAV days 1, 3) (saline IT days 17, 20) | 1.000 | 0.128 | 0.146 |
| Group 3 (AAV IT days 1, 3) (1.5 mg/kg AC003100 IT days 15, 18) | 0.372 | 0.040 | 0.045 |
| Group 4 (AAV IT days 1, 3) (1.5 mg/kg AC003371 IT days 15, 18) | 0.363 | 0.108 | 0.154 |
| Group 5 (AAV IT days 1, 3) (1.5 mg/kg AC003372 IT days 15, 18) | 0.474 | 0.086 | 0.105 |
| Group 6 (AAV IT days 1, 3) (1.5 mg/kg AC003373 IT days 15, 18) | 0.750 | 0.126 | 0.151 |
| Group 7 (AAV IT days 1, 3) (1.5 mg/kg AC003374 IT days 15, 18) | 0.335 | 0.037 | 0.041 |
| Group 8 (AAV IT days 1, 3) (1.5 mg/kg AC003375 IT days 15, 18) | 0.329 | 0.048 | 0.056 |
| Group 9 (AAV IT days 1, 3) (1.5 mg/kg AC003376 IT days 15, 18) | 0.395 | 0.086 | 0.109 |
| Group 10 (AAV IT days 1, 3) (1.5 mg/kg AC003377 IT days 15, 18) | 0.432 | 0.064 | 0.074 |
| Group 11 (AAV IT days 1, 3) (1.5 mg/kg AC003378 IT days 15, 18) | 0.411 | 0.060 | 0.070 |
| Group 12 (AAV IT days 1, 3) (1.5 mg/kg AC003379 IT days 15, 18) | 0.327 | 0.046 | 0.053 |

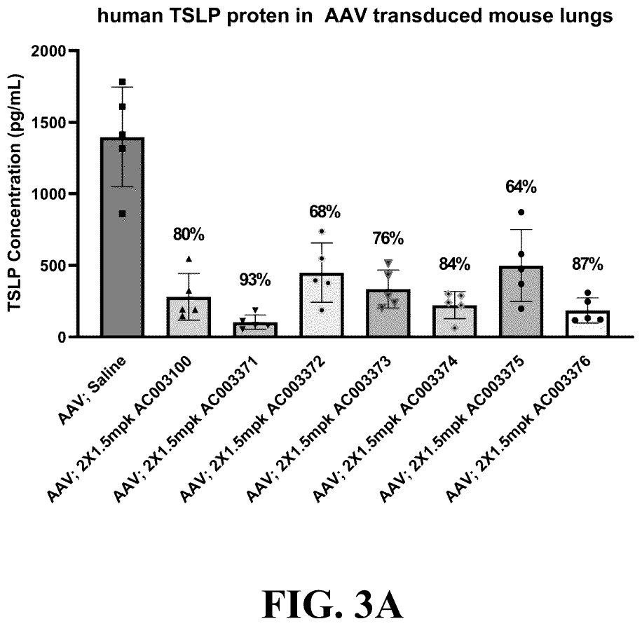

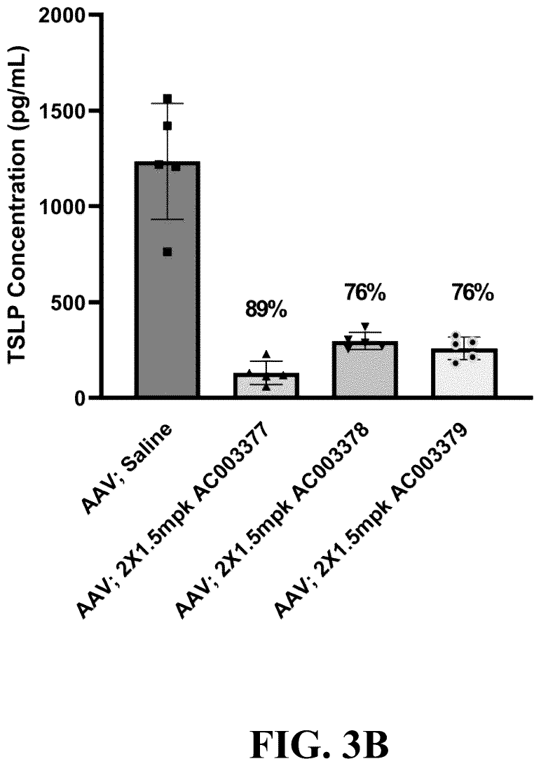

As shown in Table 25, above, each of the TSLP RNAi agents tested showed reductions in hTSLP expression compared to control. In particular, several of the TSLP RNAi agents targeting the TSLP transcript at position 571 achieved more than 60% hTSLP mRNA inhibition, with AC003371 achieving 64% knockdown (Group 4, 0.363), AC003374 achieving 66% knockdown (Group 7, 0.335), and AC003375 achieving 67% knockdown (Group 8, 0.329) mRNA. Further, hTSLP protein expression was measured for each of the dosing groups by MSD assay from the lower right lobe of the mouse lung tissues collected, and the data from certain of the samples are shown in FIGS. 3A and 3B. As shown in FIGS. 3A and 3B, greater than 90% reductions hTSLP protein was seen from Group 4 (3 mg/kg AC003371), which targeted position 571 of the TSLP gene; substantial reductions in hTSLP protein were also evidenced with each of the other Groups.

### Example 8. In Vivo Inhaled Aerosolized Administration of Rat-Specific TSLP RNAI Agents in Rats

On study day 1, male Sprague Dawley rats were administered a single targeted deposited dose of 1.5 mg/kg of the rat-specific RNAi agent AC001714, the chemical structure of which is set forth in Example 2, or a single dose of isotonic saline.

Using a jet nebulizer (Misty Max 10), aerosol was delivered to a rodent single-tier flow-past nose-only inhalation exposure chamber (CH Technologies). One of the ports was equipped with a filter housing so that RNAi agent aerosol concentration could be assessed. Using an assumed respiratory minute volume allometrically scaled to rodent body weight, along with aerosol concentration determined from filter collection and RNAi agent quantification, exposure times were adjusted to target the dose level at 1.5 mg/kg. The actual pulmonary deposited doses (PDD) are listed in Table 26:

TABLE 26
Rat-specific TSLP RNAi Agent and Dosing for Example 8.

| | AC | Animals | Harvest/ |
|---|---|---|---|
| | Duplex | per | Sacrifice |
| Group ID | Number | Group | Day |
| Group 1 (saline PDD day 1) | N/A | 5 | Day 28 |
| Group 2 (PDD dose 1.66 mg/kg AC001714 on day 1) | AC001714 | 5 | Day 28 |
| Group 3 (saline PDD day 1) | N/A | 5 | Day 56 |
| Group 4 (PDD dose 1.66 mg/kg AC001714 on day 1) | AC001714 | 5 | Day 56 |
| Group 5 (saline PDD day 1) | N/A | 5 | Day 84 |
| Group 6 (PDD dose 1.66 mg/kg AC001714 on day 1) | AC001714 | 5 | Day 84 |
| Group 7 (saline PDD day 1) | N/A | 5 | Day 112 |
| Group 8 (PDD dose 1.82 mg/kg AC001714 on day 1) | AC001714 | 5 | Day 112 |
| Group 9 (saline PDD day 1) | N/A | 5 | Day 140 |
| Group 10 (PDD dose 1.82 mg/kg AC001714 on day 1) | AC001714 | 5 | Day 140 |
| Group 11 (saline PDD day 1) | N/A | 5 | Day 168 |
| Group 12 (PDD dose 1.82 mg/kg AC001714 on day 1) | AC001714 | 5 | Day 168 |

Five (5) rats were dosed per group. Rats were sacrificed in accordance with Table 26, and total RNA was isolated from both lungs following collection and homogenization. Rat TSLP mRNA expression was quantitated by probe-based quantitative PCR, normalized to rat B2M expression, and expressed as fraction of vehicle control group (geometric mean, +/−95% confidence interval).

TABLE 27
Average Relative Rat TSLP mRNA Expression at Sacrifice in Example 8

| | Average Relative rTSLP | Low | High |
|---|---|---|---|
| Group ID | mRNA Expression | (error) | (error) |
| Group 1 (isotonic saline; day 28 sacrifice) | 1.000 | 0.242 | 0.292 |
| Group 2 (1.5 mg/kg AC001714; day 28 sacrifice) | 0.657 | 0.097 | 0.114 |
| Group 3 (isotonic saline; day 56 sacrifice) | 1.000 | 0.173 | 0.142 |
| Group 4 (1.5 mg/kg AC001714; day 56 sacrifice) | 0.729 | 0.119 | 0.142 |
| Group 5 (isotonic saline; day 84 sacrifice) | 0.798 | 0.107 | 0.160 |
| Group 6 (1.5 mg/kg AC001714; day 84 sacrifice) | 0.572 | 0.064 | 0.047 |
| Group 7 (isotonic saline; day 112 sacrifice) | 0.882 | 0.262 | 0.397 |
| Group 8 (1.5 mg/kg AC001714; day 112 sacrifice) | 0.726 | 0.082 | 0.095 |
| Group 9 (isotonic saline; day 140 sacrifice) | 0.853 | 0.274 | 0.391 |
| Group 10 (1.5 mg/kg AC001714; day 140 sacrifice) | 1.171 | 0.494 | 0.990 |
| Group 11 (isotonic saline; day 168 sacrifice) | 0.909 | 0.165 | 0.282 |
| Group 12 (1.5 mg/kg AC001714; day 168 sacrifice) | 1.017 | 0.175 | 0.174 |

As shown in the data in Table 27 above, even when administered by inhalation, meaningful inhibition of TSLP gene expression was evident by this particular rat-specific RNAi agent tool that employed an integrin-targeting ligand (AC001714) through at least day 84.

### Example 9. In Vivo Intratracheal Administration of Rat-Speck TSLP RNAI Agents in Rats

On study day 1 and day 3, male Sprague Dawley rats were administered 200 microliters via a microsprayer device (Penn Century, Philadelphia, PA) suitable for intratracheal (IT) administration of (i) isotonic saline, or (ii) 5 mg/kg of the rat-specific RNAi agent AC001714, the chemical structure of which is set forth in Example 2, or (iii) a “RISC-blocked” RNAi trigger, which include a construct similar to AC001714, including the same targeting ligand, but included chemical modifications designed to prevent the loading of the antisense strand into RISC, thus serving as a negative control, in accordance with the following Table 28:

TABLE 28
Rat-specific TSLP RNAi Agent and Dosing for Example 9.

| | AC | Animals | Harvest/ |
|---|---|---|---|
| | Duplex | per | Sacrifice |
| Group ID | Number | Group | Day |
| Group 1 (saline IT days 1, 3) | N/A | 5 | Day 15 |
| Group 2 (saline IT days 1, 3) | N/A | 5 | Day 29 |
| Group 3 (saline IT days 1, 3) | N/A | 5 | Day 43 |
| Group 4 (saline IT days 1, 3) | N/A | 5 | Day 57 |
| Group 5 (IT dose 5.0 mg/kg AC001714 on days 1,3) | AC001714 | 5 | Day 15 |
| Group 6 (IT dose 5.0 mg/kg RISC-blocked negative | AC001714 | 5 | Day 29 |
| control RNAi trigger on days 1, 3) | | | |
| Group 7 (IT dose 5.0 mg/kg AC001714 on days 1, 3) | AC001714 | 5 | Day 29 |
| Group 8 (IT dose 5.0 mg/kg AC001714 on days 1, 3) | AC001714 | 5 | Day 43 |
| Group 9 (IT dose 5.0 mg/kg AC001714 on days 1, 3) | AC001714 | 5 | Day 57 |

Five (5) rats were dosed per group. Rats were sacrificed in accordance with Table 28, and total RNA was isolated from both lungs following collection and homogenization. Rat TSLP mRNA expression was quantitated by probe-based quantitative PCR, normalized to rat B2M expression, and expressed as fraction of vehicle control group (geometric mean, +/−95% confidence interval).

TABLE 29
Average Relative Rat TSLP mRNA Expression at Sacrifice in Example 9

| | Average Relative rTSLP | Low | High |
|---|---|---|---|
| Group ID | mRNA Expression | (error) | (error) |
| Group 1 (saline IT days 1, 3; day 15 sacrifice) | 1.000 | 0.137 | 0.159 |
| Group 2 (saline IT days 1, 3; day 29 sacrifice) | 1.000 | 0.139 | 0.162 |
| Group 3 (saline IT days 1, 3; day 43 sacrifice) | 1.000 | 0.207 | 0.261 |
| Group 4 (saline IT days 1, 3; day 57 sacrifice) | 1.000 | 0.227 | 0.294 |
| Group 5 (IT dose 5.0 mg/kg AC001714 on days 1,3; | 0.506 | 0.088 | 0.106 |
| day 15 sacrifice) | | | |
| Group 6 (IT dose 5.0 mg/kg RISC-blocked negative | 0.640 | 0.223 | 0.342 |
| control RNAi trigger on days 1, 3; day 29 sacrifice) | | | |
| Group 7 (IT dose 5.0 mg/kg AC001714 on days 1, 3; | 0.495 | 0.067 | 0.078 |
| day 29 sacrifice) | | | |
| Group 8 (IT dose 5.0 mg/kg AC001714 on days 1, 3; | 0.394 | 0.146 | 0.232 |
| day 43 sacrifice) | | | |
| Group 9 (IT dose 5.0 mg/kg AC001714 on days 1, 3; | 0.371 | 0.132 | 0.204 |
| day 57 sacrifice) | | | |

As shown in the data in Table 29 above, meaningful inhibition of TSLP gene expression was evident by this particular rat-specific RNAi agent tool that employed an integrin-targeting ligand (AC001714; Groups 5, 7, 8 and 9) through at least day 57.

### Example 10 In Vivo Intratracheal Administration of Rat Specific TSLP RNAi Agents in Rats

On study day 1 and day 3, male Brown Norway rats were administered 200 microliters via a microsprayer device (Penn Century, Philadelphia, PA) suitable for intratracheal (IT) administration of (i) isotonic saline, or (ii) 5 mg/kg of the rat-specific RNAi agent AC001714, the chemical structure of which is set forth in Example 2, or (iii) a “RISC-blocked” RNAi trigger, which include a construct similar to AC001714, including the same targeting ligand, but included chemical modifications designed to prevent the loading of the antisense strand into RISC, thus serving as a negative control, in accordance with the following Table 30:

TABLE 30
Rat-specific TSLP RNAi Agent and Dosing for Example 10.

| | AC | Animals | |
|---|---|---|---|
| | Duplex | per | Harvest/ |
| Group ID | Number | Group | Sacrifice Day |
| Group 1 (saline IT days 1, 3) (PBS IT | N/A | 6 | Day 13 |
| day 13) | | | |
| Group 2 (saline IT days 1, 3) (Alternaria | N/A | 7 | Day 13 |
| IT day 13) | | | (2 hr post Alternaria) |
| Group 3 (saline IT days 1, 3) (Alternaria | N/A | 7 | Day 14 |
| IT day 13) | | | (24 hr post Alternaria) |
| Group 4 (IT dose 5.0 mg/kg RISC- | N/A | 7 | Day 13 |
| blocked trigger on days 1, 3)/ | | | (2 hr post Alternaria) |
| (Alternaria IT day 13) | | | |
| Group 5 (IT dose 5.0 mg/kg AC001714 | AC001714 | 7 | Day 13 |
| on days 1, 3)/(Alternaria IT day 13) | | | (2 hr post Alternaria) |
| Group 6 (IT dose 5.0 mg/kg RISC- | N/A | 7 | Day 14 |
| blocked trigger on days 1, 3)/ | | | (24 hr post Alternaria) |
| (Alternaria IT day 13) | | | |
| Group 7 (IT dose 5.0 mg/kg AC001714 | AC001714 | 7 | Day 14 |
| on days 1, 3)/(Alternaria IT day 13) | | | (24 hr post Alternaria) |

On day 13, rats were challenged with a single intra-tracheal dose of 500 μg/rat of Alternaria alternata prepared in phosphate buffered saline (PBS). Rats in Group 1 were administered only with PBS as control.

After either 2 or 24 hours post-administration of the Alternaria (i.e., either day 13 or 14), rats were anesthetized with isoflurane/02, blood was drawn, and were then humanely euthanized by exsanguination. Days of sacrifice/euthanasia are shown in Table 30 above. Trachea was canulated and bronchoalveolar lavage (BAL) collected after washing with 2×5 mL of ice-cold PBS. BAL samples were spun down, cells resuspended with 1 mL of ice-cold PBS, and aliquot was mixed with Turk's solution (ratio 1:1), and total cell counted via hemocytomers. Cytospins were prepared, stained and differential cell counting performed. Supernatant was used for cytokine measurements. Right lung lobes were used to determine rTSLP mRNA expression and left lung lobes were collected in 4% PFA/PBS for histology (Trichrome and Sirius Red Staining, RNAscope).

Rat TSLP mRNA expression was quantitated by probe-based quantitative PCR, normalized to rat B2M expression, and expressed as fraction of vehicle control group (geometric mean, +/−95% confidence interval).

TABLE 31
Average Relative Rat TSLP mRNA Expression at
Sacrifice (i.e., Day 13 or 14) in Example 10

| | Average Relative rTSLP | Low | High |
|---|---|---|---|
| Group ID | mRNA Expression | (error) | (error) |
| Group 1 (saline IT days 1, 3) (PBS IT day 13) | 1.000 | 0.208 | 0.263 |
| Group 2 (saline IT days 1, 3) (Alternaria IT | 1.088 | 0.174 | 0.207 |
| day 13) | | | |
| Group 3 (saline IT days 1, 3) (Alternaria IT | 1.016 | 0.211 | 0.267 |
| day 13) | | | |
| Group 4 (IT dose 5.0 mg/kg RISC-blocked | 1.172 | 0.237 | 0.298 |
| trigger on days 1, 3)/(Alternaria IT day 13) | | | |
| Group 5 (IT dose 5.0 mg/kg AC001714 on | 0.538 | 0.071 | 0.082 |
| days 1, 3)/(Alternaria IT day 13) | | | |
| Group 6 (IT dose 5.0 mg/kg RISC-blocked | 0.725 | 0.106 | 0.125 |
| trigger on days 1, 3)/(Alternaria IT day 13) | | | |
| Group 7 (IT dose 5.0 mg/kg AC001714 on | 0.524 | 0.104 | 0.130 |
| days 1, 3)/(Alternaria IT day 13) | | | |

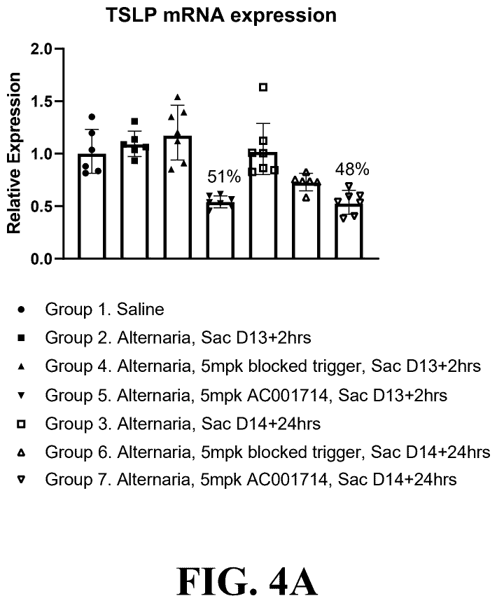

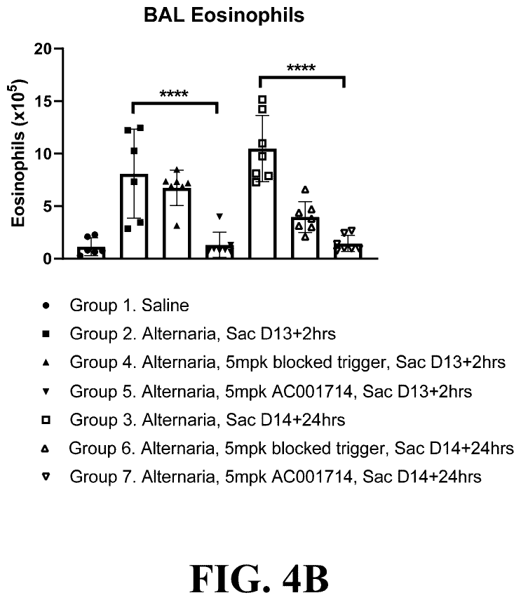

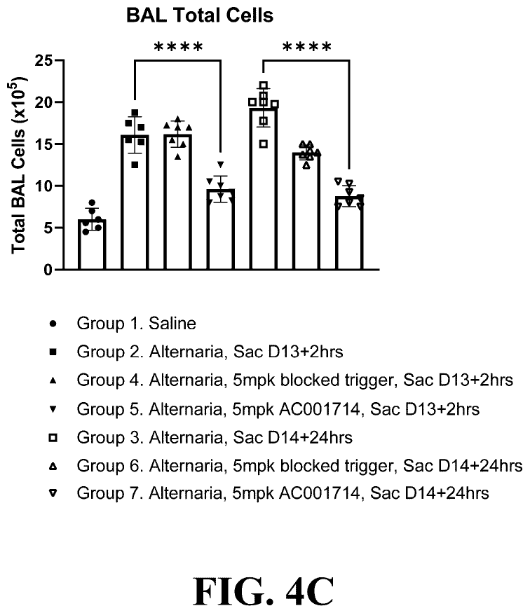

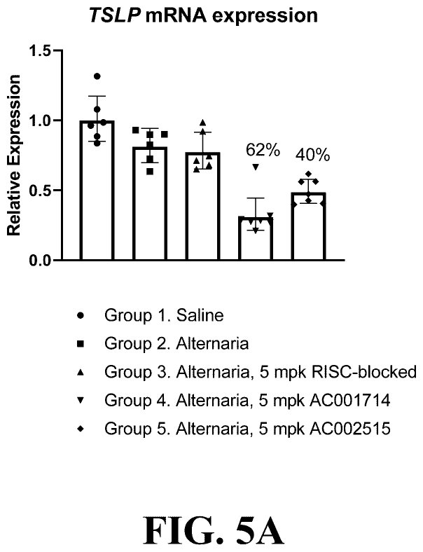

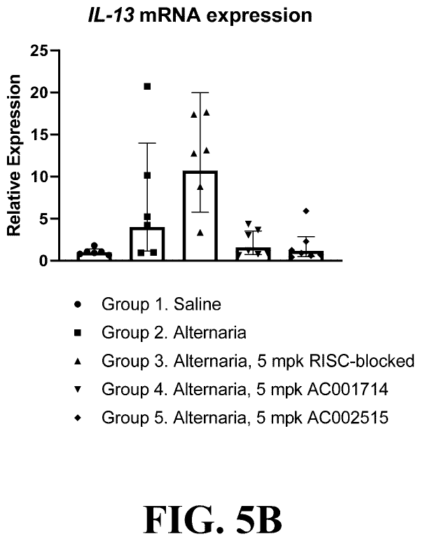

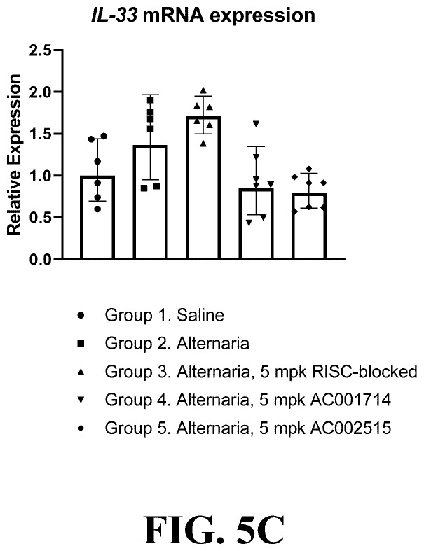

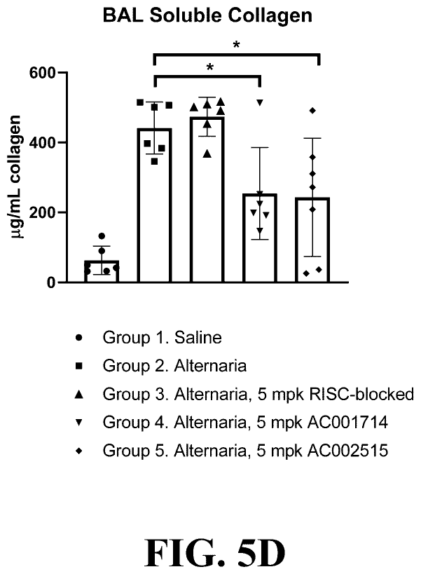

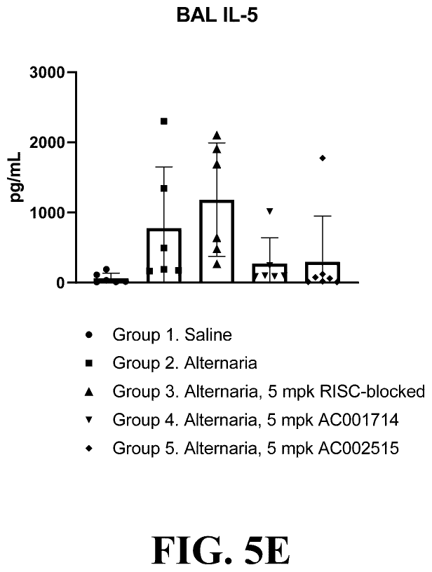

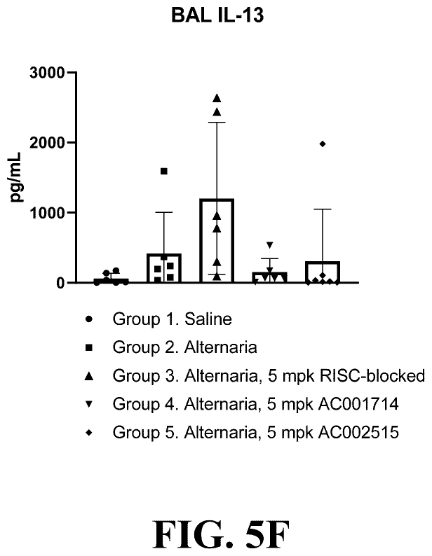

As shown in Table 31 above, the Groups administered AC001714 (i.e., Groups 5 and 7) each showed substantial reductions of rTSLP mRNA at the respective time of sacrifice relative to the respective control groups. These results are also shown in FIG. 4A.

Granulocytes, such as eosinophils, are well known markers for cellular inflammation. For the BAL samples, the total and differential cells were counted and the number of inflammatory cells were derived. The impact of rTSLP inhibition by the rat-specific TSLP RNAi agents disclosed herein on eosinophilic inflammation induced by Alternaria extract was assessed. BAL total cells and eosinophils counts are shown in FIGS. 4B and 4C. Groups 5 and 7 (treated with rat-specific TSLP RNAi agent) showed significant reductions of total BAL cell counts and eosinophils across all time points when compared to their respective control. As shown in FIG. 4B, a significant reduction of eosinophils at both 2 hours and 24 hours post Alternaria challenge (Groups 5 and 7, respectively) was observed as compared to Groups 2 and 3, respectively. Moreover, as shown in FIG. 4C, BAL total cells was significantly reduced at both 2 hour and 24 hour post Alternaria challenge (Groups 5 and 7, respectively) as compared to control Groups 2 and 3, respectively. Statistical significance is denoted **** p-value is p<0.0001.

This study provides physiological support in a rat model that a reduction in TSLP gene expression (rTSLP mRNA) can provide a phenotype improvement to reduce pulmonary inflammation, and thus can potentially treat diseases such as allergic asthma.

### Example 11. AAV9-CAG-hTSLP AAV Mouse Model

To evaluate certain TSLP RNAi agents, the same AAV9-CAG-hTSLP (Adeno-associated virus) mouse model as discussed in Example 4 was used.

The human TSLP mRNA expression was measured in the mice lung tissues by qPCR

At Day 1 and Day 5, each mouse was given an intratracheal (IT) administration of 50 μL AAV solutions containing 2e10 GC (genome copy) of AAV9-CAG-eGFP and 3e10 GC of AAV9-CAG-hTSLP in PBS, or vehicle control (PBS). At Day 20 and Day 22, each mouse was given intratracheal administration of 50 μL of 1.0 mg/kg of TSLP RNAi agents formulated in isotonic saline, or vehicle control (isotonic saline with no RNAi agent), according to the following Table 32. The mice were humanely sacrificed and harvested on Day 32.

TABLE 32
Targeted Positions and Dosing Groups of Example 11.

| | Targeted TSLP | | | |
|---|---|---|---|---|
| | Gene Position | | | |
| | (within SEQ ID | | RNAi Agent | |
| | NO: 1, GenBank | AAV dose | and Dose | |
| Group | NM_033035.5) | (Day 1, 3) | (Day 15, 18) | Dosing Regimen |
| 1 | N/A | 2e10 GC of AAV9- | Saline | IT doses of AAV on |
| | | CAG-eGFP and | | Day 1, 5; |
| | | 3e10 GC of AAV9- | | IT doses of saline on |
| | | CAG-hTSLP | | Days 20, 22 |
| 2 | 571 | 2e10 GC of AAV9- | 1.0 mg/kg | IT doses of AAV on |
| | | CAG-eGFP and | AC003374 | Day 1, 5; |
| | | 3e10 GC of AAV9- | | IT doses of RNAi |
| | | CAG-hTSLP | | agent on Days 20, 22 |
| 3 | 571 | 2e10 GC of AAV9- | 1.0 mg/kg | IT doses of AAV on |
| | | CAG-eGFP and | AC004077 | Day 1, 5; |
| | | 3e10 GC of AAV9- | | IT doses of RNAi |
| | | CAG-hTSLP | | agent on Days 20, 22 |
| 4 | 571 | 2e10 GC of AAV9- | 1.0 mg/kg | IT doses of AAV on |
| | | CAG-eGFP and | AC004078 | Day 1, 5; |
| | | 3e10 GC of AAV9- | | IT doses of RNAi |
| | | CAG-hTSLP | | agent on Days 20, 22 |
| 5 | 570 | 2e10 GC of AAV9- | 1.0 mg/kg | IT doses of AAV on |
| | | CAG-eGFP and | AC003567 | Day 1, 5; |
| | | 3e10 GC of AAV9- | | IT doses of RNAi |
| | | CAG-hTSLP | | agent on Days 20, 22 |
| 6 | 570 | 2e10 GC of AAV9- | 1.0 mg/kg | IT doses of AAV on |
| | | CAG-eGFP and | AC003511 | Day 1, 5; |
| | | 3e10 GC of AAV9- | | IT doses of RNAi |
| | | CAG-hTSLP | | agent on Days 20, 22 |
| 7 | 570 | 2e10 GC of AAV9- | 1.0 mg/kg | IT doses of AAV on |
| | | CAG-eGFP and | AC004079 | Day 1, 5; |
| | | 3e10 GC of AAV9- | | IT doses of RNAi |
| | | CAG-hTSLP | | agent on Days 20, 22 |
| 8 | 571 | 2e10 GC of AAV9- | 1.0 mg/kg | IT doses of AAV on |
| | | CAG-eGFP and | AC003602 | Day 1, 5; |
| | | 3e10 GC of AAV9- | | IT doses of RNAi |
| | | CAG-hTSLP | | agent on Days 20, 22 |

Each of the TSLP RNAi agents included modified nucleotides that were conjugated at the 5′ terminal end of the sense strand to an αvβ6 integrin targeting ligand having the modified sequences as set forth in the duplex structures herein. (See Tables 3, 4, 5, 6, 7A, 7B, 8, 9, 10, and 11 for specific modifications and structure information related to the TSLP RNAi agents, including Tri-SM6.1-αvβ6). The TSLP RNAi agents in Groups 2-8 each included nucleotide sequences that were designed to inhibit expression of a TSLP gene by targeting specific positions of TSLP mRNA as set forth in Table 32, above. (See. e.g., SEQ ID NO:1 and Table 2 for the TSLP mRNA sequence referenced.)

Five (5) mice in each group were tested (n=5) in each group. Left lobe lungs were collected in 4% PFA for histology analysis. Lower right lobes were collected for human TSLP protein measurement by Meso Scale Discovery (MSD) Assay. All remaining right lobes were collected for TSLP mRNA expression measurement by qPCR Data from the experiment are shown in the following Table 33:

TABLE 33
Average Relative TSLP Normalized to Control in AAV-hTSLP Mice from Example 11.

| | Average Relative hTSLP | Low | High |
|---|---|---|---|
| Group ID | mRNA Expression | (error) | (error) |
| Group 1 (AAV IT Days 1, 5) (Saline IT Days 17, 20) | 1.000 | 0.147 | 0.172 |
| Group 2 (AAV IT Days 1, 5) (1.0 mg/kg AC003374 IT Days 20, 22) | 0.330 | 0.071 | 0.090 |
| Group 3 (AAV IT Days 1, 5) (1.0 mg/kg AC004077 IT Days 20, 22) | 0.657 | 0.120 | 0.146 |
| Group 4 (AAV IT Days 1, 5) (1.0 mg/kg AC004078 IT Days 20, 22) | 0.476 | 0.067 | 0.078 |
| Group 5 (AAV IT Days 1, 5) (1.0 mg/kg AC003567 IT Days 20, 22) | 0.452 | 0.064 | 0.074 |
| Group 6 (AAV IT Days 1, 5) (1.0 mg/kg AC003511 IT Days 20, 22) | 0.547 | 0.101 | 0.123 |
| Group 7 (AAV IT Days 1, 5) (1.0 mg/kg AC004079 IT Days 20, 22) | 0.687 | 0.141 | 0.178 |
| Group 8 (AAV IT Days 1, 5) (1.0 mg/kg AC003602 IT Days 20, 22) | 0.380 | 0.079 | 0.100 |

As shown in Table 33, above, each of the TSLP RNAi agents tested (Groups 2-8) showed reductions in hTSLP expression compared to control (Group 1). In particular, several of the TSLP RNAi agents targeting the TSLP transcript at position 571 achieved more than 60% hTSLP mRNA inhibition, with AC003374 achieving ˜67% knockdown (Group 2, 0.330), and AC003602 achieving ˜62% knockdown (Group 8, 0.380), mRNA. These results are also shown in FIG. 6A.

Further, hTSLP protein expression was measured for each of the dosing groups by MSD assay from the lower right lobe of the mouse lung tissues collected, and the data from certain of the samples are shown in FIGS. 6B and 6C. As shown in FIG. 6B, AC003374 and AC003602 (Groups 2 and 8, respectively) achieved ˜88% and ˜77% reduction, respectively, in human TSLP protein in AAV transduced mouse lungs at 1.0 mg/kg. Furthermore, as shown in FIG. 6C, AC003374 and AC002603 (Groups 2 and 8, respectively) both achieved ˜81% reduction in human TSLP protein in serum of AAV transduced mice.

### Example 12. TSLP RNAi Agents in AAV9-CAG-hTSLP AAV Mouse Model

To evaluate certain TSLP RNAi agents, the same AAV9-CAG-hTSLP (Adeno-associated virus) mouse model as discussed in Example 4 was used.

The human TSLP mRNA expression was measured in the mice lung tissues by qPCR

At Day 1 and Day 3, each mouse (female C57Bl/6) was given an intratracheal (IT) administration of 50 μL AAV solutions containing 2e10 GC (genome copy) of AAV9-CAG-eGFP and 3e10 GC of AAV9-CAG-hTSLP in PBS. At Day 14 and Day 17, each mouse was given intratracheal administration of 50 μL of 0.4 mg/kg, 0.75 mg/kg, or 1.5 mg/kg of TSLP RNAi agents formulated in isotonic saline, or vehicle control (isotonic saline with no RNAi agent), according to the following Table 34. The mice were humanely sacrificed and harvested on Day 28.

TABLE 34
Targeted Positions and Dosing Groups of Example 12.

| | Targeted TSLP | | | |
|---|---|---|---|---|
| | Gene Position | | | |
| | (within SEQ ID | | RNAi Agent | |
| | NO: 1, GenBank | AAV dose | and Dose | |
| Group | NM_033035.5) | (Day 1, 3) | (Day 15, 18) | Dosing Regimen |
| 1 | N/A | 2e10 GC of AAV9- | Saline | IT doses of AAV on |
| | | CAG-eGFP and | | Day 1, 3; |
| | | 3e10 GC of AAV9- | | IT doses of saline on |
| | | CAG-hTSLP | | Days 14, 17 |
| 2 | 571 | 2e10 GC of AAV9- | 0.4 mg/kg | IT doses of AAV on |
| | | CAG-eGFP and | AC003374 | Day 1, 3; |
| | | 3e10 GC of AAV9- | | IT doses of RNAi |
| | | CAG-hTSLP | | agent on Days 14, 17 |
| 3 | 571 | 2e10 GC of AAV9- | 0.75 mg/kg | IT doses of AAV on |
| | | CAG-eGFP and | AC003374 | Day 1, 3; |
| | | 3e10 GC of AAV9- | | IT doses of RNAi |
| | | CAG-hTSLP | | agent on Days 14, 17 |
| 4 | 571 | 2e10 GC of AAV9- | 1.5 mg/kg | IT doses of AAV on |
| | | CAG-eGFP and | AC003374 | Day 1, 3; |
| | | 3e10 GC of AAV9- | | IT doses of RNAi |
| | | CAG-hTSLP | | agent on Days 14, 17 |
| 5 | 520 | 2e10 GC of AAV9- | 0.4 mg/kg | IT doses of AAV on |
| | | CAG-eGFP and | AC003456 | Day 1, 3; |
| | | 3e10 GC of AAV9- | | IT doses of RNAi |
| | | CAG-hTSLP | | agent on Days 14, 17 |
| 6 | 520 | 2e10 GC of AAV9- | 0.75 mg/kg | IT doses of AAV on |
| | | CAG-eGFP and | AC003456 | Day 1, 3; |
| | | 3e10 GC of AAV9- | | IT doses of RNAi |
| | | CAG-hTSLP | | agent on Days 14, 17 |
| 7 | 520 | 2e10 GC of AAV9- | 1.5 mg/kg | IT doses of AAV on |
| | | CAG-eGFP and | AC003456 | Day 1, 3; |
| | | 3e10 GC of AAV9- | | IT doses of RNAi |
| | | CAG-hTSLP | | agent on Days 14, 17 |
| 8 | 520 | 2e10 GC of AAV9- | 0.75 mg/kg | IT doses of AAV on |
| | | CAG-eGFP and | AC003342 | Day 1, 3; |
| | | 3e10 GC of AAV9- | | IT doses of RNAi |
| | | CAG-hTSLP | | agent on Days 14, 17 |

Each of the TSLP RNAi agents included modified nucleotides that were conjugated at the 5′ terminal end of the sense strand to an αvβ6 integrin targeting ligand having the modified sequences as set forth in the duplex structures herein. (See Tables 3, 4, 5, 6, 7A, 7B, 8, 9, 10, and 11 for specific modifications and structure information related to the TSLP RNAi agents, including Tri-SM6.1-αvβ6). The TSLP RNAi agents in Groups 2-8 each included nucleotide sequences that were designed to inhibit expression of a TSLP gene by targeting specific positions of TSLP mRNA as set forth in Table 34, above. (See. e.g., SEQ ID NO:1 and Table 2 for the TSLP mRNA sequence referenced.)

Five (5) mice were tested (n=5) for each group. Left lobe lungs were collected in 4% PFA for histology analysis. Lower right lobes were collected for human TSLP protein measurement by Meso Scale Discovery (MSD) Assay. All remaining right lobes were collected for TSLP mRNA expression measurement by qPCR Data from the experiment are shown in the following Table 35:

TABLE 35
Average Relative TSLP Normalized to Control in AAV-hTSLP Mice from Example 12.

| | Average Relative hTSLP | Low | High |
|---|---|---|---|
| Group ID | mRNA Expression | (error) | (error) |
| Group 1 (AAV IT Days 1, 3) (Saline IT Days 14, 17) | 1.000 | 0.189 | 0.233 |
| Group 2 (AAV IT Days 1, 3) (0.4 mg/kg AC003374 Days 14, 17) | 0.550 | 0.110 | 0.138 |
| Group 3 (AAV IT Days 1, 3) (0.75 mg/kg AC003374 Days 14, 17) | 0.382 | 0.052 | 0.060 |
| Group 4 (AAV IT Days 1, 3) (1.5 mg/kg AC003374 Days 14, 17) | 0.376 | 0.065 | 0.078 |
| Group 5 (AAV IT Days 1, 3) (0.4 mg/kg AC003456 Days 14, 17) | 0.544 | 0.124 | 0.160 |
| Group 6 (AAV IT Days 1, 3) (0.75 mg/kg AC003456 Days 14, 17) | 0.614 | 0.120 | 0.150 |
| Group 7 (AAV IT Days 1, 3) (1.5 mg/kg AC003456 Days 14, 17) | 0.583 | 0.105 | 0.128 |
| Group 8 (AAV IT Days 1, 3) (0.75 mg/kg AC003342 Days 14, 17) | 0.576 | 0.137 | 0.180 |

As shown in Table 35, above, each of the TSLP RNAi agents tested (Groups 2-8) showed reductions in hTSLP expression compared to control (Group 1). In particular, AC003374 (targeting position 571) achieved ˜62% inhibition (0.376) of TSLP mRNA at 1.5 mg/kg.

### Example 13. TSLP RNAi Agents in AAV9-CAG-hTSLP AAV Mouse Model

To evaluate certain TSLP RNAi agents, the same AAV9-CAG-hTSLP (Adeno-associated virus) mouse model as discussed in Example 4 was used.

The human TSLP CRNA expression was measured in the mice lung tissues by PCR

At Day 1 and Day 3, each mouse (female C57Bl/6) was given an intratracheal (IT) administration of 50 μL AAV solutions containing 2e10 GC (genome copy) of AAV9-CAG-eGFP and 3e10 GC of AAV9-CAG-hTSLP in PBS. At Day 17 and Day 20, each mouse was given intratracheal administration of 50 μL of 0.5 mg/kg or 1.0 mg/kg of TSLP RNAi agents formulated in isotonic saline, or vehicle control (isotonic saline with no RNAi agent), according to the following Table 36. The mice were humanely sacrificed and harvested on Day 29.

TABLE 36
Targeted Positions and Dosing Groups of Example 13.

| | Targeted TSLP | | | |
|---|---|---|---|---|
| | Gene Position | | | |
| | (within SEQ ID | | RNAi Agent | |
| | NO: 1, GenBank | AAV dose | and Dose | |
| Group | NM_033035.5) | (Day 1, 3) | (Day 15, 18) | Dosing Regimen |
| 1 | N/A | 2e10 GC of AAV9- | Saline | IT doses of AAV on |
| | | CAG-eGFP and | | Day 1, 3; |
| | | 3e10 GC of AAV9- | | IT doses of saline on |
| | | CAG-hTSLP | | Days 17, 20 |
| 2 | 571 | 2e10 GC of AAV9- | 0.5 mg/kg | IT doses of AAV on |
| | | CAG-eGFP and | AC003374 | Day 1, 3; |
| | | 3e10 GC of AAV9- | | IT doses of RNAi |
| | | CAG-hTSLP | | agent on Days 17, 20 |
| 3 | 571 | 2e10 GC of AAV9- | 1.0 mg/kg | IT doses of AAV on |
| | | CAG-eGFP and | AC003374 | Day 1, 3; |
| | | 3e10 GC of AAV9- | | IT doses of RNAi |
| | | CAG-hTSLP | | agent on Days 17, 20 |
| 4 | 571 | 2e10 GC of AAV9- | 0.5 mg/kg | IT doses of AAV on |
| | | CAG-eGFP and | AC003376 | Day 1, 3; |
| | | 3e10 GC of AAV9- | | IT doses of RNAi |
| | | CAG-hTSLP | | agent on Days 17, 20 |
| 5 | 571 | 2e10 GC of AAV9- | 1.0 mg/kg | IT doses of AAV on |
| | | CAG-eGFP and | AC003376 | Day 1, 3; |
| | | 3e10 GC of AAV9- | | IT doses of RNAi |
| | | CAG-hTSLP | | agent on Days 17, 20 |
| 6 | 571 | 2e10 GC of AAV9- | 0.5 mg/kg | IT doses of AAV on |
| | | CAG-eGFP and | AC003602 | Day 1, 3; |
| | | 3e10 GC of AAV9- | | IT doses of RNAi |
| | | CAG-hTSLP | | agent on Days 17, 20 |
| 7 | 571 | 2e10 GC of AAV9- | 1.0 mg/kg | IT doses of AAV on |
| | | CAG-eGFP and | AC003602 | Day 1, 3; |
| | | 3e10 GC of AAV9- | | IT doses of RNAi |
| | | CAG-hTSLP | | agent on Days 17, 20 |
| 8 | 571 | 2e10 GC of AAV9- | 0.5 mg/kg | IT doses of AAV on |
| | | CAG-eGFP and | AC003601 | Day 1, 3; |
| | | 3e10 GC of AAV9- | | IT doses of RNAi |
| | | CAG-hTSLP | | agent on Days 17, 20 |

Each of the TSLP RNAi agents included modified nucleotides that were conjugated at the 5′ terminal end of the sense strand to an αvβ6 integrin targeting ligand having the modified sequences as set forth in the duplex structures herein. (See Tables 3, 4, 5, 6, 7A, 7B, 8, 9, 10, and 11 for specific modifications and structure information related to the TSLP RNAi agents, including Tri-SM6.1-αvβ6). The TSLP RNAi agents in Groups 2-8 each included nucleotide sequences that were designed to inhibit expression of a TSLP gene by targeting specific positions of TSLP mRNA as set forth in Table 36, above. (See. e.g., SEQ ID NO:1 and Table 2 for the TSLP mRNA sequence referenced.)

Five (5) mice were tested (n=5) for each group. Left lobe lungs were collected in 4% PFA for histology analysis. Lower right lobes were collected for human TSLP protein measurement by Meso Scale Discovery (MSD) Assay. All remaining right lobes were collected for TSLP mRNA expression measurement by qPCR Data from the experiment are shown in the following Table 37:

TABLE 37
Average Relative TSLP Normalized to Control in AAV-hTSLP Mice from Example 13.

| | Average Relative hTSLP | Low | High |
|---|---|---|---|
| Group ID | mRNA Expression | (error) | (error) |
| Group 1 (AAV IT Days 1, 3) (Saline IT Days 17, 20) | 1.000 | 0.102 | 0.114 |
| Group 2 (AAV IT Days 1, 3) (0.5 mg/kg AC003374 IT Days 17, 20) | 0.969 | 0.108 | 0.121 |
| Group 3 (AAV IT Days 1, 3) (1.0 mg/kg AC003374 IT Days 17, 20) | 0.435 | 0.059 | 0.068 |
| Group 4 (AAV IT Days 1, 3) (0.5 mg/kg AC003376 IT Days 17, 20) | 0.697 | 0.120 | 0.144 |
| Group 5 (AAV IT Days 1, 3) (1.0 mg/kg AC003376 IT Days 17, 20) | 0.709 | 0.118 | 0.141 |
| Group 6 (AAV IT Days 1, 3) (0.5 mg/kg AC003602 IT Days 17, 20) | 0.733 | 0.204 | 0.283 |
| Group 7 (AAV IT Days 1, 3) (1.0 mg/kg AC003602 IT Days 17, 20) | 0.440 | 0.092 | 0.117 |
| Group 8 (AAV IT Days 1, 3) (0.5 mg/kg AC003601 IT Days 17, 20) | 0.748 | 0.184 | 0.245 |

As shown in Table 37, above, each of the TSLP RNAi agents tested (Groups 2-8) showed reductions in hTSLP expression compared to control (Group 1). In particular, AC003602 achieved ˜56% inhibition (0.440) of TSLP mRNA at 1.0 mg/kg.

### Example 14. TSLP RNAi Agents in AAV9-CAG-hTSLP AAV Mouse Model

To evaluate certain TSLP RNAi agents, the same AAV9-CAG-hTSLP (Adeno-associated virus) mouse model as discussed in Example 4 was used.

The human TSLP mRNA expression was measured in the mice lung tissues by qPCR

At Day 1 and Day 3, each mouse (female C57B6) was given an intratracheal (IT) administration of 50 μL AAV solutions containing 2e10 GC (genome copy) of AAV9-CAG-eGFP and 2e10 GC of AAV9-CAG-hTSLP in PBS. At Day 17 and Day 21, each mouse was given intratracheal administration of 50 μL of 1.5 mg/kg of TSLP RNAi agents formulated in isotonic saline, or vehicle control (isotonic saline with no RNAi agent), according to the following Table 38. The mice were humanely sacrificed and harvested on Day 31.

TABLE 38
Targeted Positions and Dosing Groups of Example 14.

| | Targeted TSLP | | | |
|---|---|---|---|---|
| | Gene Position | | | |
| | (within SEQ ID | | RNAi Agent | |
| | NO: 1, GenBank | AAV dose | and Dose | |
| Group | NM_033035.5) | (Day 1, 3) | (Day 15, 18) | Dosing Regimen |
| 1 | N/A | 2e10 GC of AAV9- | Saline | IT doses of AAV on |
| | | CAG-eGFP and | | Day 1, 3; |
| | | 2e10 GC of AAV9- | | IT doses of saline on |
| | | CAG-hTSLP | | Days 17, 21 |
| 2 | 571 | 2e10 GC of AAV9- | 1.5 mg/kg | IT doses of AAV on |
| | | CAG-eGFP and | AC003374 | Day 1, 3; |
| | | 2e10 GC of AAV9- | | IT doses of RNAi |
| | | CAG-hTSLP | | agent on Days 17, 21 |
| 3 | 571 | 2e10 GC of AAV9- | 1.5 mg/kg | IT doses of AAV on |
| | | CAG-eGFP and | AC003602 | Day 1, 3; |
| | | 2e10 GC of AAV9- | | IT doses of RNAi |
| | | CAG-hTSLP | | agent on Days 17, 21 |
| 4 | 398 | 2e10 GC of AAV9- | 1.5 mg/kg | IT doses of AAV on |
| | | CAG-eGFP and | AC003096 | Day 1, 3; |
| | | 2e10 GC of AAV9- | | IT doses of RNAi |
| | | CAG-hTSLP | | agent on Days 17, 21 |
| 5 | 515 | 2e10 GC of AAV9- | 1.5 mg/kg | IT doses of AAV on |
| | | CAG-eGFP and | AC003098 | Day 1, 3; |
| | | 2e10 GC of AAV9- | | IT doses of RNAi |
| | | CAG-hTSLP | | agent on Days 17, 21 |
| 6 | 413 | 2e10 GC of AAV9- | 1.5 mg/kg | IT doses of AAV on |
| | | CAG-eGFP and | AC003129 | Day 1, 3; |
| | | 2e10 GC of AAV9- | | IT doses of RNAi |
| | | CAG-hTSLP | | agent on Days 17, 21 |
| 7 | 568 | 2e10 GC of AAV9- | 1.5 mg/kg | IT doses of AAV on |
| | | CAG-eGFP and | AC003101 | Day 1, 3; |
| | | 2e10 GC of AAV9- | | IT doses of RNAi |
| | | CAG-hTSLP | | agent on Days 17, 21 |
| 8 | 571 | 2e10 GC of AAV9- | 1.5 mg/kg | IT doses of AAV on |
| | | CAG-eGFP and | AC004361 | Day 1, 3; |
| | | 2e10 GC of AAV9- | | IT doses of RNAi |
| | | CAG-hTSLP | | agent on Days 17, 21 |

Each of the TSLP RNAi agents included modified nucleotides that were conjugated at the 5′ terminal end of the sense strand to an αvβ6 integrin targeting ligand having the modified sequences as set forth in the duplex structures herein. (See Tables 3, 4, 5, 6, 7A, 7B, 8, 9, 10, and 11 for specific modifications and structure information related to the TSLP RNAi agents, including Tri-SM6.1-αvβ6). The TSLP RNAi agents in Groups 2-8 each included nucleotide sequences that were designed to inhibit expression of a TSLP gene by targeting specific positions of TSLP mRNA as set forth in Table 38, above. (See. e.g., SEQ ID NO:1 and Table 2 for the TSLP mRNA sequence referenced.)

Five (5) mice were tested (n=5) for each group. Left lobe lungs were collected in 4% PFA for histology analysis. Lower right lobes were collected for human TSLP protein measurement by Meso Scale Discovery (MSD) Assay. All remaining right lobes were collected for TSLP mRNA expression measurement by qPCR Data from the experiment are shown in the following Table 39:

TABLE 39
Average Relative TSLP Normalized to Control in AAV-hTSLP Mice from Example 14.

| | Average Relative hTSLP | Low | High |
|---|---|---|---|
| Group ID | mRNA Expression | (error) | (error) |
| Group 1 (AAV IT Days 1, 3) (Saline IT Days 17, 21) | 1.000 | 0.373 | 0.595 |
| Group 2 (AAV IT Days 1, 3) (1.5 mg/kg AC003374 IT Days 17, 21) | 0.402 | 0.129 | 0.191 |
| Group 3 (AAV IT Days 1, 3) (1.5 mg/kg AC003602 IT Days 17, 21) | 0.325 | 0.090 | 0.124 |
| Group 4 (AAV IT Days 1, 3) (1.5 mg/kg AC003096 IT Days 17, 21) | 0.525 | 0.116 | 0.149 |
| Group 5 (AAV IT Days 1, 3) (1.5 mg/kg AC003098 IT Days 17, 21) | 0.370 | 0.133 | 0.207 |
| Group 6 (AAV IT Days 1, 3) (1.5 mg/kg AC003129 IT Days 17, 21) | 0.451 | 0.098 | 0.125 |
| Group 7 (AAV IT Days 1, 3) (1.5 mg/kg AC003101 IT Days 17, 21) | 0.378 | 0.179 | 0.339 |
| Group 8 (AAV IT Days 1, 3) (1.5 mg/kg AC004361 IT Days 17, 21) | 0.280 | 0.077 | 0.107 |

As shown in Table 39, above, each of the TSLP RNAi agents tested (Groups 2-8) showed reductions in hTSLP expression compared to control (Group 1). In particular, AC003602 achieved ˜67% inhibition (0.325) of TSLP mRNA at 1.5 mg/kg, and AC004361 (also targeting position 571 of the TSLP gene) achieved ˜72% inhibition (0.280) of TSLP mRNA at 1.5 mg/kg.

### Example 15. TSLP RNAi Agents in AAV9-CAG-hTSLP AAV Mouse Model

To evaluate certain TSLP RNAi agents, the same AAV9-CAG-hTSLP (Adeno-associated virus) mouse model as discussed in Example 4 was used.

The human TSLP mRNA expression was measured in the mice lung tissues by qPCR

At Day 1 and Day 4, each mouse (female C57Bl/6) was given an intratracheal (IT) administration of 50 μL AAV solutions containing 2e10 GC (genome copy) of AAV9-CAG-eGFP and 3e10 GC of AAV9-CAG-hTSLP in PBS. At Day 20 and Day 22, each mouse was given intratracheal administration of 50 μL of 1.0 mg/kg of TSLP RNAi agents formulated in isotonic saline, or vehicle control (isotonic saline with no RNAi agent), according to the following Table 40. The mice were humanely sacrificed and harvested on Day 32.

TABLE 40
Targeted Positions and Dosing Groups of Example 15.

| | Targeted TSLP | | | |
|---|---|---|---|---|
| | Gene Position | | | |
| | (within SEQ ID | | RNAi Agent | |
| | NO: 1, GenBank | AAV dose | and Dose | |
| Group | NM_033035.5) | (Day 1, 3) | (Day 15, 18) | Dosing Regimen |
| 1 | N/A | 2e10 GC of AAV9- | Saline | IT doses of AAV on |
| | | CAG-eGFP and | | Day 1, 4; |
| | | 3e10 GC of AAV9- | | IT doses of saline on |
| | | CAG-hTSLP | | Days 20, 22 |
| 2 | 571 | 2e10 GC of AAV9- | 1.0 mg/kg | IT doses of AAV on |
| | | CAG-eGFP and | AC003602 | Day 1, 4; |
| | | 3e10 GC of AAV9- | | IT doses of RNAi |
| | | CAG-hTSLP | | agent on Days 20, 22 |
| 3 | 571 | 2e10 GC of AAV9- | 1.0 mg/kg | IT doses of AAV on |
| | | CAG-eGFP and | AC004376 | Day 1, 4; |
| | | 3e10 GC of AAV9- | | IT doses of RNAi |
| | | CAG-hTSLP | | agent on Days 20, 22 |
| 4 | 571 | 2e10 GC of AAV9- | 1.0 mg/kg | IT doses of AAV on |
| | | CAG-eGFP and | AC004363 | Day 1, 4; |
| | | 3e10 GC of AAV9- | | IT doses of RNAi |
| | | CAG-hTSLP | | agent on Days 20, 22 |
| 5 | 571 | 2e10 GC of AAV9- | 1.0 mg/kg | IT doses of AAV on |
| | | CAG-eGFP and | AC004373 | Day 1, 4; |
| | | 3e10 GC of AAV9- | | IT doses of RNAi |
| | | CAG-hTSLP | | agent on Days 20, 22 |
| 6 | 571 | 2e10 GC of AAV9- | 1.0 mg/kg | IT doses of AAV on |
| | | CAG-eGFP and | AC004374 | Day 1, 4; |
| | | 3e10 GC of AAV9- | | IT doses of RNAi |
| | | CAG-hTSLP | | agent on Days 20, 22 |
| 7 | 571 | 2e10 GC of AAV9- | 1.0 mg/kg | IT doses of AAV on |
| | | CAG-eGFP and | AC004358 | Day 1, 4; |
| | | 3e10 GC of AAV9- | | IT doses of RNAi |
| | | CAG-hTSLP | | agent on Days 20, 22 |
| 8 | 571 | 2e10 GC of AAV9- | 1.0 mg/kg | IT doses of AAV on |
| | | CAG-eGFP and | AC004361 | Day 1, 4; |
| | | 3e10 GC of AAV9- | | IT doses of RNAi |
| | | CAG-hTSLP | | agent on Days 20, 22 |

Each of the TSLP RNAi agents included modified nucleotides that were conjugated at the 5′ terminal end of the sense strand to an αvβ6 integrin targeting ligand having the modified sequences as set forth in the duplex structures herein. (See Tables 3, 4, 5, 6, 7A, 7B, 8, 9, 10, and 11 for specific modifications and structure information related to the TSLP RNAi agents, including Tri-SM6.1-αvβ6). The TSLP RNAi agents in Groups 2-8 each included nucleotide sequences that were designed to inhibit expression of a TSLP gene by targeting specific positions of TSLP mRNA as set forth in Table 40, above. (See. e.g., SEQ ID NO:1 and Table 2 for the TSLP mRNA sequence referenced.)

Five (5) mice were tested (n=5) for each group. Left lobe lungs were collected in 4% PFA for histology analysis. Lower right lobes were collected for human TSLP protein measurement by Meso Scale Discovery (MSD) Assay. All remaining right lobes were collected for TSLP mRNA expression measurement by qPCR Data from the experiment are shown in the following Table 41:

TABLE 41
Average Relative TSLP Normalized to Control in AAV-hTSLP Mice from Example 15.

| | Average Relative hTSLP | Low | High |
|---|---|---|---|
| Group ID | mRNA Expression | (error) | (error) |
| Group 1 (AAV IT Days 1, 4) (Saline IT Days 20, 22) | 1.000 | 0.182 | 0.223 |
| Group 2 (AAV IT Days 1, 4) (1.0 mg/kg AC003602 IT Days 20, 22) | 0.424 | 0.099 | 0.130 |
| Group 3 (AAV IT Days 1, 4) (1.0 mg/kg AC004376 IT Days 20, 22) | 0.371 | 0.084 | 0.108 |
| Group 4 (AAV IT Days 1, 4) (1.0 mg/kg AC004363 IT Days 20, 22) | 0.359 | 0.094 | 0.127 |
| Group 5 (AAV IT Days 1, 4) (1.0 mg/kg AC004373 IT Days 20, 22) | 0.411 | 0.091 | 0.116 |
| Group 6 (AAV IT Days 1, 4) (1.0 mg/kg AC004374 IT Days 20, 22) | 0.552 | 0.145 | 0.196 |
| Group 7 (AAV IT Days 1, 4) (1.0 mg/kg AC004358 IT Days 20, 22) | 0.383 | 0.109 | 0.152 |
| Group 8 (AAV IT Days 1, 4) (1.0 mg/kg AC004361 IT Days 20, 22) | 0.381 | 0.090 | 0.117 |

As shown in Table 41, above, each of the TSLP RNAi agents tested (Groups 2-8) showed reductions in hTSLP expression compared to control (Group 1).

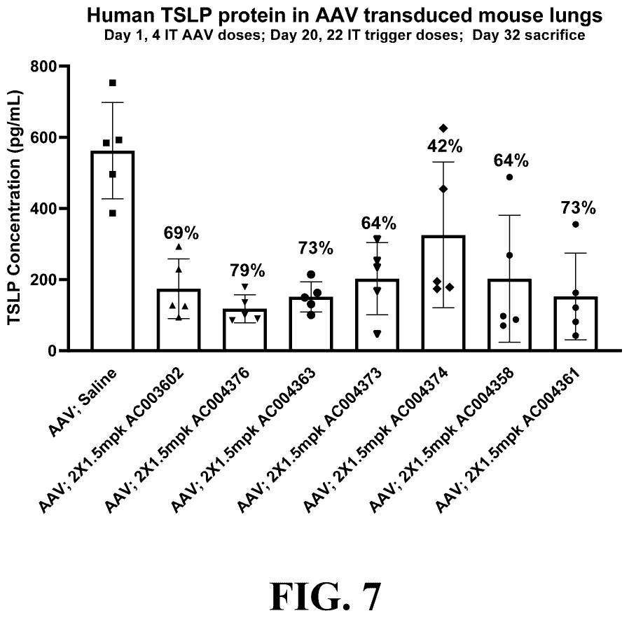

Further, hTSLP protein expression was measured for each of the dosing groups by MSD assay from the lower right lobe of the mouse lung tissues collected, and the data from certain of the samples are shown in FIG. 7. As shown in FIG. 7, AC004376 achieved ˜79% reduction, AC004363 and AC004361 both achieved 73% reduction in human TSLP protein in AAV transduced mouse lungs at 1.0 mg/kg.

### Example 16. TSLP RNAi Agents in AAV9-CAG-hTSLP AAV Mouse Model

To evaluate certain TSLP RNAi agents, the same AAV9-CAG-hTSLP (Adeno-associated virus) mouse model as discussed in Example 4 was used.

The human TSLP mRNA expression was measured in the mice lung tissues by qPCR

At Day 1 and Day 3, each mouse (female C57Bl/6) was given an intratracheal (IT) administration of 50 μL AAV solutions containing 2e10 GC (genome copy) of AAV9-CAG-eGFP and 3e10 GC of AAV9-CAG-hTSLP in PBS. At Day 17 and Day 20, each mouse was given intratracheal administration of 50 μL of 1.0 mg/kg of TSLP RNAi agents formulated in isotonic saline, or vehicle control (isotonic saline with no RNAi agent), according to the following Table 42. The mice were humanely sacrificed and harvested on Day 31.

TABLE 42
Targeted Positions and Dosing Groups of Example 16.

| | Targeted TSLP | | | |
|---|---|---|---|---|
| | Gene Position | | | |
| | (within SEQ ID | | RNAi Agent | |
| | NO: 1, GenBank | AAV dose | and Dose | |
| Group | NM_033035.5) | (Day 1, 3) | (Day 15, 18) | Dosing Regimen |
| 1 | N/A | 2e10 GC of AAV9- | Saline | IT doses of AAV on |
| | | CAG-eGFP and | | Day 1, 3; |
| | | 3e10 GC of AAV9- | | IT doses of saline on |
| | | CAG-hTSLP | | Days 17, 20 |
| 2 | 571 | 2e10 GC of AAV9- | 1.0 mg/kg | IT doses of AAV on |
| | | CAG-eGFP and | AC003602 | Day 1, 3; |
| | | 3e10 GC of AAV9- | | IT doses of RNAi |
| | | CAG-hTSLP | | agent on Days 17, 20 |
| 3 | 571 | 2e10 GC of AAV9- | 1.0 mg/kg | IT doses of AAV on |
| | | CAG-eGFP and | AC004644 | Day 1, 3; |
| | | 3e10 GC of AAV9- | | IT doses of RNAi |
| | | CAG-hTSLP | | agent on Days 17, 20 |
| 4 | 571 | 2e10 GC of AAV9- | 1.0 mg/kg | IT doses of AAV on |
| | | CAG-eGFP and | AC004645 | Day 1, 3; |
| | | 3e10 GC of AAV9- | | IT doses of RNAi |
| | | CAG-hTSLP | | agent on Days 17, 20 |
| 5 | 571 | 2e10 GC of AAV9- | 1.0 mg/kg | IT doses of AAV on |
| | | CAG-eGFP and | AC004646 | Day 1, 3; |
| | | 3e10 GC of AAV9- | | IT doses of RNAi |
| | | CAG-hTSLP | | agent on Days 17, 20 |
| 6 | 571 | 2e10 GC of AAV9- | 1.0 mg/kg | IT doses of AAV on |
| | | CAG-eGFP and | AC004647 | Day 1, 3; |
| | | 3e10 GC of AAV9- | | IT doses of RNAi |
| | | CAG-hTSLP | | agent on Days 17, 20 |
| 7 | 571 | 2e10 GC of AAV9- | 1.0 mg/kg | IT doses of AAV on |
| | | CAG-eGFP and | AC004816 | Day 1, 3; |
| | | 3e10 GC of AAV9- | | IT doses of RNAi |
| | | CAG-hTSLP | | agent on Days 17, 20 |
| 8 | 571 | 2e10 GC of AAV9- | 1.0 mg/kg | IT doses of AAV on |
| | | CAG-eGFP and | AC004363 | Day 1, 3; |
| | | 3e10 GC of AAV9- | | IT doses of RNAi |
| | | CAG-hTSLP | | agent on Days 17, 20 |

Each of the TSLP RNAi agents included modified nucleotides that were conjugated at the 5′ terminal end of the sense strand to an αvβ6 integrin targeting ligand having the modified sequences as set forth in the duplex structures herein. (See Tables 3, 4, 5, 6, 7A, 7B, 8, 9, 10, and 11 for specific modifications and structure information related to the TSLP RNAi agents, including Tri-SM6.1-αvβ6). The TSLP RNAi agents in Groups 2-8 each included nucleotide sequences that were designed to inhibit expression of a TSLP gene by targeting specific positions of TSLP mRNA as set forth in Table 42, above. (See. e.g., SEQ ID NO:1 and Table 2 for the TSLP mRNA sequence referenced.)

Five (5) mice were tested (n=5) for each group. Left lobe lungs were collected in 4% PFA for histology analysis. Lower right lobes were collected for human TSLP protein measurement by Meso Scale Discovery (MSD) Assay. All remaining right lobes were collected for TSLP mRNA expression measurement by qPCR Data from the experiment are shown in the following Table 43:

TABLE 43
Average Relative TSLP Normalized to Control in AAV-hTSLP Mice from Example 16.

| | Average Relative hTSLP | Low | High |
|---|---|---|---|
| Group ID | mRNA Expression | (error) | (error) |
| Group 1. (AAV IT Days 1, 3) (Saline IT Days 17, 20) | 1.000 | 0.157 | 0.187 |
| Group 2. (AAV IT Days 1, 3) (1.0 mg/kg AC003602 IT Days 17, 20) | 0.476 | 0.091 | 0.112 |
| Group 3. (AAV IT Days 1, 3) (1.0 mg/kg AC004644 IT Days 17, 20) | 0.404 | 0.099 | 0.131 |
| Group 4. (AAV IT Days 1, 3) (1.0 mg/kg AC004645 IT Days 17, 20) | 0.355 | 0.047 | 0.055 |
| Group 5. (AAV IT Days 1, 3) (1.0 mg/kg AC004646 IT Days 17, 20) | 0.406 | 0.067 | 0.081 |
| Group 6. (AAV IT Days 1, 3) (1.0 mg/kg AC004647 IT Days 17, 20) | 0.541 | 0.113 | 0.143 |
| Group 7. (AAV IT Days 1, 3) (1.0 mg/kg AC004816 IT Days 17, 20) | 0.509 | 0.084 | 0.100 |
| Group 8. (AAV IT Days 1, 3) (1.0 mg/kg AC004363 IT Days 17, 20) | 0.376 | 0.097 | 0.130 |

As shown in Table 43, above, each of the TSLP RNAi agents tested (Groups 2-8) showed reductions in hTSLP expression compared to control (Group 1). In particular, AC004363 achieved ˜62% inhibition of TSLP mRNA at 1.0 mg/kg.

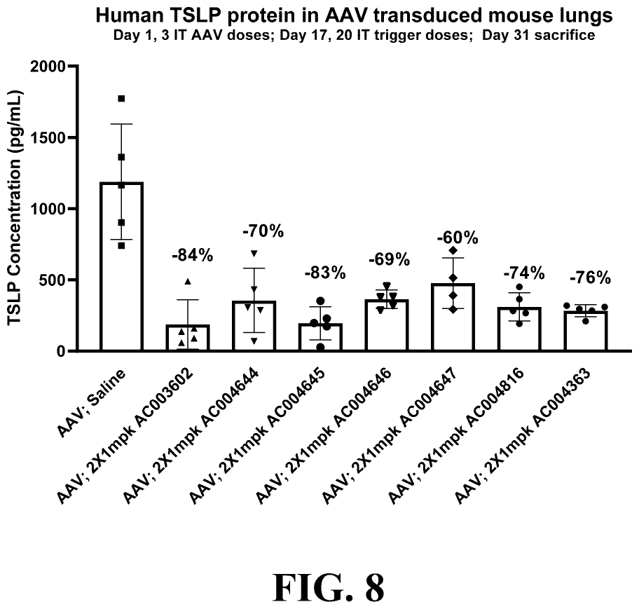

Further, hTSLP protein expression was measured for each of the dosing groups by MSD assay from the lower right lobe of the mouse lung tissues collected, and the data from certain of the samples are shown in FIG. 8. As shown in FIG. 8, AC003602 achieved ˜84% reduction, AC004645 achieved ˜83% reduction in human TSLP protein in AAV transduced mouse lungs at 1.0 mg/kg.

### Example 17. TSLP RNAi Agents in AAV9-CAG-hTSLP AAV Mouse Model

To evaluate certain TSLP RNAi agents, the same AAV9-CAG-hTSLP (Adeno-associated virus) mouse model as discussed in Example 4 was used.

The human TSLP mRNA expression was measured in the mice lung tissues by qPCR

At Day 1 and Day 4, each mouse (female C57Bl/6) was given an intratracheal (IT) administration of 50 μL AAV solutions containing 2e10 GC (genome copy) of AAV9-CAG-eGFP and 3e10 GC of AAV9-CAG-hTSLP in PBS. At Day 22 and Day 25, each mouse was given intratracheal administration of 50 μL of 1.0 mg/kg of TSLP RNAi agents formulated in isotonic saline, or vehicle control (isotonic saline with no RNAi agent), which included the Groups according to the following Table 44. The mice were humanely sacrificed and harvested on Day 36.

TABLE 44
Targeted Positions and Dosing Groups of Example 17.

| | Targeted TSLP | | | |
|---|---|---|---|---|
| | Gene Position | | | |
| | (within SEQ ID | | RNAi Agent | |
| | NO: 1, GenBank | AAV dose | and Dose | |
| Group | NM_033035.5) | (Day 1, 3) | (Day 15, 18) | Dosing Regimen |
| 1 | N/A | 2e10 GC of AAV9- | Saline | IT doses of AAV on |
| | | CAG-eGFP and | | Day 1, 4; |
| | | 3e10 GC of AAV9- | | IT doses of saline on |
| | | CAG-hTSLP | | Days 22, 25 |
| 2 | 571 | 2e10 GC of AAV9- | 1.0 mg/kg | IT doses of AAV on |
| | | CAG-eGFP and | AC003602 | Day 1, 4; |
| | | 3e10 GC of AAV9- | | IT doses of RNAi |
| | | CAG-hTSLP | | agent on Days 22, 25 |
| 3 | 571 | 2e10 GC of AAV9- | 1.0 mg/kg | IT doses of AAV on |
| | | CAG-eGFP and | AC004363 | Day 1, 4; |
| | | 3e10 GC of AAV9- | | IT doses of RNAi |
| | | CAG-hTSLP | | agent on Days 22, 25 |
| 4 | 571 | 2e10 GC of AAV9- | 1.0 mg/kg | IT doses of AAV on |
| | | CAG-eGFP and | AC004376 | Day 1, 4; |
| | | 3e10 GC of AAV9- | | IT doses of RNAi |
| | | CAG-hTSLP | | agent on Days 22, 25 |
| 5 | 571 | 2e10 GC of AAV9- | 1.0 mg/kg | IT doses of AAV on |
| | | CAG-eGFP and | AC004816 | Day 1, 4; |
| | | 3e10 GC of AAV9- | | IT doses of RNAi |
| | | CAG-hTSLP | | agent on Days 22, 25 |
| 6 | 571 | 2e10 GC of AAV9- | 1.0 mg/kg | IT doses of AAV on |
| | | CAG-eGFP and | AC004644 | Day 1, 4; |
| | | 3e10 GC of AAV9- | | IT doses of RNAi |
| | | CAG-hTSLP | | agent on Days 22, 25 |
| 7 | 571 | 2e10 GC of AAV9- | 1.0 mg/kg | IT doses of AAV on |
| | | CAG-eGFP and | AC004646 | Day 1, 4; |
| | | 3e10 GC of AAV9- | | IT doses of RNAi |
| | | CAG-hTSLP | | agent on Days 22, 25 |

Each of the TSLP RNAi agents included modified nucleotides that were conjugated at the 5′ terminal end of the sense strand to an αvβ6 integrin targeting ligand having the modified sequences as set forth in the duplex structures herein. (See Tables 3, 4, 5, 6, 7A, 7B, 8, 9, 10, and 11 for specific modifications and structure information related to the TSLP RNAi agents, including Tri-SM6.1-αvβ6). The TSLP RNAi agents in Groups 2-8 each included nucleotide sequences that were designed to inhibit expression of a TSLP gene by targeting specific positions of TSLP mRNA as set forth in Table 44, above. (See. e.g., SEQ ID NO:1 and Table 2 for the TSLP mRNA sequence referenced.)

Five (5) mice were tested (n=5) for each group. Left lobe lungs were collected in 4% PFA for histology analysis. Lower right lobes were collected for human TSLP protein measurement by Meso Scale Discovery (MSD) Assay. All remaining right lobes were collected for TSLP mRNA expression measurement by qPCR Data from the experiment are shown in the following Table 45:

TABLE 45
Average Relative TSLP Normalized to Control in AAV-hTSLP Mice from Example 17.

| | Average Relative hTSLP | Low | High |
|---|---|---|---|
| Group ID | mRNA Expression | (error) | (error) |
| Group 1. (AAV IT Days 1, 4) (Saline IT Days 22, 25) | 1.000 | 0.055 | 0.059 |
| Group 2. (AAV IT Days 1, 4) (1.0 mg/kg AC003602 IT Days 22, 25) | 0.375 | 0.052 | 0.060 |
| Group 3. (AAV IT Days 1, 4) (1.0 mg/kg AC004363 IT Days 22, 25) | 0.359 | 0.081 | 0.104 |
| Group 4. (AAV IT Days 1, 4) (1.0 mg/kg AC004376 IT Days 22, 25) | 0.324 | 0.066 | 0.083 |
| Group 5. (AAV IT Days 1, 4) (1.0 mg/kg AC004816 IT Days 22, 25) | 0.559 | 0.119 | 0.151 |
| Group 6. (AAV IT Days 1, 4) (1.0 mg/kg AC004644 IT Days 22, 25) | 0.501 | 0.078 | 0.092 |
| Group 7. (AAV IT Days 1, 4) (1.0 mg/kg AC004646 IT Days 22, 25) | 0.655 | 0.046 | 0.050 |

As shown in Table 45, above, each of the TSLP RNAi agents tested (Groups 2-8) showed reductions in hTSLP expression compared to control (Group 1). In particular, AC004376 achieved ˜67% inhibition (0.324) of TSLP mRNA at 1.0 mg/kg and AC003602 achieved ˜62% inhibition of TSLP mRNA at 1.0 mg/kg (0.375).

### Example 18. TSLP RNAi Agents in AAV9-CAG-hTSLP AAV Mouse Model

To evaluate certain TSLP RNAi agents, the same AAV9-CAG-hTSLP (Adeno-associated virus) mouse model as discussed in Example 4 was used.

The human TSLP miRNA expression was measured in the mice lung tissues by qPCR 203611 At Day 1 and Day 4, each mouse (female C57Bl/6) was given an intratracheal (IT) administration of 50 μL AAV solutions containing 2e10 GC (genome copy) of AAV9-CAG-eGFP and 2e10 GC of AAV9-CAG-hTSLP in PBS. At Day 20 and Day 22, each mouse was given intratracheal administration of 50 μL of 1.5 mg/kg of TSLP RNAi agents formulated in isotonic saline, or vehicle control (isotonic saline with no RNAi agent), according to the following Table 46. The mice were humanely sacrificed and harvested on Day 34.

TABLE 46
Targeted Positions and Dosing Groups of Example 18.

| | Targeted TSLP | | | |
|---|---|---|---|---|
| | Gene Position | | | |
| | (within SEQ ID | | RNAi Agent | |
| | NO: 1, GenBank | AAV dose | and Dose | |
| Group | NM_033035.5) | (Day 1, 3) | (Day 15, 18) | Dosing Regimen |
| 1 | N/A | 2e10 GC of AAV9- | Saline | IT doses of AAV on |
| | | CAG-eGFP and | | Day 1, 4; |
| | | 2e10 GC of AAV9- | | IT doses of saline on |
| | | CAG-hTSLP | | Days 20, 22 |
| 2 | 485 | 2e10 GC of AAV9- | 1.5 mg/kg | IT doses of AAV on |
| | | CAG-eGFP and | AC004565 | Day 1, 4; |
| | | 2e10 GC of AAV9- | | IT doses of RNAi |
| | | CAG-hTSLP | | agent on Days 20, 22 |
| 3 | 626 | 2e10 GC of AAV9- | 1.5 mg/kg | IT doses of AAV on |
| | | CAG-eGFP and | AC004566 | Day 1, 4; |
| | | 2e10 GC of AAV9- | | IT doses of RNAi |
| | | CAG-hTSLP | | agent on Days 20, 22 |
| 4 | 719 | 2e10 GC of AAV9- | 1.5 mg/kg | IT doses of AAV on |
| | | CAG-eGFP and | AC004567 | Day 1, 4; |
| | | 2e10 GC of AAV9- | | IT doses of RNAi |
| | | CAG-hTSLP | | agent on Days 20, 22 |
| 5 | 773 | 2e10 GC of AAV9- | 1.5 mg/kg | IT doses of AAV on |
| | | CAG-eGFP and | AC004568 | Day 1, 4; |
| | | 2e10 GC of AAV9- | | IT doses of RNAi |
| | | CAG-hTSLP | | agent on Days 20, 22 |
| 6 | 836 | 2e10 GC of AAV9- | 1.5 mg/kg | IT doses of AAV on |
| | | CAG-eGFP and | AC004569 | Day 1, 4; |
| | | 2e10 GC of AAV9- | | IT doses of RNAi |
| | | CAG-hTSLP | | agent on Days 20, 22 |
| 7 | 863 | 2e10 GC of AAV9- | 1.5 mg/kg | IT doses of AAV on |
| | | CAG-eGFP and | AC004570 | Day 1, 4; |
| | | 2e10 GC of AAV9- | | IT doses of RNAi |
| | | CAG-hTSLP | | agent on Days 20, 22 |
| 8 | 571 | 2e10 GC of AAV9- | 1.5 mg/kg | IT doses of AAV on |
| | | CAG-eGFP and | AC003602 | Day 1, 4; |
| | | 2e10 GC of AAV9- | | IT doses of RNAi |
| | | CAG-hTSLP | | agent on Days 20, 22 |

Each of the TSLP RNAi agents included modified nucleotides that were conjugated at the 5′ terminal end of the sense strand to an αvβ6 integrin targeting ligand having the modified sequences as set forth in the duplex structures herein. (See Tables 3, 4, 5, 6, 7A, 7B, 8, 9, 10, and 11 for specific modifications and structure information related to the TSLP RNAi agents, including Tri-SM6.1-αvβ6). The TSLP RNAi agents in Groups 2-8 each included nucleotide sequences that were designed to inhibit expression of a TSLP gene by targeting specific positions of TSLP mRNA as set forth in Table 46, above. (See. e.g., SEQ ID NO:1 and Table 2 for the TSLP mRNA sequence referenced.)

Five (5) mice were tested (n=5) for each group. Left lobe lungs were collected in 4% PFA for histology analysis. Lower right lobes were collected for human TSLP protein measurement by Meso Scale Discovery (MSD) Assay. All remaining right lobes were collected for TSLP mRNA expression measurement by qPCR Data from the experiment are shown in the following Table 47:

TABLE 47
Average Relative TSLP Normalized to Control in AAV-hTSLP Mice from Example 18.

| | Average Relative hTSLP | Low | High |
|---|---|---|---|
| Group ID | mRNA Expression | (error) | (error) |
| Group 1. (AAV IT Days 1, 4) (Saline IT Days 20, 22) | 1.000 | 0.314 | 0.458 |
| Group 2. (AAV IT Days 1, 4) (1.5 mg/kg AC004565 IT Days 20, 22) | 0.683 | 0.170 | 0.227 |
| Group 3. (AAV IT Days 1, 4) (1.5 mg/kg AC004566 IT Days 20, 22) | 0.448 | 0.125 | 0.174 |
| Group 4. (AAV IT Days 1, 4) (1.5 mg/kg AC004567 IT Days 20, 22) | 0.659 | 0.150 | 0.194 |
| Group 5. (AAV IT Days 1, 4) (1.5 mg/kg AC004568 IT Days 20, 22) | 0.400 | 0.091 | 0.097 |
| Group 6. (AAV IT Days 1, 4) (1.5 mg/kg AC004569 IT Days 20, 22) | 0.465 | 0.069 | 0.082 |
| Group 7. (AAV IT Days 1, 4) (1.5 mg/kg AC004570 IT Days 20, 22) | 0.564 | 0.160 | 0.224 |
| Group 8. (AAV IT Days 1, 4) (1.5 mg/kg AC003602 IT Days 20, 22) | 0.399 | 0.164 | 0.279 |

As shown in Table 47, above, each of the TSLP RNAi agents tested (Groups 2-8) showed reductions in hTSLP expression compared to control (Group 1). In particular, AC003602 and AC004568 achieved ˜60% inhibition of TSLP mRNA at 1.5 mg/kg (0.399 and 0.400, respectively).

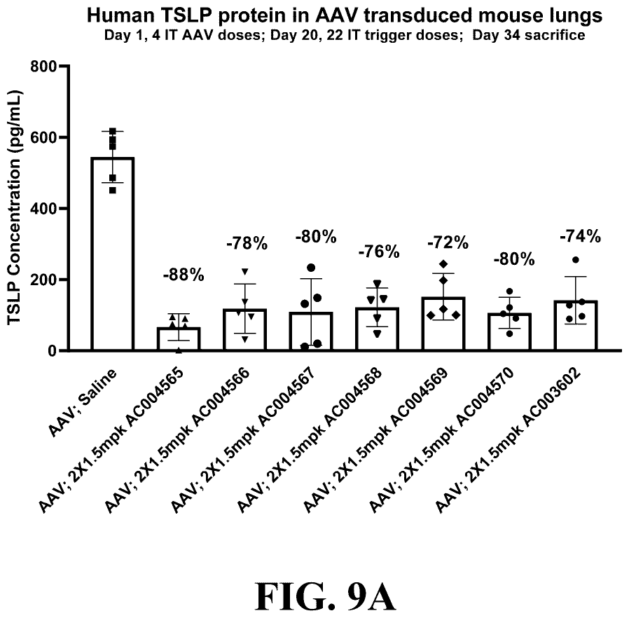

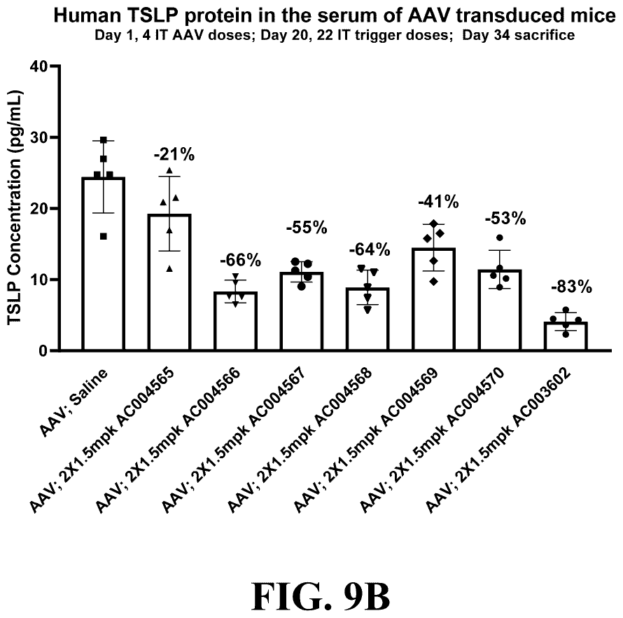

Further, hTSLP protein expression was measured for each of the dosing groups by MSD assay from the lower right lobe of the mouse lung tissues collected, and the data from certain of the samples are shown in FIGS. 9A and 9B. As shown in FIG. 9A, AC004565 achieved ˜88% reduction in human TSLP protein in AAV transduced mouse lungs at 1.5 mg/kg. As shown in FIG. 9B, AC003602 achieved ˜83% reduction, and AC004566 achieved ˜66% reduction, in human TSLP protein in AAV transduced mouse serum at 1.5 mg/kg.

### Example 19. TSLP RNAi Agents in AAV9-CAG-hTSLP AAV Mouse Model

To evaluate certain TSLP RNAi agents, the same AAV9-CAG-hTSLP (Adeno-associated virus) mouse model as discussed in Example 4 was used.

The human TSLP mRNA expression was measured in the mice lung tissues by qPCR

At Day 1 and Day 4, each mouse (female C57Bl/6) was given an intratracheal (IT) administration of 50 μL AAV solutions containing 2e10 GC (genome copy) of AAV9-CAG-eGFP and 2e10 GC of AAV9-CAG-hTSLP in PBS. At Day 18 and Day 20, each mouse was given intratracheal administration of 50 μL of 1.5 mg/kg of TSLP RNAi agents formulated in isotonic saline, or vehicle control (isotonic saline with no RNAi agent), according to the following Table 48. The mice were humanely sacrificed and harvested on Day 32.

TABLE 48
Targeted Positions and Dosing Groups of Example 19.

| | Targeted TSLP | | | |
|---|---|---|---|---|
| | Gene Position | | | |
| | (within SEQ ID | | RNAi Agent | |
| | NO: 1, GenBank | AAV dose | and Dose | |
| Group | NM_033035.5) | (Day 1, 3) | (Day 15, 18) | Dosing Regimen |
| 1 | N/A | 2e10 GC of AAV9- | Saline | IT doses of AAV on |
| | | CAG-eGFP and | | Day 1, 4; |
| | | 2e10 GC of AAV9- | | IT doses of saline on |
| | | CAG-hTSLP | | Days 18, 20 |
| 2 | 836 | 2e10 GC of AAV9- | 1.5 mg/kg | IT doses of AAV on |
| | | CAG-eGFP and | AC004569 | Day 1, 4; |
| | | 2e10 GC of AAV9- | | IT doses of RNAi |
| | | CAG-hTSLP | | agent on Days 18, 20 |
| 3 | 863 | 2e10 GC of AAV9- | 1.5 mg/kg | IT doses of AAV on |
| | | CAG-eGFP and | AC004570 | Day 1, 4; |
| | | 2e10 GC of AAV9- | | IT doses of RNAi |
| | | CAG-hTSLP | | agent on Days 18, 20 |
| 4 | 992 | 2e10 GC of AAV9- | 1.5 mg/kg | IT doses of AAV on |
| | | CAG-eGFP and | AC004571 | Day 1, 4; |
| | | 2e10 GC of AAV9- | | IT doses of RNAi |
| | | CAG-hTSLP | | agent on Days 18, 20 |
| 5 | 1021 | 2e10 GC of AAV9- | 1.5 mg/kg | IT doses of AAV on |
| | | CAG-eGFP and | AC004572 | Day 1, 4; |
| | | 2e10 GC of AAV9- | | IT doses of RNAi |
| | | CAG-hTSLP | | agent on Days 18, 20 |
| 6 | 1040 | 2e10 GC of AAV9- | 1.5 mg/kg | IT doses of AAV on |
| | | CAG-eGFP and | AC004573 | Day 1, 4; |
| | | 2e10 GC of AAV9- | | IT doses of RNAi |
| | | CAG-hTSLP | | agent on Days 18, 20 |
| 7 | 1218 | 2e10 GC of AAV9- | 1.5 mg/kg | IT doses of AAV on |
| | | CAG-eGFP and | AC004574 | Day 1, 4; |
| | | 2e10 GC of AAV9- | | IT doses of RNAi |
| | | CAG-hTSLP | | agent on Days 18, 20 |
| 8 | 571 | 2e10 GC of AAV9- | 1.5 mg/kg | IT doses of AAV on |
| | | CAG-eGFP and | AC003602 | Day 1, 4; |
| | | 2e10 GC of AAV9- | | IT doses of RNAi |
| | | CAG-hTSLP | | agent on Days 18, 20 |

Each of the TSLP RNAi agents included modified nucleotides that were conjugated at the 5′ terminal end of the sense strand to an αvβ6 integrin targeting ligand having the modified sequences as set forth in the duplex structures herein. (See Tables 3, 4, 5, 6, 7A, 7B, 8, 9, 10, and 11 for specific modifications and structure information related to the TSLP RNAi agents, including Tri-SM6.1-αvβ6). The TSLP RNAi agents in Groups 2-8 each included nucleotide sequences that were designed to inhibit expression of a TSLP gene by targeting specific positions of TSLP mRNA as set forth in Table 48, above. (See. e.g., SEQ ID NO:1 and Table 2 for the TSLP mRNA sequence referenced.)

Five (5) mice were tested (n=5) for each group. Left lobe lungs were collected in 4% PFA for histology analysis. Lower right lobes were collected for human TSLP protein measurement by Meso Scale Discovery (MSD) Assay. All remaining right lobes were collected for TSLP mRNA expression measurement by qPCR Data from the experiment are shown in the following Table 49:

TABLE 49
Average Relative TSLP Normalized to Control in AAV-hTSLP Mice from Example 19.

| | Average Relative hTSLP | Low | High |
|---|---|---|---|
| Group ID | mRNA Expression | (error) | (error) |
| Group 1. (AAV IT Days 1, 4) (Saline IT Days 18, 20) | 1.000 | 0.097 | 0.107 |
| Group 2. (AAV IT Days 1, 4) (1.5 mg/kg AC004569 IT Days 18, 20) | 0.713 | 0.103 | 0.120 |
| Group 3. (AAV IT Days 1, 4) (1.5 mg/kg AC004570 IT Days 18, 20) | 0.665 | 0.140 | 0.178 |
| Group 4. (AAV IT Days 1, 4) (1.5 mg/kg AC004571 IT Days 18, 20) | 0.506 | 0.078 | 0.093 |
| Group 5. (AAV IT Days 1, 4) (1.5 mg/kg AC004572 IT Days 18, 20) | 0.534 | 0.086 | 0.103 |
| Group 6. (AAV IT Days 1, 4) (1.5 mg/kg AC004573 IT Days 18, 20) | 0.383 | 0.086 | 0.111 |
| Group 7. (AAV IT Days 1, 4) (1.5 mg/kg AC004574 IT Days 18, 20) | 0.400 | 0.038 | 0.042 |
| Group 8. (AAV IT Days 1, 4) (1.5 mg/kg AC003602 IT Days 18, 20) | 0.404 | 0.044 | 0.049 |

As shown in Table 49, above, each of the TSLP RNAi agents tested (Groups 2-8) showed reductions in hTSLP expression compared to control (Group 1).

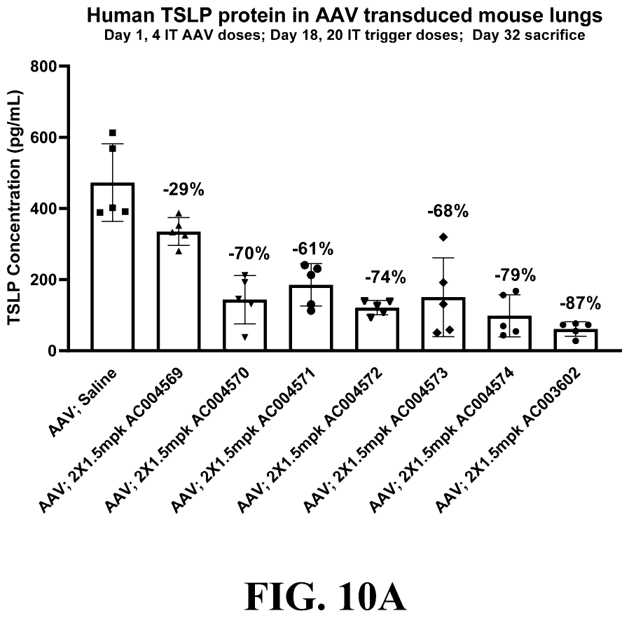

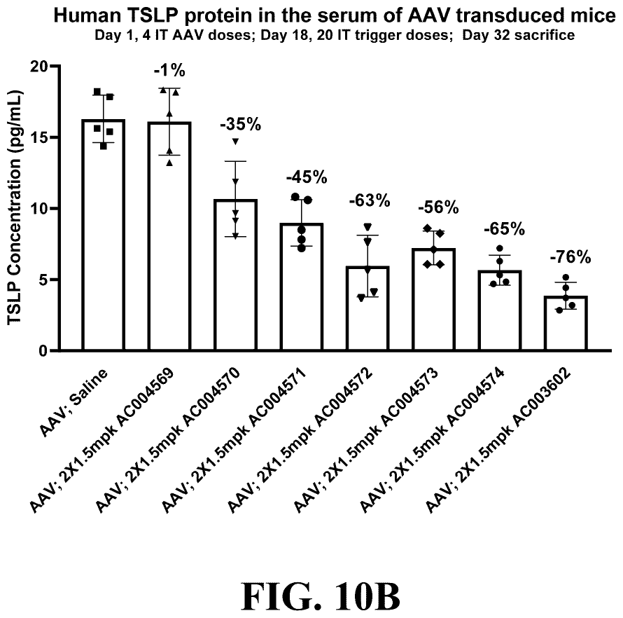

Further, hTSLP protein expression was measured for each of the dosing groups by MSD assay from the lower right lobe of the mouse lung tissues collected, and the data from certain of the samples are shown in FIGS. 10A and 10B. As shown in FIG. 10A, AC003602 achieved ˜87% reduction, AC004573 achieved ˜79% reduction, in human TSLP protein in AAV transduced mouse lungs at 1.5 mg/kg. As shown in FIG. 10B, AC003602 achieved ˜76% reduction, and AC004574 achieved ˜65% reduction, in human TSLP protein in AAV transduced mouse serum at 1.5 mg/kg.

### Example 20. TSLP-SEAP Mouse Model

To evaluate TSLP RNAi agents, a TSLP-SEAP mouse model was used. C57bl6/Albino female mice were transiently transfected in vivo with plasmid by hydrodynamic tail vein (HTV) injection. Mice were injected, via hydrodynamic tail vein (HTV) injection, with plasmid pMIR0962 containing the nucleotides 179-2610 of the TSLP cDNA sequence (GenBank NM_033035.5 (SEQ ID NO:1)) inserted into the 3′ UTR of the SEAP (secreted human placental alkaline phosphatase) reporter gene. 20 μg of the plasmid containing the TSLP cDNA in Ringer's solution in a total volume of 10% of the animal's body weight was injected, via HTV, to create TSLP-SEAP model mice. Following transfection with TSLP-SEAP, the mice were subsequently administered TSLP RNAi agents. Inhibition of TSLP gene expression by TSLP RNAi agent results in concomitant inhibition of SEAP expression. SEAP expression levels were measured by Phospha-Light™ SEAP Reporter Gene Assay System (ThermoFisher Cat #T1016). Prior to treatment, SEAP expression levels in serum were measured and the mice were grouped according to average SEAP levels.

Analyses: SEAP levels may be measured at various times, both before and after administration of TSLP RNAi agents.

i) Serum collection: Mice were anesthetized with 2-3% isoflurane and blood samples were collected from the submandibular area into serum separation tubes (Sarstedt AG & Co., Nümbrecht, Germany). Blood was allowed to coagulate at ambient temperature for 20 min. The tubes were centrifuged at 8,000×g for 3 min to separate the serum and stored at 4° C.

ii) Serum SEAP levels: Serum was collected and measured by the Phospha-Light™ SEAP Reporter Gene Assay System (ThermoFisher) according to the manufacturer's instructions. Serum SEAP levels for each animal was normalized to the control group of mice injected with saline in order to account for the non-treatment related decline in TSLP sequence expression with this model. First, the SEAP level for each animal at a time point was divided by the pre-treatment level of expression in that animal (“pre-treatment”) in order to determine the ratio of expression “normalized to pre-treatment”. Expression at a specific time point was then normalized to the control group by dividing the “normalized to pre-treatment” ratio for an individual animal by the average “normalized to pre-treatment” ratio of all mice in the normal saline control group. Alternatively, in some Examples set forth herein, the serum SEAP levels for each animal were assessed by normalizing to pre-treatment levels only.

To evaluate the activity of TSLP RNAi agents in a SEAP model as described in the Examples below, certain TSLP RNAi agents were conjugated to an N-Acetyl-galactosamine-containing targeting ligand having the chemical structure referred to as NAG37 (see Table 11 for structure information), as shown in Tables 5, 6, and 10. NAG37 is known to have high affinity to bind to asialoglycoprotein receptors that are abundantly expressed on liver cells, including hepatocytes (see International Patent Application Publication No WO2018044350A1). The use of NAG37-conjugated TSLP RNAi agents was to evaluate the expression of SEAP in the liver.

### Example 21. In Vivo Administration of TSLP RNAi Agents in TSLP-SEAP Mice

The TSLP-SEAP model described in Example 20, above, was used. On Day −21, four (n=4) female C57bl/6 albino mice animals were dosed with 20 ug pMIR0962 TSLP-SEAP hydrodynamic tail vein (HTV) injection. On Day 1, the mice test animals were dosed with either isotonic saline or TSLP RNAi agents formulated in saline (at 0.5 mg/kg, 1.0 mg/kg, or 1.5 mg/kg), via subcutaneous (SQ) injection, at 250 μL per 25 g body weight injection volume. The dosing regimen is in accordance with Table 50 below.

TABLE 50
Dosing for mice animals of Example 21.

| | | Targeted | |
|---|---|---|---|
| | | Position of | |
| | | TSLP (Seq | |
| Group | Dose (RNAi Agent) | ID No. 1) | Dosing Route |
| 1 | Saline | N/A | Day 1 SQ Injection |
| 2 | 0.5 mg/kg AC003679 | 571 | Day 1 SQ Injection |
| 3 | 1.0 mg/kg AC003679 | 571 | Day 1 SQ Injection |
| 4 | 1.5 mg/kg AC003679 | 571 | Day 1 SQ Injection |
| 5 | 0.5 mg/kg AC003989 | 520 | Day 1 SQ Injection |
| 6 | 1.0 mg/kg AC003989 | 520 | Day 1 SQ Injection |
| 7 | 1.5 mg/kg AC003989 | 520 | Day 1 SQ Injection |
| 8 | 0.5 mg/kg AC003923 | 571 | Day 1 SQ Injection |
| 9 | 1.0 mg/kg AC003923 | 571 | Day 1 SQ Injection |
| 10 | 0.5 mg/kg AC003920 | 571 | Day 1 SQ Injection |
| 11 | 1.0 mg/kg AC003920 | 571 | Day 1 SQ Injection |
| 12 | 1.5 mg/kg AC003920 | 571 | Day 1 SQ Injection |

For the purposes of evaluating efficacy of the TSLP RNAi agents and the SEAP assay of the instant Example, AC003679 is conjugated to NAG37 (see Table 11 for structure information); NAG37 is known to have high affinity to bind to asialoglycoprotein receptors that are abundantly expressed on liver cells, including hepatocytes. AC003679 was chemically modified as follows:

Modified Sense Strand (5′→3′):
(SEQ ID NO: 775)
(NAG37)s(invAb)sagucacaaCfCfAfauaaaugucus(invAb)
Modified Antisense Strand (5′→3′):
(SEQ ID NO: 652)
asGfsacauuuaUfuGfgUfuGfugacsu

For the purposes of evaluating efficacy of the TSLP RNAi agents and the SEAP assay of the instant Example, AC003989 is conjugated to NAG37 (see Table 11 for structure information); NAG37 is known to have high affinity to bind to asialoglycoprotein receptors that are abundantly expressed on liver cells, including hepatocytes. AC003989 was chemically modified as follows:

Modified Sense Strand (5′→3′):
(SEQ ID NO: 774)
(NAG37)s(invAb)sggaaacucAfGfAfuaaaugcuaas(invAb)
Modified Antisense Strand (5′→3′):
(SEQ ID NO: 628)
cPrpusUfsagCfauuUfauCfuGfaguuucsc

For the purposes of evaluating efficacy of the TSLP RNAi agents and the SEAP assay of the instant Example, AC003920 is conjugated to NAG37 (see Table 11 for structure information); NAG37 is known to have high affinity to bind to asialoglycoprotein receptors that are abundantly expressed on liver cells, including hepatocytes. AC003920 was chemically modified as follows:

Modified Sense Strand (5′→3′):
(SEQ ID NO: 760)
(NAG37)s(invAb)sggucacaaCfCfAfauaaaugucus(invAb)
Modified Antisense Strand (5′→3′):
(SEQ ID NO: 653)
asGfsacauuuaUfuGfgUfuGfugacsc

Serum was collected on Day −7, 1, 8, 15, and 22. SEAP expression levels were determined pursuant to the procedure set forth in Example 20, above. Data from the experiment are shown in the following Table 51, with average SEAP reflecting the normalized average value of SEAP.

TABLE 51
Average SEAP normalized to pre-treatment and saline
control in TSLP-SEAP mice of Example 21.

| | Day 8 | | Day 15 | | Day 22 | |
|---|---|---|---|---|---|---|
| | Avg | Std | Avg | Std | Avg | Std |
| Group ID | SEAP | Dev | SEAP | Dev | SEAP | Dev |
| 1. Saline | 1.000 | 0.377 | 1.000 | 0.520 | 1.000 | 0.681 |
| 2. 0.5 mg/kg AC003679 | 0.379 | 0.039 | 0.259 | 0.125 | 0.215 | 0.048 |
| 3. 1.0 mg/kg AC003679 | 0.277 | 0.125 | 0.307 | 0.383 | 0.167 | 0.150 |
| 4. 1.5 mg/kg AC003679 | 0.146 | 0.026 | 0.125 | 0.107 | 0.065 | 0.024 |
| 5. 0.5 mg/kg AC003989 | 0.360 | 0.093 | 0.291 | 0.152 | 0.347 | 0.282 |
| 6. 1.0 mg/kg AC003989 | 0.251 | 0.112 | 0.119 | 0.051 | 0.121 | 0.020 |
| 7. 1.5 mg/kg AC003989 | 0.127 | 0.026 | 0.123 | 0.078 | 0.102 | 0.049 |
| 8. 0.5 mg/kg AC003923 | 0.224 | 0.021 | 0.243 | 0.130 | 0.162 | 0.070 |
| 9. 1.0 mg/kg AC003923 | 0.179 | 0.020 | 0.127 | 0.028 | 0.138 | 0.027 |
| 10. 0.5 mg/kg AC003920 | 0.257 | 0.069 | 0.127 | 0.064 | 0.086 | 0.055 |
| 11. 1.0 mg/kg AC003920 | 0.196 | 0.011 | 0.130 | 0.025 | 0.100 | 0.029 |
| 12. 1.5 mg/kg AC003920 | 0.130 | 0.005 | 0.041 | 0.014 | 0.025 | 0.025 |

Groups 2-12 showed reduction in SEAP-TSLP at all time points (Day 8, 15, and 22) compared to the saline control Group 1. More specifically, AC003920 achieved ˜97% inhibition at 1.5 mg/kg on Day 22 in this model.

### Example 22. In Vivo Anti-Inflammatory Effect of TSLP Knock-Down in Rat Model of Airway Inflammation, Delivery Via Intra-Tracheal Microsprayer

On study day 1 and day 3, male Brown-Norway rats were administered a dose of 5 mg/kg of a rat-specific RNAi agent linked to a Tri-SM6.1-αvβ6 integrin targeting ligand (referred to as AC001714 or AC002515), or saline vehicle. Additionally, a “RISC-blocked” RNAi trigger was used, which include a construct similar to AC001714, including the same targeting ligand, but included chemical modifications designed to prevent the loading of the antisense strand into RISC, thus serving as a negative control. Volume of 200 μL was loaded into a syringe that was connected to a microsprayer device (Penn Century, Philadelphia, PA) for intra-tracheal administration.

AC001714 and AC002515 include a rat-specific sequence designed to target the rat TSLP transcript (NCBI GenBank XM_008772052.2) and does not have homology with the human TSLP gene, the chemical structure of which is shown above in Example 2 and 3.

On day 15, rats were challenged with a single intra-tracheal dose of 500 μg/rat of Alternaria alternata prepared in PBS. Rats in Group 1 were administered only with PBS as a control.

TABLE 52
Rat-specific TSLP RNAi Agent and Dosing for Example 22.

| | AC | Animals | Harvest/ |
|---|---|---|---|
| | Duplex | per | Sacrifice |
| Group ID | Number | Group | Day |
| Group 1 (saline IT days 1, 3) (PBS IT day 15) | N/A | 5 | Day 16 |
| Group 2 (saline IT days 1, 3) (Alternaria | N/A | 5 | Day 16 |
| IT day 15) | | | |
| Group 3 (saline IT days 1, 3) (IT dose 5.0 | RISC-blocked | 5 | Day 16 |
| mg/kg RISC-blocked trigger on days 1, 3)/ | RNAi Trigger | | |
| (Alternaria IT day 15) | | | |
| Group 4 (IT dose 5.0 mg/kg AC001714 on days | AC001714 | 5 | Day 16 |
| 1, 3)/(Alternaria IT day 15) | | | |
| Group 5 (IT dose 5.0 mg/kg AC002515 on days | AC002515 | 5 | Day 16 |
| 1, 3)/(Alternaria IT day 15) | | | |

After 24 hours post-administration of the Alternaria (i.e., day 16), rats were anesthetized with isoflurane/02, blood was drawn, and were euthanized by exsanguination. Days of sacrifice/euthanasia are shown in Table 14 above. Trachea was canulated and bronchoalveolar lavage (BAL) collected after washing with 2×5 mL of ice-cold PBS. BAL samples were spun down, cells resuspended with 1 mL of ice-cold PBS, and aliquot was mixed with Turk's solution (ratio 1:1), and total cell counted via hemocytomers. Cytospins were prepared, stained and differential cell counting performed. Supernatant was used for cytokine measurements. Right lung lobes were used to determine rTSLP mRNA expression and left lung lobes were collected in 4% PFA/PBS for histology (Trichrome and Sirius Red Staining, RNAscope).

The rat lungs were inflated, fixed in 4% PFA and processed for mRNA in situ hybridization and immunohistochemistry. TSLP RNAscope shows TSLP is expressed in airway and alveolar. Z-stack confocal scan images show TSLP transcript retained in the nucleus, showing that silencing of cytoplasmic TSLP mRNA does not reduce pre-mRNA retained in the nucleus.

### Example 23. Passive Uptake of TSLP RNAI Agents in Human Precision Cut Lung Slices (PCLS)

Precision cut tissue slices (PCLS) represent an ex vivo model and tool for studying the structure and function of the lung in its native 3D environment, allowing for examination of the natural interactions between cells, molecules, and the extracellular matrix (ECM) ex vivo (Alsafadi H. N. et al, Am J Respir Cell Mol Biol 62(6): 681-691 (2020)). PCLS can be generated from various anatomical locations of the lung (distal and proximal), and from different species, including rodents, pigs, monkeys, and humans. To validate the RNAi agent potency for silencing human TSLP mRNA, fresh agarose inflated lung slices from one (1) healthy human donor were used for examination.

Saline or TSLP RNAi agents were added to cell media, with daily media changes. The PCLS were cultured in the media from Day 1 to Day 7 and harvested at Day 8. PCLS were cultured and dosed with TSLP RNAi agents in accordance with the following Table 53.

RNAi agent AC003609, a “RISC-blocked” RNAi agent, includes chemical modifications designed to prevent the loading of the antisense strand into RISC, thus serving as a negative control.

TABLE 53
Dosing groups and dosing regimen of
TSLP RNAi agents from Example 23.

| | Dosing | # PCLS Samples |
|---|---|---|
| Group ID | Regimen | (n=) per Group |
| Group 1. Saline | Treatment daily in the | n = 6 |
| | cell media for 7 days | |
| Group 2. AC003374 10 μM | Treatment daily in the | n = 6 |
| | cell media for 7 days | |
| Group 3. AC003374 1 μM | Treatment daily in the | n = 6 |
| | cell media for 7 days | |
| Group 4. AC003374 0.1 μM | Treatment daily in the | n = 6 |
| | cell media for 7 days | |
| Group 5. AC003602 10 μM | Treatment daily in the | n = 6 |
| | cell media for 7 days | |
| Group 6. AC003602 1 μM | Treatment daily in the | n = 6 |
| | cell media for 7 days | |
| Group 7. AC003602 0.1 μM | Treatment daily in the | n = 6 |
| | cell media for 7 days | |
| Group 8. AC003546 10 μM | Treatment daily in the | n = 6 |
| | cell media for 7 days | |
| Group 9. AC003546 1 μM | Treatment daily in the | n = 6 |
| | cell media for 7 days | |
| Group 10. AC003546 0.1 μM | Treatment daily in the | n = 6 |
| | cell media for 7 days | |
| Group 11. AC003609 10 μM | Treatment daily in the | n = 6 |
| | cell media for 7 days | |

TSLP miRNA expression was quantitated by qPCR, using PPIA as endogenous control gene, and normalized to Group 1 samples dosed with saline. qPCR relative expression data are shown in the following Table 54.

TABLE 54
Relative TSLP expression in PCLS, normalized
to vehicle control, of Example 23.

| | Day 8 | | |
|---|---|---|---|
| | Rel. | Error | Error |
| Group ID | Exp. | Low | High |
| 1. Saline | 1.000 | 0.189 | 0.233 |
| 2. AC003374 10 μM | 0.256 | 0.087 | 0.132 |
| 3. AC003374 1 μM | 0.320 | 0.095 | 0.135 |
| 4. AC003374 0.1 μM | 0.453 | 0.116 | 0.156 |
| 5. AC003602 10 μM | 0.325 | 0.067 | 0.085 |
| 6. AC003602 1 μM | 0.405 | 0.100 | 0.132 |
| 7. AC003602 0.1 μM | 0.524 | 0.104 | 0.130 |
| 8. AC003546 10 μM | 0.198 | 0.070 | 0.107 |
| 9. AC003546 1 μM | 0.279 | 0.047 | 0.057 |
| 10. AC003546 0.1 μM | 0.288 | 0.060 | 0.076 |
| 11. AC003609 10 μM | 0.830 | 0.155 | 0.190 |

Effective passive uptake of TSLP RNAi agents was observed. PCLS cultures treated with TSLP RNAi agents showed significant silencing of hTSLP mRNA. Groups 2-10 showed inhibition of TSLP at Day 8. More specifically, AC003546 at 10 μM achieved ˜80% inhibition (0.198) on Day 8. Furthermore, dose response was observed for AC003374, AC003602, and AC003546.

### Example 24. Passive Uptake of TSLP RNAI Agents in Human Precision Cut Lung Slices (PCLS)

Precision cut tissue slices (PCLS) represent an ex vivo model and tool for studying the structure and function of the lung in its native 3D environment, allowing for examination of the natural interactions between cells, molecules, and the extracellular matrix (ECM) ex vivo (Alsafadi H. N. et al, Am J Respir Cell Mol Biol 62(6): 681-691 (2020)). PCLS can be generated from various anatomical locations of the lung (distal and proximal), and from different species, including rodents, pigs, monkeys, and humans. To validate the RNAi agent potency for silencing human TSLP mRNA, fresh agarose inflated lung slices from asthmatic patient donor were used for examination.

Saline or TSLP RNAi agents were added to cell media, with daily media changes. The PCLS were cultured in the media from Day 1 to Day 7 and harvested at Day 8. PCLS were cultured and dosed with TSLP RNAi agents in accordance with the following Table 55.

TABLE 55
Dosing groups and dosing regimen of
TSLP RNAi agents from Example 24.

| | Dosing | # PCLS Samples |
|---|---|---|
| Group ID | Regimen | (n=) per Group |
| Group 1. Saline | Treatment daily in the | n = 6 |
| | cell media for 7 days | |
| Group 2. AC003253 10 μM | Treatment daily in the | n = 6 |
| | cell media for 7 days | |
| Group 3. AC003253 1 μM | Treatment daily in the | n = 6 |
| | cell media for 7 days | |
| Group 4. AC003253 0.1 μM | Treatment daily in the | n = 6 |
| | cell media for 7 days | |
| Group 5. AC003374 10 μM | Treatment daily in the | n = 6 |
| | cell media for 7 days | |
| Group 6. AC003374 1 μM | Treatment daily in the | n = 6 |
| | cell media for 7 days | |
| Group 7. AC003374 0.1 μM | Treatment daily in the | n = 6 |
| | cell media for 7 days | |
| Group 8. AC003546 10 μM | Treatment daily in the | n = 6 |
| | cell media for 7 days | |
| Group 9. AC003546 1 μM | Treatment daily in the | n = 6 |
| | cell media for 7 days | |
| Group 10. AC003546 0.1 μM | Treatment daily in the | n = 6 |
| | cell media for 7 days | |
| Group 11. AC001651 10 μM | Treatment daily in the | n = 6 |
| | cell media for 7 days | |

AC001651 is an RNAi agent designed to initiate RISC and inhibit gene expression of a different gene, and not targeted to the hTSLP gene.

TSLP mRNA expression was quantitated by qPCR, using B2M as endogenous control gene, and normalized to Group 1 samples dosed with saline. qPCR relative expression data are shown in the following Table 56.

TABLE 56
Relative TSLP expression in PCLS, normalized
to vehicle control, of Example 24.

| | Day 8 | | |
|---|---|---|---|
| | Rel. | Error | Error |
| Group ID | Exp. | Low | High |
| 1. Saline | 1.000 | 0.190 | 0.234 |
| 2. AC003253 10 μM | 0.555 | 0.182 | 0.270 |
| 3. AC003253 1 μM | 0.675 | 0.339 | 0.681 |
| 4. AC003253 0.1 μM | 0.790 | 0.143 | 0.175 |
| 5. AC003374 10 μM | 0.362 | 0.082 | 0.107 |
| 6. AC003374 1 μM | 0.531 | 0.157 | 0.223 |
| 7. AC003374 0.1 μM | 0.918 | 0.352 | 0.570 |
| 8. AC003546 10 μM | 0.489 | 0.174 | 0.270 |
| 9. AC003546 1 μM | 0.403 | 0.097 | 0.127 |
| 10. AC003546 0.1 μM | 0.652 | 0.228 | 0.350 |
| 11. AC001651 10 μM | 0.913 | 0.211 | 0.274 |

Effective passive uptake of TSLP RNAi agents was observed. PCLS cultures treated with TSLP RNAi agents showed silencing of hTSLP mRNA. Groups 2-6 and 8-10 showed inhibition of TSLP at Day 8. Groups 7 and 11 showed negligible inhibition. More specifically, AC003374 at 10 μM achieved ˜63% inhibition (0.362) on Day 8. Furthermore, dose response was observed for AC003253 and AC003374.

### Example 25. TSLP RNAi Agents in AAV9-CAG-hTSLP AAV Mouse Model

To evaluate certain TSLP RNAi agents, the same AAV9-CAG-hTSLP (Adeno-associated virus) mouse model as discussed in Example 4 was used.

The human TSLP mRNA expression was measured in the mice lung tissues by qPCR

At Day −17 and Day −14, each mouse (female C57Bl/6) was given an intratracheal (IT) administration of 50 μL AAV solutions containing 2e10 GC (genome copy) of AAV9-CAG-eGFP and 2e10 GC of AAV9-CAG-hTSLP in PBS. At Day 1 and Day 3, each mouse was given intratracheal administration of 50 μL (at 0.75, 1.5, or 3.0 mg/kg) of TSLP RNAi agents formulated in isotonic saline, or vehicle control (isotonic saline with no RNAi agent), according to the following Table 57. The mice were humanely sacrificed and harvested on Day 15.

TABLE 57
Dosing Groups of Example 25.

| | | RNAi Agent | | # |
|---|---|---|---|---|
| | AAV dose | and Dose | | Animals |
| Group | (Day −17, −14) | (Day 1, 3) | Dosing Regimen | (n=) |
| 1 | 2e10 GC of | Saline | IT doses of AAV | n = 5 |
| | AAV9-CAG- | | on Day −17, −14; | |
| | eGFP and 2e10 | | IT doses of saline | |
| | GC of AAV9- | | on Days 1, 3 | |
| | CAG-hTSLP | | | |
| 2 | 2e10 GC of | 0.75 mg/kg | IT doses of AAV | n = 5 |
| | AAV9-CAG- | AC003374 | on Day −17, −14; | |
| | eGFP and 2e10 | | IT doses of RNAi | |
| | GC of AAV9- | | agent on Days 1, 3 | |
| | CAG-hTSLP | | | |
| 3 | 2e10 GC of | 1.5 mg/kg | IT doses of AAV | n = 5 |
| | AAV9-CAG- | AC003374 | on Day −17, −14; | |
| | eGFP and 2e10 | | IT doses of RNAi | |
| | GC of AAV9- | | agent on Days 1, 3 | |
| | CAG-hTSLP | | | |
| 4 | 2e10 GC of | 3.0 mg/kg | IT doses of AAV | n = 5 |
| | AAV9-CAG- | AC003374 | on Day −17, −14; | |
| | eGFP and 2e10 | | IT doses of RNAi | |
| | GC of AAV9- | | agent on Days 1, 3 | |
| | CAG-hTSLP | | | |
| 5 | 2e10 GC of | 0.75 mg/kg | IT doses of AAV | n = 5 |
| | AAV9-CAG- | AC004361 | on Day −17, −14; | |
| | eGFP and 2e10 | | IT doses of RNAi | |
| | GC of AAV9- | | agent on Days 1, 3 | |
| | CAG-hTSLP | | | |
| 6 | 2e10 GC of | 1.5 mg/kg | IT doses of AAV | n = 5 |
| | AAV9-CAG- | AC004361 | on Day −17, −14; | |
| | eGFP and 2e10 | | IT doses of RNAi | |
| | GC of AAV9- | | agent on Days 1, 3 | |
| | CAG-hTSLP | | | |
| 7 | 2e10 GC of | 3.0 mg/kg | IT doses of AAV | n = 5 |
| | AAV9-CAG- | AC004361 | on Day −17, −14; | |
| | eGFP and 2e10 | | IT doses of RNAi | |
| | GC of AAV9- | | agent on Days 1, 3 | |
| | CAG-hTSLP | | | |
| 8 | 2e10 GC of | 1.5 mg/kg | IT doses of AAV | n = 5 |
| | AAV9-CAG- | AC005945 | on Day −17, −14; | |
| | eGFP and 2e10 | | IT doses of RNAi | |
| | GC of AAV9- | | agent on Days 1, 3 | |
| | CAG-hTSLP | | | |

Each of the TSLP RNAi agents included modified nucleotides that were conjugated at the 5′ terminal end of the sense strand to an αvβ6 integrin targeting ligand having the modified sequences as set forth in the duplex structures herein. (See Tables 3, 4, 5, 6, 7A, 7B, 8, 9, 10, and 11 for specific modifications and structure information related to the TSLP RNAi agents, including Tri-SM6.1-αvβ6).

Five (5) mice were tested (n=5) for each group. Serum samples and lower right lobes were collected for human TSLP protein measurement by Meso Scale Discovery (MSD) Assay. Left lobe and all remaining right lobes were collected for TSLP mRNA expression measurement by qPCR, using eGFP as endogenous control gene, and normalized to Group 1. Data from the experiment are shown in the following Table 58:

TABLE 58
Average Relative TSLP Normalized to Control in AAV-hTSLP Mice from Example 25.

| | Average Relative hTSLP | Low | High |
|---|---|---|---|
| Group ID | mRNA Expression | (error) | (error) |
| Group 1. (AAV IT Days −17, −14) (Saline IT Days 1, 3) | 1.000 | 0.130 | 0.150 |
| Group 2. (AAV IT Days −17, −14) (0.75 mg/kg AC003374 IT Days 1, 3) | 0.351 | 0.042 | 0.047 |
| Group 3. (AAV IT Days −17, −14) (1.5 mg/kg AC003374 IT Days 1, 3) | 0.385 | 0.038 | 0.042 |
| Group 4. (AAV IT Days −17, −14) (3.0 mg/kg AC003374 IT Days 1, 3) | 0.340 | 0.068 | 0.086 |
| Group 5. (AAV IT Days −17, −14) (0.75 mg/kg AC004361 IT Days 1, 3) | 0.412 | 0.084 | 0.106 |
| Group 6. (AAV IT Days −17, −14) (1.5 mg/kg AC004361 IT Days 1, 3) | 0.397 | 0.072 | 0.088 |
| Group 7. (AAV IT Days −17, −14) (3.0 mg/kg AC004361 IT Days 1, 3) | 0.300 | 0.048 | 0.058 |
| Group 8. (AAV IT Days −17, −14) (1.5 mg/kg AC005945 IT Days 1, 3) | 0.397 | 0.051 | 0.058 |

As shown in Table 58, above, each of the TSLP RNAi agents tested (Groups 2-8) showed reductions in hTSLP expression compared to control (Group 1). Dose response was also observed for AC004361.

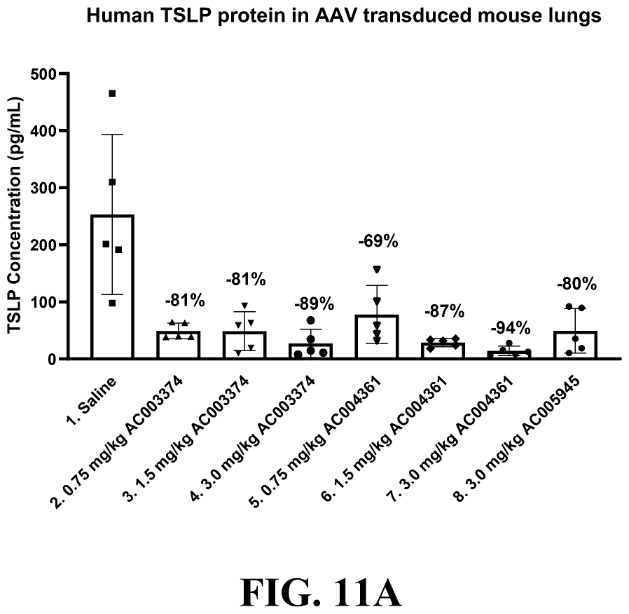

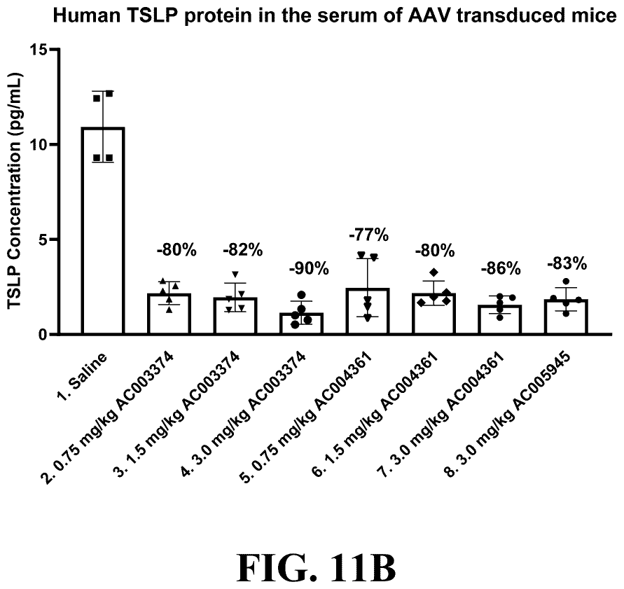

Further, hTSLP protein expression was measured for each of the dosing groups by MSD assay from the lower right lobe of the mouse lung tissues collected, and the data from certain of the samples are shown in FIGS. 11A and 11B. As shown in FIG. 11A, AC003374 achieved ˜89% reduction at 2×3.0 mg/kg dose, and AC004361 achieved ˜94% reduction at 2×3.0 mg/kg dose, of human TSLP protein in AAV transduced mouse lungs. Dose response was observed in AC004361 in mouse lungs. As shown in FIG. 11B, AC003374 achieved ˜90% reduction at 2×3.0 mg/kg dose, and AC004361 achieved ˜86% reduction at 2×3.0 mg/kg dose, of human TSLP protein in AAV transduced mouse serum. Dose response was observed in both AC003374 and AC004361 in mouse serum.

### Example 26. TSLP RNAi Agents in AAV9-CAG-hTSLP AAV Mouse Model

To evaluate certain TSLP RNAi agents, the same AAV9-CAG-hTSLP (Adeno-associated virus) mouse model as discussed in Example 4 was used.

The human TSLP mRNA expression was measured in the mice lung tissues by qPCR.

At Day −17 and Day −14, each mouse (female C57Bl/6) was given an intratracheal (IT) administration of 50 μL AAV solutions containing 2e10 GC (genome copy) of AAV9-CAG-eGFP and 2e10 GC of AAV9-CAG-hTSLP in PBS. At Day 1 and Day 3, each mouse was given intratracheal administration of 50 μL (at 0.75, 1.5, or 3.0 mg/kg) of TSLP RNAi agents formulated in isotonic saline, or vehicle control (isotonic saline with no RNAi agent), according to the following Table 59. The mice were humanely sacrificed and harvested on Day 15.

RNAi agent AC005329, a “RISC-blocked” RNAi agent, includes chemical modifications designed to prevent the loading of the antisense strand into RISC, thus serving as a negative control.

TABLE 59
Dosing Groups of Example 26.

| | | RNAi Agent | | # |
|---|---|---|---|---|
| | AAV dose | and Dose | | Animals |
| Group | (Day −17, −14) | (Day 1, 3) | Dosing Regimen | (n=) |
| 1 | 2e10 GC of AAV9-CAG-eGFP and | Saline | IT doses of AAV on Day −17, −14; | n = 5 |
| | 2e10 GC of AAV9-CAG-hTSLP | | IT doses of saline on Days 1, 3 | |
| 2 | 2e10 GC of AAV9-CAG-eGFP and | 0.75 mg/kg AC003374 | IT doses of AAV on Day −17, −14; | n = 5 |
| | 2e10 GC of AAV9-CAG-hTSLP | | IT doses of RNAi agent on Days 1, 3 | |
| 3 | 2e10 GC of AAV9-CAG-eGFP and | 1.5 mg/kg AC003374 | IT doses of AAV on Day −17, −14; | n = 5 |
| | 2e10 GC of AAV9-CAG-hTSLP | | IT doses of RNAi agent on Days 1, 3 | |
| 4 | 2e10 GC of AAV9-CAG-eGFP and | 3.0 mg/kg AC003374 | IT doses of AAV on Day −17, −14; | n = 5 |
| | 2e10 GC of AAV9-CAG-hTSLP | | IT doses of RNAi agent on Days 1, 3 | |
| 5 | 2e10 GC of AAV9-CAG-eGFP and | 0.75 mg/kg AC004361 | IT doses of AAV on Day −17, −14; | n = 5 |
| | 2e10 GC of AAV9-CAG-hTSLP | | IT doses of RNAi agent on Days 1, 3 | |
| 6 | 2e10 GC of AAV9-CAG-eGFP and | 1.5 mg/kg AC004361 | IT doses of AAV on Day −17, −14; | n = 5 |
| | 2e10 GC of AAV9-CAG-hTSLP | | IT doses of RNAi agent on Days 1, 3 | |
| 7 | 2e10 GC of AAV9-CAG-eGFP and | 3.0 mg/kg AC004361 | IT doses of AAV on Day −17, −14; | n = 5 |
| | 2e10 GC of AAV9-CAG-hTSLP | | IT doses of RNAi agent on Days 1, 3 | |
| 8 | 2e10 GC of AAV9-CAG-eGFP and | 3.0 mg/kg AC005329 | IT doses of AAV on Day −17, −14; | n = 5 |
| | 2e10 GC of AAV9-CAG-hTSLP | | IT doses of RNAi agent on Days 1, 3 | |

Each of the TSLP RNAi agents included modified nucleotides that were conjugated at the 5′ terminal end of the sense strand to an αvβ6 integrin targeting ligand having the modified sequences as set forth in the duplex structures herein. (See Tables 3, 4, 5, 6, 7A, 7B, 8, 9, 10, and 11 for specific modifications and structure information related to the TSLP RNAi agents, including Tri-SM6.1-αvβ6).

Five (5) mice were tested (n=5) for each group. Serum samples and lower right lobes were collected for human TSLP protein measurement by Meso Scale Discovery (MSD) Assay. Left lobe and all remaining right lobes were collected for TSLP mRNA expression measurement by qPCR, using eGFP as endogenous control gene, and normalized to Group 1. Data from the experiment are shown in the following Table 60.

TABLE 60
Average Relative TSLP Normalized to Control in AAV-hTSLP Mice from Example 26.

| | Average Relative hTSLP | Low | High |
|---|---|---|---|
| Group ID | mRNA Expression | (error) | (error) |
| Group 1. (AAV IT Days −17, −14) (Saline IT Days 1, 3) | 1.000 | 0.224 | 0.288 |
| Group 2. (AAV IT Days −17, −14) (0.75 mg/kg AC003374 IT Days 1, 3) | 0.602 | 0.168 | 0.233 |
| Group 3. (AAV IT Days −17, −14) (1.5 mg/kg AC003374 IT Days 1, 3) | 0.521 | 0.092 | 0.112 |
| Group 4. (AAV IT Days −17, −14) (3.0 mg/kg AC003374 IT Days 1, 3) | 0.340 | 0.073 | 0.094 |
| Group 5. (AAV IT Days −17, −14) (0.75 mg/kg AC004361 IT Days 1, 3) | 0.440 | 0.046 | 0.051 |
| Group 6. (AAV IT Days −17, −14) (1.5 mg/kg AC004361 IT Days 1, 3) | 0.350 | 0.055 | 0.065 |
| Group 7. (AAV IT Days −17, −14) (3.0 mg/kg AC004361 IT Days 1, 3) | 0.366 | 0.117 | 0.171 |
| Group 8. (AAV IT Days −17, −14) (3.0 mg/kg AC005329 IT Days 1, 3) | 0.877 | 0.178 | 0.223 |

As shown in Table 60, above, RNAi agents of Groups 2-7 tested showed reductions in hTSLP expression compared to control (Group 1). Dose response was also observed for AC003374.

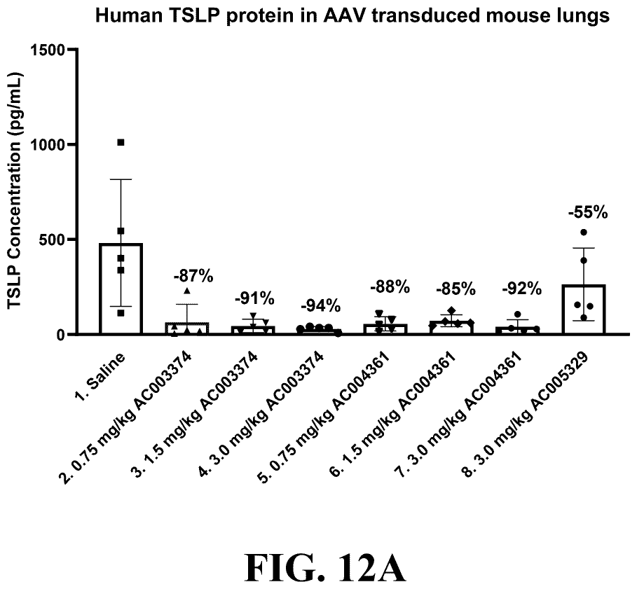

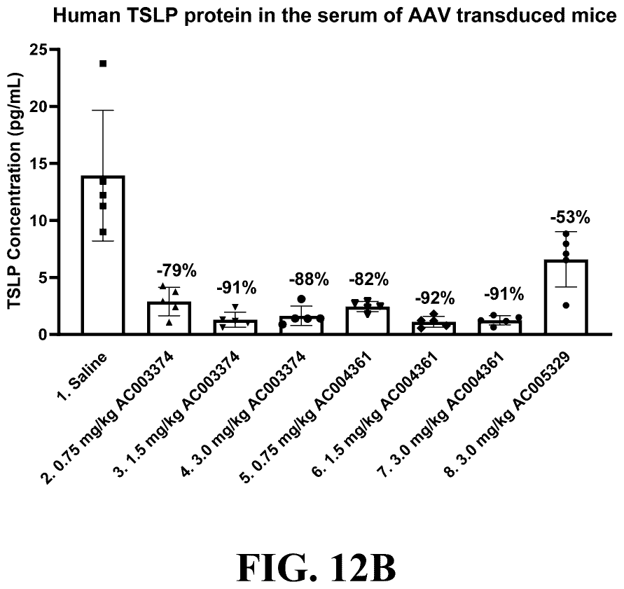

Further, hTSLP protein expression was measured for each of the dosing groups by MSD assay from the lower right lobe of the mouse lung tissues collected, and the data from certain of the samples are shown in FIGS. 12A and 12B. As shown in FIG. 12A, AC003374 achieved ˜94% reduction at 2×3.0 mg/kg dose, and AC004361 achieved ˜92% reduction at 2×3.0 mg/kg dose, of human TSLP protein in AAV transduced mouse lungs. Dose response was also observed in AC003374 in mouse lungs. As shown in FIG. 12B, AC003374 achieved ˜91% reduction at 2×1.5 mg/kg dose, and AC004361 achieved ˜92% reduction at 2×1.5 mg/kg dose, of human TSLP protein in AAV transduced mouse serum.

### Example 27. In Vivo Administration of TSLP RNAi Agents in B-hTSLP/hTSLPR Humanized TSLP Knock-In Mice

To evaluate certain TSLP RNAi agents, a B-hTSLP/hTSLPR mouse model was used. C57BL/6-Tslp^{tm1(TSLP)}Crlf2^{tm2(CRLF2)}/Bcgen (strain name), also referred to as B-hTSLP/hTSLPR mice (common name), were purchased and received from Biocytogen (Catalog #121269). The background mouse was of C57BL/6 strain. The exons 1-5 of the mouse TSLP gene that encode full-length protein were replaced by human TSLP exons 1-4, containing nucleobases 179-658 of human TSLP, in the B-hTSLP/hTSLPR mice. The extracellular and transmembrane region of human thymic stromal lymphopoietin receptor (TSLPR) gene and cytoplasmic region of mouse TSLPR gene were constructed into a chimeric CDS vector and inserted into the mouse exon 2. The mice express chimeric TSLP and TSLPR proteins, while the mouse TSLP or TSLPR will no longer express.

On Day 1 and Day 3, five (n=5) male B-hTSLP/hTSLPR mice were dosed, via intratracheal (IT) administration, with either saline (as vehicle control) or TSLP RNAi agents formulated in isotonic saline (at 5.0 mg/kg), at 50 μL dose volume. Dosing was in accordance with the following Table 61.

RNAi agent AC005329, a “RISC-blocked” RNAi agent, includes chemical modifications designed to prevent the loading of the antisense strand into RISC, thus serving as a negative control.

TABLE 61
Dosing Groups of Example 27.

| | | | Sacrifice | # Animals |
|---|---|---|---|---|
| Group | RNAi Agent | Dosing Regimen | Day | (n =) |
| 1 | Saline | Day 1 and 3: IT Administration | Day 15 | n = 5 |
| 2 | AC003374 5.0 mg/kg | Day 1 and 3: IT Administration | Day 15 | n = 5 |
| 3 | AC004361 5.0 mg/kg | Day 1 and 3: IT Administration | Day 15 | n = 5 |
| 4 | AC005329 5.0 mg/kg | Day 1 and 3: IT Administration | Day 15 | n = 5 |
| 5 | Saline | Day 1 and 3: IT Administration | Day 29 | n = 5 |
| 6 | AC003374 5.0 mg/kg | Day 1 and 3: IT Administration | Day 29 | n = 5 |
| 7 | AC004361 5.0 mg/kg | Day 1 and 3: IT Administration | Day 29 | n = 5 |
| 8 | Saline | Day 1 and 3: IT Administration | Day 43 | n = 5 |
| 9 | AC003374 5.0 mg/kg | Day 1 and 3: IT Administration | Day 43 | n = 5 |
| 10 | AC004361 5.0 mg/kg | Day 1 and 3: IT Administration | Day 43 | n = 4 |

Five (5) mice were tested (n=5) for each group. Mice test animals were humanely sacrificed and harvested on Days 15, 29, or 43. Lungs were collected for TSLP mRNA expression measurement by qPCR, using mGAPDH as endogenous control gene. Groups 2-4 were normalized to Group 1, Groups 6-7 were normalized to Group 5, and Groups 9-10 were normalized to Group 8. Data from the experiment are shown in the following Table 62:

TABLE 62
Relative TSLP expression in mice test animals of Example 27.

hTSLP

| | Rel. | Error | Error |
|---|---|---|---|
| Group ID | Exp. | Low | High |
| 1. Saline, D 15 Sac | 1.000 | 0.057 | 0.060 |
| 2. AC003374 5.0 mg/kg, D 15 Sac | 0.503 | 0.056 | 0.063 |
| 3. AC004361 5.0 mg/kg, D 15 Sac | 0.529 | 0.056 | 0.063 |
| 4. AC005329 5.0 mg/kg, D 15 Sac | 0.972 | 0.074 | 0.080 |
| 5. Saline, D 29 Sac | 1.000 | 0.087 | 0.095 |
| 6. AC003374 5.0 mg/kg, D 29 Sac | 0.635 | 0.078 | 0.089 |
| 7. AC004361 5.0 mg/kg, D 29 Sac | 0.554 | 0.073 | 0.083 |
| 8. Saline, D 43 Sac | 1.000 | 0.113 | 0.128 |
| 9. AC003374 5.0 mg/kg, D 43 Sac | 0.475 | 0.095 | 0.119 |
| 10. AC004361 5.0 mg/kg, D 43 Sac | 0.637 | 0.097 | 0.114 |

TSLP RNAi agents silenced expression of human TSLP mRNA in lungs of knock-in mice for over 6 weeks. The RISC-blocked RNAi agent AC005329 (Group 4) showed inability to silence hTSLP expression. Groups 2, 3, 6, 7, 9, and 10 showed reduction in hTSLP in the mice test animals. More specifically, AC003374 showed hTSLP inhibition, ˜52% inhibition (0.475) at 5.0 mg/kg on Day 43. Groups 9 and 10 showed hTSLP inhibition out to at least Day 43.

### Example 28. In Vivo Administration of TSLP RNAi Agents in B-hTSLP/hTSLPR Humanized TSLP Knock-In Mice

To evaluate certain TSLP RNAi agents, a B-hTSLP/hTSLPR mouse model was used. C57BL/6-Tslp^{tm1/(TSLP)}Crlf2^{tm2(CRLF2)}/Bcgen (strain name), also referred to as B-hTSLP/hTSLPR mice (common name), were purchased and received from Biocytogen (Catalog #121269). The background mouse was of C57BL/6 strain. The exons 1-5 of the mouse TSLP gene that encode full-length protein were replaced by human TSLP exons 1-4, containing nucleobases 179-658 of human TSLP, in the B-hTSLP/hTSLPR mice. The extracellular and transmembrane region of human thymic stromal lymphopoietin receptor (TSLPR) gene and cytoplasmic region of mouse TSLPR gene were constructed into a chimeric CDS vector and inserted into the mouse exon 2. The mice express chimeric TSLP and TSLPR proteins, while the mouse TSLP and TSLPR will no longer express.

On Day 1 and Day 3, five (n=5) male B-hTSLP/hTSLPR mice were dosed, via intratracheal (IT) administration, with either saline (as vehicle control) or TSLP RNAi agents formulated in isotonic saline (at 1.0, 2.5, or 5.0 mg/kg), at 50 μL dose volume. Dosing was in accordance with the following Table 63.

RNAi agents AC005329 and AC006020, “RISC-blocked” RNAi agents, include chemical modifications designed to prevent the loading of the antisense strand into RISC, thus serving as a negative control.

TABLE 63
Dosing Groups of Example 28.

| | | | Sacrifice | # Animals |
|---|---|---|---|---|
| Group | RNAi Agent | Dosing Regimen | Day | (n=) |
| 1 | Saline | Day 1 and 3: IT Administration | Day 15 | n = 5 |
| 2 | AC003374 5.0 mg/kg | Day 1 and 3: IT Administration | Day 15 | n = 5 |
| 3 | AC003374 2.5 mg/kg | Day 1 and 3: IT Administration | Day 15 | n = 5 |
| 4 | AC003374 1.0 mg/kg | Day 1 and 3: IT Administration | Day 15 | n = 5 |
| 5 | AC004361 5.0 mg/kg | Day 1 and 3: IT Administration | Day 15 | n = 5 |
| 6 | AC004361 2.5 mg/kg | Day 1 and 3: IT Administration | Day 15 | n = 5 |
| 7 | AC004361 1.0 mg/kg | Day 1 and 3: IT Administration | Day 15 | n = 5 |
| 8 | AC005329 5.0 mg/kg | Day 1 and 3: IT Administration | Day 15 | n = 5 |
| 9 | AC005329 2.5 mg/kg | Day 1 and 3: IT Administration | Day 15 | n = 5 |
| 10 | AC005329 1.0 mg/kg | Day 1 and 3: IT Administration | Day 15 | n = 5 |
| 11 | AC006020 5.0 mg/kg | Day 1 and 3: IT Administration | Day 15 | n = 4 |

Five (5) mice were tested (n=5) for each group. Mice test animals were humanely sacrificed and harvested on Day 15. Lungs were collected for TSLP mRNA expression measurement by qPCR, using mB2M as endogenous control gene. Groups 2-11 were normalized to Group 1. Data from the experiment are shown in the following Table 64:

TABLE 64
Relative TSLP expression in mice test animals of Example 28.

| | hTSLP | | |
|---|---|---|---|
| | Rel. | Error | Error |
| Group ID | Exp. | Low | High |
| 1. Saline | 1.000 | 0.182 | 0.222 |
| 2. AC003374 5.0 mg/kg | 0.506 | 0.136 | 0.186 |
| 3. AC003374 2.5 mg/kg | 0.662 | 0.132 | 0.165 |
| 4. AC003374 1.0 mg/kg | 0.733 | 0.193 | 0.262 |
| 5. AC004361 5.0 mg/kg | 0.570 | 0.104 | 0.127 |
| 6. AC004361 2.5 mg/kg | 0.723 | 0.120 | 0.144 |
| 7. AC004361 1.0 mg/kg | 0.642 | 0.104 | 0.124 |
| 8. AC005329 5.0 mg/kg | 0.962 | 0.141 | 0.165 |
| 9. AC005329 2.5 mg/kg | 1.116 | 0.138 | 0.157 |
| 10. AC005329 1.0 mg/kg | 1.041 | 0.199 | 0.246 |
| 11. AC006020 5.0 mg/kg | 0.862 | 0.151 | 0.183 |

Groups 2-7 showed reduction in hTSLP in the mice test animals. Groups 8-11 showed negligible hTSLP inhibition. More specifically, AC003374 showed hTSLP inhibition, ˜49% inhibition (0.506) at 5.0 mg/kg on Day 15. Dose response was observed for mice treated with AC003374.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An RNAi agent for inhibiting expression of a thymic stromal lymphopoietin gene, comprising:
an antisense strand comprising at least 17 contiguous nucleotides differing by 0 or 1 nucleotides from any one of the sequences provided in Table 2 or Table 3; and
a sense strand comprising a nucleotide sequence that is at least partially complementary to the antisense strand.

2. The RNAi agent of claim 1, wherein the antisense strand comprises nucleotides 2-18 of any one of the sequences provided in Table 2 or Table 3.

3. The RNAi agent of claim 1 or claim 2, wherein the sense strand comprises a nucleotide sequence of at least 17 contiguous nucleotides differing by 0 or 1 nucleotides from any one of the sequences provided in Table 2 or Table 4, and wherein the sense strand has a region of at least 85% complementarity over the 17 contiguous nucleotides to the antisense strand.

4. The RNAi agent of any one of claims 1-3, wherein at least one nucleotide of the TSLP RNAi agent is a modified nucleotide or includes a modified internucleoside linkage.

5. The RNAi agent of any one of claims 1-4, wherein all or substantially all of the nucleotides are modified nucleotides.

6. The RNAi agent of any one of claims 4-5, wherein the modified nucleotide is selected from the group consisting of: 2′-O-methyl nucleotide, 2′-fluoro nucleotide, 2′-deoxy nucleotide, 2′,3′-seco nucleotide mimic, locked nucleotide, 2′-F-arabino nucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, vinyl phosphonate-containing nucleotide, cyclopropyl phosphonate-containing nucleotide, and 3′-O-methyl nucleotide.

7. The RNAi agent of claim 5, wherein all or substantially all of the nucleotides are modified with 2′-O-methyl nucleotides, 2′-fluoro nucleotides, or combinations thereof.

8. The RNAi agent of any one of claims 1-7, wherein the antisense strand comprises the nucleotide sequence of any one of the modified sequences provided in Table 3.

9. The RNAi agent of any one of claims 1-8, wherein the sense strand comprises the nucleotide sequence of any one of the modified sequences provided in Table 4.

10. The RNAi agent of claim 1, wherein the antisense strand comprises the nucleotide sequence of any one of the modified sequences provided in Table 3 and the sense strand comprises the nucleotide sequence of any one of the modified sequences provided in Table 4.

11. The RNAi agent of any one of claims 1-10, wherein the sense strand is between 18 and 30 nucleotides in length, and the antisense strand is between 18 and 30 nucleotides in length.

12. The RNAi agent of claim 11, wherein the sense strand and the antisense strand are each between 18 and 27 nucleotides in length.

13. The RNAi agent of claim 12, wherein the sense strand and the antisense strand are each between 18 and 24 nucleotides in length.

14. The RNAi agent of claim 13, wherein the sense strand and the antisense strand are each 21 nucleotides in length.

15. The RNAi agent of claim 14, wherein the RNAi agent has two blunt ends.

16. The RNAi agent of any one of claims 1-15, wherein the sense strand comprises one or two terminal caps.

17. The RNAi agent of any one of claims 1-16, wherein the sense strand comprises one or two inverted abasic residues.

18. The RNAi agent of claim 1, wherein the RNAi agent is comprised of a sense strand and an antisense strand that form a duplex having the structure of any one of the duplexes in Table 7A, Table 7B, Table 8, Table 9, or Table 10.

19. The RNAi agent of claim 18, wherein all or substantially all of the nucleotides are modified nucleotides.

20. The RNAi agent of claim 1, comprising an antisense strand that consists of, consists essentially of, or comprises a nucleotide sequence that differs by 0 or 1 nucleotides from one of the following nucleotide sequences (5′→3′):
(SEQ ID NO: 836)
AGACAUUUAUUGGUUGUGACC;
(SEQ ID NO: 853)
AGACGUUUAUUGGUUGUGACC;
(SEQ ID NO: 837)
UGACAUUUAUUGGUUGUGACC;
(SEQ ID NO: 856)
UGACGUUUAUUGGUUGUGACC;
(SEQ ID NO: 196)
AGACAUUUAUUGGUUGUGA;
(SEQ ID NO: 197)
UGACAUUUAUUGGUUGUGA;
(SEQ ID NO: 137)
UUAGCAUUUAUCUGAGUUU;
(SEQ ID NO: 139)
UUAGCAUUUAUCUGAGUUC;
(SEQ ID NO: 192)
UACAUUUAUUGGUUGUGAC;
(SEQ ID NO: 830)
AGACAUUUAUUGGUUGUGACU;
(SEQ ID NO: 825)
UUAGCAUUUAUCUGAGUUUCC;
or
(SEQ ID NO: 826)
UACAUUUAUUGGUUGUGACUU.

21. The RNAi agent of claim 20, wherein the sense strand consists of, consists essentially of, or comprises a nucleotide sequence that differs by 0 or 1 nucleotides from one of the following nucleotide sequences (5′→3′):
(SEQ ID NO: 872)
GGUCACAACCAAUAAAUGUCU;
(SEQ ID NO: 873)
GGUCACAACCAAUAAAUGUCA;
(SEQ ID NO: 461)
UCACAACCAAUAAAUGUCU;
(SEQ ID NO: 462)
UCACAACCAAUAAAUGUCA;
(SEQ ID NO: 402)
AAACUCAGAUAAAUGCUAA;
(SEQ ID NO: 871)
G(A^{2N})ACUCAGAUAAAUGCUAA;
(SEQ ID NO: 457)
GUCACAACCAAUAAAUGUA
(SEQ ID NO: 864)
AGUCACAACCAAUAAAUGUCU;
(SEQ ID NO: 866)
GGAAACUCAGAUAAAUGCUAA;
or
(SEQ ID NO: 863)
(A^{2N})AGUCACAACCAAUAAAUGUA,
wherein (A^{2N}) represents a 2-aminoadenosine nucleotide.

22. The RNAi agent of claim 20 or 21, wherein all or substantially all of the nucleotides are modified nucleotides.

23. The RNAi agent of claim 1, comprising an antisense strand that comprises, consists of, or consists essentially of a modified nucleotide sequence that differs by 0 or 1 nucleotides from one of the following nucleotide sequences (5′→3′):
(SEQ ID NO: 649)
cPrpasGfsacauuuaUfuGfgUfuGfugacsc
(SEQ ID NO: 609)
cPrpasGfsaCfaUfuUfaUfuGfgUfuGfuGfaCfsu;
(SEQ ID NO: 611)
cPrpasGfsacauuuaUfuGfgUfuGfugacsu;
(SEQ ID NO: 681)
cPrpasGfsacguuuaUfuGfgUfuGfugacsc;
(SEQ ID NO: 612)
cPrpasGfsacauuuAfuuGfgUfuGfugacsu;
(SEQ ID NO: 603)
cPrpusUfsagcauuUfauCfuGfaGfuuucsc;
(SEQ ID NO: 606)
cPrpusUfsagcauUfuauCfuGfaGfuuucsc;
or
(SEQ ID NO: 594)
cPrpusAfscsAfuUfuAfuUfgGfuUfgUfgAfcUfsu;
wherein a represents 2′-O-methyl adenosine, c represents 2′-O-methyl cytidine, g represents 2′-O-methyl guanosine, and u represents 2′-O-methyl uridine; Af, represents 2′-fluoro adenosine, Cf represents 2′-fluoro cytidine, Gf represents 2′-fluoro guanosine, and Uf represents 2′-fluoro uridine; cPrpa represents a 5′-cyclopropyl phosphonate-2′-O-methyl adenosine; cPrpu represents a 5′-cyclopropyl phosphonate-2′-O-methyl uridine; s represents a phosphorothioate linkage; and wherein all or substantially all of the nucleotides on the sense strand are modified nucleotides.

24. The RNAi agent of claim 1, wherein the sense strand comprises, consists of, or consists essentially of a modified nucleotide sequence that differs by 0 or 1 nucleotides from one of the following nucleotide sequences (5′→3′):
(SEQ ID NO: 714)
gsgucacaaCfCfAfauaaaugucu;
(SEQ ID NO: 702)
asgucacaaCfCfAfauaaaugucu;
(SEQ ID NO: 704)
gsgaaacucAfGfAfuaaaugcuaa;
(SEQ ID NO: 701)
a_2NsagucacaAfCfCfaauaaaugua;
wherein a represents 2′-O-methyl adenosine, c represents 2′-O-methyl cytidine, g represents 2′-O-methyl guanosine, and u represents 2′-O-methyl uridine; Af, represents 2′-fluoro adenosine, Cf represents 2′-fluoro cytidine, Gf represents 2′-fluoro guanosine, and Uf represents 2′-fluoro uridine; a 2N represents 2′-O-methyl-2-aminoadenosine; s represents a phosphorothioate linkage; and wherein all or substantially all of the nucleotides on the antisense strand are modified nucleotides.

25. The RNAi agent of any one of claims 20-24, wherein the sense strand further includes inverted abasic residues at the 3′ terminal end of the nucleotide sequence, at the 5′ end of the nucleotide sequence, or at both.

26. The RNAi agent of any one of claims 1-25, wherein the RNAi agent is linked to a targeting ligand.

27. The RNAi agent of claim 26, wherein the targeting ligand has affinity for a cell receptor expressed on an epithelial cell.

28. The RNAi agent of claim 27, wherein the targeting ligand comprises an integrin targeting ligand.

29. The RNAi agent of claim 28, wherein the integrin targeting ligand is an αvβ6 integrin targeting ligand.

30. The RNAi agent of claim 29, wherein the targeting ligand comprises the structure: 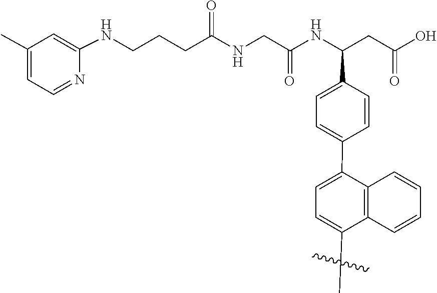 [SMILES: *c1ccc(-c2ccc([C@H](CC(=O)O)NC(=O)CNC(=O)CCCNc3cc(C)ccn3)cc2)c2ccccc12]
or a pharmaceutically acceptable salt thereof, or 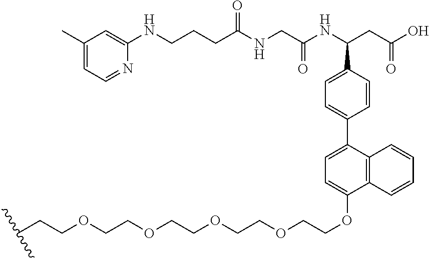 [SMILES: *CCOCCOCCOCCOCCOc1ccc(-c2ccc([C@H](CC(=O)O)NC(=O)CNC(=O)CCCNc3cc(C)ccn3)cc2)c2ccccc12]
or a pharmaceutically acceptable salt thereof,
wherein [symbol] indicates the point of connection to the RNAi agent.

31. The RNAi agent of any one of claims 26-29, wherein the targeting ligand has a structure selected from the group consisting of: 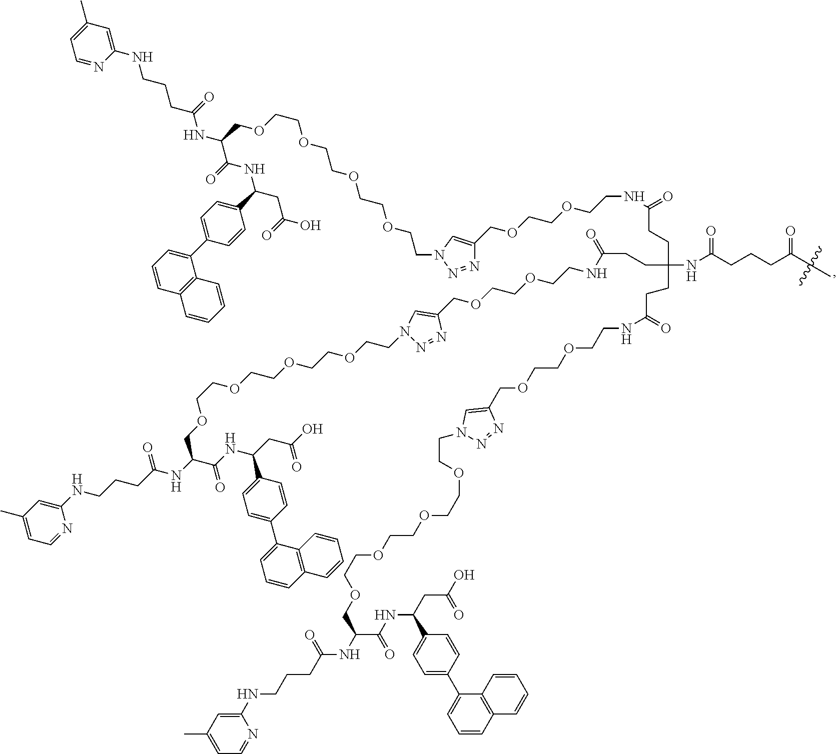 [SMILES: *C(=O)CCCC(=O)NC(CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1)(CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1)CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1] 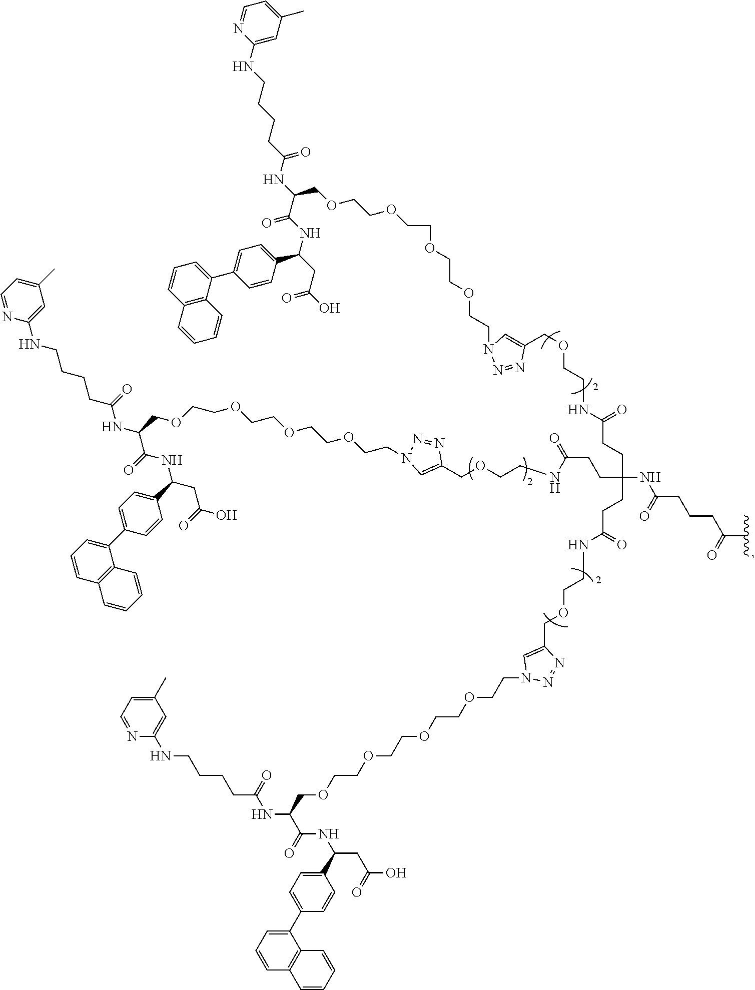 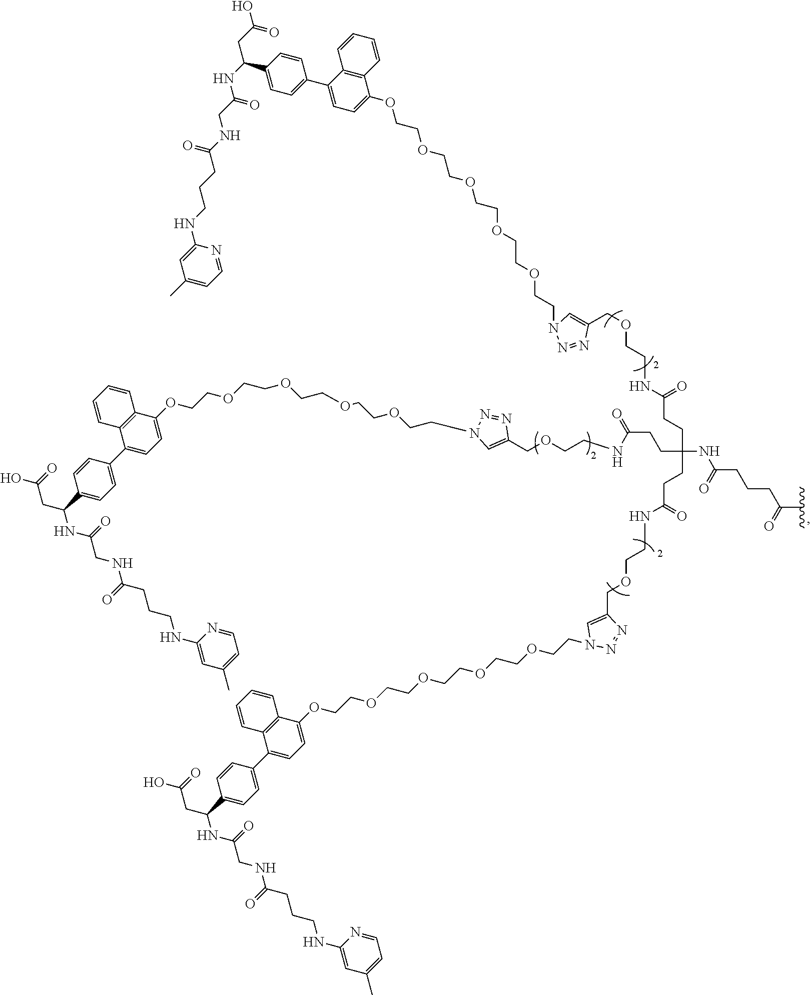 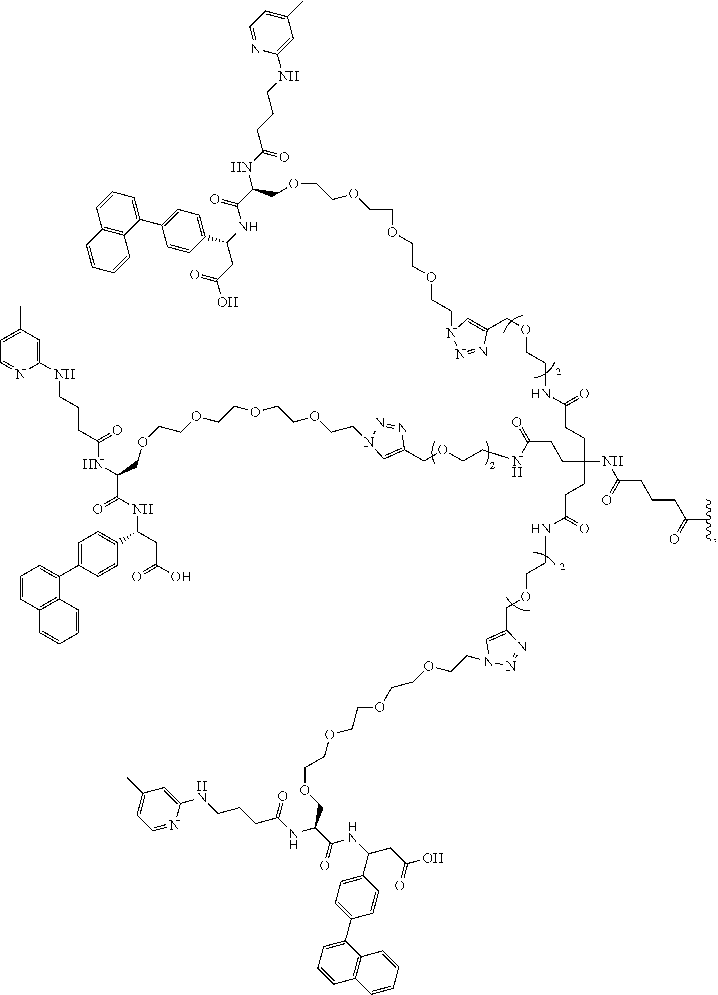 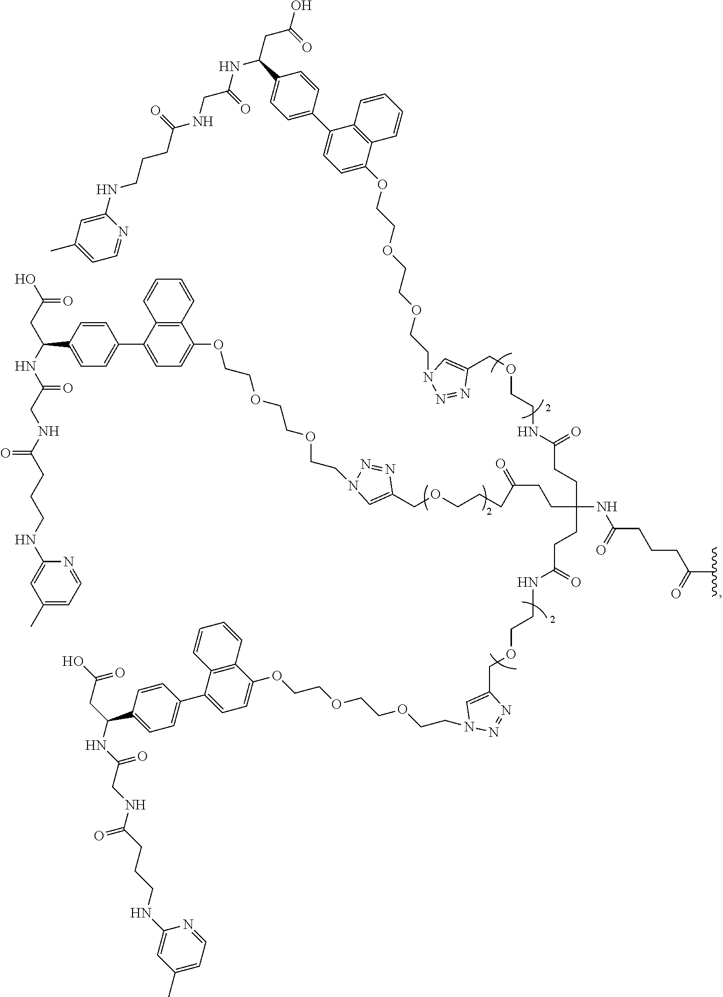 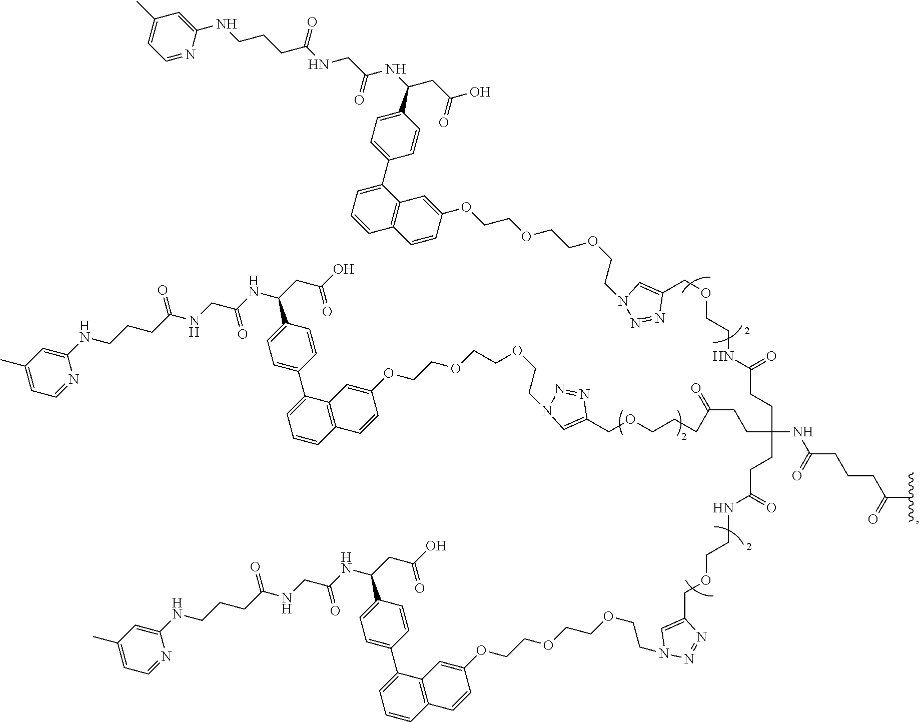 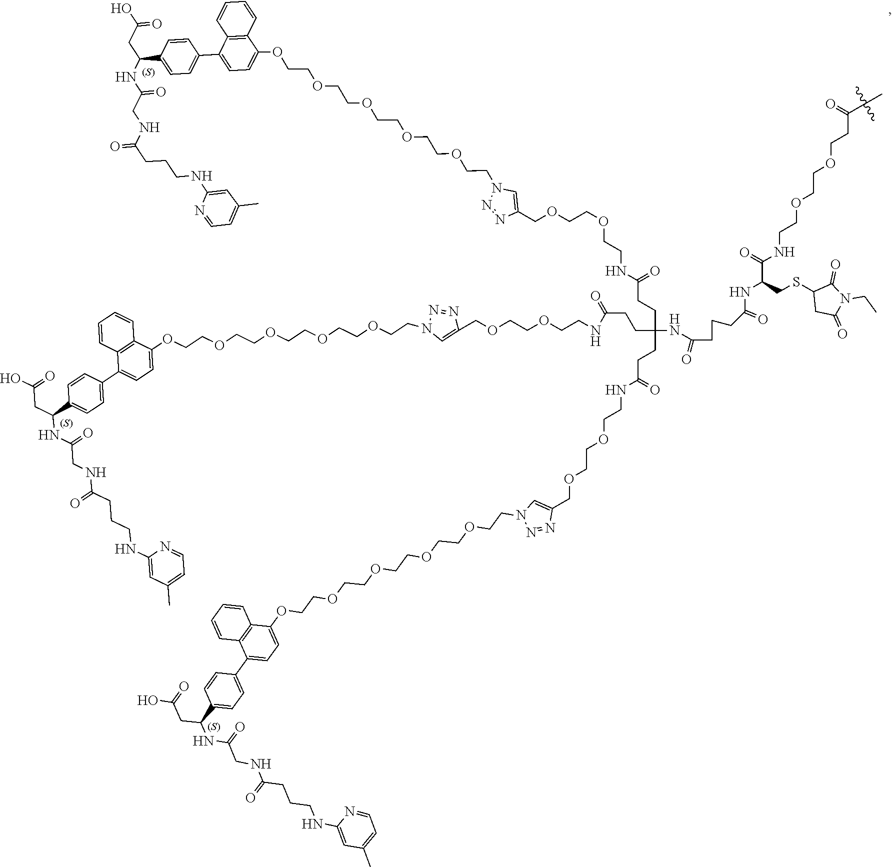 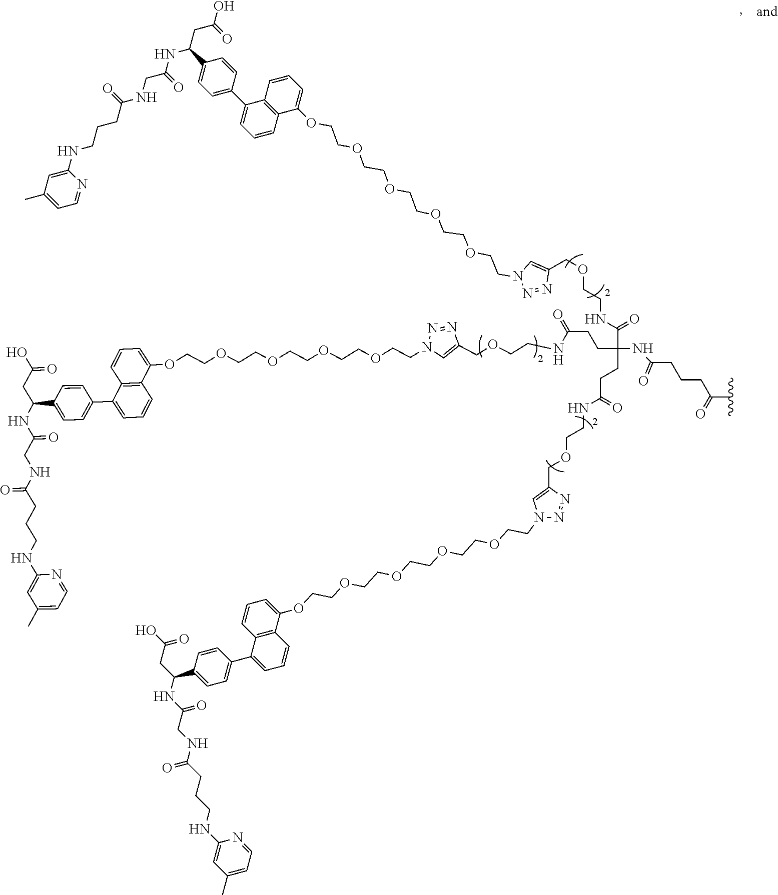 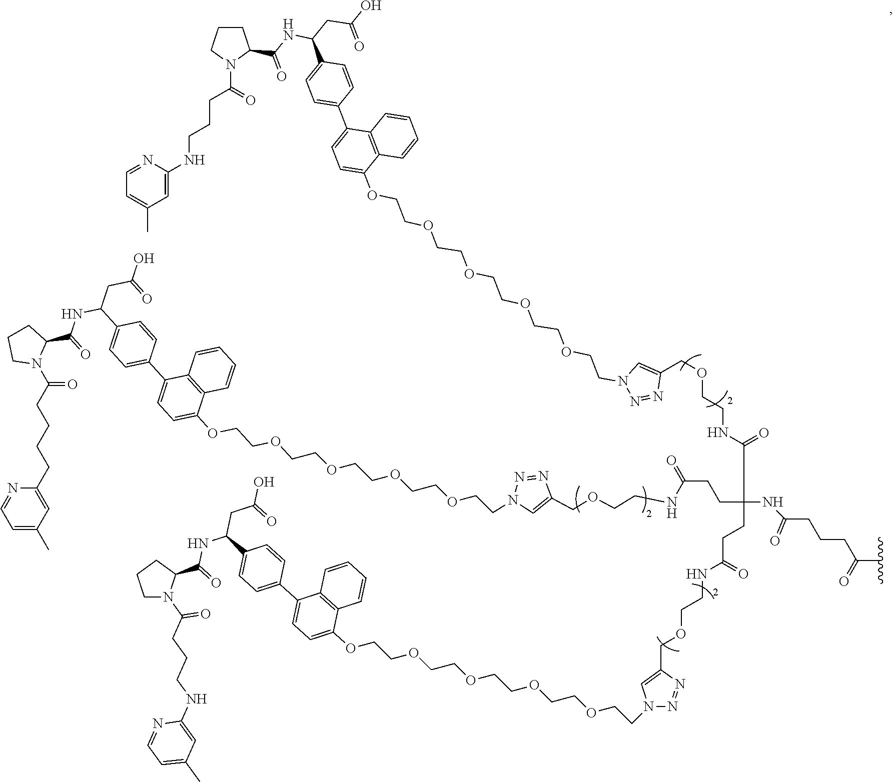
wherein [symbol] indicates the point of connection to the RNAi agent.

32. The RNAi agent of claim 31, wherein RNAi agent is conjugated to a targeting ligand having the following structure:

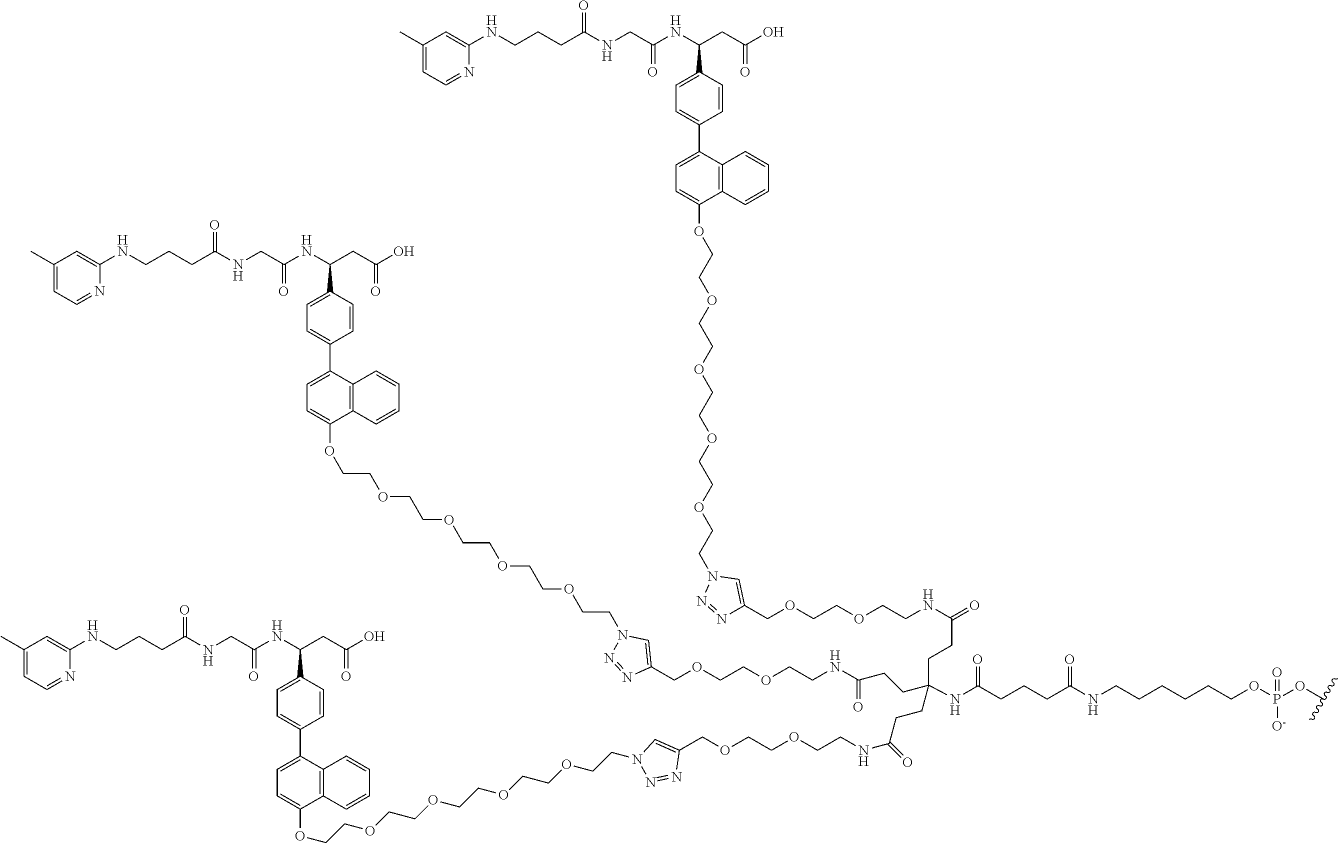 [SMILES: *OP(=O)([O-])OCCCCCCNC(=O)CCCC(=O)NC(CCC(=O)NCCOCCOCc1cn(CCOCCOCCOCCOCCOc2ccc(-c3ccc([C@H](CC(=O)O)NC(=O)CNC(=O)CCCNc4cc(C)ccn4)cc3)c3ccccc23)nn1)(CCC(=O)NCCOCCOCc1cn(CCOCCOCCOCCOCCOc2ccc(-c3ccc([C@H](CC(=O)O)NC(=O)CNC(=O)CCCNc4cc(C)ccn4)cc3)c3ccccc23)nn1)CCC(=O)NCCOCCOCc1cn(CCOCCOCCOCCOCCOc2ccc(-c3ccc([C@H](CC(=O)O)NC(=O)CNC(=O)CCCNc4cc(C)ccn4)cc3)c3ccccc23)nn1]

33. The RNAi agent of any one of claims 26-32, wherein the targeting ligand is conjugated to the sense strand.

34. The RNAi agent of claim 33, wherein the targeting ligand is conjugated to the 5′ terminal end of the sense strand.

35. The RNAi agent of any one of claims 1-34, wherein the RNAi agent is a pharmaceutically acceptable salt.

36. The RNAi agent of any one of claim 35, wherein the RNAi agent is a sodium salt.

37. A composition comprising the RNAi agent of any one of claims 1-36, wherein the composition further comprises a pharmaceutically acceptable excipient.

38. The composition of claim 37, further comprising a second RNAi agent capable of inhibiting the expression of thymic stromal lymphopoietin gene expression.

39. The composition of any one of claims 37-38, further comprising one or more additional therapeutics.

40. The composition of any one of claims 37-39, wherein the composition is formulated for administration by inhalation.

41. The composition of claim 40, wherein the composition is delivered by a metered-dose inhaler, jet nebulizer, vibrating mesh nebulizer, or soft mist inhaler.

42. The composition of any of claims 37-41, wherein the RNAi agent is a sodium salt.

43. The composition of any of claims 37-42, wherein the pharmaceutically acceptable excipient is water for injection.

44. The composition of any of claims 37-42, wherein the pharmaceutically acceptable excipient is a buffered saline solution.

45. A method for inhibiting expression of a TSLP gene in a cell, the method comprising introducing into a cell an effective amount of an RNAi agent of any one of claims 1-35 or the composition of any one of claims 37-44.

46. The method of claim 45, wherein the cell is within a subject.

47. The method of claim 46, wherein the subject is a human subject.

48. The method of any one of claims 45-47, wherein following the administration of the RNAi agent the thymic stromal lymphopoietin gene expression is inhibited by at least about 30%.

49. A method of treating one or more symptoms or diseases associated with enhanced or elevated TSLP cytokine activity levels, the method comprising administering to a human subject in need thereof a therapeutically effective amount of the composition of any one of claims 37-44.

50. The method of claim 49, wherein the disease is asthma including but not limited to allergic asthma, chronic obstructive pulmonary disease including but not limited to chronic bronchitis and emphysema, pulmonary inflammatory disorders, interstitial lung diseases (ILD), cystic fibrosis, various other types of fibrosis, infectious diseases (for example, SARS-COV-2), acute lung injury (for example, acute respiratory distress syndrome (ARDS)), pulmonary hypertension, various pulmonary cancers, chronic rhinosinutis either with or without nasal polyps, autoimmune disorders including but not limited to systemic sclerosis (SSc), and multiple inflammatory diseases including but not limited to atopic dermatitis, chronic spontaneous urticaria, and eosinophilic esophagitis.

51. The method of claim 50, wherein the disease is allergic asthma.

52. The method of any one of claims 45-51, wherein the RNAi agent is administered at a deposited dose of about 0.01 mg/kg to about 5.0 mg/kg of body weight of the subject.

53. The method of any one of claims 45-52, wherein the RNAi agent is administered at a deposited dose of about 0.03 mg/kg to about 2.0 mg/kg of body weight of the subject.

54. The method of any of claims 45-53, wherein the RNAi agent is administered in two or more doses.

55. Use of the RNAi agent of any one of claims 1-36, for the treatment of a disease, disorder, or symptom that is mediated at least in part by TSLP cytokine activity and/or TSLP gene expression.

56. Use of the composition according to any one of claims 37-44, for the treatment of a disease, disorder, or symptom that is mediated at least in part by thymic stromal lymphopoietin cytokine activity and/or thymic stromal lymphopoietin gene expression.

57. Use of the composition according to any one of claims 37-44, for the manufacture of a medicament for treatment of a disease, disorder, or symptom that is mediated at least in part by thymic stromal lymphopoietin cytokine and/or thymic stromal lymphopoietin gene expression.

58. The use of any one of claims 55-57, wherein the disease is pulmonary inflammation.

59. A method of making an RNAi agent of any one of claims 1-36, comprising annealing a sense strand and an antisense strand to form a double-stranded ribonucleic acid molecule.

60. The method of claim 59, wherein the sense strand comprises a targeting ligand.

61. The method of claim 60, comprising conjugating a targeting ligand to the sense strand.
